(19) United States
(12) Patent Application Publication
(10) Pub. No.: US 2023/0317206 A1
(43) Pub. Date: Oct. 5, 2023

(54) METHODS AND COMPOSITIONS FOR THE MOLECULAR DIAGNOSIS OF MICROSATELLITE INSTABILITY AND TREATMENTS FOR CANCER

(71) Applicant: UNIVERSITY OF WASHINGTON, Seattle, WA (US)

(72) Inventors: Stephen J. SALIPANTE, Seattle, WA (US); Adam S. WAALKES, Seattle, WA (US); Dustin LONG, Seattle, WA (US); Ronald HAUSE, Seattle, WA (US)

(73) Assignee: UNIVERSITY OF WASHINGTON, Seattle, WA (US)

(21) Appl. No.: 18/011,687

(22) PCT Filed: Jun. 23, 2021
(86) PCT No.: PCT/US2021/038672

§ 371 Date: Dec. 20, 2022

Related U.S. Application Data
(60) Provisional application No. 63/044,029, filed on Jun. 25, 2020.

(51) Int. Cl. G16B 20/10; G16B 20/20; G16B 40/20; G16H 50/20; G16H 50/70; C12Q 1/6886

(52) U.S. Cl. CPC G16B 20/10; G16B 20/20; G16B 40/20; G16H 50/20; G16H 50/70; C12Q 1/6886; C12Q 2600/156; C12Q 2600/106

(57) ABSTRACT

Described herein are methods and compositions related to treatments for cancer. Various embodiments relate to the determination of microsatellite instability as a marker for the likelihood of whether a given cancer will respond to certain therapies, including checkpoint inhibitor immunotherapies, and methods of treatment based upon those determinations.

### CROSS-REFERENCE TO RELATED APPLICATIONS

This application claims benefit under 35 U.S.C. § 119(e) of U.S. Provisional Application No. 63/044,029 filed Jun. 25, 2020, the contents of which are incorporated herein by reference in their entirety.

### GOVERNMENT SUPPORT

This invention was made with government support under Grant No. CA222344, awarded by the National Institutes of Health. The government has certain rights in the invention.

### TECHNICAL FIELD

The technology described herein relates to methods of diagnosing and treating cancer.

### BACKGROUND

Microsatellite instability (MSI) is a molecular tumor phenotype that is indicative of genomic hypermutability, usually reflecting inactivation of the mismatch repair (MMR) system. MSI is marked by spontaneous gains or losses of nucleotides from repetitive DNA tracts, resulting in new alleles of differing length that serve as the basis for its clinical diagnosis. Although classically associated with colorectal and endometrial tumors, MSI has now been recognized in most cancer types with varying prevalence and is accompanied by a generally increased rate of mutations genome-wide. Molecular diagnosis of MSI can be predictive of a patient's response to an anti-cancer therapy. However, there is an unmet need for cancer screening panels of MSI in specific cancer types and the microsatellite marker sequences vary between different types of cancers.

### SUMMARY

To better inform molecular diagnosis of MSI, over 9,000 tumor-normal exome pairs and over 900 whole genome sequence pairs were examined from 33 different cancer types and cataloged for genome-wide microsatellite instability events. Using a statistical framework, microsatellite mutations that were predictive of MSI within and across cancer types were identified. The diagnostic accuracy of different subsets of maximally informative markers was estimated computationally using a dedicated validation set. Based on these analyses, twenty-five cancer types exhibited hypermutated states consistent with MSI. Recurrently mutated microsatellites associated with MSI were identifiable in 15 cancer types, but were largely specific to individual cancer types. Cancer-specific microsatellite panels of one to seven loci were needed to attain 95% diagnostic sensitivity and specificity for 11 cancer types, and in eight of the cancer types, 100% sensitivity and specificity were achieved. In contrast, breast cancer required 800 loci to achieve comparable performance, and it was not possible to identify recurrent microsatellite mutations supporting reliable MSI diagnosis in ovarian tumors. Thus, as described further herein below, most microsatellites informative for MSI are specific to particular cancer types, requiring the use of tissue-specific loci for optimal diagnosis. Accordingly, limited numbers of markers are needed to provide accurate MSI diagnosis in most tumor types, but it is challenging to diagnose breast and ovarian cancers using pre-defined microsatellite locus panels.

Features associated with informative microsatellites were cataloged, and sets of microsatellites are ranked herein according to degree of predictive value for each microsatellite for each tumor type and underpin diagnostic, prognostic and therapeutic methods as described herein below. For each set, the number of mutated members of that set which are determinative of MSI-H is influenced by where the mutated members of the set are in the rank ordering, with higher ranked microsatellite markers carrying greater weight (i.e., lower p-value). Thus, the number of microsatellite mutations necessary for a sensitive and specific determination of MSI-H for a given cancer type can vary depending upon the ranking of the mutated markers in the set, with fewer mutations needed to assign the cancer to the MSI-H category when the mutations detected are in higher ranked microsatellites (i.e., lower p-value).

Accordingly, in one aspect, described herein is a method of predicting whether a subject's cancer will respond to checkpoint inhibitor immunotherapy, the method comprising: (a) receiving microsatellite instability data for a defined set of microsatellite repeat marker sequences in cells of the subject's cancer; and (b) processing the microsatellite instability data to output a categorical measure of microsatellite instability high (MSI-H) or microsatellite stable (MSS); wherein when the subject's cancer is determined to exhibit MSI-H, it is predicted that the cancer will respond to checkpoint inhibitor immunotherapy, and when the subject's cancer is determined to exhibit MSS, it is predicted that the cancer is less likely to respond to checkpoint inhibitor immunotherapy.

In another aspect, described herein is a method of predicting whether a subject's cancer will respond to checkpoint inhibitor immunotherapy, the method comprising: determining microsatellite instability status for a defined set of microsatellite repeat marker sequences in cells of the subject's cancer, wherein determining the microsatellite instability status of the subject's cancer comprises: (i) assaying loci in the defined set of microsatellite repeat marker sequences in cells of the subject's cancer for mutation, thereby generating microsatellite instability data; and (ii) processing the microsatellite instability data to output a categorical measure of microsatellite instability high (MSI-H) status or microsatellite stable (MSS) status for the subject's cancer; wherein when the subject's cancer is determined to exhibit MSI-H, it is predicted that the cancer will respond to checkpoint inhibitor immunotherapy, and when the subject's cancer is determined to exhibit MSS, it is predicted that the cancer is less likely to respond to checkpoint inhibitor immunotherapy.

In another aspect, described herein is a method of determining microsatellite instability (MSI) in a subject's cancer, the method comprising: assaying mutation status for a set of microsatellite repeat marker sequences in cells of the subject's cancer, wherein when the set of microsatellites exhibits mutation at or above a threshold number of microsatellites in the set, the subject's cancer is determined to exhibit high microsatellite instability (MSI-H), and when the set of microsatellites exhibits mutation below the threshold number of microsatellites in the set, the subject's cancer is determined to exhibit microsatellite stability (MSS); wherein the threshold for the set is determined using the percentage of mutated microsatellite markers in the set to calculate the area under the receiver operating characteristic (AUROC), and wherein the smallest number of markers in the set necessary to reach an AUROC value selected in the range of 0.6 to 0.99, inclusive, is the threshold.

In another aspect, described herein is a method of treating cancer in a subject in need thereof, the method comprising determining microsatellite instability status for cells of a subject's cancer by a method of described herein, and, when the microsatellite instability status is determined to be MSI-H, administering a checkpoint inhibitor, or, when the microsatellite instability status is determined to be MSS, administering a non-checkpoint inhibitor cancer therapeutic.

In another aspect, described herein is a method of treating cancer in a subject in need thereof, wherein the subject's cancer has been determined to have a microsatellite instability status of MSI-H as determined by a method as described herein, the method comprising administering an effective amount of a checkpoint inhibitor to the subject.

In another aspect, described herein is a method of predicting whether a subject's cancer will respond to checkpoint inhibitor immunotherapy, the method comprising: assaying mutation status for a set of microsatellite repeat marker sequences in cells of the subject's cancer, wherein when mutations are present in at least a threshold number of markers in the set, the set provides at least 95% sensitivity and at least 95% specificity for predicting high microsatellite instability (MSI-H) for the subject's tumor.

In another aspect, described herein is a method of determining microsatellite instability (MSI) in a subject's cancer, the method comprising: assaying mutation status for a set of microsatellite repeat marker sequences in the cells of the subject's cancer, wherein when mutations are present in at least a threshold number of markers in the set, the set provides at least 95% sensitivity and at least 95% specificity for predicting high microsatellite instability (MSI-H) for the subject's tumor.

In another aspect, described herein is a method of treating cancer in a subject in need thereof, the method comprising: assaying mutation status for a set of microsatellite repeat marker sequences in cells of the subject's cancer, wherein when mutations are present in at least a threshold number of markers in the set, the set provides at least 95% sensitivity and at least 95% specificity for predicting high microsatellite instability (MSI-H) for the subject's tumor.

In another aspect, described herein is a use of a checkpoint inhibitor for the treatment of cancer, comprising: (a) receiving microsatellite instability data for a defined set of microsatellite repeat marker sequences in cells of the subject's cancer; and (b) processing the microsatellite instability data to output a categorical measure of microsatellite instability high (MSI-H) or microsatellite stable (MSS); wherein the checkpoint inhibitor is administered when the subject's cancer is determined to exhibit MSI-H.

In another aspect, described herein is a use of a checkpoint inhibitor for the treatment of cancer, comprising: determining microsatellite instability status for a defined set of microsatellite repeat marker sequences in cells of the subject's cancer, wherein determining the microsatellite instability status of the subject's cancer comprises: (i) assaying loci in the defined set of microsatellite repeat marker sequences in cells of the subject's cancer for mutation, thereby generating microsatellite instability data; and (ii) processing the microsatellite instability data to output a categorical measure of microsatellite instability high (MSI-H) status or microsatellite stable (MSS) status for the subject's cancer; wherein the checkpoint inhibitor is administered when the subject's cancer is determined to exhibit MSI-H.

In another aspect, described herein is a diagnostic kit for determining microsatellite instability in a cancer, the kit comprising reagents that permit the detection of microsatellite mutation in a set of microsatellite repeat marker sequences in the cancer.

In another aspect, described herein is an array for detecting microsatellite instability in a cancer, the array comprising nucleic acids that permit the detection of microsatellite mutation in a set of microsatellite repeat marker sequences in the cancer, wherein the nucleic acids are linked to a solid support.

In one embodiment of any of the aspects, the set of microsatellite repeat markers comprises a plurality of up to 800 of the microsatellites set out herein or in Appendix A of U.S. Provisional Application No. 63/044,029 filed Jun. 25, 2020, the contents of which are incorporated herein by reference in their entirety.

In some embodiments of any of the aspects, when the cancer is colon adenocarcinoma (COAD), the set of microsatellite repeat marker sequences comprises a plurality up to 500 of the microsatellites set out in Table 1. In some embodiments of any of the aspects, when the cancer is esophageal carcinoma (ESCA), the set of microsatellite repeat marker sequences comprises a plurality up to 503 of the microsatellites set out in Table 2. In some embodiments of any of the aspects, when the cancer is glioblastoma multiforme (GBM), the set of microsatellite repeat marker sequences comprises a plurality up to 500 of the microsatellites set out in Table 3. In some embodiments of any of the aspects, when the cancer is lung adenocarcinoma (LUAD), the set of microsatellite repeat marker sequences comprises a plurality up to 500 of the microsatellites set out in Table 4. In some embodiments of any of the aspects, when the cancer is lung squamous cell carcinoma (LUSC), the set of microsatellite repeat marker sequences comprises a plurality up to 500 of the microsatellites set out in Table 5. In some embodiments of any of the aspects, when the cancer is rectum adenocarcinoma (READ), the set of microsatellite repeat marker sequences comprises a plurality up to 501 of the microsatellites set out in Table 6. In some embodiments of any of the aspects, when the cancer is stomach adenocarcinoma (STAD), the set of microsatellite repeat marker sequences comprises a plurality up to 500 of the microsatellites set out in Table 7. In some embodiments of any of the aspects, when the cancer is brain lower grade glioma (LGG), the set of microsatellite repeat marker sequences comprises a plurality up to 266 of the microsatellites set out in Table 8. In some embodiments of any of the aspects, when the cancer is prostate adenocarcinoma (PRAD), the set of microsatellite repeat marker sequences comprises a plurality up to 500 of the microsatellites set out in Table 9. In some embodiments of any of the aspects, when the cancer is cervical squamous cell carcinoma (CESC), the set of microsatellite repeat marker sequences comprises a plurality up to 500 of the microsatellites set out in Table 10. In some embodiments of any of the aspects, when the cancer is lymphoid neoplasm diffuse large B-cell lymphoma (DLBC), the set of microsatellite repeat marker sequences comprises a plurality up to 212 of the microsatellites set out in Table 11. In some embodiments of any of the aspects, when the cancer is uterine corpus endometrial carcinoma (UCEC), the set of microsatellite repeat marker sequences comprises a plurality up to 500 of the microsatellites set out in Table 12. In some embodiments of any of the aspects, when the cancer is kidney renal clear cell carcinoma (KIRC), the set of microsatellite repeat marker sequences comprises a plurality up to 508 of the microsatellites set out in Table 13. In some embodiments of any of the aspects, when the cancer is breast invasive carcinoma (BRCA), the set of microsatellite repeat marker sequences comprises a plurality up to 500 of the microsatellites set out in Table 14A. In some embodiments of any of the aspects, when the cancer is breast invasive carcinoma (BRCA), the set of microsatellite repeat marker sequences comprises a plurality up to 800 of the microsatellites set out in Tables 14A-14B. In some embodiments of any of the aspects, when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.

In another embodiment of any of the aspects, cancer that exhibits greater than or equal to the threshold number of mutations for that tumor type in the set is determined to be MSI-H, and cancer that exhibits fewer than the threshold number of mutations for that tumor type in the set, the subject's cancer is determined to be microsatellite stable or not MSI-H.

In some embodiments of any of the aspects, when the cancer is colon adenocarcinoma (COAD), the microsatellite repeat marker sequences comprise a plurality of microsatellite repeat markers in Table 1 and the threshold number is 50%. In some embodiments of any of the aspects, when the cancer is esophageal carcinoma (ESCA), the microsatellite repeat marker sequences comprise a plurality of microsatellite repeat markers in Table 2 and the threshold number is 50%. In some embodiments of any of the aspects, when the cancer is glioblastoma multiforme (GBM), the microsatellite repeat marker sequences comprise a plurality of microsatellite repeat markers in Table 3 and the threshold number is 50%. In some embodiments of any of the aspects, when the cancer is lung adenocarcinoma (LUAD), the microsatellite repeat marker sequences comprise a plurality of microsatellite repeat markers in Table 4 and the threshold number is 50%. In some embodiments of any of the aspects, when the cancer is lung squamous cell carcinoma (LUSC), the microsatellite repeat marker sequences comprise a plurality of microsatellite repeat markers in Table 5 and the threshold number is 50%. In some embodiments of any of the aspects, when the cancer is rectum adenocarcinoma (READ), the microsatellite repeat marker sequences comprise a plurality of microsatellite repeat markers in Table 6 and the threshold number is 50%. In some embodiments of any of the aspects, when the cancer is stomach adenocarcinoma (STAD), the microsatellite repeat marker sequences comprise a plurality of microsatellite repeat markers in Table 7 and the threshold number is 25%. In some embodiments of any of the aspects, when the cancer is brain lower grade glioma (LGG), the microsatellite repeat marker sequences comprise a plurality of microsatellite repeat markers in Table 8 and the threshold number is 12.5%. In some embodiments of any of the aspects, when the cancer is prostate adenocarcinoma (PRAD), the microsatellite repeat marker sequences comprise a plurality of microsatellite repeat markers in Table 9 and the threshold number is 16.7%. In some embodiments of any of the aspects, when the cancer is cervical squamous cell carcinoma (CESC), the microsatellite repeat marker sequences comprise a plurality of microsatellite repeat markers in Table 10 and the threshold number is 45.8%. In some embodiments of any of the aspects, when the cancer is lymphoid neoplasm diffuse large B-cell lymphoma (DLBC), the microsatellite repeat marker sequences comprise a plurality of microsatellite repeat markers in Table 11 and the threshold number is 25%. In some embodiments of any of the aspects, when the cancer is uterine corpus endometrial carcinoma (UCEC), the microsatellite repeat marker sequences comprise a plurality of microsatellite repeat markers in Table 12 and the threshold number is 12.9%. In some embodiments of any of the aspects, when the cancer is kidney renal clear cell carcinoma (KIRC), the microsatellite repeat marker sequences comprise a plurality of microsatellite repeat markers in Table 13 and the threshold number is 7.5%. In some embodiments of any of the aspects, when the cancer is breast invasive carcinoma (BRCA), the microsatellite repeat marker sequences comprise a plurality of microsatellite repeat markers in Table 14A and the threshold number is 3.6%. In some embodiments of any of the aspects, when the cancer is uterine corpus endometrial carcinoma (UCEC), colon adenocarcinoma (COAD), rectum adenocarcinoma (READ), or stomach adenocarcinoma (STAD), the microsatellite repeat marker sequences comprise a plurality of microsatellite repeat markers in Table 21 and the threshold number is at least 12.9% (e.g., 12.9%, 25%, 50%).

In another embodiment of any of the aspects, the quantitative model is selected from a continuous measure, regression or weighted scoring of markers, a combinatorial or decision-tree model, and a machine learning model. In another embodiment of any of the aspects, the quantitative model comprises a random-forest model or a deep neural network machine learning model.

In some embodiments of any of the aspects, information from individual markers can be combined to produce a final measure, non-limiting examples of which include a classification, score, probability, or prediction of MSI (e.g., stable, unstable, intermediate, indeterminate, or a continuous measure or probability of instability). Methods for analyzing information from individual markers to produce such a measure can include but are not limited to: additive scoring systems with or without use of weighting of individual markers (see e.g., Mehta et al. J Clin Epidemiol. 2016 November, 79:22-28; Austin et al. Stat Med. 2016, 35(22):4056-4072, published correction appears in Stat Med. 2018 Apr. 15, 37(8):1405; Avila et al. BMC Res Notes 8, 612 (2015)); traditional statistical methods such as linear, logistic, or Lasso regression (see e.g., Nick and Campbell (2007) Logistic Regression, in: Ambrosius W. T. (eds) Topics in Biostatistics, Methods in Molecular Biology™, vol 404, Humana Press; Eberly (2007) Multiple Linear Regression, in: Ambrosius W. T. (eds) Topics in Biostatistics, Methods in Molecular Biology™, vol 404, Humana Press; Tibshirani, Journal of the Royal Statistical Society, Series B (Methodological), vol. 58, no. 1, 1996, pp. 267-288); inverse probability-weighted estimation (see e.g., Curtis et al. Medical Care, vol. 45, no. 10, 2007, pp. 5103-5107); Bayesian methods (see e.g., Eberly 2007, supra); classifiers such as support vector machine (see e.g., Cortes and Vapnik, Mach Learn 20, 273-297 (1995)); principal component analysis (see e.g., Lever et al. Nat Methods 14, 641-642 (2017)); hierarchical clustering (see e.g., Rokach et al. Data mining and knowledge discovery handbook, Springer US, 2005, 321-352); decision trees (see e.g., Safavian and Landgrebe, IEEE Transactions on Systems, Man, and Cybernetics, vol. 21, no. 3, pp. 660-674, May-June 1991); random forest models (see e.g., Breiman, Random Forests. Machine Learning 45, 5-32 (2001)); gradient boosting methods (see e.g., Hastie et al. (2009), “10. Boosting and Additive Trees,” The Elements of Statistical Learning (2nd ed.), ISBN 978-0-387-84857-0, New York: Springer, pp. 337-384; Kobayashi and Yoshida, Environ Res. 2021 May, 196:110363); or machine learning approaches such as deep neural networks (see e.g., LeCun et al. Nature 521, 436-444 (2015)). In some embodiments of any of the aspects, missing data for individual markers can be handled intrinsically by models capable of utilizing missing data or can be accounted for using supplementary approaches to account for missing data such as imputation (see e.g., Eberly 2007, supra). The contents of each of the abovementioned references are incorporated herein by reference in their entireties.

In another embodiment of any of the aspects, the quantitative model evaluates or incorporates consideration of one or more test characteristics selected from the group consisting of sensitivity, accuracy, correlation, probability, specificity, false-positive rate, false negative rate, positive predictive value, negative predictive value and area under the receiver-operator characteristic (AUROC). In another embodiment of any of the aspects, the continuous measure comprises the proportion of unmutated or stable loci to mutated or unstable loci detected in the set.

In another embodiment of any of the aspects, thresholds for the one or more test characteristics indicative of MSI-H or MSS are defined within parameters of the known test characteristics for a given clinical application. In another embodiment of any of the aspects, the processing of MSI-H or MSS is determined by a threshold value for the set of microsatellite marker sequences. In another embodiment of any of the aspects, the threshold for the set of microsatellite marker sequences is determined using the percentage of mutated microsatellite markers in the set to calculate the area under the receiver operating characteristic (AUROC). In another embodiment of any of the aspects, the smallest number of markers in the set necessary to reach a selected AUROC value is the threshold. In another embodiment of any of the aspects, the selected AUROC value is a value between 0.6 and 0.99, inclusive. In another embodiment of any of the aspects, the selected AUROC value is 0.9 or more.

In another embodiment of any of the aspects, the set of microsatellite repeat marker sequences comprises a plurality up to 200 of the sequences set out in the respective Table for the subject's cancer type. In another embodiment of any of the aspects, the set of microsatellite repeat marker sequences comprises a plurality up to 300 of the sequences set out in the respective Table for the subject's cancer type. In another embodiment of any of the aspects, the set of microsatellite repeat marker sequences comprises a plurality up to the first 100 of the sequences set out in the respective Table for the subject's cancer type. In another embodiment of any of the aspects, the set of microsatellite repeat marker sequences comprises a plurality up to the first 200 of the sequences set out in the respective Table for the subject's cancer type. In another embodiment of any of the aspects, the set of microsatellite repeat marker sequences comprises a plurality up to the first 300 of the sequences set out in the respective Table for the subject's cancer type.

In another embodiment of any of the aspects, the checkpoint inhibitor immunotherapy comprises a checkpoint inhibitor antibody. In another embodiment of any of the aspects, the checkpoint inhibitor immunotherapy is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7). In another embodiment of any of the aspects, the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®)

In another embodiment of any of the aspects, the non-checkpoint inhibitor cancer therapy comprises one or more of angiostatin K1-3, DL-α-Difluoromethyl-ornithine, endostatin, fumagillin, genistein, minocycline, staurosporine, and (±)-thalidomide; a DNA intercalator/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, Dolastatin 15, Nocodazole, Paclitaxel, Podophyllotoxin, Rhizoxin, Vinblastine, Vincristine, Vindesine, and Vinorelbine (Navelbine); a neoantigen; and an unclassified antitumor agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatinib dimaleate (Gilotrif®), ceritinib (LDK378/Zykadia), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), a targeted therapy such as toremifene (Fareston®), fulvestrant (Faslodex®), anastrozole (Arimidex®), exemestane (Aromasin®), letrozole (Ferrara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®), an epigenetic targeted drug such as HDAC inhibitors, azacitidine (Vidaza®), decitabine (Dacogen®), vorinostat (Zolinza®), romidepsin (Istodax®), ruxolitinib (Jakafi®), kinase inhibitors, DNA methyltransferase inhibitors, histone demethylase inhibitors, or histone methylation inhibitors, and any derivative thereof.

In another embodiment of any of the aspects, assaying loci in the defined set of microsatellite repeat marker sequences in cells of the subject's cancer for mutation comprises polynucleotide sequencing, measurement of total mutational burden, immunohistochemical analysis, and/or polymerase chain reaction. In another embodiment of any of the aspect, polynucleotide sequencing comprises whole genome sequencing, whole exome sequencing, or targeted gene capture sequencing.

In another embodiment of any of the aspects, microsatellite mutations correspond to those identified in reference human genome GRCh37/hg19 translated to a different build of the human reference genome.

In another embodiment of any of the aspects, the kit comprises an array of reagents that permit the detection of microsatellite mutation on a solid support. In another embodiment of any of the aspects, the reagents comprise PCR primers that permit amplification of members of the set of microsatellite repeat marker sequences.

### BRIEF DESCRIPTION OF THE DRAWINGS

FIG. 1 is a dot plot showing the relative burden of microsatellite instability events across cancer types. Percentage of total microsatellite loci found to be mutated are reported per tumor specimen, as stratified by cancer type. Each point corresponds to an individual tumor, and its coloration indicates the corresponding MSI classification. TCGA abbreviations for cancer types are as described in the Table 16 footnote.

FIG. 2 is a heatmap showing that informative markers for diagnosing MSI vary across cancer types. The 2,000 most informative microsatellites (rows) for discriminating MSI positive from MSI negative tumors from each of the indicated cancer types (columns) are displayed. Heatmap coloration indicates the association of individual loci with the MSI-H phenotype, normalized on a per-cancer type basis from zero (least significant) to one (most significant). Hierarchical clustering of cancer types according to similarity in microsatellite mutation patterns is indicated at top. TCGA abbreviations for cancer types are as described in the Table 16 footnote.

FIG. 3A-3B is a series of line graphs showing the performance characteristics of variously sized tissue-specific panels for MSI diagnosis. Area under the receiver operating characteristic (AUROC) is shown as a function of the number of most highly informative markers examined for various cancer types. FIG. 3A shows the results for the training set from which informative microsatellites were initially identified. FIG. 3B shows the results for an independent validation set for each tumor type. TCGA abbreviations for cancer types are as described in the Table 16 footnote.

FIG. 4A-4C shows the properties of informative microsatellites. The ridgeline plots show the density of −log₁₀ transformed p values for specified features among microsatellites that are informative for diagnosing MSI. A shift to the right in the placement of distributions corresponds to increased association with locus instability in MSI. FIG. 4A shows repeat type. FIG. 4B shows genomic context. FIG. 4C shows the number of repeats in a microsatellite.

FIG. 5 shows the relative burden of microsatellite instability events across cancer types, inferred from whole genome sequence data. Percentage of total microsatellite loci found to be mutated are reported per tumor specimen, as stratified by cancer type. Each point corresponds to an individual tumor, and its shade of grey indicates the corresponding MSI classification. TCGA abbreviations for cancer types are as described in the Table 16 footnote.

### DETAILED DESCRIPTION

The methods and compositions described herein relate, in part, to the discovery of microsatellite repeat marker sequences that predict whether a subject will respond to checkpoint inhibitor immunotherapy for each cancer type. The methods comprise assaying mutation status for a set of microsatellite repeat marker sequences in cells of the subject's cancer, wherein when the set of microsatellites exhibits mutation at or above a threshold number of microsatellites in the set, the subject's cancer is determined to exhibit high microsatellite instability (MSI-H). The microsatellite repeat marker sequences for each cancer type that are most predictive of a subject's response to checkpoint inhibitor immunotherapy are provided below, e.g., in Tables 1-15. In Tables 1-15, chromosome is abbreviated as “ch.” The chromosome sequences of GRCh37/hg19 (also referred to herein as hg37) can be found at the following accession numbers indicated in Table 18 below.

TABLE 18
GRCh37/hg19 Reference Sequences; the indicated
chromosome sequences are incorporated herein
by reference in their entireties

| Molecule name | GenBank sequence | RefSeq sequence |
|---|---|---|
| Chromosome 1 | CM000663.1 | NC_000001.10 |
| Chromosome 2 | CM000664.1 | NC_000002.11 |
| Chromosome 3 | CM000665.1 | NC_000003.11 |
| Chromosome 4 | CM000666.1 | NC_000004.11 |
| Chromosome 5 | CM000667.1 | NC_000005.9 |
| Chromosome 6 | CM000668.1 | NC_000006.11 |
| Chromosome 7 | CM000669.1 | NC_000007.13 |
| Chromosome 8 | CM000670.1 | NC_000008.10 |
| Chromosome 9 | CM000671.1 | NC_000009.11 |
| Chromosome 10 | CM000672.1 | NC_000010.10 |
| Chromosome 11 | CM000673.1 | NC_000011.9 |
| Chromosome 12 | CM000674.1 | NC_000012.11 |
| Chromosome 13 | CM000675.1 | NC_000013.10 |
| Chromosome 14 | CM000676.1 | NC_000014.8 |
| Chromosome 15 | CM000677.1 | NC_000015.9 |
| Chromosome 16 | CM000678.1 | NC_000016.9 |
| Chromosome 17 | CM000679.1 | NC_000017.10 |
| Chromosome 18 | CM000680.1 | NC_000018.9 |
| Chromosome 19 | CM000681.1 | NC_000019.9 |
| Chromosome 20 | CM000682.1 | NC_000020.10 |
| Chromosome 21 | CM000683.1 | NC_000021.8 |
| Chromosome 22 | CM000684.1 | NC_000022.10 |
| Chromosome X | CM000685.1 | NC_000023.10 |
| Chromosome Y | CM000686.1 | NC_000024.9 |

In some embodiments of any of the aspects, a set of microsatellite repeat marker sequences are selected from the respective Table for the subject's cancer type (e.g., at least one of Tables 1-15 or Table 21). In some embodiments of any of the aspects, a set of microsatellite repeat marker sequences comprises a plurality up to 800 of the microsatellites set out in the respective Table for the subject's cancer type (e.g., at least one of Tables 1-15 or Table 21). In some embodiments of any of the aspects, a set of microsatellite repeat marker sequences comprises a plurality of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, at least 249, at least 250, at least 251, at least 252, at least 253, at least 254, at least 255, at least 256, at least 257, at least 258, at least 259, at least 260, at least 261, at least 262, at least 263, at least 264, at least 265, at least 266, at least 267, at least 268, at least 269, at least 270, at least 271, at least 272, at least 273, at least 274, at least 275, at least 276, at least 277, at least 278, at least 279, at least 280, at least 281, at least 282, at least 283, at least 284, at least 285, at least 286, at least 287, at least 288, at least 289, at least 290, at least 291, at least 292, at least 293, at least 294, at least 295, at least 296, at least 297, at least 298, at least 299, at least 300, at least 301, at least 302, at least 303, at least 304, at least 305, at least 306, at least 307, at least 308, at least 309, at least 310, at least 311, at least 312, at least 313, at least 314, at least 315, at least 316, at least 317, at least 318, at least 319, at least 320, at least 321, at least 322, at least 323, at least 324, at least 325, at least 326, at least 327, at least 328, at least 329, at least 330, at least 331, at least 332, at least 333, at least 334, at least 335, at least 336, at least 337, at least 338, at least 339, at least 340, at least 341, at least 342, at least 343, at least 344, at least 345, at least 346, at least 347, at least 348, at least 349, at least 350, at least 351, at least 352, at least 353, at least 354, at least 355, at least 356, at least 357, at least 358, at least 359, at least 360, at least 361, at least 362, at least 363, at least 364, at least 365, at least 366, at least 367, at least 368, at least 369, at least 370, at least 371, at least 372, at least 373, at least 374, at least 375, at least 376, at least 377, at least 378, at least 379, at least 380, at least 381, at least 382, at least 383, at least 384, at least 385, at least 386, at least 387, at least 388, at least 389, at least 390, at least 391, at least 392, at least 393, at least 394, at least 395, at least 396, at least 397, at least 398, at least 399, at least 400, at least 401, at least 402, at least 403, at least 404, at least 405, at least 406, at least 407, at least 408, at least 409, at least 410, at least 411, at least 412, at least 413, at least 414, at least 415, at least 416, at least 417, at least 418, at least 419, at least 420, at least 421, at least 422, at least 423, at least 424, at least 425, at least 426, at least 427, at least 428, at least 429, at least 430, at least 431, at least 432, at least 433, at least 434, at least 435, at least 436, at least 437, at least 438, at least 439, at least 440, at least 441, at least 442, at least 443, at least 444, at least 445, at least 446, at least 447, at least 448, at least 449, at least 450, at least 451, at least 452, at least 453, at least 454, at least 455, at least 456, at least 457, at least 458, at least 459, at least 460, at least 461, at least 462, at least 463, at least 464, at least 465, at least 466, at least 467, at least 468, at least 469, at least 470, at least 471, at least 472, at least 473, at least 474, at least 475, at least 476, at least 477, at least 478, at least 479, at least 480, at least 481, at least 482, at least 483, at least 484, at least 485, at least 486, at least 487, at least 488, at least 489, at least 490, at least 491, at least 492, at least 493, at least 494, at least 495, at least 496, at least 497, at least 498, at least 499, at least 500, at least 501, at least 502, at least 503, at least 504, at least 505, at least 506, at least 507, or at least 508, at least 509, at least 510, at least 515, at least 520, at least 525, at least 530, at least 535, at least 540, at least 545, at least 550, at least 555, at least 560, at least 565, at least 570, at least 575, at least 580, at least 585, at least 590, at least 595, at least 600, at least 605, at least 610, at least 615, at least 620, at least 625, at least 630, at least 635, at least 640, at least 645, at least 650, at least 655, at least 660, at least 665, at least 670, at least 675, at least 680, at least 685, at least 690, at least 695, at least 700, at least 705, at least 710, at least 715, at least 720, at least 725, at least 730, at least 735, at least 740, at least 745, at least 750, at least 755, at least 760, at least 765, at least 770, at least 775, at least 780, at least 785, at least 790, at least 795, or at least 800 or more of the microsatellites set out in the respective Table for the subject's cancer type (e.g., at least one of Tables 1-15 or Table 21).

In some embodiments of any of the aspects, a set of microsatellite repeat marker sequences comprises a plurality of at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, at most 250, at most 251, at most 252, at most 253, at most 254, at most 255, at most 256, at most 257, at most 258, at most 259, at most 260, at most 261, at most 262, at most 263, at most 264, at most 265, at most 266, at most 267, at most 268, at most 269, at most 270, at most 271, at most 272, at most 273, at most 274, at most 275, at most 276, at most 277, at most 278, at most 279, at most 280, at most 281, at most 282, at most 283, at most 284, at most 285, at most 286, at most 287, at most 288, at most 289, at most 290, at most 291, at most 292, at most 293, at most 294, at most 295, at most 296, at most 297, at most 298, at most 299, at most 300, at most 301, at most 302, at most 303, at most 304, at most 305, at most 306, at most 307, at most 308, at most 309, at most 310, at most 311, at most 312, at most 313, at most 314, at most 315, at most 316, at most 317, at most 318, at most 319, at most 320, at most 321, at most 322, at most 323, at most 324, at most 325, at most 326, at most 327, at most 328, at most 329, at most 330, at most 331, at most 332, at most 333, at most 334, at most 335, at most 336, at most 337, at most 338, at most 339, at most 340, at most 341, at most 342, at most 343, at most 344, at most 345, at most 346, at most 347, at most 348, at most 349, at most 350, at most 351, at most 352, at most 353, at most 354, at most 355, at most 356, at most 357, at most 358, at most 359, at most 360, at most 361, at most 362, at most 363, at most 364, at most 365, at most 366, at most 367, at most 368, at most 369, at most 370, at most 371, at most 372, at most 373, at most 374, at most 375, at most 376, at most 377, at most 378, at most 379, at most 380, at most 381, at most 382, at most 383, at most 384, at most 385, at most 386, at most 387, at most 388, at most 389, at most 390, at most 391, at most 392, at most 393, at most 394, at most 395, at most 396, at most 397, at most 398, at most 399, at most 400, at most 401, at most 402, at most 403, at most 404, at most 405, at most 406, at most 407, at most 408, at most 409, at most 410, at most 411, at most 412, at most 413, at most 414, at most 415, at most 416, at most 417, at most 418, at most 419, at most 420, at most 421, at most 422, at most 423, at most 424, at most 425, at most 426, at most 427, at most 428, at most 429, at most 430, at most 431, at most 432, at most 433, at most 434, at most 435, at most 436, at most 437, at most 438, at most 439, at most 440, at most 441, at most 442, at most 443, at most 444, at most 445, at most 446, at most 447, at most 448, at most 449, at most 450, at most 451, at most 452, at most 453, at most 454, at most 455, at most 456, at most 457, at most 458, at most 459, at most 460, at most 461, at most 462, at most 463, at most 464, at most 465, at most 466, at most 467, at most 468, at most 469, at most 470, at most 471, at most 472, at most 473, at most 474, at most 475, at most 476, at most 477, at most 478, at most 479, at most 480, at most 481, at most 482, at most 483, at most 484, at most 485, at most 486, at most 487, at most 488, at most 489, at most 490, at most 491, at most 492, at most 493, at most 494, at most 495, at most 496, at most 497, at most 498, at most 499, at most 500, at most 501, at most 502, at most 503, at most 504, at most 505, at most 506, at most 507, at most 508, at most 509, at most 510, at most 515, at most 520, at most 525, at most 530, at most 535, at most 540, at most 545, at most 550, at most 555, at most 560, at most 565, at most 570, at most 575, at most 580, at most 585, at most 590, at most 595, at most 600, at most 605, at most 610, at most 615, at most 620, at most 625, at most 630, at most 635, at most 640, at most 645, at most 650, at most 655, at most 660, at most 665, at most 670, at most 675, at most 680, at most 685, at most 690, at most 695, at most 700, at most 705, at most 710, at most 715, at most 720, at most 725, at most 730, at most 735, at most 740, at most 745, at most 750, at most 755, at most 760, at most 765, at most 770, at most 775, at most 780, at most 785, at most 790, at most 795, at most 800 of the microsatellites set out in the respective Table for the subject's cancer type (e.g., at least one of Tables 1-15 or Table 21).

The microsatellite repeat marker sequences in Tables 1-15 are listed in rank order; i.e., they are ordered by significance, from the highest significance (i.e., lowest p-value) in the first data row, to the lowest significance (i.e., highest p-value) in the last data row. In some embodiments of any of the aspects, the set of microsatellite repeat marker sequences comprises a plurality up to the first 5, 10, 20, 30, 40, 50, 60, 70, 80, 90, 100, 150, 200, 250, 300, 350, 400, 450, 500, 600, 700, or 800 or more of the sequences set out in the respective Table for the subject's cancer type e.g., at least one of Tables 1-15).

In some embodiments of any of the aspects, the set of microsatellite repeat marker sequences comprises a plurality comprising sequences, each with a p-value of no more than 1E-48, no more than 1E-47, no more than 1E-46, no more than 1E-45, no more than 1E-44, no more than 1E-43, no more than 1E-42, no more than 1E-41, no more than 1E-40, no more than 1E-39, no more than 1E-38, no more than 1E-37, no more than 1E-36, no more than 1E-35, no more than 1E-34, no more than 1E-33, no more than 1E-32, no more than 1E-31, no more than 1E-30, no more than 1E-29, no more than 1E-28, no more than 1E-27, no more than 1E-26, no more than 1E-25, no more than 1E-24, no more than 1E-23, no more than 1E-22, no more than 1E-21, no more than 1E-20, no more than 1E-19, no more than 1E-18, no more than 1E-17, no more than 1E-16, no more than 1E-15, no more than 1E-14, no more than 1E-13, no more than 1E-12, no more than 1E-11, no more than 1E-10, no more than 1E-9, no more than 1E-8, no more than 1E-7, no more than 1E-6, no more than 1E-5, no more than 1E-4, no more than 1E-3, no more than 0.01, no more than 0.02, no more than 0.03, no more than 0.04, or no more than 0.05, as set out in the respective Table for the subject's cancer type e.g., at least one of Tables 1-15).

TABLE 1
Microsatellite Repeat Marker Sequences for Colon Adenocarcinoma (COAD).
TABLE 1 - COAD

| ch | start_pos | end_pos | | COAD |
|---|---|---|---|---|
| (hg37) | (hg37) | (hg37) | gene | p-value |
| 9 | 133498229 | 133498245 | FUBP3 | 1.97444E−42 |
| 22 | 30051705 | 30051722 | NF2 | 4.2723E−40 |
| 17 | 29508819 | 29508836 | NF1 | 7.41122E−39 |
| 18 | 649879 | 649895 | TYMSOS(NM_001012716: c.*120_*104del0) | 1.18183E−38 |
| 13 | 28133956 | 28133972 | LNX2 | 5.27268E−38 |
| 6 | 111329221 | 111329239 | RPF2 | 6.2912E−38 |
| 1 | 33402334 | 33402352 | RNF19B(NM_001127361: c.*555_*537del0, | 7.77278E−38 |
| | | | NM_153341: c.*73_*55del0, | |
| | | | NM_001300826: c.*73_*55del0) | |
| 7 | 92900502 | 92900527 | CCDC132 | 1.12934E−37 |
| 10 | 112360315 | 112360333 | SMC3 | 1.17805E−37 |
| 6 | 89638988 | 89639004 | RNGTT | 1.3471E−37 |
| 5 | 86679495 | 86679514 | RASA1 | 1.90078E−37 |
| 4 | 146031236 | 146031253 | ABCE1 | 3.46974E−37 |
| 2 | 99936265 | 99936280 | TXNDC9 | 1.18581E−36 |
| 3 | 32571823 | 32571841 | DYNC1LI1 | 1.85388E−36 |
| X | 106092450 | 106092473 | TBCID8B | 2.91268E−36 |
| 1 | 32381592 | 32381607 | PTP4A2 | 5.75413E−36 |
| 4 | 115586972 | 115586988 | UGT8 | 6.55335E−36 |
| 2 | 62063093 | 62063111 | FAM161A | 6.90573E−36 |
| 6 | 32147291 | 32147312 | RNF5P1 | 8.98174E−36 |
| 16 | 75674252 | 75674271 | KARS | 1.567E−35 |
| 5 | 172578549 | 172578567 | BNIP1 | 5.32873E−35 |
| 5 | 94842718 | 94842730 | TTC37 | 1.08708E−34 |
| 12 | 110834030 | 110834049 | ANAPC7 | 1.84502E−34 |
| 14 | 73959703 | 73959720 | C14orf169(NM_024644: c.*56_*73del0) | 2.1326E−34 |
| 2 | 15536509 | 15536532 | NBAS | 2.20429E−34 |
| 1 | 94964139 | 94964155 | ABCD3 | 3.83708E−34 |
| 20 | 35695535 | 35695562 | RBL1 | 4.26031E−34 |
| 13 | 79216356 | 79216381 | RNF219 | 6.25174E−34 |
| 5 | 172421760 | 172421776 | ATP6V0E1 | 8.57225E−34 |
| 2 | 48734331 | 48734349 | PPP1R21 | 1.27747E−33 |
| 1 | 150676674 | 150676692 | HORMAD1 | 1.37307E−33 |
| 3 | 196088809 | 196088826 | UBXN7 | 1.58786E−33 |
| 8 | 103287850 | 103287864 | UBR5 | 1.60937E−33 |
| 3 | 138248009 | 138248026 | CEP70 | 1.88658E−33 |
| 9 | 30689085 | 30689104 | LINC01242(dist = 280633), ACO1(dist = 1695497) | 3.97068E−33 |
| 6 | 157495951 | 157495966 | ARID1B | 4.13998E−33 |
| 2 | 74089266 | 74089281 | STAMBP | 5.7002E−33 |
| 2 | 61308569 | 61308585 | KIAA1841 | 8.51011E−33 |
| 6 | 49415351 | 49415368 | MUT | 8.71316E−33 |
| 1 | 47123898 | 47123914 | ATPAF1 | 2.42202E−32 |
| 1 | 113159364 | 113159384 | ST7L | 2.59998E−32 |
| 6 | 36452603 | 36452620 | KCTD20 | 2.82618E−32 |
| 5 | 50122645 | 50122660 | PARP8 | 2.91483E−32 |
| 14 | 59787325 | 59787338 | DAAM1 | 3.69262E−32 |
| 2 | 201778717 | 201778733 | ORC2 | 3.824E−32 |
| 1 | 223156384 | 223156419 | DISP1 | 4.72047E−32 |
| 16 | 47399670 | 47399687 | ITFG1 | 6.48005E−32 |
| 7 | 36465606 | 36465622 | ANLN | 8.85541E−32 |
| 14 | 58825825 | 58825847 | ARID4A | 8.8991E−32 |
| 7 | 54819993 | 54820005 | SEC61G(NM_014302: c.*111_*99del0, | 9.41642E−32 |
| | | | NM_001012456: c.*111_*99del0) | |
| 21 | 37706065 | 37706077 | MORC3 | 1.05742E−31 |
| 1 | 165712555 | 165712571 | TMCO1 | 1.70961E−31 |
| 1 | 212217944 | 212217961 | DTL | 2.36848E−31 |
| 2 | 55883509 | 55883523 | PNPT1 | 3.31975E−31 |
| 16 | 90141288 | 90141302 | PRDM7 | 3.78188E−31 |
| 1 | 63044645 | 63044661 | DOCK7 | 5.63554E−31 |
| X | 96171416 | 96171435 | DIAPH2 | 6.17561E−31 |
| 11 | 110128925 | 110128941 | RDX | 7.14912E−31 |
| 3 | 132169705 | 132169718 | DNAJC13 | 7.16803E−31 |
| 8 | 53543090 | 53543106 | RB1CC1 | 8.99963E−31 |
| 4 | 128814425 | 128814449 | PLK4 | 1.82148E−30 |
| 3 | 71008341 | 71008362 | FOXP1(NM_001244816: c._*57_*36del0, | 1.84404E−30 |
| | | | NM_001244812: c.*57_*36del0, | |
| | | | NM_001244813: c.*57_*36del0, | |
| | | | NM_001244808: c.*57_*36del0, | |
| | | | NM_001244815: c.*57_*36del0, | |
| | | | NM_001244814: c.*57_*36del0, | |
| | | | NM_032682: c.*57_*36del0, | |
| | | | NM_001244810: c.*57_*36del0) | |
| 4 | 39501722 | 39501754 | MIR1273H | 2.16074E−30 |
| 9 | 163927 | 163943 | CBWD1 | 2.29365E−30 |
| 10 | 30605169 | 30605182 | MTPAP | 2.4128E−30 |
| X | 18800531 | 18800548 | PPEF1 | 3.27994E−30 |
| 18 | 19119813 | 19119828 | ESCO1 | 3.29335E−30 |
| 17 | 30293149 | 30293164 | SUZ12 | 3.31796E−30 |
| 13 | 103257289 | 103257311 | TPP2 | 3.66206E−30 |
| 2 | 109371849 | 109371865 | RANBP2 | 4.87581E−30 |
| 12 | 89853387 | 89853405 | POC1B | 5.24509E−30 |
| 18 | 33058318 | 33058335 | INO80C | 7.15262E−30 |
| 6 | 100988266 | 100988280 | ASCC3 | 7.50578E−30 |
| 2 | 61577667 | 61577680 | USP34 | 7.77574E−30 |
| 3 | 58351660 | 58351690 | PXK | 8.33089E−30 |
| 14 | 68582259 | 68582279 | RAD51B | 8.94048E−30 |
| 1 | 197053561 | 197053577 | ASPM | 9.31752E−30 |
| 3 | 169992958 | 169992994 | PRKCI | 9.86781E−30 |
| 8 | 3611416 | 3611429 | CSMD1 | 1.83694E−29 |
| 2 | 32117217 | 32117230 | MEMO1 | 1.85212E−29 |
| 13 | 39608335 | 39608360 | PROSER1 | 1.88642E−29 |
| X | 84523274 | 84523298 | ZNF711 | 2.1074E−29 |
| 12 | 109510086 | 109510099 | USP30 | 2.14945E−29 |
| 15 | 50596341 | 50596355 | GABPB1 | 2.77078E−29 |
| 12 | 110006724 | 110006738 | MMAB | 2.92762E−29 |
| 12 | 76780051 | 76780066 | OSBPL8 | 2.92907E−29 |
| 4 | 128812230 | 128812245 | PLK4 | 3.02816E−29 |
| 1 | 222904869 | 222904881 | BROX | 4.84008E−29 |
| 12 | 104714847 | 104714867 | TXNRD1 | 5.16677E−29 |
| 9 | 125655308 | 125655324 | RC3H2 | 5.31074E−29 |
| 5 | 36123065 | 36123135 | LMBRD2 | 5.75649E−29 |
| 2 | 112854563 | 112854576 | TMEM87B | 7.87714E−29 |
| 2 | 203676382 | 203676403 | ICA1L | 1.02838E−28 |
| 4 | 128817905 | 128817919 | PLK4 | 1.03158E−28 |
| 6 | 24515330 | 24515350 | ALDH5A1 | 1.04464E−28 |
| 16 | 15159022 | 15159039 | PDXDC1, RRN3 | 1.07089E−28 |
| 20 | 35672436 | 35672456 | RBL1 | 1.7126E−28 |
| 8 | 30933816 | 30933829 | WRN | 1.79545E−28 |
| 1 | 117605130 | 117605145 | TTF2 | 2.02249E−28 |
| 7 | 16676082 | 16676097 | ANKMY2 | 2.14328E−28 |
| 9 | 133498017 | 133498047 | FUBP3 | 2.59338E−28 |
| 19 | 54603097 | 54603118 | OSCAR | 2.90262E−28 |
| 2 | 172582088 | 172582125 | DYNC1I2 | 3.16686E−28 |
| 2 | 103343275 | 103343295 | MFSD9 | 3.5448E−28 |
| 6 | 70840124 | 70840137 | COL19A1 | 3.86566E−28 |
| 2 | 234916771 | 234916787 | TRPM8 | 4.34333E−28 |
| 5 | 14485161 | 14485175 | TRIO | 4.43695E−28 |
| 14 | 35242702 | 35242726 | BAZ1A | 4.48519E−28 |
| 16 | 10567751 | 10567778 | ATF7IP2 | 5.3796E−28 |
| 1 | 33087567 | 33087584 | ZBTB8OS | 5.39418E−28 |
| X | 19037666 | 19037686 | GPR64 | 5.53603E−28 |
| 4 | 76997157 | 76997173 | ART3 | 5.75346E−28 |
| 19 | 15511205 | 15511221 | AKAP8L | 6.00927E−28 |
| 2 | 179548695 | 179548712 | TTN | 6.50856E−28 |
| 6 | 20758808 | 20758822 | CDKAL1 | 7.44263E−28 |
| 8 | 100160282 | 100160300 | VPS13B | 7.5375E−28 |
| 5 | 118500981 | 118501000 | DMXL1 | 9.02536E−28 |
| 3 | 132173004 | 132173026 | DNAJC13 | 1.1257E−27 |
| 3 | 33626219 | 33626236 | CLASP2 | 1.17044E−27 |
| 1 | 183481943 | 183481964 | SMG7 | 1.25607E−27 |
| 5 | 96093391 | 96093408 | CAST | 1.38902E−27 |
| 20 | 58497481 | 58497497 | SYCP2(NM_014258: c.-13_-29del0) | 1.66216E−27 |
| 9 | 19299209 | 19299224 | DENND4C | 1.73078E−27 |
| 3 | 108224685 | 108224701 | MYH15 | 1.92096E−27 |
| X | 48214185 | 48214200 | SSX3 | 2.22276E−27 |
| 3 | 44846477 | 44846493 | KIF15 | 2.29722E−27 |
| 1 | 213009178 | 213009196 | SPATA45 | 2.31418E−27 |
| 2 | 239241445 | 239241460 | TRAF3IP1 | 2.65192E−27 |
| 3 | 113222099 | 113222114 | SPICE1 | 2.67655E−27 |
| 20 | 47649496 | 47649525 | ARFGEF2 | 2.85041E−27 |
| 3 | 111873902 | 111873917 | SLC9C1 | 3.0073E−27 |
| 2 | 91887905 | 91887919 | LOC654342(dist = 39930), GGT8P(dist = 75449) | 3.20197E−27 |
| 1 | 150297334 | 150297351 | PRPF3 | 3.3667E−27 |
| 12 | 123703074 | 123703086 | MPHOSPH9 | 3.86064E−27 |
| 14 | 39591613 | 39591627 | GEMIN2 | 4.36301E−27 |
| 10 | 35314130 | 35314161 | CUL2 | 4.55769E−27 |
| 2 | 48032740 | 48032754 | MSH6 | 4.79768E−27 |
| 8 | 96069973 | 96069988 | NDUFAF6 | 4.87356E−27 |
| 1 | 57249981 | 57250000 | RSPRY1 | 5.37964E−27 |
| 7 | 74608740 | 74608754 | GTF2IP1, LOC100093631 | 6.05012E−27 |
| 17 | 28811834 | 28811848 | GOSR1 | 6.24449E−27 |
| 6 | 11714639 | 11714665 | ADTRP(NM_001143948: c.*72_*46del0, | 6.32705E−27 |
| | | | NM_032744: c.*72_*46del0) | |
| 12 | 49091972 | 49092009 | CCNT1 | 7.01103E−27 |
| 2 | 70456452 | 70456465 | TIA1 | 8.78721E−27 |
| 4 | 39851058 | 39851077 | PDS5A | 1.001E−26 |
| 11 | 108058769 | 108058785 | NPAT | 1.01853E−26 |
| 5 | 52358761 | 52358775 | ITGA2 | 1.22448E−26 |
| 2 | 173435578 | 173435593 | PDK1 | 1.27764E−26 |
| 6 | 20548763 | 20548778 | CDKAL1 | 1.30837E−26 |
| 18 | 66367726 | 66367742 | TMX3 | 1.57507E−26 |
| 2 | 152424933 | 152424962 | NEB | 1.75375E−26 |
| 3 | 180320897 | 180320910 | TTC14 | 1.92782E−26 |
| X | 44386435 | 44386460 | FUNDC1 | 2.11618E−26 |
| 15 | 30875074 | 30875089 | ULK4P1, ULK4P2 | 2.23317E−26 |
| 9 | 114341239 | 114341255 | PTGR1 | 2.25943E−26 |
| 13 | 102235764 | 102235780 | ITGBL1 | 2.28436E−26 |
| 16 | 15162143 | 15162165 | PDXDC1, RRN3 | 2.44732E−26 |
| 2 | 203079025 | 203079039 | SUMO1 | 2.47405E−26 |
| 4 | 84519775 | 84519790 | AGPAT9 | 2.54004E−26 |
| 15 | 55841195 | 55841209 | PYGO1 | 2.55373E−26 |
| 22 | 41545024 | 41545039 | EP300 | 2.65914E−26 |
| 20 | 31954819 | 31954834 | CDK5RAP1 | 2.94342E−26 |
| 2 | 120714401 | 120714418 | PTPN4 | 2.97068E−26 |
| 11 | 8486375 | 8486389 | STK33 | 3.0217E−26 |
| 2 | 108520601 | 108520629 | RGPD4(dist = 11601), SLC5A7(dist = 82366) | 3.17888E−26 |
| 11 | 59368218 | 59368235 | OSBP | 3.72002E−26 |
| 20 | 46266543 | 46266558 | NCOA3 | 3.73849E−26 |
| 2 | 236877066 | 236877084 | AGAP1 | 3.85222E−26 |
| 11 | 118884009 | 118884025 | CCDC84 | 5.08727E−26 |
| 1 | 65099701 | 65099716 | CACHD1 | 5.33943E−26 |
| 14 | 32142544 | 32142557 | NUBPL | 5.75709E−26 |
| 14 | 96770942 | 96770967 | ATG2B | 6.43549E−26 |
| 16 | 19063220 | 19063235 | TMC7 | 6.75772E−26 |
| 12 | 21644428 | 21644447 | RECQL | 6.91293E−26 |
| 14 | 53513439 | 53513452 | DDHD1(NM_001160148: c.*47_*34del0, | 7.32716E−26 |
| | | | NM_001160147: c.*47_*34del0, | |
| | | | NM_030637: c.*47_*34del0) | |
| 14 | 36218039 | 36218056 | RALGAPA1P | 8.15888E−26 |
| 5 | 95224700 | 95224719 | ELL2 | 8.88189E−26 |
| 3 | 71739332 | 71739345 | EIF4E3 | 9.69231E−26 |
| 11 | 88068256 | 88068270 | CTSC | 9.73243E−26 |
| 5 | 41909830 | 41909846 | C5orf51 | 9.79141E−26 |
| 15 | 82517622 | 82517671 | EFTUD1 | 1.0184E−25 |
| 16 | 24185815 | 24185831 | PRKCB | 1.09299E−25 |
| 9 | 86395372 | 86395398 | GKAP1 | 1.13366E−25 |
| 4 | 95508108 | 95508128 | PDLIM5 | 1.13711E−25 |
| 3 | 69112658 | 69112688 | UBA3 | 1.17295E−25 |
| 16 | 3808052 | 3808066 | CREBBP | 1.28767E−25 |
| 1 | 92763091 | 92763128 | GLMN | 1.36845E−25 |
| 3 | 121202433 | 121202476 | POLQ | 1.37521E−25 |
| 8 | 142161666 | 142161681 | DENND3 | 1.45121E−25 |
| 17 | 1265304 | 1265326 | YWHAE | 1.67543E−25 |
| 9 | 34017005 | 34017043 | UBAP2 | 1.77445E−25 |
| X | 20204548 | 20204563 | RPS6KA3 | 1.85E−25 |
| 4 | 44691795 | 44691810 | GUF1 | 1.92631E−25 |
| 17 | 35979746 | 35979761 | DDX52 | 2.09281E−25 |
| 17 | 54981571 | 54981588 | TRIM25 | 2.23713E−25 |
| 9 | 116044905 | 116044921 | PRPF4 | 2.27948E−25 |
| 8 | 109247189 | 109247210 | EIF3E | 2.33007E−25 |
| 13 | 48664556 | 48664569 | MED4 | 2.37233E−25 |
| 8 | 89499214 | 89499230 | MMP16(dist = 159497), LOC101929709(dist = 1230397) | 2.53324E−25 |
| 20 | 7921106 | 7921120 | HAO1 | 2.64395E−25 |
| 1 | 112305406 | 112305423 | DDX20 | 2.66171E−25 |
| 6 | 152740681 | 152740695 | SYNE1 | 2.84733E−25 |
| 17 | 19749952 | 19749969 | ULK2 | 2.863E−25 |
| 1 | 14057425 | 14057439 | PRDM2 | 2.98632E−25 |
| 5 | 37027560 | 37027590 | NIPBL | 3.02598E−25 |
| 14 | 20857306 | 20857321 | TEP1 | 3.15633E−25 |
| 14 | 36096255 | 36096278 | RALGAPA1P | 3.59889E−25 |
| 14 | 60616914 | 60616937 | DHRS7 | 3.78632E−25 |
| 12 | 48360947 | 48360961 | TMEM106C | 3.99918E−25 |
| X | 44918221 | 44918236 | KDM6A | 4.03992E−25 |
| 14 | 35899480 | 35899493 | NFKBIA(dist = 25520), INSM2(dist = 103755) | 4.3902E−25 |
| 3 | 113169372 | 113169386 | SPICE1 | 4.40181E−25 |
| X | 2343183 | 2343213 | DHRSX | 4.47914E−25 |
| 9 | 79968285 | 79968309 | VPS13A | 4.49841E−25 |
| 2 | 32095023 | 32095056 | MEMO1 | 4.58282E−25 |
| 4 | 89352469 | 89352487 | HERC6 | 5.3032E−25 |
| 2 | 64323751 | 64323763 | PELI1 | 5.65312E−25 |
| 11 | 122928926 | 122928953 | HSPA8 | 6.23598E−25 |
| 7 | 114303617 | 114303633 | FOXP2 | 6.55004E−25 |
| 5 | 87494728 | 87494767 | TMEM161B | 6.60555E−25 |
| 11 | 14539315 | 14539332 | PSMA1 | 7.15944E−25 |
| 1 | 159032486 | 159032497 | AIM2 | 7.94894E−25 |
| 1 | 155701055 | 155701071 | DAP3 | 8.04399E−25 |
| 18 | 59942586 | 59942600 | KIAA1468 | 9.06981E−25 |
| 6 | 24412431 | 24412445 | MRS2 | 1.02893E−24 |
| 5 | 178030941 | 178030955 | CLK4 | 1.07799E−24 |
| 21 | 27136460 | 27136473 | GABPA | 1.13104E−24 |
| 12 | 45771901 | 45771918 | ANO6 | 1.13312E−24 |
| 2 | 47635523 | 47635537 | MSH2 | 1.16329E−24 |
| 2 | 120620092 | 120620108 | PTPN4 | 1.26988E−24 |
| 2 | 111406968 | 111406984 | BUB1 | 1.28181E−24 |
| 2 | 54146387 | 54146400 | PSME4 | 1.33261E−24 |
| 5 | 180481216 | 180481233 | BTNL9 | 1.42643E−24 |
| 2 | 64139831 | 64139844 | VPS54 | 1.44374E−24 |
| 1 | 173490547 | 173490571 | SLC9C2 | 1.46066E−24 |
| 1 | 185120927 | 185120940 | TRMT1L | 1.57643E−24 |
| 17 | 57937792 | 57937807 | TUBD1 | 1.60716E−24 |
| 3 | 52696310 | 52696322 | PBRM1 | 1.83711E−24 |
| X | 134025421 | 134025436 | MOSPD1 | 2.09041E−24 |
| 2 | 157370018 | 157370033 | GPD2 | 2.09277E−24 |
| 4 | 55976947 | 55976961 | KDR | 2.393E−24 |
| 18 | 40503735 | 40503751 | RIT2 | 2.48943E−24 |
| 2 | 230656987 | 230657001 | TRIP12 | 2.7448E−24 |
| 3 | 20189528 | 20189540 | KAT2B | 2.76665E−24 |
| 20 | 37191391 | 37191408 | RALGAPB | 3.21935E−24 |
| 7 | 138434066 | 138434081 | ATP6V0A4 | 3.23489E−24 |
| 2 | 64806544 | 64806556 | AFTPH | 3.26179E−24 |
| 2 | 158275111 | 158275135 | CYTIP | 3.52321E−24 |
| 1 | 210411266 | 210411281 | SERTAD4 | 3.58194E−24 |
| 2 | 86683539 | 86683575 | KDM3A | 3.72631E−24 |
| 1 | 42776699 | 42776711 | FOXJ3 | 3.76862E−24 |
| 8 | 87751841 | 87751867 | CNGB3 | 3.95052E−24 |
| 5 | 134086670 | 134086684 | CAMLG(NM_001745: c.*30_*44del0) | 4.26748E−24 |
| 8 | 101933023 | 101933037 | YWHAZ | 4.82347E−24 |
| 7 | 81695842 | 81695859 | CACNA2D1 | 4.95872E−24 |
| 1 | 79383748 | 79383762 | ELTD1 | 5.04093E−24 |
| 3 | 5249962 | 5249976 | EDEM1 | 5.09083E−24 |
| 11 | 33374613 | 33374626 | HIPK3 | 5.35266E−24 |
| 1 | 158584103 | 158584116 | SPTA1 | 5.62348E−24 |
| 8 | 23712066 | 23712089 | STC1(NM_003155: c.-30_-53del0) | 5.72444E−24 |
| 5 | 14388797 | 14388816 | TRIO | 5.80373E−24 |
| 3 | 38257551 | 38257583 | OXSR1 | 6.714E−24 |
| 18 | 29435778 | 29435795 | TRAPPC8 | 6.89026E−24 |
| 1 | 54395673 | 54395692 | HSPB11 | 6.93225E−24 |
| 5 | 50055456 | 50055473 | PARP8 | 8.17131E−24 |
| 6 | 149846372 | 149846387 | PPIL4 | 9.48992E−24 |
| 15 | 56946703 | 56946733 | ZNF280D | 1.11368E−23 |
| 20 | 48562797 | 48562813 | RNF114 | 1.12626E−23 |
| 2 | 131883287 | 131883301 | PLEKHB2 | 1.2191E−23 |
| 10 | 21461410 | 21461435 | NEBL | 1.31105E−23 |
| 13 | 73409508 | 73409522 | PIBF1 | 1.34259E−23 |
| 15 | 43430749 | 43430765 | TMEM62 | 1.35865E−23 |
| 6 | 15651642 | 15651656 | DTNBP1 | 1.41445E−23 |
| 2 | 135887514 | 135887528 | RAB3GAP1 | 1.44056E−23 |
| 14 | 21964623 | 21964635 | TOX4 | 1.48711E−23 |
| 1 | 151384052 | 151384067 | POGZ | 1.49637E−23 |
| 12 | 10205420 | 10205435 | CLEC9A | 1.58202E−23 |
| 20 | 271204 | 271226 | C20orf96 | 1.59463E−23 |
| 10 | 70056619 | 70056632 | PBLD | 1.623E−23 |
| 20 | 37146131 | 37146146 | RALGAPB | 1.67662E−23 |
| 13 | 48828102 | 48828118 | ITM2B | 1.68847E−23 |
| 10 | 27333121 | 27333134 | ANKRD26 | 1.85758E−23 |
| X | 138864932 | 138864946 | ATP11C | 1.9878E−23 |
| 2 | 9558905 | 9558924 | ITGB1BP1 | 2.0684E−23 |
| 2 | 101885656 | 101885675 | CNOT11 | 2.15963E−23 |
| 9 | 130127691 | 130127703 | GARNL3 | 2.16385E−23 |
| 8 | 59518439 | 59518456 | NSMAF | 2.19278E−23 |
| 6 | 145095545 | 145095558 | UTRN | 2.50911E−23 |
| 5 | 96342077 | 96342093 | LNPEP | 2.60313E−23 |
| 12 | 97306603 | 97306631 | NEDD1 | 2.62054E−23 |
| 6 | 17675386 | 17675402 | NUP153 | 2.824E−23 |
| 22 | 44083327 | 44083345 | EFCAB6 | 2.89856E−23 |
| 5 | 10394911 | 10394930 | MARCH6 | 3.025E−23 |
| 6 | 71571688 | 71571707 | SMAP1(NM_001281439: c.*1751_*1770del0, | 3.21041E−23 |
| | | | NM_021940: c.*1651_*1670del0, | |
| | | | NM_001044305: c.*1651_*1670del0, | |
| | | | NM_001281440: c.*1651_*1670del0) | |
| 3 | 113377481 | 113377493 | KIAA2018 | 3.24595E−23 |
| 3 | 41877473 | 41877487 | ULK4 | 3.47059E−23 |
| 14 | 60587879 | 60587900 | PCNXL4 | 3.52279E−23 |
| 12 | 100496478 | 100496498 | UHRF1BP1L | 3.56689E−23 |
| 8 | 80831393 | 80831407 | MRPS28 | 3.57504E−23 |
| 12 | 125587478 | 125587491 | AACS | 3.90401E−23 |
| X | 131233588 | 131233600 | FRMD7 | 4.16916E−23 |
| 10 | 103567665 | 103567680 | MGEA5 | 4.21675E−23 |
| 12 | 21639528 | 21639542 | RECQL | 4.37998E−23 |
| 9 | 131715001 | 131715017 | NUP188 | 4.43058E−23 |
| 5 | 76785406 | 76785444 | WDR41 | 4.83785E−23 |
| X | 23693084 | 23693098 | PRDX4 | 4.90381E−23 |
| 3 | 33893929 | 33893949 | PDCD6IP | 5.05744E−23 |
| 1 | 89449508 | 89449520 | RBMXL1 | 5.68388E−23 |
| 2 | 61566824 | 61566839 | USP34 | 5.82621E−23 |
| 10 | 88834244 | 88834265 | GLUD1 | 5.94791E−23 |
| 2 | 219341717 | 219341730 | USP37 | 5.98493E−23 |
| 16 | 5129689 | 5129703 | ALG1 | 6.07055E−23 |
| 19 | 14857187 | 14857203 | EMR2 | 6.33804E−23 |
| 7 | 95775848 | 95775863 | SLC25A13 | 6.5168E−23 |
| 1 | 46739974 | 46739989 | RAD54L | 6.88557E−23 |
| 10 | 74653468 | 74653479 | OIT3(NM_152635: c.-89_-78del-) | 6.96623E−23 |
| 3 | 48491343 | 48491366 | ATRIP | 6.97912E−23 |
| 20 | 8665502 | 8665518 | PLCB1 | 7.52397E−23 |
| 6 | 30573637 | 30573656 | PPP1R10 | 7.53381E−23 |
| 16 | 9010826 | 9010844 | USP7 | 7.59901E−23 |
| 7 | 102727051 | 102727067 | ARMC10 | 8.04084E−23 |
| 9 | 103027226 | 103027243 | INVS | 8.9062E−23 |
| X | 48156078 | 48156097 | SSX1(dist = 29199), SSX9(dist = 4888) | 9.26142E−23 |
| 12 | 28412398 | 28412416 | CCDC91 | 9.31126E−23 |
| 14 | 31191658 | 31191682 | SCFD1 | 9.73138E−23 |
| 9 | 97555228 | 97555242 | C9orf3 | 9.8621E−23 |
| 7 | 6064470 | 6064488 | EIF2AK1 | 1.0058E−22 |
| 1 | 10357206 | 10357224 | KIF1B | 1.03062E−22 |
| 8 | 38940143 | 38940157 | ADAM9 | 1.05909E−22 |
| 8 | 97296205 | 97296220 | PTDSS1 | 1.1449E−22 |
| 6 | 17669262 | 17669277 | NUP153 | 1.16335E−22 |
| 1 | 176050430 | 176050447 | RFWD2 | 1.30874E−22 |
| 12 | 50152601 | 50152618 | TMBIM6 | 1.3139E−22 |
| 9 | 19078116 | 19078132 | HAUS6 | 1.38134E−22 |
| 5 | 70837260 | 70837275 | BDP1 | 1.64155E−22 |
| 8 | 96260054 | 96260070 | C8orf37 | 1.66262E−22 |
| 8 | 86180906 | 86180924 | CA13 | 1.72829E−22 |
| 8 | 28707622 | 28707636 | INTS9 | 1.75063E−22 |
| 1 | 155686739 | 155686757 | DAP3 | 1.86544E−22 |
| 19 | 6026288 | 6026302 | RFX2 | 1.90685E−22 |
| 20 | 34435246 | 34435270 | PHF20 | 2.02382E−22 |
| 14 | 91372685 | 91372700 | RPS6KA5 | 2.2959E−22 |
| 3 | 93754286 | 93754317 | ARL13B | 2.37681E−22 |
| 1 | 172577864 | 172577879 | SUCO | 2.46375E−22 |
| 6 | 42626564 | 42626585 | UBR2 | 2.59638E−22 |
| 8 | 62550924 | 62550938 | ASPH | 2.76163E−22 |
| 5 | 150846858 | 150846882 | SLC36A1 | 2.76634E−22 |
| 6 | 84649715 | 84649747 | CYB5R4 | 2.77451E−22 |
| 2 | 99721804 | 99721820 | TSGA10 | 2.99882E−22 |
| 12 | 105538270 | 105538287 | KIAA1033 | 3.07075E−22 |
| 4 | 76528928 | 76528945 | CDKL2 | 3.13103E−22 |
| 1 | 214825289 | 214825312 | CENPF | 3.15603E−22 |
| 8 | 33356825 | 33356839 | MAK16(NM_032509: c.*678_*692del0) | 3.18837E−22 |
| 3 | 150282051 | 150282075 | EIF2A | 3.20711E−22 |
| 15 | 34380216 | 34380230 | EMC7 | 3.20711E−22 |
| 6 | 46636556 | 46636581 | SLC25A27 | 3.26649E−22 |
| 1 | 247729206 | 247729223 | GCSAML | 3.32916E−22 |
| X | 107918007 | 107918043 | COL4A5 | 3.52105E−22 |
| 22 | 41551231 | 41551249 | EP300 | 3.736E−22 |
| 1 | 202990110 | 202990128 | TMEM183A, TMEM183B | 3.84037E−22 |
| 7 | 5239305 | 5239336 | WIPI2 | 3.87028E−22 |
| 2 | 102785158 | 102785171 | IL1R1 | 4.12245E−22 |
| 14 | 55629650 | 55629664 | DLGAP5 | 4.17582E−22 |
| 3 | 122471407 | 122471434 | HSPBAP1 | 4.31879E−22 |
| 12 | 107237602 | 107237626 | RIC8B | 4.3971E−22 |
| 6 | 7413094 | 7413107 | RIOK1 | 4.65995E−22 |
| 1 | 93691855 | 93691881 | CCDC18 | 4.70356E−22 |
| 22 | 46679850 | 46679864 | TTC38 | 4.7248E−22 |
| 4 | 113568126 | 113568140 | LARP7 | 4.85516E−22 |
| 9 | 91083260 | 91083278 | SPIN1 | 4.93324E−22 |
| 16 | 24574691 | 24574710 | RBBP6 | 5.7931E−22 |
| 13 | 77792099 | 77792113 | MYCBP2 | 5.87631E−22 |
| 2 | 8822892 | 8822918 | ID2 | 6.24993E−22 |
| 13 | 77644856 | 77644877 | MYCBP2 | 6.40289E−22 |
| 3 | 42660489 | 42660508 | LOC101928323 | 6.54694E−22 |
| 5 | 137282031 | 137282059 | FAM13B | 6.61715E−22 |
| 19 | 36145409 | 36145425 | COX6B1 | 7.51746E−22 |
| 1 | 74901739 | 74901760 | FPGT-TNNI3K, TNNI3K | 7.52798E−22 |
| 17 | 44409890 | 44409927 | LRRC37A | 7.54899E−22 |
| 13 | 43491778 | 43491796 | EPSTI1 | 7.6162E−22 |
| 3 | 158526942 | 158526966 | MFSD1 | 7.78369E−22 |
| 8 | 38839302 | 38839316 | HTRA4 | 8.40849E−22 |
| 9 | 37887570 | 37887587 | SLC25A51 | 8.53361E−22 |
| 7 | 152144203 | 152144216 | FABP5P3(dist = 4103), LINC01003(dist = 16993) | 8.74701E−22 |
| 16 | 71808499 | 71808519 | AP1G1 | 8.85343E−22 |
| 4 | 9710633 | 9710651 | MIR548I2(dist = 152696), DRD5(dist = 72607) | 9.2485E−22 |
| 17 | 29195444 | 29195460 | ATAD5 | 9.29572E−22 |
| 12 | 53861636 | 53861650 | PCBP2 | 9.30239E−22 |
| 1 | 231094050 | 231094067 | TTC13 | 9.4254E−22 |
| 17 | 39033672 | 39033684 | KRT20 | 9.49511E−22 |
| 5 | 151180439 | 151180475 | G3BP1 | 9.53371E−22 |
| 19 | 37721404 | 37721419 | ZNF383 | 9.63E−22 |
| 7 | 7572355 | 7572381 | COL28A1 | 9.95204E−22 |
| 12 | 104077096 | 104077126 | STAB2 | 1.06863E−21 |
| 8 | 131193153 | 131193166 | ASAP1 | 1.0715E−21 |
| 6 | 90503754 | 90503779 | MDN1 | 1.07569E−21 |
| 2 | 211521213 | 211521243 | CPS1 | 1.12102E−21 |
| 15 | 50782416 | 50782446 | USP8 | 1.13234E−21 |
| 12 | 57006059 | 57006072 | BAZ2A | 1.29023E−21 |
| 9 | 86354663 | 86354679 | GKAP1 | 1.29873E−21 |
| 10 | 17279211 | 17279223 | VIM | 1.31339E−21 |
| 13 | 115011550 | 115011564 | CDC16 | 1.33337E−21 |
| 15 | 81187286 | 81187297 | CEMIP | 1.34615E−21 |
| 14 | 36334905 | 36334921 | BRMS1L | 1.36334E−21 |
| X | 24745018 | 24745047 | POLA1 | 1.42309E−21 |
| 3 | 195022926 | 195022944 | ACAP2 | 1.4349E−21 |
| 2 | 172187261 | 172187296 | METTL8 | 1.46E−21 |
| 4 | 17841869 | 17841881 | NCAPG | 1.60179E−21 |
| 1 | 168165767 | 168165781 | TIPRL | 1.63795E−21 |
| 4 | 1701673 | 1701686 | SLBP | 1.63997E−21 |
| 2 | 152359276 | 152359297 | NEB | 1.70334E−21 |
| X | 23724675 | 23724698 | ACOT9 | 1.7347E−21 |
| 13 | 50586051 | 50586064 | DLEU2 | 1.73805E−21 |
| 15 | 52239617 | 52239629 | LEO1 | 1.81414E−21 |
| 12 | 110454085 | 110454114 | ANKRD13A | 1.82095E−21 |
| 6 | 38905667 | 38905680 | LOC100131047 | 1.87314E−21 |
| 6 | 36569717 | 36569737 | SRSF3 | 1.96053E−21 |
| 10 | 89720622 | 89720649 | PTEN | 1.98143E−21 |
| 8 | 96047806 | 96047820 | NDUFAF6 | 1.99857E−21 |
| 13 | 25073399 | 25073415 | PARP4 | 2.01E−21 |
| 10 | 32575773 | 32575785 | EPC1 | 2.07141E−21 |
| 14 | 102550069 | 102550088 | HSP90AA1 | 2.09435E−21 |
| 16 | 21396850 | 21396873 | SNX29P1 | 2.10082E−21 |
| 22 | 38766061 | 38766082 | LOC400927 | 2.22577E−21 |
| 1 | 86435957 | 86435971 | COL24A1 | 2.31018E−21 |
| 11 | 5687320 | 5687338 | TRIM5 | 2.3563E−21 |
| 22 | 39079920 | 39079936 | TOMM22(NM_020243: c.*74_*90del0) | 2.44863E−21 |
| 4 | 141448595 | 141448610 | ELMOD2 | 2.45085E−21 |
| 1 | 175116046 | 175116071 | TNN | 2.50258E−21 |
| 15 | 59806473 | 59806486 | FAM81A | 2.71323E−21 |
| 3 | 186522293 | 186522308 | RFC4 | 2.71473E−21 |
| 1 | 245021291 | 245021305 | HNRNPU | 2.83883E−21 |
| X | 52674318 | 52674335 | SSX7 | 2.90848E−21 |
| 1 | 78432506 | 78432521 | FUBP1 | 2.98254E−21 |
| 21 | 35094849 | 35094878 | ITSN1 | 2.99288E−21 |
| 8 | 63948170 | 63948202 | GGH | 3.24232E−21 |
| 1 | 93584864 | 93584885 | MTF2 | 3.29637E−21 |
| 3 | 10103903 | 10103946 | FANCD2 | 3.31269E−21 |
| 2 | 162081128 | 162081140 | TANK | 3.32769E−21 |
| 1 | 185269100 | 185269122 | IVNSIABP | 3.36381E−21 |
| 11 | 125763610 | 125763624 | PUS3(NM_001271985: c.*70_*56del0, | 3.45862E−21 |
| | | | NM_031307: c.*70_*56del0) | |
| 5 | 70818329 | 70818354 | BDP1 | 3.46045E−21 |
| 15 | 52561891 | 52561907 | MYO5C | 3.6701E−21 |
| 17 | 74736999 | 74737016 | MFSD11 | 3.70727E−21 |
| 13 | 52726707 | 52726730 | NEK3 | 3.87017E−21 |
| 4 | 164440461 | 164440474 | TMA16 | 3.97441E−21 |
| X | 19380975 | 19380993 | MAP3K15 | 4.15548E−21 |
| 3 | 123332875 | 123332892 | MYLK-AS1 | 4.19178E−21 |
| 14 | 104040415 | 104040450 | APOPT1 | 4.40224E−21 |
| 15 | 48717850 | 48717884 | FBN1 | 4.61042E−21 |
| X | 24084241 | 24084254 | EIF2S3 | 5.86793E−21 |
| 19 | 1825786 | 1825805 | REXO1 | 5.94048E−21 |
| 11 | 68338491 | 68338520 | PPP6R3 | 6.43655E−21 |
| 9 | 79820192 | 79820224 | VPS13A | 6.44865E−21 |
| 21 | 34117067 | 34117080 | PAXBP1 | 6.5632E−21 |
| 3 | 100438778 | 100438810 | TFG | 6.56367E−21 |
| 1 | 65138787 | 65138801 | CACHD1 | 6.58731E−21 |
| 9 | 131456159 | 131456172 | SET | 6.67017E−21 |
| 1 | 10177680 | 10177693 | UBE4B | 6.7488E−21 |
| 11 | 18111872 | 18111889 | SAAL1 | 7.01393E−21 |
| 5 | 78416973 | 78416990 | BHMT | 7.10922E−21 |
| 1 | 176105710 | 176105723 | RFWD2 | 8.04665E−21 |
| X | 100793432 | 100793449 | ARMCX4(dist = 42634), ARMCX1(dist = 12065) | 8.31019E−21 |
| 6 | 20548795 | 20548808 | CDKAL1 | 8.33373E−21 |
| 5 | 54710072 | 54710091 | SKIV2L2 | 8.43081E−21 |
| 2 | 61145498 | 61145512 | REL | 8.68532E−21 |
| 12 | 45269439 | 45269452 | NELL2(NM_001145109: c.103_98delins-) | 9.40033E−21 |
| 2 | 98907089 | 98907103 | VWA3B | 9.97931E−21 |
| 3 | 37360696 | 37360708 | GOLGA4 | 1.00925E−20 |
| 11 | 118883880 | 118883892 | CCDC84 | 1.03126E−20 |
| 8 | 30464719 | 30464734 | GTF2E2 | 1.05094E−20 |
| 2 | 64195997 | 64196012 | VPS54 | 1.07643E−20 |
| 17 | 58289521 | 58289533 | USP32 | 1.17896E−20 |
| 18 | 43445730 | 43445748 | EPG5 | 1.20239E−20 |
| 14 | 20344373 | 20344394 | OR4K2 | 1.21947E−20 |
| 4 | 146046123 | 146046135 | ABCE1 | 1.23246E−20 |
| 2 | 139318332 | 139318366 | SPOPL | 1.35372E−20 |
| 4 | 141448696 | 141448718 | ELMOD2 | 1.4123E−20 |
| 12 | 56504143 | 56504159 | PA2G4 | 1.42391E−20 |
| 9 | 135773000 | 135773019 | TSC1 | 1.54202E−20 |
| X | 129267464 | 129267478 | AIFM1 | 1.54347E−20 |
| 3 | 37550027 | 37550040 | ITGA9 | 1.62302E−20 |
| 6 | 17779922 | 17779935 | KIF13A | 1.69313E−20 |
| 14 | 52957731 | 52957750 | TXNDC16 | 1.69585E−20 |
| 4 | 39233719 | 39233734 | MIR1273H | 1.73752E−20 |
| 8 | 68140383 | 68140394 | ARFGEF1 | 1.75581E−20 |
| 15 | 30010328 | 30010349 | TJP1 | 1.78485E−20 |
| 1 | 53267474 | 53267488 | ZYG11B | 1.85235E−20 |
| 1 | 109525421 | 109525434 | WDR47 | 1.88444E−20 |
| 11 | 16339918 | 16339934 | SOX6 | 1.91217E−20 |
| 3 | 160144007 | 160144039 | SMC4 | 1.95037E−20 |
| 4 | 119666217 | 119666241 | SEC24D | 1.9873E−20 |
| X | 52654536 | 52654550 | SSX8 | 2.03068E−20 |
| 3 | 30691871 | 30691882 | TGFBR2 | 2.06284E−20 |
| 12 | 14599855 | 14599867 | ATF7IP | 2.20883E−20 |
| 13 | 31722620 | 31722638 | HSPH1 | 2.25682E−20 |
| 2 | 135703650 | 135703664 | CCNT2 | 2.31976E−20 |
| 15 | 51768755 | 51768768 | DMXL2 | 2.36357E−20 |
| 10 | 21536939 | 21536956 | NEBL-AS1(dist = 73087), CASC10(dist = 246465) | 2.38852E−20 |
| 2 | 87122096 | 87122121 | ANAPC1P1 | 2.39126E−20 |

TABLE 2
Microsatellite Repeat Marker Sequences for Esophageal Carcinoma (ESCA).
TABLE 2 - ESCA

| ch | start_pos | end_pos | | ESCA |
|---|---|---|---|---|
| (hg37) | (hg37) | (hg37) | gene | p-value |
| 1 | 209974528 | 209974550 | IRF6 | 0.000249688 |
| 1 | 245025697 | 245025717 | HNRNPU | 0.000301114 |
| 1 | 33087567 | 33087584 | ZBTB8OS | 0.000333 |
| 2 | 166758447 | 166758460 | TTC21B | 0.000333 |
| 1 | 184692999 | 184693020 | EDEM3 | 0.000341763 |
| 7 | 102727051 | 102727067 | ARMC10 | 0.000350877 |
| 3 | 132169705 | 132169718 | DNAJC13 | 0.000370233 |
| 13 | 28133956 | 28133972 | LNX2 | 0.000370233 |
| 3 | 129168854 | 129168881 | IFT122 | 0.000370233 |
| 20 | 33033065 | 33033083 | ITCH | 0.000370233 |
| 14 | 31191658 | 31191682 | SCFD1 | 0.000380518 |
| 20 | 58497481 | 58497497 | SYCP2(NM_014258: c.-13_-29del0) | 0.000380518 |
| 15 | 91303325 | 91303352 | BLM(NM_001287248: c.-90_-63del-) | 0.000380518 |
| 1 | 153617510 | 153617526 | CHTOP | 0.000391236 |
| 7 | 102825987 | 102826001 | DPY19L2P2 | 0.000391236 |
| 4 | 146046123 | 146046135 | ABCE1 | 0.000391236 |
| 10 | 33496676 | 33496688 | NRP1 | 0.000391236 |
| 7 | 16722392 | 16722403 | BZW2 | 0.000391236 |
| 10 | 101510104 | 101510123 | CUTC | 0.000402414 |
| 5 | 14678762 | 14678776 | OTULIN | 0.000402414 |
| 2 | 99614595 | 99614609 | TSGA10(NM_025244: c.*65_*51del0, | 0.000402414 |
| | | | NM_182911: c.*65_*51del0) | |
| 4 | 104066176 | 104066191 | CENPE | 0.000402414 |
| 9 | 131456159 | 131456172 | SET | 0.000414079 |
| 2 | 162081128 | 162081140 | TANK | 0.000414079 |
| 18 | 19119813 | 19119828 | ESCO1 | 0.000414079 |
| 2 | 179548695 | 179548712 | TTN | 0.000414079 |
| 7 | 54819993 | 54820005 | SEC61G(NM_014302: c.*111_*99del0, | 0.000414079 |
| | | | NM_001012456: c*111_*99del0) | |
| 20 | 37191391 | 37191408 | RALGAPB | 0.000426257 |
| 4 | 146031236 | 146031253 | ABCE1 | 0.000426257 |
| 13 | 79216356 | 79216381 | RNF219 | 0.000426257 |
| 6 | 7413094 | 7413107 | RIOK1 | 0.000426257 |
| 7 | 16676082 | 16676097 | ANKMY2 | 0.000426257 |
| 9 | 86395372 | 86395398 | GKAP1 | 0.000426257 |
| 6 | 53138203 | 53138233 | ELOVL5 | 0.000426257 |
| 1 | 100542702 | 100542718 | HIAT1 | 0.000426257 |
| 5 | 122940980 | 122941019 | CSNKIG3 | 0.000438982 |
| 8 | 23712066 | 23712089 | STC1(NM_003155: c.-30_-53del0) | 0.000438982 |
| 12 | 89853387 | 89853405 | POC1B | 0.000438982 |
| 8 | 107704921 | 107704955 | OXR1 | 0.000452284 |
| 12 | 45771901 | 45771918 | ANO6 | 0.000452284 |
| 7 | 74608740 | 74608754 | GTF2IP1, LOC100093631 | 0.000452284 |
| 5 | 33546113 | 33546143 | ADAMTS12 | 0.000452284 |
| 5 | 74226391 | 74226403 | FAM169A(dist = 63728), GCNT4(dist = 96886) | 0.000452284 |
| 7 | 2406737 | 2406755 | EIF3B | 0.0004662 |
| 14 | 21964623 | 21964635 | TOX4 | 0.0004662 |
| 2 | 120714401 | 120714418 | PTPN4 | 0.0004662 |
| 6 | 111329221 | 111329239 | RPF2 | 0.0004662 |
| 2 | 197531518 | 197531530 | CCDC150 | 0.0004662 |
| 3 | 30691871 | 30691882 | TGFBR2 | 0.0004662 |
| 3 | 67559216 | 67559229 | SUCLG2 | 0.0004662 |
| 7 | 131113756 | 131113779 | MKLN1 | 0.000480769 |
| 4 | 115586972 | 115586988 | UGT8 | 0.000480769 |
| 5 | 118576096 | 118576110 | DMXL1 | 0.000480769 |
| 1 | 185834851 | 185834865 | HMCN1 | 0.000480769 |
| 17 | 35696841 | 35696853 | ACACA | 0.000480769 |
| 9 | 97555228 | 97555242 | C9orf3 | 0.000496032 |
| 3 | 48491343 | 48491366 | ATRIP | 0.000496032 |
| 2 | 189926219 | 189926240 | COL5A2 | 0.000496032 |
| 11 | 107518175 | 107518187 | ELMOD1 | 0.000512033 |
| 10 | 74874082 | 74874112 | NUDT13 | 0.000512033 |
| 15 | 52561891 | 52561907 | MYO5C | 0.000512033 |
| 12 | 88482779 | 88482791 | CEP290 | 0.000512033 |
| 15 | 69714622 | 69714658 | KIF23 | 0.000512033 |
| 20 | 37146131 | 37146146 | RALGAPB | 0.000528821 |
| 8 | 134125650 | 134125662 | TG | 0.000528821 |
| 12 | 25678995 | 25679008 | LMNTD1 | 0.000528821 |
| 17 | 66033401 | 66033412 | KPNA2 | 0.000546448 |
| 1 | 41456808 | 41456823 | CTPS1 | 0.000546448 |
| 2 | 201778717 | 201778733 | ORC2 | 0.000546448 |
| 11 | 118626215 | 118626227 | DDX6 | 0.000546448 |
| 2 | 64112697 | 64112719 | UGP2 | 0.000564972 |
| 2 | 37487341 | 37487353 | PRKD3 | 0.000564972 |
| 2 | 197654578 | 197654593 | GTF3C3 | 0.000564972 |
| 17 | 39522910 | 39522922 | KRT33B | 0.000564972 |
| 1 | 144918011 | 144918022 | NBPF20, NBPF9, PDE4DIP | 0.000564972 |
| 7 | 5087800 | 5087822 | RBAK, RBAK-RBAKDN | 0.000584454 |
| 4 | 169612452 | 169612484 | PALLD | 0.000604961 |
| 12 | 109510086 | 109510099 | USP30 | 0.000604961 |
| 9 | 98703711 | 98703723 | ERCC6L2 | 0.000604961 |
| 5 | 41909830 | 41909846 | C5orf51 | 0.000626566 |
| X | 19037666 | 19037686 | GPR64 | 0.000626566 |
| 3 | 150282051 | 150282075 | EIF2A | 0.000626566 |
| 12 | 124242458 | 124242472 | ATP6V0A2 | 0.000649351 |
| 15 | 82517622 | 82517671 | EFTUD1 | 0.000649351 |
| 10 | 71055448 | 71055460 | HK1 | 0.000649351 |
| 5 | 14485161 | 14485175 | TRIO | 0.000649351 |
| 3 | 160144007 | 160144039 | SMC4 | 0.000649351 |
| 14 | 53513439 | 53513452 | DDHD1(NM_001160148: c.*47_*34del0, | 0.000649351 |
| | | | NM_001160147: c.*47_*34del0, | |
| | | | NM_030637: c.*47_*34del0) | |
| 2 | 15536509 | 15536532 | NBAS | 0.000649351 |
| 12 | 62972198 | 62972223 | MON2 | 0.000673401 |
| 17 | 47076449 | 47076462 | IGF2BP1 | 0.000673401 |
| 6 | 25452453 | 25452466 | LRRC16A | 0.000673401 |
| 19 | 51919434 | 51919447 | LOC100129083, SIGLEC10 | 0.000673401 |
| 6 | 7595241 | 7595255 | SNRNP48 | 0.000673401 |
| 1 | 9633387 | 9633400 | SLC25A33 | 0.000673401 |
| 9 | 376186 | 376197 | DOCK8 | 0.000673401 |
| 16 | 3350676 | 3350699 | TIGD7(NM_033208: c.-62_-85del0) | 0.000673401 |
| 12 | 8813345 | 8813362 | MFAP5 | 0.000686342 |
| 2 | 47635523 | 47635537 | MSH2 | 0.000698812 |
| 15 | 45725103 | 45725138 | C15orf48 | 0.000698812 |
| 16 | 74372596 | 74372618 | LOC283922 | 0.000698812 |
| 6 | 149846372 | 149846387 | PPIL4 | 0.000698812 |
| 16 | 70176311 | 70176336 | PDPR | 0.000698812 |
| 8 | 17504626 | 17504637 | MTUS1 | 0.000698812 |
| 2 | 39240584 | 39240596 | SOS1 | 0.000725689 |
| 2 | 178364328 | 178364342 | AGPS | 0.000725689 |
| 3 | 107910327 | 107910342 | IFT57 | 0.000725689 |
| 17 | 66141017 | 66141032 | LINC00674(dist = 8947), LOC440461(dist = 53769) | 0.000725689 |
| 3 | 180320897 | 180320910 | TTC14 | 0.000754148 |
| 17 | 30293149 | 30293164 | SUZ12 | 0.000754148 |
| 16 | 70595514 | 70595525 | SF3B3 | 0.000754148 |
| 10 | 101992923 | 101992938 | CWF19L1(NM_018294: c.*61_*46del0) | 0.000754148 |
| 18 | 44424651 | 44424667 | PLAS2 | 0.000754148 |
| 6 | 100006295 | 100006309 | CCNC | 0.000754148 |
| 2 | 66796098 | 66796109 | MEIS1 | 0.000784314 |
| 15 | 65273147 | 65273161 | SPG21 | 0.000784314 |
| X | 148582573 | 148582585 | IDS | 0.000784314 |
| 10 | 116196143 | 116196156 | ABLIM1 | 0.000784314 |
| 11 | 58381663 | 58381676 | ZFP91-CNTF | 0.000784314 |
| 12 | 99060155 | 99060173 | APAF1 | 0.000784314 |
| 16 | 68155860 | 68155874 | NFATC3 | 0.000816327 |
| 1 | 237965133 | 237965146 | RYR2 | 0.000816327 |
| 15 | 23333801 | 23333815 | HERC2P2 | 0.000816327 |
| 7 | 26247984 | 26247997 | CBX3 | 0.00085034 |
| 12 | 50867692 | 50867704 | LARP 4 | 0.00085034 |
| 22 | 30772228 | 30772244 | KIAA1656 | 0.000881575 |
| 7 | 98639904 | 98639917 | SMURF1 | 0.000886525 |
| 7 | 152144203 | 152144216 | FABP5P3(dist = 4103), LINC01003(dist = 16993) | 0.000886525 |
| 1 | 221875661 | 221875686 | DUSP10(NM_007207: c.*93_*68del0) | 0.000886525 |
| 10 | 70669942 | 70669958 | DDX50 | 0.000886525 |
| 3 | 135806702 | 135806713 | PPP2R3A | 0.000886525 |
| 12 | 95488339 | 95488354 | FGD6 | 0.000903342 |
| 2 | 88875981 | 88875993 | EIF2AK3 | 0.000925069 |
| 18 | 66367726 | 66367742 | TMX3 | 0.000966184 |
| 3 | 132166148 | 132166162 | DNAJC13 | 0.000966184 |
| 10 | 35314130 | 35314161 | CUL2 | 0.00097371 |
| 8 | 67759411 | 67759438 | C8orf44-SGK3, SGK3 | 0.001010101 |
| 14 | 76638065 | 76638087 | GPATCH2L | 0.001052632 |
| 5 | 78936847 | 78936860 | PAPD4 | 0.001057082 |
| 12 | 21791410 | 21791426 | LDHB | 0.001057082 |
| 4 | 113178966 | 113178979 | AP1AR | 0.001057082 |
| 12 | 64491002 | 64491019 | SRGAP1 | 0.001081081 |
| 16 | 67384060 | 67384075 | LRRC36 | 0.00110742 |
| 14 | 56078699 | 56078713 | KTN1 | 0.00110742 |
| 2 | 120620092 | 120620108 | PTPN4 | 0.0011107 |
| 4 | 141448595 | 141448610 | ELMOD2 | 0.001141553 |
| 17 | 20639183 | 20639199 | LOC100287072 | 0.001141553 |
| 12 | 97306603 | 97306631 | NEDD1 | 0.001207243 |
| 7 | 129680648 | 129680677 | ZC3HC1 | 0.001207243 |
| 1 | 89289954 | 89289966 | PKN2 | 0.001242236 |
| 12 | 69042454 | 69042468 | RAP1B | 0.001242236 |
| 11 | 86198529 | 86198542 | ME3 | 0.001242236 |
| 1 | 212241691 | 212241730 | DTL | 0.001278772 |
| 2 | 61577667 | 61577680 | USP34 | 0.001316945 |
| 8 | 65528842 | 65528854 | CYP7B1 | 0.001316945 |
| 9 | 34017005 | 34017043 | UBAP2 | 0.001316945 |
| X | 96171416 | 96171435 | DIAPH2 | 0.001316945 |
| 18 | 12680595 | 12680617 | CEP76, PSMG2 | 0.001316945 |
| 1 | 1920434 | 1920449 | CFAP74 | 0.001316945 |
| 14 | 101506158 | 101506169 | MIR376A1, MIR376A2, MIR376B, MIR654; MIR376C | 0.001349528 |
| 12 | 122017848 | 122017866 | KDM2B | 0.001356852 |
| 12 | 51752085 | 51752103 | GALNT6 | 0.001356852 |
| 2 | 70066571 | 70066582 | GMCL1 | 0.001356852 |
| 19 | 9361740 | 9361752 | OR7E24 | 0.001398601 |
| 13 | 25355954 | 25355978 | RNF17 | 0.001422475 |
| 3 | 143235916 | 143235929 | SLC9A9 | 0.001422475 |
| 6 | 20548795 | 20548808 | CDKAL1 | 0.001442308 |
| X | 48214185 | 48214200 | SSX3 | 0.001442308 |
| 7 | 102912317 | 102912336 | DPY19L2P2 | 0.001442308 |
| 1 | 155583131 | 155583149 | MSTO2P | 0.001442308 |
| 11 | 87013273 | 87013284 | TMEM135 | 0.001442308 |
| 6 | 102124470 | 102124483 | GRIK2 | 0.001488095 |
| 5 | 137451361 | 137451372 | NME5 | 0.001501502 |
| 3 | 20189528 | 20189540 | KAT2B | 0.001536098 |
| 3 | 113377481 | 113377493 | KIAA2018 | 0.001586462 |
| 15 | 59094494 | 59094508 | FAM63B | 0.001587302 |
| 2 | 167138320 | 167138331 | SCN9A | 0.001639344 |
| 2 | 191155233 | 191155245 | HIBCH | 0.001680672 |
| 6 | 36452603 | 36452620 | KCTD20 | 0.001680672 |
| 8 | 124382376 | 124382403 | ATAD2 | 0.001680672 |
| 2 | 64806544 | 64806556 | AFTPH | 0.001680672 |
| 1 | 63270823 | 63270838 | ATG4C | 0.001694915 |
| 3 | 160395089 | 160395102 | ARL14(NM_025047: c.-46_-33del-) | 0.001694915 |
| 1 | 53267474 | 53267488 | ZYG11B | 0.001753361 |
| X | 2343183 | 2343213 | DHRSX | 0.00176315 |
| 1 | 113657018 | 113657033 | LRIG2 | 0.001782531 |
| 12 | 48360947 | 48360961 | TMEM106C | 0.001814882 |
| 1 | 150676674 | 150676692 | HORMAD1 | 0.001814882 |
| 14 | 32142544 | 32142557 | NUBPL | 0.001814882 |
| 13 | 25911034 | 25911067 | NUPL1 | 0.001879699 |
| 12 | 104714847 | 104714867 | TXNRD1 | 0.001948052 |
| 18 | 180211 | 180228 | USP14 | 0.001948052 |
| 8 | 100160282 | 100160300 | VPS13B | 0.001948052 |
| X | 79991590 | 79991604 | BRWD3 | 0.001948052 |
| 1 | 182812410 | 182812425 | DHX9 | 0.001948052 |
| 1 | 211545442 | 211545459 | TRAF5 | 0.001998002 |
| 9 | 75420448 | 75420463 | TMC1 | 0.002020202 |
| 3 | 33626219 | 33626236 | CLASP2 | 0.002020202 |
| 1 | 114963973 | 114963986 | TRIM33 | 0.002020202 |
| 7 | 95775848 | 95775863 | SLC25A13 | 0.002096436 |
| 2 | 27463091 | 27463105 | CAD | 0.002096436 |
| 10 | 95549836 | 95549852 | LGI1 | 0.002105263 |
| 2 | 196762491 | 196762509 | DNAH7 | 0.002162162 |
| 9 | 131715001 | 131715017 | NUP188 | 0.002177068 |
| 17 | 57278908 | 57278920 | PRR11 | 0.002221399 |
| 7 | 130139660 | 130139674 | MEST | 0.002262443 |
| 3 | 100569452 | 100569464 | ABI3BP | 0.002298851 |
| 2 | 11913727 | 11913739 | LPIN1 | 0.002298851 |
| 11 | 88068256 | 88068270 | CTSC | 0.002352941 |
| 10 | 112211000 | 112211025 | SMNDC1(dist = 146293), DUSP5(dist = 46600) | 0.00244898 |
| 8 | 101933023 | 101933037 | YWHAZ | 0.002463054 |
| 5 | 59901692 | 59901704 | DEPDC1B | 0.002463054 |
| 4 | 88986745 | 88986761 | PKD2 | 0.002463054 |
| 3 | 179298584 | 179298597 | ACTL6A | 0.002484472 |
| 8 | 96069973 | 96069988 | NDUFAF6 | 0.00255102 |
| 17 | 40850552 | 40850587 | CNTNAP1 | 0.00255102 |
| 9 | 80343587 | 80343612 | GNAQ | 0.002557545 |
| 1 | 217784431 | 217784441 | GPATCH2 | 0.002645503 |
| X | 70594925 | 70594942 | BCYRN1 | 0.002645503 |
| 1 | 86435957 | 86435971 | COL24A1 | 0.002659574 |
| 11 | 71548312 | 71548339 | DEFB108B | 0.002775208 |
| 15 | 101550861 | 101550877 | LRRK1 | 0.002797203 |
| 2 | 236817336 | 236817351 | AGAP1 | 0.002884615 |
| 1 | 95615737 | 95615761 | TMEM56, TMEM56-RWDD3 | 0.003030303 |
| 2 | 120366057 | 120366070 | CFAP221 | 0.003030303 |
| 9 | 19334957 | 19334970 | DENND4C | 0.003278689 |
| 6 | 80910611 | 80910635 | BCKDHB | 0.003322259 |
| 6 | 32166160 | 32166174 | NOTCH4 | 0.003389831 |
| 9 | 133498229 | 133498245 | FUBP3 | 0.003417635 |
| 1 | 173797451 | 173797462 | DARS2 | 0.003484321 |
| 15 | 80460576 | 80460591 | FAH | 0.003506721 |
| 3 | 196215451 | 196215463 | RNF168 | 0.003658537 |
| 6 | 20758808 | 20758822 | CDKAL1 | 0.003759398 |
| 4 | 119674051 | 119674062 | SEC24D | 0.003759398 |
| 1 | 158326447 | 158326459 | CD1E | 0.003759398 |
| 3 | 122471407 | 122471434 | HSPBAP1 | 0.003805175 |
| 9 | 163927 | 163943 | CBWD1 | 0.003846154 |
| 5 | 33561022 | 33561043 | ADAMTS12 | 0.004040404 |
| 17 | 44409890 | 44409927 | LRRC37A | 0.004048583 |
| 4 | 15839700 | 15839712 | CD38 | 0.004140787 |
| 5 | 96116095 | 96116125 | ERAP1 | 0.004192872 |
| 15 | 63571279 | 63571293 | APH1B | 0.004192872 |
| 6 | 24589046 | 24589057 | KIAA0319 | 0.004267425 |
| 5 | 70809269 | 70809285 | BDP1 | 0.004354136 |
| 15 | 51017381 | 51017393 | SPPL2A | 0.004354136 |
| 6 | 152728079 | 152728093 | SYNE1 | 0.004354136 |
| 17 | 65074354 | 65074364 | HELZ(NM_014877: c.*14_*4del0) | 0.004761905 |
| 19 | 22836873 | 22836886 | ZNF492 | 0.004807692 |
| 14 | 64443258 | 64443278 | MIR548AZ | 0.005288207 |
| 7 | 117867960 | 117867983 | ANKRD7 | 0.005288207 |
| 3 | 185155430 | 185155442 | MAP3K13 | 0.005347594 |
| 11 | 125763610 | 125763624 | PUS3(NM_001271985: c.*70_*56del0, | 0.005347594 |
| | | | NM_031307: c.*70_*56del0) | |
| 22 | 16404838 | 16404857 | POTEH(dist = 116901), OR11H1(dist = 43967) | 0.005745554 |
| 6 | 152740681 | 152740695 | SYNE1 | 0.005844535 |
| 8 | 17100456 | 17100471 | CNOT7 | 0.006049607 |
| 4 | 38930985 | 38930995 | FAM114A1 | 0.006060606 |
| 19 | 22585521 | 22585535 | ZNF98 | 0.006265664 |
| 12 | 53861636 | 53861650 | PCBP2 | 0.006342495 |
| 22 | 44083327 | 44083345 | EFCAB6 | 0.006393862 |
| X | 79937622 | 79937634 | BRWD3 | 0.006451613 |
| 2 | 43778857 | 43778869 | THADA | 0.006493506 |
| 5 | 14481270 | 14481282 | TRIO | 0.006535948 |
| X | 20204548 | 20204563 | RPS6KA3 | 0.006535948 |
| 15 | 40005793 | 40005806 | FSIP1 | 0.006584723 |
| 1 | 47325299 | 47325313 | CYP4Z2P | 0.006993007 |
| 6 | 70040500 | 70040513 | BAI3 | 0.007211538 |
| X | 1464126 | 1464139 | IL3RA | 0.007389163 |
| 12 | 16713487 | 16713501 | LMO3 | 0.008097166 |
| 6 | 133138067 | 133138082 | RPS12 | 0.008534851 |
| 16 | 57249981 | 57250000 | RSPRY1 | 0.008547009 |
| 14 | 96916232 | 96916246 | AK7 | 0.009398496 |
| 17 | 18708830 | 18708843 | TVP23B | 0.009661836 |
| 7 | 23761159 | 23761171 | STK31 | 0.01 |
| 2 | 7154826 | 7154836 | RNF144A | 0.01 |
| 2 | 230705516 | 230705530 | TRIP12 | 0.01010101 |
| 13 | 33637913 | 33637928 | KL | 0.010989011 |
| 11 | 125488247 | 125488261 | STT3A | 0.010989011 |
| 8 | 131859768 | 131859803 | ADCY8 | 0.011363636 |
| 16 | 90141288 | 90141302 | PRDM7 | 0.011614402 |
| 13 | 115002097 | 115002111 | CDC16 | 0.011904762 |
| 7 | 36465606 | 36465622 | ANLN | 0.012048193 |
| 6 | 15593319 | 15593334 | DTNBP1 | 0.012048193 |
| X | 106092450 | 106092473 | TBC1D8B | 0.012195122 |
| 11 | 116629734 | 116629748 | BUD13 | 0.012195122 |
| 10 | 102035182 | 102035193 | BLOC1S2(NM_001282439: c.*37_*26del0, | 0.012345679 |
| | | | NM_001282438: c.*37_*26del0, | |
| | | | NM_001001342: c.*37_*26del0, | |
| | | | NM_173809: c.*37_*26del0, | |
| | | | NM_001282436: c.*37_*26del0, | |
| | | | NM_001282437: c.*37_*26del0) | |
| 19 | 34951356 | 34951371 | UBA2 | 0.0125 |
| 12 | 108104315 | 108104351 | PWP1 | 0.0125 |
| 4 | 54265883 | 54265894 | FIP1L1 | 0.0125 |
| 16 | 21623935 | 21623964 | METTL9 | 0.012658228 |
| 6 | 126249747 | 126249771 | NCOA7 | 0.012658228 |
| 1 | 112305406 | 112305423 | DDX20 | 0.012658228 |
| 2 | 44934524 | 44934535 | CAMKMT | 0.012820513 |
| 15 | 76191743 | 76191755 | UBE2Q2 | 0.012820513 |
| 7 | 149543201 | 149543232 | ZNF862 | 0.012987013 |
| 17 | 1326754 | 1326769 | CRK(NM_005206: c.*183_*168del0, | 0.012987013 |
| | | | NM_016823: c.*53_*38del0) | |
| 15 | 41663884 | 41663900 | NUSAP1 | 0.012987013 |
| 1 | 64022708 | 64022725 | EFCAB7 | 0.013157895 |
| 20 | 34430474 | 34430486 | PHF20 | 0.013333333 |
| 1 | 114186351 | 114186362 | MAGI3 | 0.013333333 |
| 1 | 39929162 | 39929178 | MACF1 | 0.013333333 |
| 2 | 271204 | 271226 | C20orf96 | 0.013333333 |
| 1 | 210536178 | 210536190 | HHAT | 0.013333333 |
| 3 | 93754080 | 93754099 | ARL13B | 0.013333333 |
| 2 | 111406968 | 111406984 | BUB1 | 0.013513514 |
| 11 | 16117460 | 16117478 | SOX6 | 0.013513514 |
| 1 | 173490547 | 173490571 | SLC9C2 | 0.013513514 |
| 3 | 125179711 | 125179723 | SNX4 | 0.013513514 |
| 20 | 2552802 | 2552817 | TMC2 | 0.013513514 |
| 19 | 35758789 | 35758801 | LSR(NM_015925: c.*27_*39del0, | 0.01369863 |
| | | | NM_205835: c.*27_*39del0, | |
| | | | NM_205834: c.*27_*39del0, | |
| | | | NM_001260490: c.*27_*39del0, | |
| | | | NM_001260489: c.*27_*39del0) | |
| 12 | 97339669 | 97339680 | NEDD1 | 0.01369863 |
| 7 | 131130509 | 131130538 | MKLN1 | 0.01369863 |
| 1 | 100684334 | 100684355 | DBT | 0.01369863 |
| 1 | 20441896 | 20441907 | PLA2G2D | 0.01369863 |
| 14 | 68582259 | 68582279 | RAD51B | 0.013888889 |
| 6 | 117725383 | 117725399 | ROS1 | 0.013888889 |
| 3 | 142402827 | 142402838 | PLS1 | 0.013888889 |
| 8 | 22053163 | 22053178 | BMP1 | 0.013888889 |
| 7 | 155099925 | 155099936 | INSIG1 | 0.013888889 |
| 6 | 18258299 | 18258311 | DEK | 0.014084507 |
| 1 | 155701055 | 155701071 | DAP3 | 0.014084507 |
| 15 | 81427705 | 81427719 | C15orf26 | 0.014084507 |
| 20 | 13140617 | 13140637 | SPTLC3 | 0.014084507 |
| 3 | 160131236 | 160131250 | SMC4 | 0.014084507 |
| 2 | 204156968 | 204156980 | CYP20A1 | 0.014084507 |
| 10 | 104449572 | 104449619 | ARL3 | 0.014084507 |
| 4 | 151683003 | 151683021 | LRBA | 0.014084507 |
| 6 | 146256315 | 146256344 | SHPRH | 0.014285714 |
| 22 | 46679850 | 46679864 | TTC38 | 0.014285714 |
| 5 | 37051861 | 37051873 | NIPBL | 0.014285714 |
| 2 | 192228419 | 192228444 | MYO1B | 0.014285714 |
| 5 | 14607339 | 14607351 | FAM105A | 0.014285714 |
| 4 | 25777985 | 25777996 | SEL1L3 | 0.014285714 |
| 9 | 99408079 | 99408094 | AAED1 | 0.014492754 |
| 1 | 149900985 | 149901002 | MTMR11(NM_001145862: c.*36_*19del0) | 0.014492754 |
| 3 | 52696310 | 52696322 | PBRM1 | 0.014492754 |
| 10 | 90362299 | 90362324 | LIPJ | 0.014492754 |
| 7 | 5239305 | 5239336 | WIPI2 | 0.014492754 |
| 2 | 236877066 | 236877084 | AGAP1 | 0.014492754 |
| 6 | 108214754 | 108214783 | SEC63 | 0.014492754 |
| 10 | 74899343 | 74899356 | ECD | 0.014705882 |
| 8 | 53543090 | 53543106 | RB1CC1 | 0.014705882 |
| 17 | 30320860 | 30320882 | SUZ12 | 0.014705882 |
| 14 | 74426284 | 74426298 | COQ6 | 0.014705882 |
| 1 | 220284112 | 220284124 | RNU5F-1 | 0.014705882 |
| 12 | 56380950 | 56380964 | RAB5B | 0.014705882 |
| 5 | 137451460 | 137451472 | NME5 | 0.014705882 |
| 17 | 33288433 | 33288445 | CCT6B(NM_001193530: c.-21_-33del0, | 0.014705882 |
| | | | NM_001193529: c.-21_-33del0, | |
| | | | NM_006584: c.-21_-33del0) | |
| 4 | 15826415 | 15826426 | CD38 | 0.014705882 |
| 20 | 29449706 | 29449717 | NONE(dist = NONE), FRG1B(dist = 162162) | 0.014705882 |
| 6 | 160237662 | 160237680 | PNLDCI | 0.014705882 |
| 12 | 101598350 | 101598362 | SLC5A8 | 0.014925373 |
| 3 | 186522293 | 186522308 | RFC4 | 0.014925373 |
| 1 | 237060945 | 237060970 | MTR(NM_000254: c.*1_*26del0, | 0.014925373 |
| | | | NM_001291939: c.*1_*26del0, | |
| | | | NM_001291940: c.*1_*26del0) | |
| 4 | 79754716 | 79754728 | BMP2K | 0.014925373 |
| 13 | 98634737 | 98634749 | IPO5 | 0.014925373 |
| 10 | 64022582 | 64022593 | RTKN2 | 0.014925373 |
| 14 | 21702046 | 21702071 | HNRNPC | 0.014925373 |
| 9 | 33886999 | 33887011 | UBE2R2 | 0.014925373 |
| 15 | 59961570 | 59961607 | BNIP2 | 0.014925373 |
| 2 | 158275111 | 158275135 | CYTIP | 0.014925373 |
| 11 | 108141955 | 108141971 | ATM | 0.014925373 |
| 20 | 58587783 | 58587794 | CDH26 | 0.014925373 |
| 16 | 21559162 | 21559174 | SLC7A5P2(dist = 27397), LOC101927814(dist = 41845) | 0.014925373 |
| 13 | 45781539 | 45781550 | GTF2F2 | 0.014925373 |
| 4 | 77276593 | 77276604 | CCDC158 | 0.014925373 |
| 6 | 15651642 | 15651656 | DTNBP1 | 0.015151515 |
| 17 | 62855003 | 62855017 | LRRC37A3 | 0.015151515 |
| 10 | 116203903 | 116203928 | ABLIM1 | 0.015151515 |
| 9 | 135523692 | 135523704 | DDX31 | 0.015151515 |
| 8 | 42295130 | 42295156 | SLC20A2 | 0.015151515 |
| 4 | 145659167 | 145659178 | HHIP(NM_022475: c.*58_*69del0) | 0.015151515 |
| 7 | 120590802 | 120590830 | ING3(NM_019071: c.-134_-121delins-, | 0.015151515 |
| | | | NM_198267: c.-134_-121delins-) | |
| 2 | 191523855 | 191523879 | NAB1 | 0.015151515 |
| 4 | 5630470 | 5630482 | EVC2 | 0.015239478 |
| 19 | 327260 | 327277 | MIER2 | 0.015384615 |
| 15 | 81187286 | 81187297 | CEMIP | 0.015384615 |
| 14 | 45716018 | 45716030 | MIS18BP1 | 0.015384615 |
| 20 | 3565277 | 3565289 | ATRN | 0.015384615 |
| 12 | 94575201 | 94575219 | PLXNC1 | 0.015384615 |
| 12 | 31435608 | 31435620 | FAM60A(NM_001135811: c.*38_*26del0, | 0.015384615 |
| | | | NM_001135812: c.*38_*26del0, | |
| | | | NM_021238: c.*38_*26del0) | |
| 9 | 19299209 | 19299224 | DENND4C | 0.015384615 |
| 6 | 136429995 | 136430013 | PDE7B | 0.015384615 |
| 6 | 17669303 | 17669328 | NUP153 | 0.015384615 |
| 7 | 81695842 | 81695859 | CACNA2D1 | 0.015384615 |
| 13 | 26151184 | 26151196 | ATP8A2 | 0.015384615 |
| 17 | 1265304 | 1265326 | YWHAE | 0.015384615 |
| 19 | 21350520 | 21350533 | ZNF431 | 0.015384615 |
| 17 | 27816599 | 27816619 | TAOK1 | 0.015384615 |
| 12 | 105260159 | 105260194 | SLC41A2 | 0.015384615 |
| 8 | 38880851 | 38880862 | ADAM9 | 0.015384615 |
| 16 | 9186735 | 9186764 | C16orf72 | 0.015384615 |
| 5 | 77745712 | 77745752 | SCAMP1 | 0.015625 |
| 4 | 128621144 | 128621158 | INTU | 0.015625 |
| 12 | 42835328 | 42835365 | PPHLN1(NM_201439: c.*17_*54del0, | 0.015625 |
| | | | NM_201440: c.*17_*54del0, | |
| | | | NM_001143788: c.*17_*54del0, | |
| | | | NM_001143789: c.*17_*54del0, | |
| | | | NM_201515: c.*17_*54del0) | |
| 2 | 160092703 | 160092716 | WDSUB1 | 0.015625 |
| 19 | 34302673 | 34302689 | KCTD15 | 0.015625 |
| 2 | 24521686 | 24521697 | ITSN2 | 0.015625 |
| 21 | 34655334 | 34655346 | IL10RB | 0.015625 |
| 21 | 37706065 | 37706077 | MORC3 | 0.015625 |
| 5 | 153174310 | 153174322 | GRIA1 | 0.015625 |
| X | 134033226 | 134033239 | MOSPD1 | 0.015625 |
| 7 | 75155143 | 75155157 | PMS2P3 | 0.015625 |
| 20 | 7976762 | 7976773 | TMX4 | 0.015625 |
| 2 | 64335290 | 64335301 | PELI1 | 0.015625 |
| 11 | 27412757 | 27412806 | LGR4 | 0.015625 |
| 19 | 14827076 | 14827089 | ZNF333 | 0.015625 |
| 21 | 40871690 | 40871717 | SH3BGR | 0.015625 |
| 14 | 39788501 | 39788513 | CTAGE5 | 0.015625 |
| 9 | 132595849 | 132595861 | C9orf78 | 0.015873016 |
| 3 | 67058329 | 67058357 | KBTBD8 | 0.015873016 |
| 6 | 89638988 | 89639004 | RNGTT | 0.015873016 |
| 2 | 170062201 | 170062216 | LRP2 | 0.015873016 |
| 17 | 39659874 | 39659890 | KRT13 | 0.015873016 |
| 12 | 88926290 | 88926302 | KITLG | 0.015873016 |
| 5 | 150856066 | 150856091 | SLC36A1 | 0.015873016 |
| 1 | 22033369 | 22033411 | USP48 | 0.015873016 |
| 5 | 38958956 | 38958971 | RICTOR | 0.015873016 |
| X | 11793084 | 11793105 | MSL3 | 0.015873016 |
| 15 | 58974349 | 58974361 | ADAM10 | 0.015873016 |
| 15 | 64231569 | 64231591 | DAPK2 | 0.015873016 |
| 8 | 121502680 | 121502691 | MTBP | 0.015873016 |
| 16 | 72050826 | 72050853 | DHODH | 0.015873016 |
| 17 | 75187248 | 75187259 | SEC14L1I | 0.015873016 |
| 9 | 33364821 | 33364833 | NFX1(NM_147133: c.*13_*25del0) | 0.015873016 |
| 1 | 35826959 | 35826972 | ZMYM4 | 0.015873016 |
| X | 30726149 | 30726160 | GK | 0.015873016 |
| 10 | 88753099 | 88753110 | AGAP11 | 0.015873016 |
| 2 | 234178624 | 234178640 | ATG16L1 | 0.016129032 |
| 1 | 173795803 | 173795821 | DARS2 | 0.016129032 |
| 3 | 33725814 | 33725828 | CLASP2 | 0.016129032 |
| 6 | 128330423 | 128330435 | PTPRK | 0.016129032 |
| 8 | 33356825 | 33356839 | MAK16(NM_032509: c.*678_*692del0) | 0.016129032 |
| 2 | 32366959 | 32366971 | SPAST | 0.016129032 |
| 3 | 132363752 | 132363765 | NPHP3-ACAD11 | 0.016129032 |
| 9 | 123886190 | 123886202 | CNTRL | 0.016129032 |
| 14 | 21876732 | 21876743 | CHD8 | 0.016129032 |
| 2 | 39505509 | 39505528 | MAP4K3 | 0.016129032 |
| 4 | 128996166 | 128996178 | LARP1B | 0.016129032 |
| 1 | 64017377 | 64017388 | EFCAB7 | 0.016129032 |
| 22 | 45828137 | 45828148 | RIBC2 | 0.016129032 |
| 2 | 37152389 | 37152408 | STRN | 0.016129032 |
| 14 | 92258905 | 92258916 | TC2N | 0.016393443 |
| 1 | 12398426 | 12398437 | VPS13D | 0.016393443 |
| 11 | 118885668 | 118885701 | CCDC84 | 0.016393443 |
| 2 | 70463334 | 70463348 | TIA1 | 0.016393443 |
| 4 | 8472785 | 8472805 | TRMT44 | 0.016393443 |
| 11 | 67209167 | 67209194 | CORO1B | 0.016393443 |
| 2 | 45645684 | 45645695 | SRBD1 | 0.016393443 |
| 15 | 57545438 | 57545449 | TCF12 | 0.016393443 |
| 1 | 52912109 | 52912134 | ZCCHC11 | 0.016393443 |
| 5 | 60170512 | 60170537 | ERCC8 | 0.016393443 |
| 9 | 32986030 | 32986069 | APTX | 0.016393443 |
| 10 | 121675238 | 121675251 | SEC23IP | 0.016393443 |
| 5 | 130535204 | 130535217 | LYRM7 | 0.016393443 |
| 11 | 117152291 | 117152314 | RNF214 | 0.016666667 |
| 2 | 211521213 | 211521243 | CPS1 | 0.016666667 |
| 17 | 59857599 | 59857611 | BRIP1 | 0.016666667 |
| 19 | 47700438 | 47700453 | SAE1 | 0.016666667 |
| 4 | 159618683 | 159618709 | ETFDH | 0.016666667 |
| 2 | 179325203 | 179325214 | MIR548N | 0.016666667 |
| 8 | 130874614 | 130874639 | FAM49B | 0.016666667 |
| 9 | 123922400 | 123922412 | CNTRL | 0.016666667 |
| 11 | 60182851 | 60182862 | MS4A14 | 0.016666667 |
| 3 | 9102112 | 9102129 | SRGAP3 | 0.016666667 |
| 11 | 17526254 | 17526267 | USH1C | 0.016666667 |
| 8 | 22138812 | 22138831 | PIWIL2 | 0.016666667 |
| 14 | 72941317 | 72941328 | RGS6 | 0.016666667 |
| 20 | 49354351 | 49354368 | PARD6B | 0.016666667 |
| 10 | 48980563 | 48980577 | GLUD1P7 | 0.016666667 |
| 15 | 56736722 | 56736733 | MNS1 | 0.016666667 |
| 12 | 66716618 | 66716630 | HELB | 0.016666667 |
| 2 | 179363909 | 179363925 | MIR548N | 0.016666667 |
| 15 | 59399501 | 59399518 | CCNB2 | 0.016666667 |
| 19 | 1364472 | 1364496 | MUM1 | 0.016666667 |
| 5 | 86659147 | 86659159 | RASA1 | 0.016666667 |
| 4 | 95170783 | 95170794 | SMARCAD1 | 0.016666667 |
| 5 | 71500888 | 71500899 | MAP1B | 0.016666667 |
| 17 | 30538050 | 30538061 | RHOT1 | 0.016666667 |
| 16 | 66884343 | 66884355 | CA7 | 0.016666667 |
| 8 | 68140383 | 68140394 | ARFGEF1 | 0.016949153 |
| 3 | 121515940 | 121515953 | IQCB1 | 0.016949153 |
| 10 | 34573008 | 34573020 | PARD3 | 0.016949153 |
| 15 | 86278759 | 86278785 | AKAP13 | 0.016949153 |
| 16 | 71718538 | 71718563 | PHLPP2 | 0.016949153 |
| 15 | 101019693 | 101019705 | CERS3 | 0.016949153 |
| 3 | 160138723 | 160138736 | SMC4 | 0.016949153 |
| 2 | 61622374 | 61622387 | USP34 | 0.016949153 |
| 3 | 4669375 | 4669386 | ITPR1 | 0.016949153 |
| 4 | 89352469 | 89352487 | HERC6 | 0.016949153 |
| 17 | 73515055 | 73515068 | TSEN54 | 0.016949153 |
| 21 | 34117067 | 34117080 | PAXBP1 | 0.016949153 |
| 3 | 108175734 | 108175745 | MYH15 | 0.016949153 |
| 7 | 76883792 | 76883819 | CCDC146 | 0.016949153 |
| 5 | 118500981 | 118501000 | DMXL1 | 0.016949153 |
| 20 | 33338325 | 33338339 | NCOA6 | 0.016949153 |
| 1 | 46739974 | 46739989 | RAD54L | 0.016949153 |
| 3 | 16636093 | 16636108 | DAZL | 0.016949153 |
| 6 | 123545159 | 123545186 | TRDN | 0.016949153 |
| 2 | 165971883 | 165971895 | SCN3A | 0.016949153 |
| 7 | 23347410 | 23347421 | MALSU1 | 0.016949153 |
| 15 | 94841380 | 94841419 | MCTP2 | 0.016949153 |
| 7 | 56052489 | 56052500 | GBAS | 0.016949153 |
| 16 | 15141966 | 15141977 | NTAN1, PDXDC1 | 0.016949153 |

TABLE 3
Microsatellite Repeat Marker Sequences for Glioblastoma Multiforme (GBM).
TABLE 3 - GBM

| ch | start_pos | end_pos | | |
|---|---|---|---|---|
| (hg37) | (hg37) | (hg37) | gene | GBM p-value |
| 20 | 37146131 | 37146146 | RALGAPB | 6.35006E−10 |
| 17 | 18785823 | 18785842 | PRPSAP2 | 9.26872E−10 |
| 19 | 327260 | 327277 | MIER2 | 1.05996E−09 |
| 14 | 95999676 | 95999689 | SNHG10 | 2.51832E−09 |
| 12 | 48360947 | 48360961 | TMEM106C | 4.44836E−09 |
| 11 | 117152291 | 117152314 | RNF214 | 6.01553E−09 |
| 12 | 110720351 | 110720363 | ATP2A2 | 1.34407E−08 |
| 10 | 74899343 | 74899356 | ECD | 1.83372E−08 |
| 17 | 74729588 | 74729600 | METTL23 | 2.66069E−08 |
| 14 | 51348281 | 51348299 | ABHD12B | 3.46368E−08 |
| 15 | 44057632 | 44057645 | PDIA3 | 3.70668E−08 |
| 7 | 86522420 | 86522432 | KIAA1324L | 3.92669E−08 |
| 2 | 47635523 | 47635537 | MSH2 | 4.73369E−08 |
| 12 | 104461711 | 104461723 | HCFC2 | 5.95548E−08 |
| 10 | 76349020 | 76349033 | ADK | 1.01211E−07 |
| 16 | 58585178 | 58585191 | CNOTI | 1.03024E−07 |
| 1 | 215931901 | 215931935 | USH2A | 1.2498E−07 |
| 8 | 141293914 | 141293943 | TRAPPC9 | 1.39278E−07 |
| 2 | 61308569 | 61308585 | KIAA1841 | 2.13551E−07 |
| 15 | 83808031 | 83808046 | HDGFRP3 | 2.6195E−07 |
| 13 | 31722620 | 31722638 | HSPH1 | 2.81205E−07 |
| 4 | 113568126 | 113568140 | LARP7 | 3.49196E−07 |
| 1 | 86435957 | 86435971 | COL24A1 | 3.61688E−07 |
| 1 | 78432506 | 78432521 | FUBP1 | 3.70987E−07 |
| 17 | 39871551 | 39871566 | GAST | 4.77824E−07 |
| 2 | 27708007 | 27708031 | IFT172 | 5.77336E−07 |
| 9 | 98230924 | 98230947 | LOC100507346 | 7.01064E−07 |
| 6 | 136710669 | 136710685 | MAP7 | 7.40825E−07 |
| 8 | 90933269 | 90933296 | OSGIN2 | 7.52016E−07 |
| 7 | 121699789 | 121699802 | PTPRZ1 | 8.68727E−07 |
| 4 | 128812230 | 128812245 | PLK4 | 9.60345E−07 |
| 1 | 220355521 | 220355533 | RAB3GAP2 | 1.15674E−06 |
| 7 | 135099044 | 135099059 | CNOT4 | 1.2157E−06 |
| 16 | 15133783 | 15133795 | NTAN1, PDXDC1 | 1.30846E−06 |
| 12 | 8693215 | 8693257 | CLEC4E | 1.32055E−06 |
| 7 | 91714092 | 91714106 | AKAP9 | 1.33108E−06 |
| 4 | 57897508 | 57897530 | IGFBP7 | 1.40045E−06 |
| 5 | 77745712 | 77745752 | SCAMP1 | 1.50091E−06 |
| X | 15607586 | 15607598 | ACE2 | 1.55681E−06 |
| 2 | 211179765 | 211179777 | MYL1 | 1.59051E−06 |
| 2 | 62063093 | 62063111 | FAM161A | 1.59119E−06 |
| 7 | 106888946 | 106888972 | COG5 | 2.32965E−06 |
| 1 | 235938388 | 235938400 | LYST | 2.56714E−06 |
| 6 | 42611936 | 42611951 | UBR2 | 2.59603E−06 |
| 4 | 52928352 | 52928371 | SPATA18 | 2.62044E−06 |
| 1 | 201754396 | 201754428 | IPO9-AS1, RNU6-79P | 2.63645E−06 |
| 7 | 95775848 | 95775863 | SLC25A13 | 2.79898E−06 |
| 6 | 56504477 | 56504489 | RNU6-71P | 2.83185E−06 |
| 4 | 128621144 | 128621158 | INTU | 3.40001E−06 |
| 12 | 102512024 | 102512037 | NUP37 | 3.9454E−06 |
| 12 | 989800 | 989815 | WNK1 | 4.05912E−06 |
| 12 | 3018675 | 3018690 | TULP3 | 4.43747E−06 |
| 1 | 223156384 | 223156419 | DISP1 | 4.63349E−06 |
| 3 | 5024415 | 5024435 | BHLHE40 | 5.17572E−06 |
| 12 | 56213978 | 56213991 | ORMDL2 | 5.49765E−06 |
| 7 | 66463195 | 66463206 | TYW1 | 5.49765E−06 |
| 4 | 41015553 | 41015566 | APBB2 | 5.71317E−06 |
| 20 | 31954819 | 31954834 | CDK5RAP1 | 5.895E−06 |
| 10 | 105752705 | 105752727 | SLK | 7.91627E−06 |
| 9 | 19334957 | 19334970 | DENND4C | 7.94569E−06 |
| 19 | 46299146 | 46299180 | RSPH6A | 8.58205E−06 |
| 2 | 64112697 | 64112719 | UGP2 | 9.31961E−06 |
| 21 | 42823193 | 42823206 | MX1 | 9.91146E−06 |
| 12 | 57882764 | 57882777 | MARS | 1.16881E−05 |
| 8 | 107704921 | 107704955 | OXR1 | 1.17173E−05 |
| 9 | 125655308 | 125655324 | RC3H2 | 1.22888E−05 |
| 4 | 39233719 | 39233734 | MIR1273H | 1.3431E−05 |
| 5 | 67576330 | 67576346 | PIK3R1 | 1.46604E−05 |
| 3 | 197702875 | 197702887 | LMLN | 1.48703E−05 |
| 4 | 108935768 | 108935822 | HADH | 1.49272E−05 |
| 21 | 37787572 | 37787585 | CHAF1B | 1.52013E−05 |
| 3 | 44399683 | 44399706 | TCAIM | 1.57816E−05 |
| 16 | 84121078 | 84121113 | MBTPS1 | 1.63044E−05 |
| 1 | 172577864 | 172577879 | SUCO | 1.67014E−05 |
| 9 | 112225552 | 112225564 | PTPN3 | 1.73801E−05 |
| 5 | 10405809 | 10405822 | MARCH6 | 1.77786E−05 |
| 11 | 700526 | 700541 | TMEM80 | 1.78668E−05 |
| 15 | 73418741 | 73418756 | NEO1 | 1.9685E−05 |
| 22 | 35719378 | 35719394 | TOM1 | 2.0203E−05 |
| 10 | 98325237 | 98325262 | TM9SF3 | 2.32964E−05 |
| 9 | 100997485 | 100997498 | TBC1D2 | 2.56891E−05 |
| 12 | 104995800 | 104995813 | CHST11 | 2.68605E−05 |
| 3 | 180320897 | 180320910 | TTC14 | 2.73657E−05 |
| 19 | 7995974 | 7996007 | TIMM44 | 2.8612E−05 |
| 6 | 88853530 | 88853542 | CNR1(NM_001160259: c.*45_*33del0, | 2.87436E−05 |
| | | | NM_001160226: c.*45_*33del0, | |
| | | | NM_001160258: c.*45_*33del0, | |
| | | | NM_033181: c.*45_*33del0, | |
| | | | NM_016083: c.*45_*33del0) | |
| 1 | 16248728 | 16248740 | SPEN | 3.07607E−05 |
| 16 | 56535193 | 56535208 | BBS2 | 3.1448E−05 |
| 6 | 10926514 | 10926528 | SYCP2L | 3.29636E−05 |
| 8 | 68140383 | 68140394 | ARFGEF1 | 3.40205E−05 |
| 17 | 16056594 | 16056626 | NCOR1 | 4.15863E−05 |
| 16 | 50368677 | 50368692 | BRD7 | 4.18375E−05 |
| 10 | 114154662 | 114154673 | ACSL5 | 4.23827E−05 |
| 4 | 42592935 | 42592965 | ATP8A1 | 4.35132E−05 |
| 17 | 53014442 | 53014457 | TOM1L1 | 4.39359E−05 |
| 8 | 17882845 | 17882857 | PCM1 | 4.46847E−05 |
| 7 | 115891736 | 115891750 | TES | 4.48083E−05 |
| 12 | 72067966 | 72067980 | THAP2 | 4.51671E−05 |
| 9 | 98009613 | 98009626 | FANCC | 4.87757E−05 |
| 20 | 36431202 | 36431215 | CTNNBL1 | 5.05109E−05 |
| 8 | 67969596 | 67969610 | COPS5 | 5.42673E−05 |
| 14 | 103915242 | 103915254 | MARK3 | 5.45559E−05 |
| 1 | 23636874 | 23636886 | HNRNPR(NM_001102397: c.*73_*61del0, | 5.45607E−05 |
| | | | NM_001102398: c.*73_*61del0, | |
| | | | NM_001102399: c.*73_*61del0, | |
| | | | NM_001297621: c.*73_*61del0, | |
| | | | NM_005826: c.*73_*61del0, | |
| | | | NM_001297622: c.*73_*61del0, | |
| | | | NM_001297620: c.*73_*61del0) | |
| 7 | 2406737 | 2406755 | EIF3B | 5.85864E−05 |
| 16 | 75665791 | 75665803 | KARS | 5.8725E−05 |
| 6 | 31624922 | 31624937 | APOM | 6.44903E−05 |
| 15 | 45968237 | 45968250 | SQRDL | 6.59546E−05 |
| 9 | 102730697 | 102730712 | STX17 | 6.83924E−05 |
| 4 | 94693160 | 94693173 | GRID2 | 6.94493E−05 |
| 3 | 160249220 | 160249234 | KPNA4 | 6.94493E−05 |
| 3 | 69111084 | 69111116 | UBA3 | 7.08642E−05 |
| 11 | 16339918 | 16339934 | SOX6 | 7.26811E−05 |
| 2 | 211521213 | 211521243 | CPS1 | 7.35759E−05 |
| 6 | 100957763 | 100957779 | ASCC3 | 7.74931E−05 |
| 15 | 32393486 | 32393502 | CHRNA7 | 7.87517E−05 |
| 3 | 113376110 | 113376147 | KIAA2018 | 7.96718E−05 |
| 10 | 122630877 | 122630889 | MIR5694 | 7.98148E−05 |
| 11 | 49196555 | 49196567 | FOLH1 | 8.00293E−05 |
| X | 18323348 | 18323361 | SCML2 | 8.25982E−05 |
| 2 | 228159652 | 228159677 | LOC654841 | 8.41236E−05 |
| 11 | 57094842 | 57094869 | SSRP1 | 8.51209E−05 |
| 3 | 19959707 | 19959719 | EFHB | 8.74126E−05 |
| X | 109507669 | 109507681 | AMMECR1 | 8.9845E−05 |
| 17 | 15517060 | 15517073 | CDRT1 | 9.13109E−05 |
| 9 | 131743701 | 131743715 | NUP188 | 9.38174E−05 |
| 8 | 103287850 | 103287864 | UBR5 | 9.56814E−05 |
| 1 | 65138787 | 65138801 | CACHD1 | 9.65764E−05 |
| 2 | 202494630 | 202494642 | TMEM237 | 9.79643E−05 |
| 5 | 77745862 | 77745874 | SCAMP1 | 9.93093E−05 |
| 10 | 102035182 | 102035193 | BLOCIS2(NM_001282439: c.*37_*26del0, | 0.000101906 |
| | | | NM_001282438: c.*37_*26del0, | |
| | | | NM_001001342: c.*37_*26del0, | |
| | | | NM_173809: c.*37_*26del0, | |
| | | | NM_001282436: c.*37_*26del0, | |
| | | | NM_001282437: c.*37_*26del0) | |
| 5 | 74011541 | 74011556 | HEXB | 0.000104264 |
| 9 | 86588185 | 86588199 | HNRNPK | 0.000107448 |
| 12 | 107398862 | 107398876 | CRY1 | 0.000107969 |
| 16 | 74920075 | 74920089 | WDR59 | 0.000112918 |
| 7 | 139097271 | 139097295 | C7orf55-LUC7L2, LUC7L2 | 0.000116128 |
| 6 | 137325901 | 137325917 | IL20RA | 0.000116206 |
| 14 | 39597463 | 39597482 | GEMIN2 | 0.000122455 |
| 4 | 184570723 | 184570735 | RWDD4 | 0.000123392 |
| 9 | 17464479 | 17464491 | CNTLN | 0.000127857 |
| 1 | 53255587 | 53255601 | ZYG11B | 0.000131496 |
| 19 | 34872355 | 34872367 | GPI | 0.000134548 |
| 21 | 38498341 | 38498356 | TTC3 | 0.000137592 |
| 9 | 33912102 | 33912114 | UBE2R2 | 0.000137741 |
| 6 | 137193301 | 137193332 | PEX7 | 0.000138002 |
| 7 | 151884279 | 151884292 | KMT2C | 0.000141755 |
| 4 | 48381682 | 48381704 | SLAIN2 | 0.000147362 |
| 3 | 170715672 | 170715694 | SLC2A2 | 0.00015519 |
| 2 | 203817257 | 203817270 | CARF | 0.000166879 |
| 1 | 167889145 | 167889160 | MPC2 | 0.00017307 |
| 10 | 96108988 | 96109000 | NOC3L | 0.000173085 |
| 10 | 50686600 | 50686614 | ERCC6 | 0.000174192 |
| 3 | 52727189 | 52727200 | GNL3 | 0.000182448 |
| 11 | 76157922 | 76157934 | C11orf30 | 0.000182549 |
| 4 | 83970297 | 83970312 | COPS4 | 0.000186059 |
| 3 | 121515940 | 121515953 | IQCB1 | 0.000189973 |
| 2 | 175246545 | 175246557 | CIR1 | 0.000196227 |
| 4 | 128724672 | 128724734 | HSPA4L | 0.00019802 |
| 1 | 211280724 | 211280748 | KCNH1 | 0.000199584 |
| 13 | 51522081 | 51522094 | RNASEH2B | 0.000200337 |
| 5 | 14481270 | 14481282 | TRIO | 0.000202029 |
| 17 | 5044640 | 5044653 | USP6 | 0.000206143 |
| 6 | 152539326 | 152539345 | SYNE1 | 0.00021198 |
| 8 | 134125650 | 134125662 | TG | 0.000229417 |
| 11 | 55035809 | 55035822 | TRIM48 | 0.000229473 |
| 19 | 11565819 | 11565831 | ELAVL3 | 0.000234907 |
| 11 | 118247264 | 118247277 | UBE4A | 0.000240341 |
| 1 | 206904644 | 206904665 | MAPKAPK2 | 0.000243546 |
| 2 | 112854563 | 112854576 | TMEM87B | 0.000251362 |
| 12 | 62261077 | 62261088 | FAM19A2 | 0.000256725 |
| 15 | 89533168 | 89533181 | MFGE8(dist = 76505), ABHD2(dist = 98200) | 0.00025686 |
| X | 1464126 | 1464139 | IL3RA | 0.000265236 |
| 13 | 37602245 | 37602257 | SUPT20H | 0.0002725 |
| 17 | 8417006 | 8417020 | MYH10 | 0.000274591 |
| X | 129267464 | 129267478 | AIFM1 | 0.000282137 |
| 2 | 228680153 | 228680168 | CCL20 | 0.000299587 |
| 16 | 4559351 | 4559367 | HMOX2 | 0.000308196 |
| 5 | 94842718 | 94842730 | TTC37 | 0.000319434 |
| 7 | 130139660 | 130139674 | MEST | 0.00032029 |
| 1 | 153955601 | 153955614 | RAB13 | 0.000322915 |
| 17 | 39976721 | 39976731 | FKBP10 | 0.000335345 |
| 2 | 99721804 | 99721820 | TSGA10 | 0.000336818 |
| 14 | 56104386 | 56104405 | KTN1 | 0.000341763 |
| 5 | 122940980 | 122941019 | CSNK1G3 | 0.000345412 |
| 10 | 34573008 | 34573020 | PARD3 | 0.000358836 |
| X | 148048207 | 148048223 | AFF2 | 0.00036314 |
| 14 | 92258905 | 92258916 | TC2N | 0.000370233 |
| 15 | 50920509 | 50920523 | TRPM7 | 0.00038346 |
| 1 | 103471456 | 103471487 | COL11A1 | 0.000391752 |
| 11 | 1944050 | 1944071 | TNNT3 | 0.000391937 |
| 1 | 204234196 | 204234213 | PLEKHA6 | 0.000398144 |
| 17 | 8166113 | 8166126 | PFAS | 0.000406449 |
| 5 | 132408905 | 132408918 | HSPA4 | 0.000419566 |
| 6 | 154489255 | 154489267 | IPCEF1, OPRM1 | 0.000435414 |
| 6 | 64419024 | 64419039 | PHF3 | 0.000438237 |
| 12 | 107237602 | 107237626 | RIC8B | 0.00045684 |
| 17 | 2323953 | 2323979 | METTL16 | 0.000460066 |
| 2 | 98872570 | 98872582 | VWA3B | 0.000476677 |
| 1 | 179271948 | 179271961 | SOAT1 | 0.000480031 |
| 3 | 185155430 | 185155442 | MAP3K13 | 0.000482959 |
| 6 | 167416662 | 167416676 | FGFR1OP | 0.000500417 |
| 9 | 86354663 | 86354679 | GKAP1 | 0.000509295 |
| 12 | 105464316 | 105464342 | ALDH1L2 | 0.000509684 |
| 4 | 146076632 | 146076644 | OTUD4 | 0.000513526 |
| 10 | 127789605 | 127789616 | ADAM12 | 0.000529606 |
| 6 | 144758647 | 144758660 | UTRN | 0.000529801 |
| 17 | 17039561 | 17039595 | MPRIP | 0.000531476 |
| 3 | 47129781 | 47129808 | SETD2 | 0.000536913 |
| 2 | 232025952 | 232025967 | PSMD1 | 0.000539084 |
| 7 | 31132235 | 31132249 | ADCYAP1R1 | 0.000558102 |
| X | 135095655 | 135095677 | SLC9A6 | 0.000564972 |
| 8 | 74890927 | 74890949 | TMEM70 | 0.000564972 |
| 18 | 649879 | 649895 | TYMSOS(NM_001012716: c.*120_*104del0) | 0.000566472 |
| 11 | 46665805 | 46665827 | ATG13 | 0.00056684 |
| 12 | 73012649 | 73012663 | TRHDE | 0.000581636 |
| 2 | 152236045 | 152236060 | TNFAIP6 | 0.000582411 |
| 9 | 19328009 | 19328021 | DENND4C | 0.000584454 |
| 4 | 110433282 | 110433303 | SEC24B | 0.000590958 |
| 11 | 14316128 | 14316148 | RRAS2 | 0.000607903 |
| 1 | 46650191 | 46650201 | TSPAN1 | 0.000641849 |
| X | 24084241 | 24084254 | EIF2S3 | 0.000656689 |
| 17 | 47696764 | 47696775 | SPOP | 0.000657895 |
| 15 | 35149313 | 35149327 | AQR | 0.00066335 |
| 4 | 39511530 | 39511543 | MIR1273H | 0.000702272 |
| 15 | 81187286 | 81187297 | CEMIP | 0.000710773 |
| 1 | 235826382 | 235826396 | LYST | 0.000730997 |
| 12 | 90005161 | 90005173 | ATP2B1 | 0.000738712 |
| 11 | 108121410 | 108121426 | ATM | 0.000743496 |
| 5 | 56546749 | 56546777 | GPBP1 | 0.000745956 |
| 6 | 31646746 | 31646758 | LY6G5C | 0.000746911 |
| 10 | 70056619 | 70056632 | PBLD | 0.000770337 |
| 12 | 21958998 | 21959011 | ABCC9 | 0.000806257 |
| 17 | 7811876 | 7811889 | CHD3 | 0.000807723 |
| 3 | 196532148 | 196532160 | PAK2 | 0.000812898 |
| 15 | 55841195 | 55841209 | PYGO1 | 0.00081389 |
| 1 | 213180434 | 213180459 | ANGEL2 | 0.000837872 |
| 19 | 35758789 | 35758801 | LSR(NM_015925: c.*27_*39del0, | 0.000839024 |
| | | | NM_205835: c.*27_*39del0, | |
| | | | NM_205834: c.*27_*39del0, | |
| | | | NM_001260490: c.*27_*39del0, | |
| | | | NM_001260489: c._*27_*39del0) | |
| 6 | 138628598 | 138628611 | KIAA1244 | 0.000840336 |
| 10 | 7755088 | 7755101 | ITIH2 | 0.000869187 |
| 20 | 54823684 | 54823699 | MC3R | 0.00087108 |
| 6 | 90347394 | 90347406 | LYRM2 | 0.000881368 |
| 18 | 57684454 | 57684466 | PMAIP1(dist = 112916), MC4R(dist = 354098) | 0.000883002 |
| X | 11200070 | 11200086 | ARHGAP6 | 0.000911521 |
| X | 129203198 | 129203214 | ELF4 | 0.000912545 |
| 6 | 160157277 | 160157288 | WTAP | 0.000948509 |
| 17 | 60374173 | 60374189 | TBC1D3P2(dist = 21157), EFCAB3(dist = 73390) | 0.001000834 |
| 21 | 43770926 | 43770938 | TFF2 | 0.001010001 |
| 12 | 62785259 | 62785270 | USP15 | 0.001012146 |
| 1 | 89289954 | 89289966 | PKN2 | 0.001038422 |
| 19 | 33579248 | 33579262 | GPATCH1 | 0.001042644 |
| 6 | 111702448 | 111702460 | REV3L | 0.001045033 |
| 6 | 76728583 | 76728598 | IMPG1 | 0.001045033 |
| 1 | 20749284 | 20749296 | THUMPD1 | 0.001076625 |
| 17 | 73519258 | 73519269 | TSEN54 | 0.001085541 |
| 6 | 131999103 | 131999115 | ENPP3 | 0.001101425 |
| 15 | 56946703 | 56946733 | ZNF280D | 0.001120239 |
| 21 | 33312539 | 33312554 | HUNK | 0.001122209 |
| 12 | 69214081 | 69214094 | MDM2 | 0.00112225 |
| 3 | 127820493 | 127820505 | RUVBL1 | 0.001134301 |
| 5 | 176729736 | 176729749 | RAB24 | 0.001160656 |
| 2 | 56611386 | 56611398 | CCDC85A | 0.00116898 |
| 6 | 20548795 | 20548808 | CDKAL1 | 0.001179485 |
| 4 | 152065328 | 152065339 | SH3D19 | 0.001185771 |
| 6 | 128385949 | 128385967 | LOC101928140 | 0.001207243 |
| 21 | 30339205 | 30339217 | LTN1 | 0.001212121 |
| 6 | 102124470 | 102124483 | GRIK2 | 0.001212573 |
| X | 150573322 | 150573335 | VMA21 | 0.001232724 |
| 18 | 43445730 | 43445748 | EPG5 | 0.001240695 |
| 18 | 19110456 | 19110477 | ESCO1 | 0.001244912 |
| 16 | 3433714 | 3433726 | ZSCAN32 | 0.001307076 |
| 5 | 147475501 | 147475520 | SPINK5 | 0.001318571 |
| 15 | 45028919 | 45028931 | TRIM69 | 0.00132632 |
| 1 | 84956161 | 84956173 | RPF1 | 0.001360421 |
| 12 | 69090573 | 69090587 | NUP107 | 0.001376994 |
| 1 | 155671899 | 155671929 | DAP3 | 0.001380474 |
| 1 | 150310549 | 150310566 | PRPF3 | 0.001388981 |
| 4 | 25315637 | 25315653 | ZCCHC4 | 0.001394783 |
| 15 | 50935484 | 50935496 | TRPM7 | 0.001414874 |
| 2 | 131883287 | 131883301 | PLEKHB2 | 0.0014171 |
| 6 | 131948816 | 131948831 | MED23 | 0.001456876 |
| 13 | 76134860 | 76134871 | UCHL3 | 0.001488095 |
| 16 | 30665449 | 30665465 | PRR14 | 0.00151747 |
| 5 | 50122645 | 50122660 | PARP8 | 0.001541624 |
| 8 | 75157416 | 75157442 | JPH1 | 0.001552554 |
| 2 | 98866980 | 98866995 | VWA3B | 0.001610133 |
| 21 | 37706087 | 37706100 | MORC3 | 0.001639344 |
| 6 | 86387036 | 86387048 | SNORD50A | 0.001683313 |
| 17 | 35979746 | 35979761 | DDX52 | 0.001731836 |
| 11 | 78154810 | 78154823 | NARS2 | 0.001734906 |
| 5 | 63803508 | 63803522 | RGS7BP | 0.001738051 |
| 11 | 94230106 | 94230131 | ANKRD49 | 0.00174216 |
| 17 | 59857599 | 59857611 | BRIP1 | 0.001759531 |
| 12 | 97339669 | 97339680 | NEDD1 | 0.001761597 |
| 15 | 64717845 | 64717857 | TRIP4 | 0.001763357 |
| 14 | 58690427 | 58690451 | ACTR10 | 0.001780898 |
| 9 | 15458080 | 15458092 | SNAPC3 | 0.001797187 |
| 1 | 36888302 | 36888316 | OSCP1 | 0.001807693 |
| 2 | 186692859 | 186692872 | FSIP2 | 0.001818182 |
| 1 | 67154730 | 67154748 | SGIP1 | 0.001822564 |
| 12 | 124144572 | 124144584 | GTF2H3 | 0.001831502 |
| 2 | 98907089 | 98907103 | VWA3B | 0.001831502 |
| 1 | 233295992 | 233296007 | PCNXL2 | 0.001845472 |
| 12 | 31820564 | 31820578 | METTL20 | 0.001845472 |
| 2 | 20526998 | 20527011 | PUM2 | 0.001851852 |
| X | 122598635 | 122598648 | GRIA3 | 0.001854305 |
| 4 | 169612452 | 169612484 | PALLD | 0.001854305 |
| 12 | 124242458 | 124242472 | ATP6V0A2 | 0.001858494 |
| 12 | 122966016 | 122966028 | ZCCHC8 | 0.001867959 |
| X | 153357614 | 153357628 | MECP2 | 0.001904589 |
| 10 | 75214137 | 75214149 | PPP3CB | 0.001905732 |
| 12 | 110924566 | 110924578 | FAM216A | 0.001923077 |
| 13 | 23808732 | 23808745 | SGCG | 0.001930502 |
| 15 | 78836661 | 78836676 | PSMA4 | 0.001935484 |
| 13 | 39435552 | 39435564 | FREM2 | 0.001950297 |
| 5 | 88018369 | 88018402 | MEF2C(NM_001193350: c.*52_*19del0, | 0.001980198 |
| | | | NM_001131005: c.*52_*19del0, | |
| | | | NM_002397: c.*52_*19del0, | |
| | | | NM_001193347: c.*52_*19del0, | |
| | | | NM_001193348: c.*52_*19del0, | |
| | | | NM_001193349: c.*52_*19del0) | |
| 12 | 53685372 | 53685383 | ESPL1 | 0.002016129 |
| 14 | 88407716 | 88407730 | GALC | 0.002030991 |
| X | 15373247 | 15373259 | PIR-FIGF | 0.002059661 |
| 1 | 150297334 | 150297351 | PRPF3 | 0.002060535 |
| 11 | 124007701 | 124007714 | VWA5A | 0.002087963 |
| 5 | 179665269 | 179665281 | MAPK9 | 0.002094868 |
| 13 | 100913132 | 100913161 | PCCA | 0.002097693 |
| 5 | 68524006 | 68524019 | MRPS36 | 0.002131035 |
| 1 | 181752769 | 181752784 | CACNA1E | 0.002134098 |
| 8 | 120856021 | 120856034 | DSCC1 | 0.002136448 |
| 17 | 4646533 | 4646547 | ZMYND15 | 0.002158342 |
| 3 | 52676065 | 52676079 | PBRM1 | 0.002162162 |
| 2 | 139318332 | 139318366 | SPOPL | 0.002191132 |
| 14 | 58681903 | 58681915 | ACTR10 | 0.002192982 |
| 14 | 45716018 | 45716030 | MIS18BP1 | 0.002203442 |
| 5 | 6623405 | 6623418 | NSUN2 | 0.002229122 |
| 5 | 76707435 | 76707447 | PDE8B | 0.002235584 |
| 4 | 107133861 | 107133873 | TBCK | 0.002237835 |
| 6 | 76618182 | 76618195 | MY06 | 0.002243985 |
| 4 | 113107829 | 113107845 | C4orf32 | 0.002248876 |
| X | 153714027 | 153714038 | UBL4A | 0.0022829 |
| 12 | 31289662 | 31289674 | DDX11(dist = 31937), FAM60A(dist = 143846) | 0.002337541 |
| 3 | 172046695 | 172046709 | FNDC3B | 0.002337541 |
| 4 | 16362275 | 16362287 | TAPT1-AS1(dist = 102465), LDB2(dist = 140878) | 0.002340183 |
| 11 | 5775903 | 5775915 | OR52N4 | 0.002356638 |
| 11 | 94212930 | 94212942 | MRE11A | 0.002370417 |
| 16 | 53496428 | 53496451 | RBL2 | 0.00238776 |
| X | 118679315 | 118679331 | CXorf56 | 0.002392344 |
| X | 106028279 | 106028291 | RNF128 | 0.002392344 |
| 16 | 31196218 | 31196237 | FUS | 0.002407981 |
| 6 | 17602786 | 17602812 | FAM8A1 | 0.002414487 |
| 6 | 110048489 | 110048501 | FIG4 | 0.002432596 |
| 7 | 23823176 | 23823194 | STK31 | 0.002442002 |
| 12 | 30887772 | 30887832 | CAPRIN2 | 0.002451559 |
| 11 | 65268479 | 65268492 | MALAT1 | 0.002466236 |
| 13 | 114514907 | 114514927 | TMEM255B | 0.002466526 |
| 9 | 99408079 | 99408094 | AAED1 | 0.002504472 |
| 19 | 8438454 | 8438467 | ANGPTL4 | 0.002504472 |
| 3 | 113377481 | 113377493 | KIAA2018 | 0.002505593 |
| 5 | 38954825 | 38954836 | RICTOR | 0.00255102 |
| 16 | 67286352 | 67286366 | SLC9A5 | 0.002574003 |
| 2 | 39240584 | 39240596 | SOS1 | 0.002579296 |
| 10 | 17646048 | 17646060 | PTPLA | 0.002583661 |
| 1 | 237619875 | 237619890 | RYR2 | 0.002612179 |
| 5 | 137507104 | 137507121 | BRD8 | 0.002617217 |
| 22 | 41550984 | 41550996 | EP300 | 0.002621356 |
| 16 | 58200464 | 58200481 | CSNK2A2 | 0.002645036 |
| 1 | 22837586 | 22837604 | ZBTB40 | 0.002675318 |
| 6 | 121412191 | 121412203 | TBC1D32 | 0.002681549 |
| 16 | 4835778 | 4835794 | SEPT12 | 0.002721712 |
| 18 | 47438344 | 47438355 | MYO5B | 0.002747253 |
| 1 | 227218085 | 227218097 | CDC42BPA | 0.002757078 |
| 4 | 166388811 | 166388838 | CPE | 0.002757806 |
| X | 32472757 | 32472777 | DMD | 0.002758873 |
| 5 | 145888687 | 145888714 | TCERG1 | 0.002830189 |
| 10 | 64975001 | 64975012 | JMJD1C | 0.002863632 |
| 1 | 196642969 | 196642979 | CFH | 0.002884615 |
| X | 153216185 | 153216195 | HCFC1 | 0.002898551 |
| 19 | 47700438 | 47700453 | SAE1 | 0.002919404 |
| 4 | 159618683 | 159618709 | ETFDH | 0.002919404 |
| 18 | 32917176 | 32917189 | ZNF24(NM_006965: c.*20_*7del0) | 0.002926233 |
| 17 | 62748076 | 62748093 | LOC146880 | 0.002941372 |
| 2 | 48032740 | 48032754 | MSH6 | 0.002943848 |
| 9 | 131456159 | 131456172 | SET | 0.002962023 |
| 3 | 113955055 | 113955069 | ZNF80(NM_007136: c.*45_*31del0) | 0.002965434 |
| 1 | 245021291 | 245021305 | HNRNPU | 0.00299578 |
| 3 | 3216976 | 3216988 | CRBN | 0.003005582 |
| 2 | 38527397 | 38527412 | ATL2 | 0.003042156 |
| 5 | 130939025 | 130939037 | RAPGEF6 | 0.003055768 |
| 13 | 101727043 | 101727094 | NALCN | 0.00305771 |
| 15 | 50570999 | 50571024 | GABPB1 | 0.003072197 |
| 4 | 89772341 | 89772364 | FAM13A | 0.00308642 |
| 2 | 88484805 | 88484815 | THNSL2 | 0.00310517 |
| 9 | 113530024 | 113530036 | MUSK | 0.003142184 |
| 12 | 9706558 | 9706571 | DDX12P(dist = 105790), KLRB1(dist = 41299) | 0.003164557 |
| 1 | 19683355 | 19683368 | CAPZB | 0.003193033 |
| 9 | 132595849 | 132595861 | C9orf78 | 0.003260363 |
| 6 | 110426583 | 110426595 | WASF1 | 0.003301246 |
| 17 | 36477914 | 36477929 | MRPL45 | 0.003318584 |
| 8 | 24348425 | 24348437 | LOC101929294, LOC101929315 | 0.003339296 |
| 1 | 12398426 | 12398437 | VPS13D | 0.003342246 |
| 18 | 12352952 | 12352964 | AFG3L2 | 0.003378378 |
| 9 | 15433520 | 15433532 | SNAPC3 | 0.003378378 |
| 2 | 120712718 | 120712731 | PTPN4 | 0.003401039 |
| 10 | 96025381 | 96025392 | PLCE1 | 0.003418803 |
| 3 | 138193025 | 138193038 | ESYT3 | 0.003478027 |
| X | 108636043 | 108636070 | GUCY2F | 0.003496503 |
| 7 | 73666736 | 73666750 | RFC2 | 0.003496503 |
| 6 | 55739711 | 55739726 | BMP5(NM_021073: c.-48_-63del0) | 0.003499563 |
| 2 | 66796098 | 66796109 | MEIS1 | 0.003506311 |
| 5 | 180482303 | 180482317 | BTNL9 | 0.003516352 |
| 2 | 102979238 | 102979250 | IL18R1 | 0.003551745 |
| 6 | 110113763 | 110113776 | FIG4 | 0.003596044 |
| 4 | 123109034 | 123109048 | KIAA1109 | 0.003611449 |
| 12 | 88566358 | 88566370 | TMTC3 | 0.003634476 |
| 4 | 119950975 | 119950989 | SYNPO2 | 0.003653617 |
| 1 | 10468005 | 10468021 | PGD | 0.00367165 |
| 7 | 120739962 | 120739978 | CPED1 | 0.003703051 |
| 11 | 63149670 | 63149682 | SLC22A9 | 0.003753519 |
| 16 | 71808499 | 71808519 | AP1G1 | 0.003773585 |
| 13 | 99461532 | 99461565 | DOCK9 | 0.003805175 |
| 16 | 70693872 | 70693884 | IL34 | 0.003834364 |
| 21 | 47662899 | 47662934 | MCM3AP-AS1 | 0.003843748 |
| 8 | 7217010 | 7217023 | ZNF705G | 0.00385058 |
| 4 | 122590883 | 122590899 | ANXA5 | 0.003851516 |
| 4 | 79460647 | 79460664 | FRAS1 | 0.003856749 |
| 3 | 58792080 | 58792092 | C3orf67 | 0.003864319 |
| 1 | 154227292 | 154227307 | UBAP2L | 0.003900525 |
| 14 | 31762500 | 31762511 | HEATR5A | 0.003912363 |
| 8 | 30569609 | 30569622 | GSR | 0.003923495 |
| 5 | 10397473 | 10397485 | MARCH6 | 0.004048583 |
| 15 | 86278759 | 86278785 | AKAP13 | 0.004058369 |
| 2 | 153004816 | 153004839 | STAM2 | 0.004126142 |
| 2 | 79314062 | 79314073 | REG1B | 0.004158416 |
| 2 | 103281540 | 103281552 | SLC9A2 | 0.004158416 |
| 5 | 139936680 | 139936694 | SRA1 | 0.004162596 |
| 6 | 158322872 | 158322901 | SNX9 | 0.004172571 |
| 12 | 72019989 | 72020018 | ZFC3H1 | 0.004242424 |
| 15 | 65412325 | 65412337 | PDCD7 | 0.004418843 |
| 8 | 17100456 | 17100471 | CNOT7 | 0.004418843 |
| 3 | 179083024 | 179083036 | MFN1 | 0.004450908 |
| 8 | 54742095 | 54742107 | ATP6V1H | 0.004508408 |
| 3 | 19498221 | 19498253 | KCNH8 | 0.00452284 |
| X | 119675354 | 119675368 | CUL4B | 0.004559271 |
| 14 | 91633483 | 91633498 | C14orf159 | 0.004586405 |
| 9 | 86584222 | 86584234 | HNRNPK(NM_002140: c.*100_*88del0, | 0.004605263 |
| | | | NM_031263: c.*100_*88del0, | |
| | | | NM_031262: c.*43_*31del0) | |
| 19 | 16101139 | 16101166 | OR10H4(dist = 40371), LINC00661(dist = 25278) | 0.004617828 |
| X | 19024426 | 19024440 | GPR64 | 0.004662005 |
| X | 48214185 | 48214200 | SSX3 | 0.004667052 |
| 1 | 86246998 | 86247012 | COL24A1 | 0.004734984 |
| 2 | 37487341 | 37487353 | PRKD3 | 0.0049217 |
| 9 | 8525217 | 8525230 | PTPRD | 0.004980316 |
| 1 | 224318148 | 224318164 | FBXO28 | 0.005078803 |
| 17 | 4712563 | 4712580 | PLD2 | 0.005126452 |
| 14 | 50605267 | 50605280 | SOS2 | 0.005202381 |
| 10 | 99118165 | 99118176 | RRP12 | 0.005202381 |
| 1 | 113657018 | 113657033 | LRIG2 | 0.005254919 |
| 7 | 129028857 | 129028882 | AHCYL2 | 0.005254919 |
| 15 | 43307839 | 43307852 | UBR1 | 0.005362615 |
| 11 | 89395303 | 89395315 | FOLH1B | 0.005580911 |
| 1 | 233225957 | 233225969 | PCNXL2 | 0.005656566 |
| 11 | 107518175 | 107518187 | ELMOD1 | 0.005689001 |
| 3 | 67058329 | 67058357 | KBTBD8 | 0.005714286 |
| 1 | 215914883 | 215914896 | USH2A | 0.005745554 |
| 19 | 14153179 | 14153202 | IL27RA | 0.005806452 |
| 12 | 121004564 | 121004583 | RNF10 | 0.00586178 |
| 1 | 150201305 | 150201316 | ANP32E | 0.005888848 |
| 14 | 73465027 | 73465043 | ZFYVE1 | 0.005905042 |
| 5 | 70754289 | 70754302 | BDP1 | 0.00593692 |
| 17 | 10543281 | 10543296 | MYH3 | 0.005961252 |
| 4 | 54319247 | 54319262 | FIP1L1 | 0.00599448 |
| 2 | 73635899 | 73635911 | ALMS1 | 0.006036217 |
| 2 | 179478746 | 179478757 | MIR548N, TTN-AS1 | 0.006171026 |
| 1 | 236902532 | 236902560 | ACTN2 | 0.006240292 |
| 8 | 99054836 | 99054849 | RPL30 | 0.00625077 |
| 14 | 53217376 | 53217395 | STYX | 0.006263261 |
| 12 | 55354876 | 55354889 | TESPA1 | 0.006270479 |
| 19 | 11447769 | 11447783 | RAB3D | 0.006302521 |
| 13 | 41947712 | 41947725 | NAA16 | 0.006341945 |
| 14 | 73079138 | 73079150 | RGS6(dist = 45900), DPF3(dist = 6854) | 0.006348087 |
| 8 | 104930646 | 104930658 | RIMS2 | 0.00643897 |
| 1 | 9770690 | 9770741 | PIK3CD | 0.00647753 |
| 11 | 55656437 | 55656451 | TRIM51 | 0.006588811 |
| 7 | 74172385 | 74172400 | GTF2I | 0.006593407 |
| 1 | 114224782 | 114224799 | MAGI3 | 0.006672895 |
| X | 100793432 | 100793449 | ARMCX4(dist = 42634), ARMCX1(dist = 12065) | 0.006746627 |
| 15 | 58891938 | 58891962 | ADAM10 | 0.006754862 |
| 12 | 99117397 | 99117414 | APAF1 | 0.006820148 |
| 6 | 163235158 | 163235170 | PACRG | 0.00689138 |
| 4 | 48529683 | 48529699 | FRYL | 0.006896613 |
| 16 | 67470052 | 67470095 | HSD11B2 | 0.006962785 |
| 5 | 68396764 | 68396793 | SLC30A5 | 0.006993007 |
| 4 | 68416393 | 68416409 | CENPC(dist = 5137), STAP1(dist = 8037) | 0.007063882 |
| 2 | 207027356 | 207027368 | EEF1 B2 | 0.007225164 |
| 4 | 123374811 | 123374832 | IL2 | 0.007482453 |
| 17 | 31324395 | 31324414 | SPACA3 | 0.007484766 |
| 4 | 120423686 | 120423697 | PDE5A | 0.007541478 |
| 1 | 15890342 | 15890359 | DNAJC16 | 0.007545932 |
| 12 | 68690765 | 68690777 | MDM1 | 0.007545932 |
| 2 | 271998 | 272015 | ACP1 | 0.007548299 |
| 12 | 101598350 | 101598362 | SLC5A8 | 0.007843137 |
| 6 | 42559866 | 42559879 | UBR2 | 0.007928691 |
| X | 53113895 | 53113905 | TSPYL2 | 0.0080453 |
| 15 | 42536227 | 42536238 | TMEM87A | 0.0080453 |
| 17 | 58289521 | 58289533 | USP32 | 0.008064516 |
| 1 | 92846489 | 92846501 | RPAP2 | 0.008130081 |
| 6 | 7410735 | 7410747 | RIOK1 | 0.008130081 |

TABLE 4
Microsatellite Repeat Marker Sequences for is Lung Adenocarcinoma (LUAD).
TABLE 4 - LUAD

| ch | start_pos | end_pos | | LUAD |
|---|---|---|---|---|
| (hg37) | (hg37) | (hg37) | gene | p-value |
| 15 | 101929632 | 101929652 | PCSK6 | 2.5301E−08 |
| 12 | 107237602 | 107237626 | RIC8B | 3.53475E−06 |
| 20 | 37146131 | 37146146 | RALGAPB | 3.66148E−06 |
| 11 | 73984657 | 73984669 | P4HA3 | 4.18157E−06 |
| 7 | 106888946 | 106888972 | COG5 | 5.12958E−06 |
| 3 | 48636506 | 48636516 | UQCRC1(NM_003365: c.*55_*45del0) | 5.64524E−06 |
| 1 | 222904869 | 222904881 | BROX | 6.74941E−06 |
| 9 | 131456159 | 131456172 | SET | 7.61383E−06 |
| 12 | 63964640 | 63964653 | DPY19L2 | 8.90139E−06 |
| 5 | 145647187 | 145647210 | RBM27 | 9.24667E−06 |
| 5 | 68524006 | 68524019 | MRPS36 | 1.05359E−05 |
| 14 | 23495610 | 23495622 | PSMB5 | 1.11579E−05 |
| 12 | 49397291 | 49397302 | PRKAG1 | 1.11957E−05 |
| 10 | 116919785 | 116919796 | ATRNL1 | 1.32179E−05 |
| 7 | 121699789 | 121699802 | PTPRZ1 | 2.46924E−05 |
| 1 | 218610662 | 218610680 | TGFB2 | 2.55728E−05 |
| 10 | 26825151 | 26825168 | APBB1IP | 3.78529E−05 |
| X | 53113895 | 53113905 | TSPYL2 | 3.84423E−05 |
| 2 | 231940295 | 231940307 | PSMD1 | 4.64511E−05 |
| 8 | 104922508 | 104922534 | RIMS2 | 4.86669E−05 |
| 2 | 99721804 | 99721820 | TSGA10 | 5.43603E−05 |
| 6 | 24589046 | 24589057 | KIAA0319 | 5.56948E−05 |
| 5 | 140812775 | 140812788 | PCDHGA12 | 6.20732E−05 |
| 2 | 170062201 | 170062216 | LRP2 | 6.30112E−05 |
| 1 | 82187543 | 82187554 | LPHN2 | 7.21449E−05 |
| 4 | 71693494 | 71693508 | GRSF1 | 7.25128E−05 |
| X | 129267464 | 129267478 | AIFM1 | 7.67575E−05 |
| 12 | 73012649 | 73012663 | TRHDE | 9.1847E−05 |
| 6 | 55739711 | 55739726 | BMP5(NM_021073: c.-48_-63del0) | 0.000100547 |
| 14 | 96800846 | 96800858 | ATG2B | 0.000106372 |
| 11 | 14501267 | 14501282 | COPB1 | 0.000108932 |
| 16 | 74372596 | 74372618 | LOC283922 | 0.000112931 |
| 3 | 158526942 | 158526966 | MFSD1 | 0.000116955 |
| 2 | 102979238 | 102979250 | IL18R1 | 0.000120115 |
| 2 | 10269544 | 10269555 | RRM2(NM_001165931: c.*31_*42del0, | 0.000135483 |
| | | | NM_001034: c.*31_*42del0) | |
| 11 | 46703002 | 46703014 | ARHGAP1 | 0.000140706 |
| 12 | 28459652 | 28459664 | CCDC91 | 0.000143472 |
| 12 | 94575201 | 94575219 | PLXNC1 | 0.000143472 |
| 1 | 211280724 | 211280748 | KCNH1 | 0.000146282 |
| 22 | 31985385 | 31985398 | SFI1 | 0.000149254 |
| 9 | 8525217 | 8525230 | PTPRD | 0.000151061 |
| 14 | 73079138 | 73079150 | RGS6(dist = 45900), DPF3(dist = 6854) | 0.000152277 |
| 1 | 92846489 | 92846501 | RPAP2 | 0.000163613 |
| 15 | 20666397 | 20666411 | HERC2P3 | 0.000165548 |
| 5 | 70837260 | 70837275 | BDP1 | 0.000167701 |
| 1 | 11134470 | 11134485 | EXOSC10 | 0.000216372 |
| X | 107464327 | 107464339 | COL4A6 | 0.000216372 |
| 12 | 95488339 | 95488354 | FGD6 | 0.000221484 |
| 17 | 57762402 | 57762414 | CLTC | 0.00022445 |
| 2 | 176995682 | 176995693 | HOXD8 | 0.000235849 |
| X | 24084241 | 24084254 | EIF2S3 | 0.000236183 |
| X | 48214185 | 48214200 | SSX3 | 0.000246812 |
| 13 | 113965002 | 113965017 | LAMP1 | 0.000251351 |
| 14 | 45587186 | 45587199 | FKBP3 | 0.000251362 |
| 10 | 8111345 | 8111359 | GATA3 | 0.000255363 |
| 5 | 77745712 | 77745752 | SCAMP1 | 0.000256016 |
| 5 | 52358761 | 52358775 | ITGA2 | 0.000268264 |
| 3 | 132173004 | 132173026 | DNAJC13 | 0.000270819 |
| 12 | 8692327 | 8692338 | CLEC4E | 0.000273411 |
| 8 | 80831393 | 80831407 | MRPS28 | 0.00027604 |
| 7 | 150918586 | 150918602 | ABCF2 | 0.000281413 |
| X | 135481956 | 135481970 | GPR112 | 0.00028342 |
| 21 | 42598164 | 42598175 | BACE2 | 0.000284158 |
| 6 | 56504477 | 56504489 | RNU6-71P | 0.000287356 |
| 2 | 128263363 | 128263377 | IWS1 | 0.00029264 |
| 9 | 86354663 | 86354679 | GKAP1 | 0.000295479 |
| 2 | 27597190 | 27597204 | SNX17 | 0.000299303 |
| 9 | 97555228 | 97555242 | C9orf3 | 0.000302746 |
| 12 | 133237753 | 133237768 | POLE | 0.000306373 |
| 1 | 207753824 | 207753846 | CR1 | 0.000307645 |
| 7 | 98639904 | 98639917 | SMURF1 | 0.000308642 |
| 2 | 39240584 | 39240596 | SOS1 | 0.000316196 |
| 8 | 101933023 | 101933037 | YWHAZ | 0.00031736 |
| 7 | 75185990 | 75186002 | HIP1 | 0.000318725 |
| 9 | 127287158 | 127287183 | NR6A1 | 0.000340101 |
| 10 | 25140413 | 25140425 | PRTFDC1 | 0.000342924 |
| 3 | 180320897 | 180320910 | TTC14 | 0.000345781 |
| 13 | 99890849 | 99890862 | MIR548AN | 0.000348675 |
| 2 | 119739127 | 119739142 | MARCO | 0.000349824 |
| 4 | 89352469 | 89352487 | HERC6 | 0.000351605 |
| 18 | 649879 | 649895 | TYMSOS(NM_001012716: c.*120_*104del0) | 0.000357577 |
| 6 | 102337480 | 102337497 | GRIK2 | 0.000357782 |
| 12 | 50867692 | 50867704 | LARP4 | 0.000363372 |
| 11 | 128350396 | 128350418 | ETS1 | 0.000364277 |
| 2 | 207013834 | 207013846 | NDUFS1 | 0.000369987 |
| 5 | 10405809 | 10405822 | MARCH6 | 0.000369987 |
| 4 | 39233719 | 39233734 | MIR1273H | 0.000372456 |
| 15 | 81427705 | 81427719 | C15orf26 | 0.000383054 |
| 12 | 56213978 | 56213991 | ORMDL2 | 0.000385208 |
| 1 | 167871297 | 167871312 | ADCY10 | 0.00038961 |
| 8 | 24348425 | 24348437 | LOC101929294, LOC101929315 | 0.000396825 |
| 14 | 96848565 | 96848580 | GSKIP | 0.00039984 |
| 14 | 21960425 | 21960451 | TOX4 | 0.000442087 |
| 2 | 211179765 | 211179777 | MYL1 | 0.000452515 |
| 5 | 68662287 | 68662298 | AK6, TAF9 | 0.000454442 |
| 10 | 34573008 | 34573020 | PARD3 | 0.000455982 |
| 1 | 165712555 | 165712571 | TMCO1 | 0.000463177 |
| 1 | 113159364 | 113159384 | ST7L | 0.000469339 |
| 1 | 237060945 | 237060970 | MTR(NM_000254: c.*1_*26del0, | 0.000470544 |
| | | | NM_001291939: c.*1_*26del0, | |
| | | | NM_001291940: c.*1_*26del0) | |
| 5 | 38954825 | 38954836 | RICTOR | 0.000471698 |
| 16 | 5129689 | 5129703 | ALG1 | 0.000474169 |
| 8 | 134125650 | 134125662 | TG | 0.000474293 |
| 1 | 150799125 | 150799138 | ARNT | 0.000477669 |
| 19 | 36564438 | 36564455 | WDR62 | 0.000477669 |
| 1 | 116655183 | 116655197 | MAB21L3(NM_152367: c.-54_-40del-) | 0.000479074 |
| 1 | 12330939 | 12330953 | VPS13D | 0.000482625 |
| 1 | 173795803 | 173795821 | DARS2 | 0.000489115 |
| 2 | 61308569 | 61308585 | KIAA1841 | 0.000489115 |
| 12 | 90005161 | 90005173 | ATP2B1 | 0.000492587 |
| 11 | 124007701 | 124007714 | VWA5A | 0.000493197 |
| 6 | 110426583 | 110426595 | WASF1 | 0.000500417 |
| 1 | 116940481 | 116940497 | ATP1A1-AS1 | 0.000500581 |
| 5 | 118451836 | 118451847 | DMXL1 | 0.000502513 |
| 15 | 50782416 | 50782446 | USP8 | 0.00050478 |
| 16 | 4873743 | 4873761 | GLYR1 | 0.000514282 |
| 20 | 36431202 | 36431215 | CTNNBL1 | 0.000514386 |
| 17 | 73515055 | 73515068 | TSEN54 | 0.000517974 |
| 11 | 119145463 | 119145476 | CBL | 0.000517974 |
| 15 | 64717845 | 64717857 | TRIP4 | 0.000523013 |
| 3 | 132169705 | 132169718 | DNAJC13 | 0.000532611 |
| 17 | 45105631 | 45105644 | RPRML(dist = 49017), ARL17A(dist = 3308) | 0.000551572 |
| 2 | 175246545 | 175246557 | CIR1 | 0.000556567 |
| 17 | 39659874 | 39659890 | KRT13 | 0.000560119 |
| 11 | 124624705 | 124624721 | ESAM | 0.000570633 |
| 14 | 35497274 | 35497286 | SRP54 | 0.000574581 |
| 1 | 85575838 | 85575853 | WDR63 | 0.000589251 |
| 21 | 34117067 | 34117080 | PAXBP1 | 0.000603819 |
| 2 | 47635523 | 47635537 | MSH2 | 0.000613834 |
| 6 | 25452453 | 25452466 | LRRC16A | 0.000639987 |
| 2 | 101581294 | 101581308 | NPAS2 | 0.000652032 |
| 2 | 95783701 | 95783713 | MRPS5 | 0.000662468 |
| 17 | 61958954 | 61958979 | GH2 | 0.000678841 |
| 1 | 65138787 | 65138801 | CACHD1 | 0.000684265 |
| 12 | 132496182 | 132496193 | EP400 | 0.000686342 |
| 5 | 112903585 | 112903598 | YTHDC2 | 0.000693963 |
| 9 | 73164596 | 73164610 | TRPM3 | 0.000697569 |
| 3 | 172080401 | 172080412 | FNDC3B | 0.00071286 |
| 14 | 31191658 | 31191682 | SCFD1 | 0.000716363 |
| 15 | 40951461 | 40951477 | CASC5 | 0.000716675 |
| 19 | 36572251 | 36572263 | WDR62 | 0.000750912 |
| X | 108636043 | 108636070 | GUCY2F | 0.000756305 |
| 1 | 241954005 | 241954024 | WDR64 | 0.000761589 |
| 3 | 129155547 | 129155558 | MBD4 | 0.000766088 |
| 11 | 65268479 | 65268492 | MALAT1 | 0.000770331 |
| 6 | 96982111 | 96982125 | UFL1 | 0.000783699 |
| 1 | 153955601 | 153955614 | RAB13 | 0.000807275 |
| 11 | 117152291 | 117152314 | RNF214 | 0.000812898 |
| 19 | 14104688 | 14104703 | RFX1(NM_002918: c.-33_-48del0) | 0.000844736 |
| 6 | 36452603 | 36452620 | KCTD20 | 0.000844736 |
| 1 | 159166612 | 159166624 | CADM3-AS1 | 0.000855178 |
| 12 | 109510086 | 109510099 | USP30 | 0.000889522 |
| 11 | 16339918 | 16339934 | SOX6 | 0.000889522 |
| 2 | 118854094 | 118854108 | INSIG2(NM_016133: c.-39_-25del-) | 0.000964295 |
| 7 | 73279233 | 73279260 | WBSCR28 | 0.000964721 |
| 3 | 33725814 | 33725828 | CLASP2 | 0.00098736 |
| 11 | 63149670 | 63149682 | SLC22A9 | 0.000995419 |
| 1 | 50663196 | 50663209 | ELAVL4 | 0.001003578 |
| 13 | 28133956 | 28133972 | LNX2 | 0.001011837 |
| 19 | 9771286 | 9771300 | ZNF562 | 0.001034752 |
| 6 | 163235158 | 163235170 | PACRG | 0.001037235 |
| 10 | 81036869 | 81036879 | ZMIZ1 | 0.001042644 |
| 17 | 30320860 | 30320882 | SUZ12 | 0.001081742 |
| 14 | 95999676 | 95999689 | SNHG10 | 0.001084735 |
| 19 | 35758789 | 35758801 | LSR(NM_015925: c.*27_*39del0, | 0.001084735 |
| | | | NM_205835: c.*27_*39del0, | |
| | | | NM_205834: c.*27_*39del0, | |
| | | | NM_001260490: c.*27_*39del0, | |
| | | | NM_001260489: c.*27_*39del0) | |
| 9 | 80343587 | 80343612 | GNAQ | 0.001090986 |
| 4 | 8472785 | 8472805 | TRMT44 | 0.001096742 |
| 5 | 176729736 | 176729749 | RAB24 | 0.001096987 |
| 9 | 86588185 | 86588199 | HNRNPK | 0.001149027 |
| 16 | 19481220 | 19481235 | TMC5 | 0.001256808 |
| 2 | 120714401 | 120714418 | PTPN4 | 0.001312605 |
| 11 | 134118382 | 134118401 | THYN1 | 0.001385696 |
| 7 | 92129161 | 92129183 | PEX1 | 0.001438858 |
| 5 | 67576330 | 67576346 | PIK3R1 | 0.001438858 |
| 10 | 17110577 | 17110588 | CUBN | 0.001441708 |
| 15 | 52561891 | 52561907 | MYO5C | 0.001448641 |
| 12 | 31820564 | 31820578 | METTL20 | 0.00149231 |
| 12 | 121004564 | 121004583 | RNF10 | 0.001492773 |
| 6 | 157495951 | 157495966 | ARID1B | 0.001501185 |
| X | 100530312 | 100530326 | TAF7L | 0.001569607 |
| 17 | 39871551 | 39871566 | GAST | 0.001571151 |
| 6 | 111702448 | 111702460 | REV3L | 0.001574357 |
| 5 | 14485161 | 14485175 | TRIO | 0.001577546 |
| 11 | 71548312 | 71548339 | DEFB108B | 0.001585786 |
| 10 | 43199666 | 43199677 | LINC01518(dist = 12459), BMS1(dist = 78277) | 0.00160422 |
| 2 | 172314644 | 172314656 | DCAF17 | 0.001624915 |
| 14 | 76524970 | 76524982 | IFT43 | 0.001632604 |
| 5 | 179150788 | 179150800 | CANX | 0.001635852 |
| 13 | 113818805 | 113818818 | PROZ | 0.001661959 |
| 11 | 59368218 | 59368235 | OSBP | 0.001754454 |
| 1 | 99164246 | 99164260 | SNX7 | 0.001763357 |
| 2 | 183960133 | 183960155 | DUSP19 | 0.001796025 |
| 15 | 101019693 | 101019705 | CERS3 | 0.001812178 |
| 11 | 118369265 | 118369290 | KMT2A | 0.001823505 |
| 4 | 101337123 | 101337134 | EMCN | 0.001842536 |
| 6 | 133093231 | 133093247 | SLC18B1 | 0.00193585 |
| 2 | 173848186 | 173848214 | RAPGEF4 | 0.001996891 |
| 17 | 74286032 | 74286047 | QRICH2 | 0.002005228 |
| 19 | 11565819 | 11565831 | ELAVL3 | 0.002018712 |
| 7 | 134626034 | 134626049 | CALD1 | 0.002060535 |
| 1 | 112303077 | 112303090 | DDX20 | 0.002062152 |
| 14 | 68582259 | 68582279 | RAD51B | 0.002093745 |
| 4 | 79754716 | 79754728 | BMP2K | 0.002144873 |
| 17 | 4646533 | 4646547 | ZMYND15 | 0.002219289 |
| 2 | 206911191 | 206911211 | INO80D | 0.002253229 |
| 20 | 60963346 | 60963359 | RPS21 | 0.002321725 |
| 9 | 52625 | 52641 | FAM138C(dist = 16761), PGM5P3-AS1(dist = 20049) | 0.002502187 |
| 7 | 95442405 | 95442420 | DYNC1I1 | 0.002512967 |
| 15 | 55972660 | 55972674 | PRTG | 0.002598137 |
| 2 | 179616770 | 179616786 | TTN | 0.00268172 |
| 14 | 91639611 | 91639627 | C14orf159 | 0.002742018 |
| 1 | 234565078 | 234565090 | TARBP1 | 0.002836165 |
| X | 100793432 | 100793449 | ARMCX4(dist = 42634), ARMCX1(dist = 12065) | 0.002877698 |
| 2 | 21226218 | 21226230 | APOB | 0.003019997 |
| 7 | 81641570 | 81641584 | LOC101927356 | 0.003075233 |
| 12 | 27841925 | 27841938 | PPFIBP1 | 0.003110914 |
| 1 | 235621851 | 235621865 | B3GALNT2 | 0.003236992 |
| 15 | 41770639 | 41770652 | RTF1 | 0.00328121 |
| 15 | 40005793 | 40005806 | FSIP1 | 0.003332107 |
| 11 | 55656437 | 55656451 | TRIM51 | 0.003399405 |
| 6 | 43021976 | 43021989 | MRPL2(NM_015950: c.*36_*23del0, | 0.003422944 |
| | | | NM_001300848: c.*202_*189del0) | |
| 12 | 99117397 | 99117414 | APAF1 | 0.003618497 |
| 6 | 136710669 | 136710685 | MAP7 | 0.00367396 |
| 7 | 26883761 | 26883776 | SKAP2 | 0.003678084 |
| 3 | 15613279 | 15613304 | HACL1 | 0.003762378 |
| 6 | 20758808 | 20758822 | CDKAL1 | 0.003922319 |
| 17 | 74721967 | 74721983 | JMJD6 | 0.003984394 |
| 1 | 63044645 | 63044661 | DOCK7 | 0.004034497 |
| 6 | 133138067 | 133138082 | RPS12 | 0.004087252 |
| 13 | 33637913 | 33637928 | KL | 0.004100523 |
| 6 | 121412191 | 121412203 | TBC1D32 | 0.004148696 |
| 5 | 134015250 | 134015288 | SEC24A | 0.004255319 |
| 1 | 109856836 | 109856854 | SORT1 | 0.004274795 |
| 9 | 5811331 | 5811349 | ERMP1 | 0.00456621 |
| 12 | 110720351 | 110720363 | ATP2A2 | 0.004598134 |
| 12 | 53861636 | 53861650 | PCBP2 | 0.00460591 |
| 14 | 64443258 | 64443278 | MIR548AZ | 0.004728293 |
| 8 | 75944409 | 75944422 | CRISPLD1 | 0.004777562 |
| 2 | 238925144 | 238925166 | UBE2F-SCLY | 0.004830918 |
| 1 | 117911178 | 117911189 | MAN1A2 | 0.004830918 |
| 22 | 44368728 | 44368741 | SAMM50 | 0.004871187 |
| X | 100666907 | 100666921 | HNRNPH2, RPL36A-HNRNPH2 | 0.004878049 |
| 3 | 42599049 | 42599064 | SEC22C | 0.004933759 |
| 5 | 54552380 | 54552391 | DHX29 | 0.004950495 |
| 7 | 36465606 | 36465622 | ANLN | 0.005071541 |
| 1 | 115262366 | 115262389 | CSDE1 | 0.005076142 |
| 2 | 10928694 | 10928709 | PDIA6 | 0.005164222 |
| 9 | 96209987 | 96209999 | FAM120AOS | 0.005265236 |
| 20 | 6077429 | 6077449 | FERMT1 | 0.005299223 |
| 13 | 78137932 | 78137946 | SCEL | 0.005541183 |
| X | 135630678 | 135630689 | VGLL1 | 0.005552794 |
| 14 | 39594134 | 39594146 | GEMIN2 | 0.005813953 |
| 7 | 84671609 | 84671620 | SEMA3D | 0.005813953 |
| 4 | 39511530 | 39511543 | MIR1273H | 0.005999859 |
| 12 | 48360947 | 48360961 | TMEM106C | 0.006000513 |
| 15 | 101550861 | 101550877 | LRRK1 | 0.006104998 |
| 1 | 54692854 | 54692865 | SSBP3 | 0.00617284 |
| 4 | 41015553 | 41015566 | APBB2 | 0.006231221 |
| 6 | 126075709 | 126075734 | HEY2 | 0.00627802 |
| 5 | 74722305 | 74722316 | COL4A3BP | 0.006289308 |
| 19 | 55438923 | 55438944 | NLRP7 | 0.006369045 |
| 2 | 88484805 | 88484815 | THNSL2 | 0.006410256 |
| 3 | 188242333 | 188242358 | LPP | 0.006410256 |
| 2 | 238419543 | 238419568 | MLPH | 0.006530381 |
| 4 | 39871117 | 39871154 | PDS5A | 0.006530381 |
| 2 | 54125185 | 54125196 | PSME4 | 0.006622517 |
| 4 | 84519775 | 84519790 | AGPAT9 | 0.006843973 |
| 5 | 131924346 | 131924374 | RAD50 | 0.006896552 |
| X | 154456644 | 154456657 | VBP1 | 0.006994412 |
| 14 | 51348281 | 51348299 | ABHD12B | 0.007009585 |
| 1 | 109617803 | 109617815 | TAF13 | 0.007042254 |
| 12 | 113645152 | 113645163 | IQCD | 0.007142857 |
| 12 | 57032216 | 57032235 | ATP5B | 0.00729927 |
| 5 | 75858199 | 75858210 | IQGAP2 | 0.007352941 |
| 20 | 46270911 | 46270923 | NCOA3 | 0.007575758 |
| 17 | 30500816 | 30500828 | RHOT1 | 0.007604563 |
| 1 | 179561673 | 179561709 | TDRD5(NM_001199091: c.-78_-42del-) | 0.007633588 |
| 17 | 74736999 | 74737016 | MFSD11 | 0.007756119 |
| 1 | 91402917 | 91402929 | ZNF644 | 0.007784946 |
| 7 | 107336262 | 107336274 | SLC26A4 | 0.0078125 |
| 2 | 111881263 | 111881275 | BCL2L11 | 0.0078125 |
| 10 | 93601945 | 93601955 | TNKS2 | 0.007874016 |
| 17 | 62855003 | 62855017 | LRRC37A3 | 0.008 |
| X | 154009644 | 154009656 | MPP1 | 0.008130081 |
| X | 19606999 | 19607014 | SH3KBP1 | 0.008333333 |
| 14 | 92549604 | 92549623 | ATXN3 | 0.008583691 |
| 2 | 43768233 | 43768244 | THADA | 0.00862069 |
| 17 | 37657460 | 37657471 | CDK12 | 0.008624591 |
| 4 | 15642515 | 15642535 | FBXL5 | 0.008733624 |
| 2 | 189859434 | 189859447 | COL3A1 | 0.00877193 |
| 4 | 146076632 | 146076644 | OTUD4 | 0.00878035 |
| X | 110494528 | 110494543 | CAPN6 | 0.008849558 |
| 13 | 73409508 | 73409522 | PIBF1 | 0.008866553 |
| 10 | 12215870 | 12215884 | NUDT5 | 0.00904258 |
| 8 | 68139326 | 68139338 | ARFGEF1 | 0.009049774 |
| 8 | 124382376 | 124382403 | ATAD2 | 0.009090909 |
| 19 | 34687462 | 34687485 | LSM14A | 0.009259259 |
| 17 | 67178976 | 67178992 | ABCA10 | 0.009421209 |
| 4 | 38937316 | 38937329 | FAM114A1 | 0.009478673 |
| 1 | 243736210 | 243736226 | AKT3 | 0.009508655 |
| 17 | 2323953 | 2323979 | METTL16 | 0.00952381 |
| 10 | 93841262 | 93841273 | CPEB3 | 0.009661836 |
| 2 | 238271786 | 238271797 | COL6A3 | 0.009674113 |
| 18 | 21293834 | 21293881 | LAMA3 | 0.009708738 |
| X | 31200799 | 31200843 | DMD | 0.009860334 |
| 21 | 19653342 | 19653355 | TMPRSS15 | 0.009896847 |
| 19 | 54400979 | 54401003 | PRKCG | 0.00990099 |
| 1 | 47123898 | 47123914 | ATPAF1 | 0.009979494 |
| 16 | 15133783 | 15133795 | NTAN1, PDXDC1 | 0.010032747 |
| 1 | 46726867 | 46726890 | RAD54L | 0.010050251 |
| 5 | 70797500 | 70797521 | BDP1 | 0.01010101 |
| 11 | 46666872 | 46666883 | ATG13 | 0.010152284 |
| 6 | 149718577 | 149718593 | TAB2 | 0.010362694 |
| 6 | 18258299 | 18258311 | DEK | 0.010638298 |
| 12 | 120741303 | 120741326 | SIRT4 | 0.010695187 |
| 11 | 132179950 | 132179979 | NTM | 0.010752688 |
| X | 35944077 | 35944088 | CXorf22 | 0.010752688 |
| 12 | 26816832 | 26816843 | ITPR2 | 0.010752688 |
| 11 | 104879686 | 104879697 | CASP5 | 0.010810811 |
| 1 | 31766005 | 31766017 | SNRNP40 | 0.010928962 |
| 8 | 38153186 | 38153199 | WHSC1L1 | 0.010928962 |
| 2 | 167138320 | 167138331 | SCN9A | 0.010935187 |
| 11 | 86120270 | 86120294 | CCDC81 | 0.011049724 |
| 7 | 8110836 | 8110848 | GLCCI1 | 0.011049724 |
| 12 | 53470811 | 53470838 | SPRYD3 | 0.011049724 |
| 17 | 16056594 | 16056626 | NCOR1 | 0.011049724 |
| 5 | 131927539 | 131927561 | RAD50 | 0.011111111 |
| 2 | 111397237 | 111397251 | BUB1 | 0.011114006 |
| 15 | 51768755 | 51768768 | DMXL2 | 0.011173184 |
| 18 | 14214318 | 14214331 | ANKRD20A5P | 0.011363636 |
| 15 | 66794298 | 66794311 | RPL4 | 0.011363636 |
| 7 | 112555294 | 112555307 | C7orf60 | 0.011406844 |
| 7 | 38282130 | 38282151 | STARD3NL(dist = 11858), TARP(dist = 17093) | 0.011433093 |
| 6 | 28876873 | 28876904 | TRIM27 | 0.011450382 |
| 12 | 97051927 | 97051940 | CDK17(dist = 257561), NEDD1(dist = 249061) | 0.011466421 |
| 17 | 41256074 | 41256122 | BRCA1 | 0.01146862 |
| 6 | 17292448 | 17292460 | RBM24(NM_001143942: c.*98_*110del0, | 0.011494253 |
| | | | NM_001143941: c.*98_*110del0, | |
| | | | NM_153020: c.*98_*110del0) | |
| 1 | 154178185 | 154178198 | C1orf189 | 0.011494253 |
| 3 | 62459813 | 62459846 | CADPS | 0.011494253 |
| 11 | 49088577 | 49088602 | TRIM64C(dist = 7913), FOLH1(dist = 79585) | 0.01150398 |
| 4 | 178357514 | 178357527 | AGA | 0.011560694 |
| 20 | 35660978 | 35660989 | RBL1 | 0.011560694 |
| 2 | 58362150 | 58362161 | VRK2 | 0.011764706 |
| X | 134025421 | 134025436 | MOSPD1 | 0.011811024 |
| 11 | 55339545 | 55339556 | OR4C16 | 0.011811024 |
| 11 | 74053485 | 74053497 | PGM2L1 | 0.011857708 |
| 7 | 103828911 | 103828922 | ORC5 | 0.011904762 |
| 15 | 52239617 | 52239629 | LEO1 | 0.011952191 |
| 15 | 34434467 | 34434479 | KATNBL1(NM_024713: c.*73_*61del0) | 0.011988012 |
| 19 | 51919434 | 51919447 | LOC100129083, SIGLEC10 | 0.012 |
| 17 | 8417006 | 8417020 | MYH10 | 0.012021927 |
| 1 | 54252939 | 54252952 | NDC1 | 0.012048193 |
| 4 | 83970297 | 83970312 | COPS4 | 0.012048193 |
| 6 | 5000724 | 5000762 | RPP40 | 0.012048193 |
| 1 | 220269426 | 220269439 | RNU5F-1 | 0.012121212 |
| 6 | 130761525 | 130761543 | TMEM200A | 0.012145749 |
| 15 | 43990706 | 43990741 | CKMT1A | 0.012195122 |
| 21 | 38564530 | 38564545 | TTC3 | 0.012195122 |
| 2 | 55863360 | 55863372 | PNPT1 | 0.012200061 |
| 11 | 47187055 | 47187087 | ARFGAP2 | 0.012244898 |
| 18 | 3597089 | 3597100 | DLGAP1-AS1 | 0.012244898 |
| 2 | 238005446 | 238005457 | COPS8 | 0.012269939 |
| 11 | 89395303 | 89395315 | FOLH1B | 0.012327142 |
| X | 48631645 | 48631660 | GLOD5 | 0.012345679 |
| 10 | 52566432 | 52566445 | A1CF(NM_001198820: c.*57_*44del0, | 0.01242236 |
| | | | NM_014576: c.*57_*44del0, | |
| | | | NM_138932: c.*57_*44del0, | |
| | | | NM_001198819: c.*57_*44del0, | |
| | | | NM_001198818: c.*57_*44del0, | |
| | | | NM138933: c.*57_*44del0) | |
| 16 | 69385403 | 69385421 | TMED6 | 0.012448133 |
| 7 | 91714092 | 91714106 | AKAP9 | 0.012448133 |
| 2 | 187466921 | 187466932 | ITGAV | 0.0125 |
| 6 | 32155249 | 32155264 | PBX2 | 0.0125 |
| 10 | 112044559 | 112044572 | MXI1 | 0.012552301 |
| 12 | 7187777 | 7187796 | C1R(NM_001733: c.*59_*40del0) | 0.012552301 |
| 9 | 113449613 | 113449633 | MUSK | 0.012578616 |
| 5 | 137507104 | 137507121 | BRD8 | 0.012586287 |
| 1 | 109349996 | 109350021 | STXBP3 | 0.012658228 |
| 7 | 137076100 | 137076123 | DGKI | 0.012711864 |
| 11 | 4150238 | 4150253 | RRM1 | 0.012738854 |
| X | 103164079 | 103164090 | MIR1256 | 0.012738854 |
| 21 | 37425988 | 37425999 | SETD4 | 0.012738854 |
| 10 | 102716310 | 102716330 | FAM178A | 0.012875536 |
| 20 | 34430474 | 34430486 | PHF20 | 0.012987013 |
| 12 | 51323672 | 51323684 | METTL7A | 0.012987013 |
| 1 | 22837586 | 22837604 | ZBTB40 | 0.013034757 |
| 2 | 152424933 | 152424962 | NEB | 0.013043478 |
| 7 | 5087800 | 5087822 | RBAK, RBAK-RBAKDN | 0.013043478 |
| 6 | 109480681 | 109480693 | CEP57L1 | 0.013043478 |
| 18 | 12955439 | 12955452 | SEH1L | 0.01310075 |
| 7 | 100319680 | 100319696 | EPO | 0.013157895 |
| 1 | 16245610 | 16245621 | SPEN | 0.013157895 |
| 10 | 115380483 | 115380497 | NRAP | 0.013215859 |
| X | 18183097 | 18183113 | BEND2(NM_153346: c.*32_*16del0) | 0.013215859 |
| 22 | 21968716 | 21968730 | UBE2L3 | 0.013215859 |
| 20 | 31294314 | 31294329 | COMMD7 | 0.013245033 |
| 14 | 50760968 | 50760980 | L2HGDH | 0.013274336 |
| 1 | 158298679 | 158298691 | CD1B | 0.013274336 |
| X | 19086800 | 19086823 | GPR64 | 0.013274336 |
| 10 | 12162338 | 12162351 | DHTKD1 | 0.013333333 |
| 2 | 79314062 | 79314073 | REG1B | 0.013333333 |
| 1 | 200080464 | 200080490 | NR5A2 | 0.013333333 |
| 2 | 98872570 | 98872582 | VWA3B | 0.013333333 |
| 7 | 73254451 | 73254472 | WBSCR27 | 0.013333333 |
| 13 | 26967446 | 26967495 | CDK8 | 0.013392857 |
| 1 | 46650191 | 46650201 | TSPAN1 | 0.013413889 |
| 5 | 108673063 | 108673074 | PJA2 | 0.013422819 |
| 3 | 10128965 | 10128985 | FANCD2, FANCD2OS | 0.013513514 |
| 5 | 72875091 | 72875102 | UTP15 | 0.013513514 |
| 17 | 62532622 | 62532635 | CEP95 | 0.013513514 |
| 1 | 197009613 | 197009644 | F13B | 0.013574661 |
| 1 | 211545442 | 211545459 | TRAF5 | 0.013590424 |
| 1 | 86590509 | 86590523 | COL24A1 | 0.013605442 |
| X | 65486260 | 65486271 | HEPH | 0.013605442 |
| 11 | 125763610 | 125763624 | PUS3(NM_001271985: c.*70_*56del0, | 0.013636364 |
| | | | NM_031307: c.*70_*56del0) | |
| 17 | 35988718 | 35988739 | DDX52 | 0.01369863 |
| 11 | 94230106 | 94230131 | ANKRD49 | 0.013793103 |
| 9 | 98009613 | 98009626 | FANCC | 0.013888889 |
| 2 | 70456452 | 70456465 | TIA1 | 0.013888889 |
| 6 | 99817699 | 99817713 | COQ3 | 0.013953488 |
| 4 | 140262240 | 140262251 | NAA15 | 0.013986014 |
| 18 | 56279052 | 56279066 | ALPK2 | 0.014018692 |
| 19 | 10097443 | 10097455 | COL5A3 | 0.014052062 |
| 9 | 2083313 | 2083338 | SMARCA2 | 0.014084507 |
| X | 129265852 | 129265866 | AIFM1 | 0.014084507 |
| 2 | 208606875 | 208606908 | CCNYL1 | 0.014084507 |
| 5 | 79369202 | 79369225 | CTD-2201I18.1 | 0.014084507 |
| 17 | 65720150 | 65720161 | NOL11 | 0.014084507 |
| 7 | 74608740 | 74608754 | GTF2IP1, LOC100093631 | 0.014150943 |
| 16 | 66855468 | 66855480 | NAE1 | 0.014184397 |
| 16 | 23486187 | 23486201 | GGA2 | 0.014184397 |
| 4 | 99214537 | 99214549 | RAP1GDS1 | 0.014285714 |
| 7 | 23761159 | 23761171 | STK31 | 0.014285714 |
| 16 | 3828847 | 3828870 | CREBBP | 0.014388489 |
| 14 | 74426284 | 74426298 | COQ6 | 0.014423077 |
| X | 153994469 | 153994482 | DKC1 | 0.014440433 |
| 1 | 7912944 | 7912955 | UTS2 | 0.014492754 |
| 5 | 158126222 | 158126236 | EBF1 | 0.014492754 |
| 20 | 34435246 | 34435270 | PHF20 | 0.014492754 |
| 5 | 101795499 | 101795510 | SLCO6A1 | 0.014492754 |
| 9 | 100997485 | 100997498 | TBC1D2 | 0.014545455 |
| 1 | 236737874 | 236737886 | HEATR1 | 0.014563107 |
| 20 | 13140617 | 13140637 | SPTLC3 | 0.01459854 |
| 21 | 40181937 | 40181956 | ETS2 | 0.01459854 |
| 13 | 37011617 | 37011628 | CCNA1 | 0.014634146 |
| 6 | 163899794 | 163899807 | QKI | 0.014705882 |
| 5 | 147793666 | 147793680 | FBXO38 | 0.014705882 |
| 7 | 135304090 | 135304102 | NUP205 | 0.014705882 |
| 3 | 101575877 | 101575895 | NFKBIZ | 0.014778325 |
| X | 52654536 | 52654550 | SSX8 | 0.014778325 |
| 4 | 114176848 | 114176860 | ANK2 | 0.014787278 |
| 13 | 111340025 | 111340053 | CARS2 | 0.014801007 |
| 3 | 41877473 | 41877487 | ULK4 | 0.014851485 |
| 2 | 175246386 | 175246411 | CIR1 | 0.014851485 |
| 9 | 21861902 | 21861914 | MTAP | 0.014851485 |
| 6 | 146256315 | 146256344 | SHPRH | 0.014851485 |
| X | 108924091 | 108924121 | ACSL4 | 0.014925373 |
| 6 | 39047035 | 39047052 | GLP1R | 0.014925373 |
| 1 | 75172756 | 75172771 | CRYZ | 0.014963067 |
| 10 | 102035182 | 102035193 | BLOC1S2(NM_001282439: c.*37_*26del0, | 0.015 |
| | | | NM_001282438: c.*37_*26del0, | |
| | | | NM_001001342: c.*37_*26del0, | |
| | | | NM_173809: c.*37_*26del0, | |
| | | | NM_001282436: c.*37_*26del0, | |
| | | | NM_001282437: c.*37_*26del0) | |
| 2 | 96994050 | 96994061 | ITPRIPL1(NM_001008949: c.*13_*24del0, | 0.015 |
| | | | NM_001163523: c.*13_*24del0, | |
| | | | NM_178495: c.*13_*24del0, | |
| | | | NM_001163524: c.*13_*24del0) | |
| 6 | 157496153 | 157496197 | ARID1B | 0.015037594 |
| 2 | 48046012 | 48046035 | FBXO11 | 0.015037594 |
| 19 | 4430524 | 4430535 | CHAF1A | 0.015075377 |
| 6 | 109319962 | 109319991 | SESN1 | 0.01509434 |
| 17 | 29206408 | 29206439 | ATAD5 | 0.015209125 |
| 19 | 14153179 | 14153202 | IL27RA | 0.015267176 |
| 19 | 5727206 | 5727218 | CATSPERD | 0.015267176 |
| 14 | 35255287 | 35255316 | BAZ1A | 0.015267176 |
| X | 101409243 | 101409271 | BEX5(NM_001159560: c.-6_-34del0, | 0.01532567 |
| | | | NM_001012978: c.-6_-34del0) | |
| 13 | 79216356 | 79216381 | RNF219 | 0.015384615 |
| 1 | 36297998 | 36298033 | AGO4 | 0.015384615 |
| 6 | 34831755 | 34831773 | UHRF1BP1 | 0.015463918 |
| 15 | 90229786 | 90229798 | PEX11A | 0.015503876 |
| 11 | 108332126 | 108332138 | C11orf65 | 0.015503876 |
| 3 | 196236651 | 196236664 | SMCO1 | 0.015503876 |
| 22 | 29879265 | 29879302 | NEFH | 0.015564202 |
| X | 132888207 | 132888236 | GPC3 | 0.015564202 |
| 22 | 36872638 | 36872662 | TXN2 | 0.015625 |
| 12 | 48880078 | 48880089 | C12orf54 | 0.015625 |
| 1 | 200544650 | 200544673 | KIF14 | 0.015655213 |
| 2 | 101885656 | 101885675 | CNOT11 | 0.015686275 |
| 3 | 100438778 | 100438810 | TFG | 0.015706806 |
| 3 | 44399683 | 44399706 | TCAIM | 0.015706806 |
| 1 | 216373476 | 216373489 | USH2A | 0.015706806 |
| 3 | 3216976 | 3216988 | CRBN | 0.015716909 |
| 12 | 4645161 | 4645173 | C12orf4 | 0.015873016 |
| 14 | 91633483 | 91633498 | C14orf159 | 0.015873016 |
| 3 | 150906314 | 150906344 | MED12L | 0.015873016 |
| 3 | 38006175 | 38006186 | CTDSPL | 0.015873016 |
| 5 | 158630629 | 158630653 | RNF145 | 0.015936255 |
| 11 | 108188266 | 108188280 | ATM | 0.015936255 |
| 2 | 110925982 | 110925995 | NPHP1 | 0.015936255 |
| 8 | 53455013 | 53455024 | FAM150A | 0.015957447 |
| 3 | 121515940 | 121515953 | IQCB1 | 0.016 |
| X | 119675354 | 119675368 | CUL4B | 0.016064257 |
| 13 | 100913132 | 100913161 | PCCA | 0.016064257 |
| 5 | 122359456 | 122359480 | PPIC(NM_000943: c.*114_*90del0) | 0.016129032 |
| 1 | 197898395 | 197898415 | LHX9(NM_001014434: c.*6_*26del0, | 0.016129032 |
| | | | NM_020204: c.*6_*26del0) | |
| 17 | 35620810 | 35620826 | ACACA | 0.016129032 |
| 14 | 74875955 | 74875968 | SYNDIG1L | 0.016161417 |
| 2 | 157370018 | 157370033 | GPD2 | 0.016194332 |
| 2 | 24521686 | 24521697 | ITSN2 | 0.016260163 |
| 12 | 88482779 | 88482791 | CEP290 | 0.016260163 |
| 4 | 107037269 | 107037282 | TBCK | 0.016260163 |

TABLE 5
Microsatellite Repeat Marker Sequences for Lung Squamous Cell Carcinoma (LUSC).
TABLE 5 - LUSC

| ch | start_pos | end_pos | | LUSC |
|---|---|---|---|---|
| (hg37) | (hg37) | (hg37 | gene | p-value |
| 3 | 48636506 | 48636516 | UQCRC1(NM_003365: c.*55_*45del0) | 1.42813E−10 |
| X | 53113895 | 53113905 | TSPYL2 | 1.32824E−06 |
| 2 | 46583272 | 46583283 | EPAS1 | 2.13437E−06 |
| 5 | 67584512 | 67584525 | PIK3R1(NM_181524: c.-52_-39del-) | 4.6354E−06 |
| 2 | 228159652 | 228159677 | LOC654841 | 6.14867E−06 |
| 2 | 66796098 | 66796109 | MEIS1 | 8.7468E−06 |
| 3 | 186295417 | 186295431 | DNAJB11 | 1.30955E−05 |
| 17 | 67178976 | 67178992 | ABCA10 | 1.55218E−05 |
| 2 | 179616770 | 179616786 | TTN | 2.19677E−05 |
| 13 | 73409508 | 73409522 | PIBF1 | 2.39998E−05 |
| 1 | 155701055 | 155701071 | DAP3 | 2.42228E−05 |
| 5 | 14485161 | 14485175 | TRIO | 2.87624E−05 |
| 17 | 67178764 | 67178794 | ABCA10 | 3.10099E−05 |
| 15 | 50920509 | 50920523 | TRPM7 | 3.64511E−05 |
| 2 | 215890346 | 215890382 | ABCA12 | 4.20258E−05 |
| 18 | 43314395 | 43314412 | SLC14A1 | 4.23846E−05 |
| 8 | 89499214 | 89499230 | MMP16(dist = 159497), LOC101929709(dist = 1230397) | 4.93243E−05 |
| X | 129354254 | 129354276 | ZNF280C | 5.65448E−05 |
| 11 | 124624705 | 124624721 | ESAM | 6.02149E−05 |
| 13 | 99890849 | 99890862 | MIR548AN | 6.16333E−05 |
| 10 | 63977011 | 63977030 | RTKN2 | 6.18461E−05 |
| 1 | 169797067 | 169797078 | C1orf112 | 6.57479E−05 |
| 6 | 100403812 | 100403827 | MCHR2 | 7.86164E−05 |
| 3 | 108175734 | 108175745 | MYH15 | 8.83002E−05 |
| 1 | 213180434 | 213180459 | ANGEL2 | 9.06871E−05 |
| 9 | 20740346 | 20740366 | FOCAD | 9.7679E−05 |
| 3 | 185155430 | 185155442 | MAP3K13 | 9.81082E−05 |
| 11 | 10820663 | 10820688 | EIF4G2 | 0.000106296 |
| 6 | 43021976 | 43021989 | MRPL2(NM_015950: c.*36_*23del0, | 0.00011493 |
| | | | NM_001300848: c.*202_*189del0) | |
| 2 | 88484805 | 88484815 | THNSL2 | 0.000116482 |
| 16 | 68155860 | 68155874 | NFATC3 | 0.000133581 |
| 19 | 50911947 | 50911960 | POLD1 | 0.000140056 |
| 2 | 21226218 | 21226230 | APOB | 0.000176336 |
| X | 100530312 | 100530326 | TAF7L | 0.000179631 |
| 2 | 175246545 | 175246557 | CIR1 | 0.00019313 |
| 11 | 108121410 | 108121426 | ATM | 0.000194441 |
| 16 | 75674252 | 75674271 | KARS | 0.000201324 |
| 8 | 120800692 | 120800703 | TAF2 | 0.000210393 |
| 1 | 211545442 | 211545459 | TRAF5 | 0.000219138 |
| 20 | 4850568 | 4850578 | SLC23A2 | 0.000220942 |
| 2 | 66670211 | 66670223 | MEIS1 | 0.000221729 |
| X | 117883813 | 117883824 | IL13RA1 | 0.000223964 |
| 21 | 17238572 | 17238582 | USP25 | 0.000228781 |
| 18 | 77737714 | 77737726 | TXNL4A | 0.000230044 |
| 3 | 180679235 | 180679250 | FXR1 | 0.000230044 |
| 15 | 41770598 | 41770624 | RTF1 | 0.000230044 |
| 7 | 139097271 | 139097295 | C7orf55-LUC7L2, LUC7L2 | 0.000233238 |
| 16 | 84907499 | 84907520 | CRISPLD2 | 0.000261749 |
| 12 | 99117397 | 99117414 | APAF1 | 0.000273332 |
| 4 | 187627659 | 187627676 | FAT1 | 0.000274472 |
| 5 | 6623405 | 6623418 | NSUN2 | 0.000278707 |
| 12 | 121004564 | 121004583 | RNF10 | 0.000280306 |
| 17 | 35771517 | 35771528 | TADA2A | 0.000286862 |
| 9 | 19334957 | 19334970 | DENND4C | 0.00029264 |
| 2 | 110922055 | 110922080 | NPHP1 | 0.000304553 |
| 15 | 52185961 | 52185975 | TMOD3 | 0.000348334 |
| 10 | 27037487 | 27037499 | ABI1 | 0.000388149 |
| X | 135080623 | 135080653 | SLC9A6 | 0.000393465 |
| 19 | 11411997 | 11412010 | TSPAN16 | 0.00040328 |
| 1 | 220369745 | 220369758 | RAB3GAP2 | 0.000405725 |
| 11 | 104878040 | 104878051 | CASP5 | 0.000407647 |
| 7 | 100319680 | 100319696 | EPO | 0.000418918 |
| 1 | 236737874 | 236737886 | HEATR1 | 0.000432869 |
| 1 | 171511148 | 171511159 | PRRC2C | 0.000474083 |
| 12 | 120741303 | 120741326 | SIRT4 | 0.000477669 |
| 12 | 31435608 | 31435620 | FAM60A(NM_001135811: c.*38_*26del0, | 0.000482952 |
| | | | NM_001135812: c.*38_*26del0, | |
| | | | NM_021238: c.*38_*26del0) | |
| 12 | 125587478 | 125587491 | AACS | 0.000497512 |
| 7 | 101747593 | 101747605 | CUX1 | 0.000502638 |
| 20 | 23473522 | 23473535 | CST8 | 0.000509127 |
| 8 | 101933023 | 101933037 | YWHAZ | 0.000519211 |
| 6 | 64289938 | 64289954 | PTP4A1 | 0.000543607 |
| 19 | 17366101 | 17366122 | USHBP1 | 0.000544168 |
| 14 | 45497556 | 45497567 | FAM179B | 0.000549451 |
| 3 | 158407930 | 158407945 | GFM1 | 0.00056684 |
| 13 | 52726707 | 52726730 | NEK3 | 0.000568893 |
| 12 | 122437594 | 122437605 | WDR66 | 0.000571102 |
| 6 | 157496153 | 157496197 | ARID1B | 0.000582411 |
| 10 | 12240681 | 12240702 | CDC123 | 0.000583412 |
| 14 | 81448951 | 81448962 | TSHR | 0.000592107 |
| 15 | 42144967 | 42144979 | SPTBN5 | 0.000594059 |
| 5 | 413428 | 413452 | AHRR | 0.000609595 |
| 11 | 128350396 | 128350418 | ETS1 | 0.00061665 |
| 8 | 28707622 | 28707636 | INTS9 | 0.000622665 |
| 2 | 179478746 | 179478757 | MIR548N, TTN-AS1 | 0.000625586 |
| 12 | 107360876 | 107360887 | TMEM263 | 0.000627707 |
| 7 | 20180425 | 20180476 | MACC1(NM_182762: c.*144_*93del0) | 0.000651972 |
| 18 | 67614674 | 67614695 | CD226 | 0.000662659 |
| 17 | 39776817 | 39776829 | KRT17 | 0.000670661 |
| 8 | 103287850 | 103287864 | UBR5 | 0.000670661 |
| 15 | 66794298 | 66794311 | RPL4 | 0.000677048 |
| 12 | 95488339 | 95488354 | FGD6 | 0.000714213 |
| 3 | 47604262 | 47604281 | CSPG5 | 0.000761905 |
| 3 | 184573472 | 184573483 | VPS8 | 0.000766088 |
| 12 | 90005161 | 90005173 | ATP2B1 | 0.000766812 |
| 9 | 86588185 | 86588199 | HNRNPK | 0.000769112 |
| 7 | 104747784 | 104747796 | KMT2E | 0.000774194 |
| 3 | 108224685 | 108224701 | MYH15 | 0.000776972 |
| 20 | 16359356 | 16359369 | KIF16B | 0.000777717 |
| 9 | 98230924 | 98230947 | LOC100507346 | 0.000784902 |
| 12 | 19475610 | 19475621 | PLEKHA5 | 0.00079968 |
| 6 | 97615907 | 97615918 | MIR548H3 | 0.000820793 |
| 1 | 202736222 | 202736233 | KDM5B | 0.000826674 |
| 8 | 37704283 | 37704295 | BRF2 | 0.000826674 |
| 6 | 111685185 | 111685219 | REV3L | 0.000840336 |
| 20 | 36431202 | 36431215 | CTNNBL1 | 0.000853339 |
| 2 | 176995682 | 176995693 | HOXD8 | 0.000869187 |
| 19 | 9528423 | 9528438 | ZNF266 | 0.000883002 |
| 1 | 46650191 | 46650201 | TSPAN1 | 0.000926351 |
| 8 | 141293914 | 141293943 | TRAPPC9 | 0.000938925 |
| 15 | 28427670 | 28427687 | HERC2 | 0.00094156 |
| 13 | 50235208 | 50235218 | EBPL | 0.000949367 |
| 3 | 101575877 | 101575895 | NFKBIZ | 0.0009522 |
| 3 | 44815871 | 44815883 | KIF15 | 0.000957854 |
| 8 | 12598316 | 12598327 | LONRF1 | 0.000957854 |
| 21 | 43549808 | 43549820 | UMODL1 | 0.000964295 |
| 1 | 153617510 | 153617526 | CHTOP | 0.000964721 |
| 6 | 97000341 | 97000356 | UFL1 | 0.000975515 |
| 19 | 37155495 | 37155509 | ZNF461 | 0.000985011 |
| 11 | 107904512 | 107904524 | CUL5 | 0.000994554 |
| 12 | 64491002 | 64491019 | SRGAP1 | 0.001046296 |
| 5 | 158630629 | 158630653 | RNF145 | 0.001055276 |
| 2 | 55883509 | 55883523 | PNPT1 | 0.001056636 |
| 5 | 70809269 | 70809285 | BDP1 | 0.001083686 |
| 4 | 70280786 | 70280798 | UGT2B28(dist = 120018), UGT2B4(dist = 65085) | 0.001093534 |
| X | 3539376 | 3539390 | PRKX | 0.001098901 |
| 6 | 100988266 | 100988280 | ASCC3 | 0.001122209 |
| 6 | 151859146 | 151859161 | CCDC170 | 0.001125407 |
| 6 | 32147291 | 32147312 | RNF5P1 | 0.00113342 |
| 3 | 113377481 | 113377493 | KIAA2018 | 0.001136105 |
| 6 | 137325901 | 137325917 | IL20RA | 0.001139212 |
| 2 | 207013834 | 207013846 | NDUFS1 | 0.001145288 |
| 21 | 46602483 | 46602497 | ADARB1 | 0.001162308 |
| 7 | 102727051 | 102727067 | ARMC10 | 0.001182033 |
| 5 | 132408905 | 132408918 | HSPA4 | 0.001194172 |
| 11 | 59368218 | 59368235 | OSBP | 0.001211924 |
| 1 | 160302354 | 160302365 | COPA | 0.001220575 |
| 14 | 75199555 | 75199568 | FCF1 | 0.00122489 |
| 15 | 83808031 | 83808046 | HDGFRP3 | 0.001228555 |
| 3 | 179298584 | 179298597 | ACTL6A | 0.001236858 |
| 15 | 72244247 | 72244286 | MYO9A | 0.001240145 |
| 2 | 170917983 | 170917996 | UBR3 | 0.001249844 |
| 17 | 7726648 | 7726698 | DNAH2 | 0.001267579 |
| 10 | 13633928 | 13633944 | PRPF18 | 0.001274968 |
| 17 | 39976721 | 39976731 | FKBP10 | 0.001297641 |
| 3 | 130733046 | 130733058 | ASTE1 | 0.001300632 |
| 17 | 34151245 | 34151260 | TAF15 | 0.001360421 |
| 3 | 57850505 | 57850518 | SLMAP | 0.001393035 |
| 6 | 111702448 | 111702460 | REV3L | 0.001403153 |
| 17 | 57762402 | 57762414 | CLTC | 0.001413708 |
| 3 | 160131236 | 160131250 | SMC4 | 0.001457224 |
| 11 | 108549186 | 108549200 | DDX10 | 0.001495647 |
| 1 | 65129233 | 65129268 | CACHD1 | 0.001495647 |
| 1 | 36884530 | 36884544 | OSCP1 | 0.001534319 |
| 2 | 136527318 | 136527332 | UBXN4 | 0.001543125 |
| 3 | 38938299 | 38938322 | SCN11A | 0.001579511 |
| 15 | 50570999 | 50571024 | GABPB1 | 0.001603849 |
| 2 | 64139831 | 64139844 | VPS54 | 0.001630435 |
| 5 | 55034756 | 55034770 | DDX4 | 0.001645123 |
| 13 | 111932586 | 111932617 | ARHGEF7 | 0.001699958 |
| 11 | 134064516 | 134064540 | NCAPD3 | 0.001708817 |
| 2 | 186689258 | 186689269 | FSIP2 | 0.001708817 |
| 7 | 73666736 | 73666750 | RFC2 | 0.001715717 |
| 20 | 55206241 | 55206253 | TFAP2C | 0.001718846 |
| 7 | 56126036 | 56126047 | CCT6A | 0.00172117 |
| 19 | 53905129 | 53905144 | ZNF765 | 0.001731836 |
| 5 | 64084746 | 64084771 | CWC27 | 0.001761597 |
| 15 | 70961809 | 70961821 | UACA | 0.001805986 |
| 3 | 48491343 | 48491366 | ATRIP | 0.001806215 |
| 7 | 92129161 | 92129183 | PEX1 | 0.00181686 |
| 20 | 41306488 | 41306500 | PTPRT | 0.001854305 |
| 6 | 152539326 | 152539345 | SYNE1 | 0.001858494 |
| 15 | 50873112 | 50873154 | TRPM7 | 0.001858494 |
| 3 | 10018739 | 10018754 | EMC3 | 0.001877006 |
| 2 | 15676703 | 15676717 | NBAS | 0.00188383 |
| 22 | 46679850 | 46679864 | TTC38 | 0.001885133 |
| 12 | 66531936 | 66531947 | TMBIM4 | 0.001941371 |
| 4 | 187560860 | 187560871 | FAT1 | 0.00194742 |
| 14 | 31191658 | 31191682 | SCFD1 | 0.001970046 |
| 9 | 15458080 | 15458092 | SNAPC3 | 0.001983544 |
| 12 | 107237602 | 107237626 | RIC8B | 0.001984018 |
| 18 | 59942586 | 59942600 | KIAA1468 | 0.002011494 |
| 14 | 104145625 | 104145642 | KLC1 | 0.002063242 |
| 13 | 28133956 | 28133972 | LNX2 | 0.00212999 |
| 6 | 134494708 | 134494722 | SGK1 | 0.002307337 |
| 10 | 12215870 | 12215884 | NUDT5 | 0.002394913 |
| 17 | 9501525 | 9501545 | CFAP52 | 0.002419744 |
| 17 | 57278908 | 57278920 | PRR11 | 0.002445031 |
| 14 | 31762500 | 31762511 | HEATR5A | 0.002451559 |
| 17 | 5324558 | 5324611 | RPAIN | 0.002457002 |
| 2 | 36986088 | 36986108 | VIT | 0.002457002 |
| 10 | 81036869 | 81036879 | ZMIZ1 | 0.002596054 |
| 12 | 104714847 | 104714867 | TXNRD1 | 0.002631729 |
| 10 | 64975001 | 64975012 | JMJD1C | 0.002667088 |
| 5 | 68413042 | 68413072 | SLC30A5 | 0.002677702 |
| 2 | 56102061 | 56102072 | EFEMP1 | 0.002695418 |
| 1 | 45472798 | 45472829 | HECTD3 | 0.002700187 |
| 2 | 27463091 | 27463105 | CAD | 0.002713704 |
| 2 | 54163830 | 54163905 | PSME4 | 0.002754337 |
| 12 | 104515109 | 104515121 | NFYB | 0.002757078 |
| 7 | 103206001 | 103206013 | RELN | 0.002925697 |
| 18 | 649879 | 649895 | TYMSOS(NM_001012716: c.*120_*104del0) | 0.002963439 |
| 1 | 103471456 | 103471487 | COL11A1 | 0.003078376 |
| 2 | 207027356 | 207027368 | EEF1B2 | 0.003081827 |
| 12 | 21327652 | 21327667 | SLCO1B1 | 0.003081827 |
| 7 | 133881908 | 133881936 | LRGUK | 0.003221819 |
| 10 | 61956386 | 61956407 | ANK3 | 0.003266874 |
| 5 | 63803508 | 63803522 | RGS7BP | 0.00330263 |
| X | 135292012 | 135292028 | FHL1 | 0.003378378 |
| 17 | 5044640 | 5044653 | USP6 | 0.003392501 |
| 17 | 28773060 | 28773074 | CPD | 0.003410796 |
| 14 | 35899480 | 35899493 | NFKBIA(dist = 25520), INSM2(dist = 103755) | 0.003411857 |
| 1 | 89449508 | 89449520 | RBMXL1 | 0.003496503 |
| 8 | 37611414 | 37611428 | ERLIN2 | 0.003517546 |
| 5 | 139936680 | 139936694 | SRA1 | 0.003544037 |
| 16 | 90141288 | 90141302 | PRDM7 | 0.003704708 |
| 5 | 134032954 | 134032965 | SEC24A | 0.003764412 |
| 1 | 14057425 | 14057439 | PRDM2 | 0.003795385 |
| 21 | 34655334 | 34655346 | IL10RB | 0.003806927 |
| 15 | 55972660 | 55972674 | PRTG | 0.003850113 |
| 22 | 41545024 | 41545039 | EP300 | 0.003850165 |
| 13 | 111340025 | 111340053 | CARS2 | 0.003891377 |
| 10 | 75214137 | 75214149 | PPP3CB | 0.003894145 |
| 10 | 116203903 | 116203928 | ABLIM1 | 0.004034497 |
| 15 | 25319287 | 25319304 | SNORD116-10 | 0.004039951 |
| 5 | 94903575 | 94903589 | ARSK | 0.004058202 |
| 3 | 132179072 | 132179085 | DNAJC13 | 0.004077817 |
| 2 | 183832100 | 183832113 | NCKAP1 | 0.004104649 |
| 15 | 102287989 | 102288000 | TARSL2(dist = 23344), OR4F6(dist = 57923) | 0.004130466 |
| 1 | 170927570 | 170927588 | MROH9 | 0.004172571 |
| 1 | 173797451 | 173797462 | DARS2 | 0.004172571 |
| 5 | 137507104 | 137507121 | BRD8 | 0.004173092 |
| 3 | 113376110 | 113376147 | KIAA2018 | 0.0042042 |
| 11 | 88068256 | 88068270 | CTSC | 0.004274795 |
| 12 | 69090573 | 69090587 | NUP107 | 0.004406754 |
| 11 | 118247264 | 118247277 | UBE4A | 0.004409435 |
| 12 | 43826277 | 43826290 | ADAMTS20 | 0.004427021 |
| 10 | 28872473 | 28872498 | WAC | 0.004545455 |
| 7 | 95818864 | 95818897 | SLC25A13 | 0.004591776 |
| 6 | 24412431 | 24412445 | MRS2 | 0.004598134 |
| 6 | 46636556 | 46636581 | SLC25A27 | 0.004634401 |
| 7 | 24789116 | 24789134 | DFNA5 | 0.00466523 |
| 8 | 68139326 | 68139338 | ARFGEF1 | 0.00472565 |
| 9 | 2083313 | 2083338 | SMARCA2 | 0.004834508 |
| 8 | 110334770 | 110334782 | NUDCD1 | 0.004868549 |
| 4 | 114176848 | 114176860 | ANK2 | 0.004937786 |
| 18 | 18619416 | 18619428 | ROCK1 | 0.004970882 |
| 11 | 102272460 | 102272474 | TMEM123 | 0.005094144 |
| 4 | 48381682 | 48381704 | SLAIN2 | 0.005096981 |
| 11 | 108058769 | 108058785 | NPAT | 0.005097955 |
| 12 | 50867998 | 50868014 | LARP4 | 0.005102041 |
| 1 | 215914883 | 215914896 | USH2A | 0.005224756 |
| 2 | 86998651 | 86998676 | RMND5A | 0.005230322 |
| 7 | 20826270 | 20826284 | SP8 | 0.005235602 |
| 9 | 32485288 | 32485313 | DDX58 | 0.00544955 |
| 19 | 5739320 | 5739333 | CATSPERD | 0.005494505 |
| 15 | 74536285 | 74536301 | CCDC33 | 0.005541183 |
| 1 | 24180806 | 24180833 | FUCA1 | 0.005558934 |
| 10 | 76349020 | 76349033 | ADK | 0.005603164 |
| 7 | 5567254 | 5567271 | ACTB(NM_001101: c.*125_*108del0) | 0.005649718 |
| 16 | 22271671 | 22271685 | EEF2K | 0.00566619 |
| 5 | 81601142 | 81601155 | ATP6AP1L(NM_001017971: c.-79_-66del-) | 0.005747126 |
| X | 24084241 | 24084254 | EIF2S3 | 0.005820582 |
| 12 | 6858889 | 6858905 | MLF2 | 0.005843734 |
| 5 | 176729736 | 176729749 | RAB24 | 0.005881775 |
| 6 | 90366329 | 90366347 | LOC101929057 | 0.005894638 |
| 17 | 80443296 | 80443308 | NARF | 0.005913256 |
| 9 | 85987879 | 85987892 | FRMD3 | 0.006035135 |
| 19 | 55501348 | 55501365 | NLRP2 | 0.006565027 |
| 20 | 46266543 | 46266558 | NCOA3 | 0.006672895 |
| 10 | 101731593 | 101731631 | DNMBP | 0.006756757 |
| 15 | 48717850 | 48717884 | FBN1 | 0.006896552 |
| 16 | 67409030 | 67409054 | LRRC36 | 0.006944444 |
| 8 | 95750744 | 95750759 | DPY19L4 | 0.006944444 |
| 17 | 54981571 | 54981588 | TRIM25 | 0.007076105 |
| 7 | 75513199 | 75513231 | RHBDD2 | 0.007142857 |
| 1 | 52403174 | 52403187 | RAB3B | 0.007142857 |
| 5 | 162945387 | 162945400 | MAT2B(NM_182796: c.*18_*31del0, | 0.007188258 |
| | | | NM_013283: c.*18_*31 del0) | |
| 1 | 101194965 | 101194977 | VCAM1 | 0.007225164 |
| 14 | 95999676 | 95999689 | SNHG10 | 0.007260544 |
| 12 | 14599896 | 14599919 | ATF7IP | 0.007308175 |
| 5 | 7885782 | 7885795 | MTRR | 0.007374771 |
| 6 | 90347394 | 90347406 | LYRM2 | 0.007633588 |
| 8 | 117864952 | 117864973 | RAD21 | 0.007633588 |
| 16 | 69293926 | 69293945 | SNTB2 | 0.007664042 |
| 8 | 120580546 | 120580569 | ENPP2 | 0.007664725 |
| 1 | 67516191 | 67516202 | SLC35D1 | 0.007878914 |
| 1 | 213009178 | 213009196 | SPATA45 | 0.007959728 |
| 14 | 35497274 | 35497286 | SRP54 | 0.008049905 |
| 10 | 34985197 | 34985210 | PARD3 | 0.008080842 |
| X | 153216185 | 153216195 | HCFC1 | 0.008130081 |
| 15 | 45446084 | 45446109 | DUOX1 | 0.008151034 |
| 4 | 91549147 | 91549168 | CCSER1 | 0.00823979 |
| 8 | 139380103 | 139380130 | FAM135B | 0.008264463 |
| 10 | 26825151 | 26825168 | APBB1IP | 0.008322941 |
| 1 | 207679180 | 207679198 | CRI | 0.008474576 |
| 10 | 94366347 | 94366370 | KIF11 | 0.008522708 |
| 10 | 105990647 | 105990660 | CFAP43 | 0.00862069 |
| 5 | 158526517 | 158526550 | EBF1(NM_024007: c.-31_-64del0, | 0.008624591 |
| | | | NM_001290360: c.-31_-64del0, | |
| | | | NM_182708: c.-31_-64del0) | |
| 17 | 33998753 | 33998764 | AP2B1 | 0.00872633 |
| 13 | 20056698 | 20056716 | TPTE2 | 0.008928571 |
| 5 | 58021809 | 58021820 | RAB3C | 0.009009009 |
| 4 | 1806012 | 1806024 | FGFR3 | 0.009021513 |
| X | 40588550 | 40588561 | MED14 | 0.009250243 |
| 1 | 33135035 | 33135055 | RBBP4 | 0.009345794 |
| 2 | 203384776 | 203384799 | BMPR2 | 0.009345794 |
| 1 | 98011812 | 98011842 | DPYD | 0.00937633 |
| 1 | 181714466 | 181714481 | CACNA1E | 0.009405926 |
| X | 109416688 | 109416703 | TMEM164(NM_017698: c.*9_*24del0, | 0.009416538 |
| | | | NM_032227: c.*9_*24del0) | |
| 1 | 63096855 | 63096873 | DOCK7 | 0.009433962 |
| 20 | 29623380 | 29623389 | FRG1B | 0.00952381 |
| 17 | 45105631 | 45105644 | RPRML(dist = 49017), ARL17A(dist = 3308) | 0.009615385 |
| 7 | 86823977 | 86823988 | DMTF1 | 0.009615385 |
| 14 | 96848565 | 96848580 | GSKIP | 0.009708738 |
| 2 | 99936265 | 99936280 | TXNDC9 | 0.01 |
| X | 135328309 | 135328320 | MAP7D3 | 0.01010101 |
| 5 | 31435975 | 31435987 | DROSHA | 0.010309278 |
| 1 | 39352116 | 39352127 | RHBDL2(NM_017821: c.*60_*49del0) | 0.010309278 |
| 7 | 154745910 | 154745921 | PAXIP1 | 0.010309278 |
| 9 | 740769 | 740788 | KANK1 | 0.010356963 |
| 15 | 41060070 | 41060085 | DNAJC17(NM_018163: c.*68_*53del0) | 0.010416667 |
| 19 | 23845789 | 23845807 | ZNF675 | 0.010416667 |
| X | 15364158 | 15364186 | PIR-FIGF | 0.010416667 |
| 15 | 43784435 | 43784447 | RNU6-28P | 0.010425965 |
| 2 | 211521213 | 211521243 | CPS1 | 0.01052978 |
| 17 | 12920526 | 12920549 | ELAC2 | 0.010638298 |
| 16 | 19063220 | 19063235 | TMC7 | 0.010658937 |
| 12 | 31820564 | 31820578 | METTL20 | 0.010928962 |
| 2 | 162081128 | 162081140 | TANK | 0.010928962 |
| 7 | 138951165 | 138951177 | UBN2 | 0.011049724 |
| 12 | 27841925 | 27841938 | PPFIBP1 | 0.0110951 |
| 13 | 73636910 | 73636929 | KLF5 | 0.01110156 |
| 16 | 67470052 | 67470095 | HSD11B2 | 0.011111111 |
| 2 | 222290623 | 222290635 | EPHA4(NM_004438: c.*125_*113del0) | 0.011235955 |
| 17 | 35538304 | 35538314 | ACACA | 0.011235955 |
| 12 | 1909656 | 1909667 | CACNA2D4 | 0.011363636 |
| 18 | 19110456 | 19110477 | ESCO1 | 0.011378114 |
| 17 | 39723415 | 39723439 | KRT9 | 0.011477574 |
| 5 | 147785802 | 147785816 | FBXO38 | 0.011558576 |
| 2 | 86348737 | 86348749 | PTCD3 | 0.011560694 |
| 14 | 74426284 | 74426298 | COQ6 | 0.011976048 |
| 16 | 5129689 | 5129703 | ALG1 | 0.012121212 |
| 7 | 94042504 | 94042515 | COLLA2 | 0.012195122 |
| 2 | 10928694 | 10928709 | PDIA6 | 0.012259994 |
| 1 | 113159364 | 113159384 | ST7L | 0.012269939 |
| 2 | 178364328 | 178364342 | AGPS | 0.012577028 |
| 1 | 168205770 | 168205781 | SFT2D2 | 0.012578616 |
| 6 | 148761410 | 148761421 | SASH1 | 0.012658228 |
| 2 | 238925144 | 238925166 | UBE2F-SCLY | 0.012738854 |
| 18 | 29238076 | 29238091 | B4GALT6 | 0.012794551 |
| 22 | 26886953 | 26886982 | SRRD | 0.01282012 |
| 9 | 70993092 | 70993104 | PGM5 | 0.012866192 |
| 5 | 146773709 | 146773725 | DPYSL3 | 0.012903226 |
| 2 | 58362150 | 58362161 | VRK2 | 0.012987013 |
| 5 | 94206957 | 94206971 | MCTP1 | 0.012987013 |
| 12 | 112515954 | 112515966 | NAA25 | 0.012987013 |
| 11 | 66618396 | 66618426 | PC | 0.013071895 |
| 14 | 59790749 | 59790763 | DAAM1 | 0.013157895 |
| 11 | 102080325 | 102080341 | YAP1 | 0.013288501 |
| 8 | 131193153 | 131193166 | ASAP1 | 0.013314349 |
| 5 | 37326073 | 37326084 | NUP155 | 0.013333333 |
| 14 | 89878206 | 89878246 | FOXN3 | 0.013333333 |
| 3 | 192516152 | 192516163 | MB21D2(NM_178496: c.*23_*12del0) | 0.013333333 |
| 10 | 70252822 | 70252837 | SLC25A16 | 0.013422819 |
| 2 | 219341717 | 219341730 | USP37 | 0.013513514 |
| 5 | 179150788 | 179150800 | CANX | 0.013605442 |
| 12 | 31289662 | 31289674 | DDX11(dist = 31937), FAM60A(dist = 143846) | 0.013605442 |
| 19 | 7707445 | 7707455 | STXBP2 | 0.013753651 |
| 2 | 101581294 | 101581308 | NPAS2 | 0.013764251 |
| 15 | 54586266 | 54586278 | UNC13C | 0.013784009 |
| X | 83403148 | 83403160 | RPS6KA6 | 0.013888889 |
| 7 | 103828911 | 103828922 | ORC5 | 0.013888889 |
| 18 | 180211 | 180228 | USP14 | 0.014048048 |
| 17 | 28811834 | 28811848 | GOSR1 | 0.014084507 |
| 14 | 93686568 | 93686587 | UBR7 | 0.014182614 |
| 19 | 47656338 | 47656359 | SAE1 | 0.014184397 |
| 1 | 32119347 | 32119376 | COL16A1 | 0.014459225 |
| 9 | 8338847 | 8338866 | PTPRD | 0.014470918 |
| 10 | 111651598 | 111651609 | XPNPEP1 | 0.01459854 |
| 11 | 86120270 | 86120294 | CCDC81 | 0.014705882 |
| 12 | 989800 | 989815 | WNK1 | 0.01476888 |
| 11 | 67209167 | 67209194 | CORO1B | 0.014925373 |
| 13 | 53239754 | 53239767 | SUGT1 | 0.015037594 |
| 5 | 36123065 | 36123135 | LMBRD2 | 0.015151515 |
| 4 | 7817686 | 7817710 | AFAP1 | 0.0152262 |
| 10 | 5788110 | 5788126 | FAM208B | 0.015294739 |
| 20 | 37126168 | 37126180 | RALGAPB | 0.015384615 |
| 10 | 32327037 | 32327053 | KIF5B | 0.015503876 |
| 6 | 70966421 | 70966435 | COL9A1 | 0.015639415 |
| 6 | 89614390 | 89614408 | RNGTT | 0.015748031 |
| 8 | 124244005 | 124244016 | C8orf76, ZHX1-C8orf76 | 0.015873016 |
| 19 | 6013202 | 6013215 | LOC100128568 | 0.015873016 |
| 13 | 98634737 | 98634749 | IPO5 | 0.016042781 |
| 14 | 74443828 | 74443841 | ENTPD5 | 0.016129032 |
| 16 | 67286352 | 67286366 | SLC9A5 | 0.016393443 |
| 5 | 70805272 | 70805288 | BDP1 | 0.016445815 |
| 2 | 230656987 | 230657001 | TRIP12 | 0.016483516 |
| 1 | 211280724 | 211280748 | KCNH1 | 0.016496612 |
| 3 | 58296209 | 58296232 | RPP14 | 0.016496612 |
| 7 | 98630574 | 98630598 | LOC101927550 | 0.016528926 |
| 1 | 154144673 | 154144694 | TPM3 | 0.016666667 |
| 9 | 96209987 | 96209999 | FAM120AOS | 0.016770111 |
| 1 | 236590675 | 236590687 | EDARADD | 0.016806723 |
| 2 | 25656889 | 25656901 | DTNB | 0.016839036 |
| 5 | 32057910 | 32057923 | PDZD2 | 0.016949153 |
| 17 | 79176010 | 79176020 | CEP131 | 0.016949153 |
| 11 | 126174242 | 126174254 | DCPS | 0.016949153 |
| 12 | 111160513 | 111160526 | PPP1CC | 0.017045455 |
| 2 | 165365365 | 165365376 | GRB14 | 0.017241379 |
| 3 | 196532148 | 196532160 | PAK2 | 0.017241379 |
| 7 | 97939975 | 97939988 | BAIAP2L1 | 0.017241379 |
| 19 | 53277686 | 53277727 | ZNF600 | 0.017241379 |
| 1 | 36282435 | 36282452 | AGO4 | 0.017241379 |
| 1 | 183873947 | 183873965 | RGL1 | 0.017391304 |
| 1 | 217784431 | 217784441 | GPATCH2 | 0.017408801 |
| 5 | 50122645 | 50122660 | PARP8 | 0.01754386 |
| 2 | 10095014 | 10095031 | GRHL1 | 0.01754386 |
| 14 | 96794877 | 96794890 | ATG2B | 0.017699115 |
| 2 | 32983299 | 32983311 | TTC27 | 0.017699115 |
| 1 | 173921292 | 173921316 | RC3H1 | 0.017857143 |
| 15 | 44057632 | 44057645 | PDIA3 | 0.018018018 |
| 14 | 89037482 | 89037493 | ZC3H14 | 0.018119102 |
| 4 | 99214537 | 99214549 | RAP1GDS1 | 0.018181818 |
| 14 | 50121442 | 50121460 | POLE2 | 0.018181818 |
| 12 | 109510086 | 109510099 | USP30 | 0.018292683 |
| 2 | 11317833 | 11317856 | PQLC3 | 0.018348624 |
| 19 | 52646629 | 52646648 | ZNF616(dist = 3438), ZNF836(dist = 11477) | 0.018351539 |
| 11 | 28113094 | 28113107 | KIF18A | 0.018404908 |
| 16 | 71808499 | 71808519 | AP1G1 | 0.018404908 |
| 13 | 25839869 | 25839885 | MTMR6 | 0.018404908 |
| 12 | 14599855 | 14599867 | ATF7IP | 0.018518519 |
| 14 | 90499389 | 90499401 | TDP1 | 0.018518519 |
| 5 | 98208016 | 98208027 | CHD1 | 0.018518519 |
| 19 | 42933173 | 42933197 | LIPE-AS1 | 0.018518519 |
| 13 | 33637913 | 33637928 | KL | 0.018592703 |
| 14 | 96770942 | 96770967 | ATG2B | 0.018625096 |
| 14 | 64460536 | 64460551 | SYNE2 | 0.018630567 |
| 14 | 73959703 | 73959720 | C14orf169(NM_024644: c.*56_*73del0) | 0.018683428 |
| 20 | 35695535 | 35695562 | RBL1 | 0.018691589 |
| 3 | 121720514 | 121720530 | ILDR1 | 0.01872207 |
| 12 | 88566358 | 88566370 | TMTC3 | 0.01875 |
| 12 | 107398862 | 107398876 | CRY1 | 0.018867925 |
| 15 | 41364265 | 41364305 | INO80 | 0.018867925 |
| 14 | 39620884 | 39620896 | TRAPPC6B | 0.018867925 |
| 6 | 129955151 | 129955165 | ARHGAP18 | 0.018984235 |
| 16 | 70693872 | 70693884 | IL34 | 0.018990299 |
| 20 | 62163783 | 62163814 | PTK6 | 0.01910828 |
| 21 | 44513110 | 44513140 | U2AF1(NM_001025204: c.*102_*72del0, | 0.019230769 |
| | | | NM_006758: c.*102_*72del0, | |
| | | | NM_001025203: c.*102_*72del0) | |
| 1 | 114186351 | 114186362 | MAGI3 | 0.019230769 |
| 9 | 3937244 | 3937266 | GLIS3 | 0.019230769 |
| 13 | 108518658 | 108518714 | FAM155A | 0.019230769 |
| 3 | 111873902 | 111873917 | SLC9C1 | 0.019247877 |
| 12 | 89995053 | 89995065 | ATP2B1 | 0.019354839 |
| 21 | 47821449 | 47821460 | PCNT | 0.019417476 |
| 12 | 27803097 | 27803114 | PPFIBP1 | 0.019417476 |
| 11 | 65268479 | 65268492 | MALAT1 | 0.019480519 |
| 4 | 52928352 | 52928371 | SPATA18 | 0.019488012 |
| 14 | 69861462 | 69861490 | ERH | 0.019607843 |
| 12 | 78594396 | 78594427 | NAV3 | 0.01980198 |
| 3 | 143551065 | 143551076 | SLC9A9 | 0.01980198 |
| 12 | 57677520 | 57677531 | R3HDM2 | 0.01980198 |
| 6 | 56869464 | 56869475 | BEND6 | 0.01986755 |
| 12 | 69980447 | 69980462 | CCT2 | 0.019900498 |
| 9 | 33062183 | 33062195 | SMU1 | 0.02 |
| 5 | 88018305 | 88018317 | MEF2C(NM_001193350: c.*116_*104del0, | 0.02 |
| | | | NM_001131005: c.*116_*104del0, | |
| | | | NM_002397: c.*116_*104del0, | |
| | | | NM_001193347: c.*116_*104del0, | |
| | | | NM_001193348: c.*116_*104del0, | |
| | | | NM_001193349: c.*116_*104del0) | |
| 18 | 60034075 | 60034100 | TNFRSF11A | 0.02 |
| 1 | 100174683 | 100174709 | MIR548AA1, MIR548D1 | 0.02 |
| 16 | 58585178 | 58585191 | CNOT1 | 0.020134228 |
| 11 | 49196555 | 49196567 | FOLH1 | 0.020134228 |
| 2 | 101185539 | 101185550 | PDCL3 | 0.020134228 |
| 5 | 162884074 | 162884113 | NUDCD2 | 0.02020202 |
| 1 | 7912944 | 7912955 | UTS2 | 0.02020202 |
| 3 | 100569452 | 100569464 | ABI3BP | 0.02020202 |
| 16 | 51176119 | 51176131 | SALL1 | 0.020408163 |
| 1 | 15890342 | 15890359 | DNAJC16 | 0.020417133 |
| 7 | 35189891 | 35189905 | DPY19L2P1 | 0.020833333 |
| 15 | 42536227 | 42536238 | TMEM87A | 0.020833333 |
| 13 | 25355954 | 25355978 | RNF17 | 0.020833333 |
| 4 | 54319247 | 54319262 | FIP1L1 | 0.020833333 |
| 12 | 111701486 | 111701497 | CUX2 | 0.020933775 |
| 1 | 151380720 | 151380737 | POGZ | 0.021052632 |
| 12 | 2997058 | 2997074 | RHNO1 | 0.021052632 |
| 9 | 2088640 | 2088651 | SMARCA2 | 0.021052632 |
| 4 | 155457781 | 155457826 | PLRG1(NM_002669: c.*56_*11del0, | 0.021052632 |
| | | | NM_001201564: c.*56_*11del0) | |
| 3 | 170715672 | 170715694 | SLC2A2 | 0.021126761 |
| 3 | 24270412 | 24270424 | THRB | 0.021276596 |
| 1 | 22030891 | 22030902 | USP48 | 0.021276596 |
| 21 | 42823193 | 42823206 | MX1 | 0.021276596 |
| 6 | 144179190 | 144179202 | LTV1 | 0.021276596 |
| 2 | 45645684 | 45645695 | SRBD1 | 0.021276596 |
| 6 | 54791345 | 54791356 | FAM83B | 0.021276596 |
| 1 | 151010849 | 151010872 | BNIPL | 0.021276596 |
| 19 | 19308821 | 19308837 | RFXANK | 0.021276596 |
| 5 | 179032860 | 179032871 | RUFY1 | 0.021390374 |
| 7 | 7572355 | 7572381 | COL28A1 | 0.021390374 |
| 19 | 15511205 | 15511221 | AKAP8L | 0.021447254 |
| 18 | 30913142 | 30913153 | CCDC178 | 0.021505376 |
| 17 | 17796875 | 17796886 | TOM1L2 | 0.021505376 |
| 14 | 53213128 | 53213140 | STYX | 0.021582734 |
| 16 | 30665449 | 30665465 | PRR14 | 0.021621622 |
| 2 | 45806939 | 45806979 | SRBD1 | 0.021645022 |

TABLE 6
Microsatellite Repeat Marker Sequences for Rectum Adenocarcinoma (READ).
TABLE 6 - READ

| ch | start_pos | end_pos | | READ |
|---|---|---|---|---|
| (hg37) | (hg37 | (hg37) | gene | p-value |
| 10 | 112360315 | 112360333 | SMC3 | 8.22741E−07 |
| 16 | 3808052 | 3808066 | CREBBP | 9.18755E−07 |
| X | 19380975 | 19380993 | MAP3K15 | 9.18755E−07 |
| 11 | 118885668 | 118885701 | CCDC84 | 9.18755E−07 |
| 5 | 36123065 | 36123135 | LMBRD2 | 1.02919E−06 |
| 10 | 35314130 | 35314161 | CUL2 | 1.22792E−06 |
| 6 | 122733490 | 122733508 | HSF2 | 1.22792E−06 |
| 12 | 125587478 | 125587491 | AACS | 1.30467E−06 |
| 8 | 33356825 | 33356839 | MAK16(NM_032509: c.*678_*692del0) | 1.79261E−06 |
| 7 | 97939975 | 97939988 | BAIAP2L1 | 2.05071E−06 |
| 12 | 110834030 | 110834049 | ANAPC7 | 2.24629E−06 |
| 6 | 102130410 | 102130422 | GRIK2 | 2.3498E−06 |
| 9 | 75420448 | 75420463 | TMC1 | 2.48475E−06 |
| 13 | 79216356 | 79216381 | RNF219 | 2.53158E−06 |
| 19 | 6026288 | 6026302 | RFX2 | 2.85868E−06 |
| 17 | 29508819 | 29508836 | NF1 | 3.00528E−06 |
| 16 | 9010826 | 9010844 | USP7 | 3.1614E−06 |
| 4 | 146031236 | 146031253 | ABCE1 | 3.16205E−06 |
| X | 106092450 | 106092473 | TBC1D8B | 3.50527E−06 |
| 12 | 100496478 | 100496498 | UHRF1BP1L | 3.9732E−06 |
| 3 | 113442784 | 113442795 | NAA50 | 4.0016E−06 |
| 12 | 89853387 | 89853405 | POC1B | 4.1137E−06 |
| 5 | 172578549 | 172578567 | BNIP1 | 4.24695E−06 |
| 15 | 55841195 | 55841209 | PYGO1 | 4.34216E−06 |
| 1 | 222904869 | 222904881 | BROX | 4.59377E−06 |
| 3 | 41877473 | 41877487 | ULK4 | 4.62695E−06 |
| 12 | 85285920 | 85285938 | SLC6A15(NM_182767: c.-21 _-39del0, | 4.62695E−06 |
| | | | NM_018057: c.-21_-39del0) | |
| 10 | 64022582 | 64022593 | RTKN2 | 5.13927E−06 |
| 9 | 52625 | 52641 | FAM138C(dist = 16761), PGM5P3-AS1(dist = 20049) | 5.14597E−06 |
| 6 | 46636556 | 46636581 | SLC25A27 | 5.78368E−06 |
| 8 | 53543090 | 53543106 | RB1CC1 | 5.78368E−06 |
| 8 | 100160282 | 100160300 | VPS13B | 5.83216E−06 |
| 16 | 10567751 | 10567778 | ATF7IP2 | 6.1396E−06 |
| 2 | 8998775 | 8998788 | MBOAT2(NM_138799: c.*34_*21del0) | 6.1396E−06 |
| 1 | 149900985 | 149901002 | MTMR11(NM_001145862: c.*36_*19del0) | 6.52333E−06 |
| 2 | 141294271 | 141294299 | LRP1B | 6.52333E−06 |
| 2 | 203676382 | 203676403 | ICAIL | 6.73886E−06 |
| 6 | 49415351 | 49415368 | MUT | 6.93751E−06 |
| 2 | 32095023 | 32095056 | MEMO1 | 7.86936E−06 |
| 6 | 36452603 | 36452620 | KCTD20 | 8.39398E−06 |
| 11 | 47701629 | 47701642 | AGBL2 | 9.58121E−06 |
| 17 | 57937792 | 57937807 | TUBD1 | 9.98335E−06 |
| 16 | 75674252 | 75674271 | KARS | 1.02536E−05 |
| 4 | 120423686 | 120423697 | PDE5A | 1.02536E−05 |
| 13 | 28133956 | 28133972 | LNX2 | 1.09421E−05 |
| 9 | 79968285 | 79968309 | VPS13A | 1.0986E−05 |
| 8 | 24348425 | 24348437 | LOC101929294, LOC101929315 | 1.17849E−05 |
| 6 | 44364261 | 44364277 | CDC5L | 1.21579E−05 |
| 14 | 36218039 | 36218056 | RALGAPA1P | 1.23411E−05 |
| 4 | 76528928 | 76528945 | CDKL2 | 1.26579E−05 |
| 1 | 93687513 | 93687543 | CCDC18 | 1.30364E−05 |
| 2 | 120714401 | 120714418 | PTPN4 | 1.31447E−05 |
| 10 | 115380483 | 115380497 | NRAP | 1.36132E−05 |
| 4 | 39501722 | 39501754 | MIR1273H | 1.37813E−05 |
| 17 | 30687599 | 30687612 | ZNF207 | 1.58102E−05 |
| 3 | 33893929 | 33893949 | PDCD6IP | 1.64818E−05 |
| 15 | 55972660 | 55972674 | PRTG | 1.7411E−05 |
| 6 | 96971185 | 96971208 | UFL1 | 1.82682E−05 |
| 12 | 121618278 | 121618293 | P2RX7 | 1.84188E−05 |
| 20 | 58497481 | 58497497 | SYCP2(NM_014258: c.-13_-29del0) | 1.92694E−05 |
| 3 | 130733046 | 130733058 | ASTE1 | 1.99537E−05 |
| 3 | 33626219 | 33626236 | CLASP2 | 2.18531E−05 |
| 2 | 196762491 | 196762509 | DNAH7 | 2.27026E−05 |
| 3 | 169992958 | 169992994 | PRKCI | 2.28938E−05 |
| 12 | 129360344 | 129360381 | GLT1D1 | 2.35987E−05 |
| 7 | 137076100 | 137076123 | DGKI | 2.40015E−05 |
| 5 | 180481216 | 180481233 | BTNL9 | 2.51819E−05 |
| 2 | 207013834 | 207013846 | NDUFS1 | 2.51819E−05 |
| 21 | 37706087 | 37706100 | MORC3 | 2.51819E−05 |
| 3 | 52696310 | 52696322 | PBRM1 | 2.6441E−05 |
| 8 | 109462215 | 109462229 | EMC2 | 2.69354E−05 |
| 2 | 55883509 | 55883523 | PNPT1 | 2.77855E−05 |
| 15 | 52571270 | 52571284 | MYO5C | 2.77855E−05 |
| 14 | 104465128 | 104465144 | TDRD9 | 2.80324E−05 |
| 2 | 211527815 | 211527840 | CPS1 | 2.80324E−05 |
| 14 | 20344373 | 20344394 | OR4K2 | 2.81976E−05 |
| 9 | 91083260 | 91083278 | SPIN1 | 3.03143E−05 |
| 13 | 73409508 | 73409522 | PIBF1 | 3.06401E−05 |
| 2 | 99936265 | 99936280 | TXNDC9 | 3.07607E−05 |
| 4 | 99214537 | 99214549 | RAP1GDS1 | 3.07607E−05 |
| 15 | 59693969 | 59693985 | MYO1E(dist = 28898), FAM81A(dist = 36387) | 3.07607E−05 |
| 11 | 110306734 | 110306760 | FDX1 | 3.29579E−05 |
| 5 | 145647187 | 145647210 | RBM27 | 3.41763E−05 |
| 12 | 57032216 | 57032235 | ATP5B | 3.6075E−05 |
| 7 | 92900502 | 92900527 | CCDC132 | 3.68321E−05 |
| 19 | 12574740 | 12574762 | ZNF709(NM_152601: c.*70_*48del0) | 3.68321E−05 |
| 1 | 176050430 | 176050447 | RFWD2 | 3.8117E−05 |
| 4 | 141481203 | 141481225 | UCP1 | 3.88222E−05 |
| 12 | 109510086 | 109510099 | USP30 | 4.03161E−05 |
| 12 | 78594396 | 78594427 | NAV3 | 4.14008E−05 |
| 9 | 98009613 | 98009626 | FANCC | 4.21035E−05 |
| 12 | 123703074 | 123703086 | MPHOSPH9 | 4.26876E−05 |
| 20 | 3245219 | 3245232 | C20orf194 | 4.41424E−05 |
| 22 | 30051705 | 30051722 | NF2 | 4.51671E−05 |
| 6 | 116911413 | 116911425 | RWDD1 | 4.52489E−05 |
| X | 2343183 | 2343213 | DHRSX | 4.72337E−05 |
| 20 | 57569617 | 57569636 | NELFCD | 4.86855E−05 |
| 5 | 58270446 | 58270485 | PDE4D(NM_001197223: c.*45_*6del0, | 5.6899E−05 |
| | | | NM_001197221: c.*45_*6del0, | |
| | | | NM_001197220: c.*45_*6del0, | |
| | | | NM_001165899: c.*45_*6del0, | |
| | | | NM_001197218: c.*45_*6del0, | |
| | | | NM_001197222: c.*45_*6del0, | |
| | | | NM_001197219: c.*45_*6del0, | |
| | | | NM_006203: c.*45_*6del0, | |
| | | | NM_001104631: c.*45_*6del0) | |
| 5 | 14485161 | 14485175 | TRIO | 5.6899E−05 |
| 3 | 5249962 | 5249976 | EDEM1 | 5.71588E−05 |
| X | 118679315 | 118679331 | CXorf56 | 5.78168E−05 |
| 15 | 30010328 | 30010349 | TJP1 | 6.17055E−05 |
| 1 | 115280193 | 115280207 | CSDE1 | 6.17055E−05 |
| 4 | 95508108 | 95508128 | PDLIM5 | 6.17055E−05 |
| 14 | 36096255 | 36096278 | RALGAPA1P | 6.17055E−05 |
| 6 | 142691950 | 142691968 | GPR126 | 6.17055E−05 |
| 3 | 37176656 | 37176670 | LRRFIP2 | 6.43128E−05 |
| 6 | 89638988 | 89639004 | RNGTT | 6.43128E−05 |
| 14 | 60587879 | 60587900 | PCNXL4 | 6.43128E−05 |
| 13 | 31195409 | 31195432 | USPL1 | 6.43128E−05 |
| 2 | 197654578 | 197654593 | GTF3C3 | 6.58762E−05 |
| 17 | 40880759 | 40880775 | EZH1 | 6.99851E−05 |
| 3 | 111873902 | 111873917 | SLC9C1 | 7.1775E−05 |
| 2 | 212578379 | 212578394 | ERBB4 | 7.21521E−05 |
| 20 | 35695535 | 35695562 | RBL1 | 7.30727E−05 |
| 1 | 25679278 | 25679290 | TMEM50A | 7.30727E−05 |
| 5 | 86629039 | 86629073 | RASA1 | 7.30727E−05 |
| 12 | 56504143 | 56504159 | PA2G4 | 7.63446E−05 |
| 1 | 33402334 | 33402352 | RNF19B(NM_001127361: c.*555_*537del0, | 7.63446E−05 |
| | | | NM_153341: c.*73_*55del0, | |
| | | | NM_001300826: c.*73_*55del0) | |
| 15 | 64496611 | 64496630 | CSNK1G1 | 7.70891E−05 |
| 1 | 47123898 | 47123914 | ATPAF1 | 7.77863E−05 |
| 21 | 19653342 | 19653355 | TMPRSS15 | 7.98148E−05 |
| 16 | 47399670 | 47399687 | ITFG1 | 7.98148E−05 |
| 1 | 231094050 | 231094067 | TTC13 | 7.98148E−05 |
| 6 | 99817699 | 99817713 | COQ3 | 7.98148E−05 |
| 1 | 22033369 | 22033411 | USP48 | 7.98148E−05 |
| 22 | 42415224 | 42415240 | WBP2NL | 8.10307E−05 |
| 1 | 94964139 | 94964155 | ABCD3 | 8.24946E−05 |
| 1 | 63128836 | 63128850 | DOCK7 | 8.34986E−05 |
| 16 | 24185815 | 24185831 | PRKCB | 8.34986E−05 |
| 5 | 169020337 | 169020350 | SPDL1 | 8.34986E−05 |
| 2 | 141625834 | 141625854 | LRP1B | 8.34986E−05 |
| 4 | 166215662 | 166215692 | KLHL2 | 8.34986E−05 |
| 15 | 43170667 | 43170727 | TTBK2 | 8.34986E−05 |
| 7 | 81695842 | 81695859 | CACNA2D1 | 8.46197E−05 |
| 2 | 110922055 | 110922080 | NPHP1 | 8.7108E−05 |
| 20 | 47649496 | 47649525 | ARFGEF2 | 8.74126E−05 |
| 2 | 110325590 | 110325602 | SEPT10 | 9.15751E−05 |
| 10 | 13633928 | 13633944 | PRPF18 | 9.15751E−05 |
| 1 | 150676674 | 150676692 | HORMAD1 | 9.15751E−05 |
| 1 | 52242432 | 52242447 | OSBPL9 | 9.15751E−05 |
| 1 | 169819526 | 169819538 | C1orf112 | 9.15751E−05 |
| 11 | 49196555 | 49196567 | FOLH1 | 9.19202E−05 |
| 17 | 58289521 | 58289533 | USP32 | 9.38086E−05 |
| 1 | 216495186 | 216495197 | USH2A | 9.38086E−05 |
| 8 | 131193153 | 131193166 | ASAP1 | 9.52925E−05 |
| 5 | 178030941 | 178030955 | CLK4 | 9.60061E−05 |
| 2 | 136677957 | 136677968 | DARS | 9.60061E−05 |
| 12 | 50152601 | 50152618 | TMBIM6 | 9.77135E−05 |
| 16 | 90141288 | 90141302 | PRDM7 | 0.000100728 |
| 6 | 70459209 | 70459222 | LMBRD1 | 0.000101215 |
| 12 | 66522628 | 66522660 | LLPH | 0.000105764 |
| 15 | 43307839 | 43307852 | UBR1 | 0.000106666 |
| 4 | 146076632 | 146076644 | OTUD4 | 0.000107871 |
| 16 | 21623935 | 21623964 | METTL9 | 0.000108836 |
| 8 | 109247189 | 109247210 | EIF3E | 0.000108836 |
| 7 | 26883761 | 26883776 | SKAP2 | 0.000109421 |
| 8 | 28707622 | 28707636 | INTS9 | 0.000111142 |
| 1 | 92763091 | 92763128 | GLMN | 0.000111142 |
| 21 | 37706065 | 37706077 | MORC3 | 0.000111142 |
| 3 | 138248009 | 138248026 | CEP70 | 0.000111142 |
| 20 | 37191391 | 37191408 | RALGAPB | 0.000111142 |
| 2 | 179595571 | 179595589 | TTN | 0.000111142 |
| 9 | 103027226 | 103027243 | INVS | 0.000112918 |
| 6 | 11714639 | 11714665 | ADTRP(NM_001143948: c.*72_*46del0, | 0.000114893 |
| | | | NM_032744: c.*72_*46del0) | |
| 13 | 48664556 | 48664569 | MED4 | 0.000116891 |
| 3 | 146177594 | 146177608 | PLSCR2 | 0.000116891 |
| 5 | 33546113 | 33546143 | ADAMTS12 | 0.000116891 |
| 10 | 26802633 | 26802648 | APBB1IP | 0.000116891 |
| 6 | 133108763 | 133108784 | SLC18B1 | 0.000116891 |
| 22 | 40755427 | 40755439 | ADSL | 0.000116891 |
| 18 | 40503735 | 40503751 | RIT2 | 0.000121546 |
| 11 | 59368218 | 59368235 | OSBP | 0.000121714 |
| 3 | 129168854 | 129168881 | IFT122 | 0.000121714 |
| 5 | 38958956 | 38958971 | RICTOR | 0.000123043 |
| 7 | 105122891 | 105122924 | PUS7 | 0.000123043 |
| 14 | 64460536 | 64460551 | SYNE2 | 0.000129634 |
| 3 | 179083024 | 179083036 | MFN1 | 0.000129634 |
| 8 | 75157416 | 75157442 | JPH1 | 0.000136705 |
| 14 | 35550467 | 35550490 | LOC101927178 | 0.000136705 |
| 12 | 124281419 | 124281436 | DNAH10 | 0.000137554 |
| 20 | 10273694 | 10273715 | SNAP25 | 0.000142248 |
| 12 | 96694172 | 96694190 | CDK17 | 0.000142248 |
| 2 | 48046012 | 48046035 | FBXO11 | 0.0001443 |
| 8 | 67759411 | 67759438 | C8orf44-SGK3, SGK3 | 0.0001443 |
| 5 | 86679495 | 86679514 | RASA1 | 0.0001443 |
| 11 | 49088577 | 49088602 | TRIM64C(dist = 7913), FOLH1(dist = 79585) | 0.0001443 |
| 18 | 19119813 | 19119828 | ESCO1 | 0.000148093 |
| 5 | 150846858 | 150846882 | SLC36A1 | 0.000152468 |
| 15 | 33218472 | 33218487 | FMN1 | 0.000154264 |
| 14 | 58825825 | 58825847 | ARID4A | 0.000160511 |
| 9 | 30689085 | 30689104 | LINC01242(dist = 280633), ACO1(dist = 1695497) | 0.000160782 |
| X | 101409243 | 101409271 | BEX5(NM_001159560: c.-6_-34del0, | 0.000160782 |
| | | | NM_001012978: c.-6_-34del0) | |
| 20 | 480602 | 480627 | CSNK2A1 | 0.000161264 |
| 5 | 151180439 | 151180475 | G3BP1 | 0.000161264 |
| 8 | 141754888 | 141754905 | PTK2 | 0.000161264 |
| 2 | 15753307 | 15753321 | DDX1 | 0.000161264 |
| 16 | 53913911 | 53913925 | FTO | 0.000161264 |
| 12 | 51752085 | 51752103 | GALNT6 | 0.000164517 |
| 8 | 23712066 | 23712089 | STC1(NM_003155: c.-30_-53del0) | 0.000167673 |
| 15 | 83805209 | 83805222 | TM6SF1 | 0.000167673 |
| 10 | 12226870 | 12226882 | NUDT5 | 0.00017075 |
| 15 | 40062813 | 40062836 | FSIP1 | 0.00017075 |
| 14 | 55231286 | 55231297 | SAMD4A | 0.00017075 |
| 22 | 39079920 | 39079936 | TOMM22(NM_020243: c.*74_*90del0) | 0.000174963 |
| 16 | 20749284 | 20749296 | THUMPD1 | 0.000180995 |
| 5 | 134011834 | 134011848 | SEC24A | 0.000182256 |
| 7 | 36465606 | 36465622 | ANLN | 0.000182682 |
| 6 | 80910611 | 80910635 | BCKDHB | 0.000183284 |
| 2 | 158275111 | 158275135 | CYTIP | 0.000190862 |
| 1 | 32381592 | 32381607 | PTP4A2 | 0.000190862 |
| 4 | 141448696 | 141448718 | ELMOD2 | 0.000190862 |
| 16 | 46652286 | 46652305 | SHCBP1 | 0.000190862 |
| X | 119675354 | 119675368 | CUL4B | 0.000192077 |
| 5 | 78608348 | 78608371 | JMY | 0.000192077 |
| 8 | 133984814 | 133984834 | TG | 0.000199537 |
| 1 | 93691855 | 93691881 | CCDC18 | 0.000199537 |
| 1 | 197053561 | 197053577 | ASPM | 0.000199537 |
| 2 | 86683539 | 86683575 | KDM3A | 0.000199537 |
| 9 | 79835312 | 79835353 | VPS13A | 0.000199537 |
| 1 | 39900058 | 39900076 | MACF1 | 0.000204082 |
| 9 | 131715001 | 131715017 | NUP188 | 0.000208213 |
| 7 | 35218768 | 35218786 | DPY19L2P1 | 0.000208746 |
| 7 | 21603750 | 21603765 | DNAH11 | 0.000208746 |
| 14 | 96794877 | 96794890 | ATG2B | 0.000217108 |
| 17 | 30267579 | 30267601 | SUZ12 | 0.000217108 |
| 5 | 145888687 | 145888714 | TCERG1 | 0.000217673 |
| 7 | 92020452 | 92020465 | ANKIB1 | 0.000218531 |
| 9 | 133498017 | 133498047 | FUBP3 | 0.000218531 |
| 5 | 96093391 | 96093408 | CAST | 0.000218531 |
| 17 | 19744865 | 19744877 | ULK2 | 0.000222469 |
| 19 | 9361740 | 9361752 | OR7E24 | 0.000225258 |
| 21 | 33979904 | 33979922 | C21orf59 | 0.000229564 |
| 2 | 60988862 | 60988872 | PAPOLG | 0.000231267 |
| 9 | 88287588 | 88287615 | AGTPBP1 | 0.000231267 |
| 9 | 86354663 | 86354679 | GKAP1 | 0.000234412 |
| 12 | 104077096 | 104077126 | STAB2 | 0.000234412 |
| 2 | 103343275 | 103343295 | MFSD9 | 0.000234412 |
| 3 | 14696955 | 14696967 | CCDC174 | 0.000240015 |
| 2 | 179407763 | 179407779 | MIR548N, TTN-AS1 | 0.000246685 |
| 18 | 66367726 | 66367742 | TMX3 | 0.000246685 |
| 1 | 163313695 | 163313707 | NUF2 | 0.000246685 |
| 17 | 29195444 | 29195460 | ATAD5 | 0.000251819 |
| 12 | 12974842 | 12974853 | DDX47 | 0.000251819 |
| 5 | 10394911 | 10394930 | MARCH6 | 0.000252912 |
| 12 | 97306603 | 97306631 | NEDD1 | 0.000262895 |
| 16 | 31202698 | 31202716 | FUS | 0.000262895 |
| 3 | 113169372 | 113169386 | SPICE1 | 0.000262895 |
| 3 | 160144007 | 160144039 | SMC4 | 0.000263505 |
| 12 | 43964612 | 43964623 | ADAMTS20(dist = 18888), PUS7L(dist = 157787) | 0.000263505 |
| 3 | 97605460 | 97605472 | CRYBG3 | 0.00026441 |
| 3 | 113377481 | 113377493 | KIAA2018 | 0.00026441 |
| 8 | 48732074 | 48732096 | PRKDC | 0.00026441 |
| 3 | 183558468 | 183558512 | PARL | 0.00026441 |
| 2 | 201778717 | 201778733 | ORC2 | 0.000273411 |
| 2 | 157352734 | 157352747 | GPD2 | 0.000276853 |
| 2 | 219341717 | 219341730 | USP37 | 0.000277855 |
| 2 | 128854948 | 128854963 | UGGTI | 0.000277855 |
| 3 | 180320897 | 180320910 | TTC14 | 0.000281889 |
| 1 | 207237233 | 207237262 | PFKFB2 | 0.000281889 |
| 12 | 112119387 | 112119420 | BRAP | 0.000284495 |
| 4 | 113506885 | 113506900 | ZGRF1 | 0.000289019 |
| 14 | 55636060 | 55636079 | DLGAP5 | 0.000289019 |
| 1 | 245025697 | 245025717 | HNRNPU | 0.000292227 |
| 5 | 67584512 | 67584525 | PIK3R1(NM_181524: c.-52_-39del-) | 0.000301908 |
| 16 | 23457290 | 23457308 | COG7 | 0.000301908 |
| X | 48214185 | 48214200 | SSX3 | 0.000302024 |
| 8 | 120856021 | 120856034 | DSCC1 | 0.00030525 |
| 10 | 88458996 | 88459025 | LDB3 | 0.00030525 |
| 5 | 37027560 | 37027590 | NIPBL | 0.000307607 |
| 6 | 149846372 | 149846387 | PPIL4 | 0.000315576 |
| 5 | 35068814 | 35068826 | PRLR | 0.000315576 |
| 6 | 157495951 | 157495966 | ARID1B | 0.000316456 |
| 13 | 48828102 | 48828118 | ITM2B | 0.000321564 |
| 1 | 175087683 | 175087694 | TNN | 0.000324086 |
| 20 | 13140617 | 13140637 | SPTLC3 | 0.000324123 |
| 6 | 165809963 | 165809979 | PDE10A | 0.000324123 |
| 7 | 2406737 | 2406755 | EIF3B | 0.000324123 |
| 14 | 103915242 | 103915254 | MARK3 | 0.000324123 |
| 14 | 35242702 | 35242726 | BAZ1A | 0.000335345 |
| 6 | 36569717 | 36569737 | SRSF3 | 0.000335345 |
| 2 | 64139831 | 64139844 | VPS54 | 0.000341763 |
| 6 | 33408890 | 33408917 | SYNGAP1 | 0.00034188 |
| 14 | 96770942 | 96770967 | ATG2B | 0.000343013 |
| 4 | 76997157 | 76997173 | ART3 | 0.000345488 |
| 2 | 239241445 | 239241460 | TRAF3IP1 | 0.000345488 |
| 10 | 89720622 | 89720649 | PTEN | 0.000345488 |
| 20 | 33033065 | 33033083 | ITCH | 0.000348432 |
| 5 | 61745737 | 61745750 | IPO11 | 0.000348432 |
| 12 | 73012649 | 73012663 | TRHDE | 0.000349926 |
| 5 | 172421760 | 172421776 | ATP6V0E1 | 0.000359749 |
| 1 | 33087567 | 33087584 | ZBTB8OS | 0.000361869 |
| 14 | 102550069 | 102550088 | HSP90AA1 | 0.000381723 |
| 11 | 48970767 | 48970782 | OR4A47(dist = 459493), TRIM49B(dist = 82370) | 0.000381723 |
| 5 | 65372793 | 65372805 | ERBB2IP | 0.000392454 |
| 16 | 15159022 | 15159039 | PDXDC1, RRN3 | 0.000397573 |
| 1 | 170927570 | 170927588 | MROH9 | 0.000397875 |
| 12 | 94575162 | 94575177 | PLXNC1 | 0.000399074 |
| 4 | 119674051 | 119674062 | SEC24D | 0.000403161 |
| 21 | 40650627 | 40650652 | BRWD1 | 0.000403161 |
| 2 | 66796098 | 66796109 | MEIS1 | 0.000403161 |
| 5 | 36629801 | 36629815 | SLC1A3 | 0.000404858 |
| 2 | 91887905 | 91887919 | LOC654342(dist = 39930), GGT8P(dist = 75449) | 0.000404858 |
| 5 | 162884074 | 162884113 | NUDCD2 | 0.000414079 |
| 16 | 69293926 | 69293945 | SNTB2 | 0.000417493 |
| 11 | 108141955 | 108141971 | ATM | 0.000417679 |
| 6 | 75890933 | 75890959 | COL12A1 | 0.000428961 |
| 3 | 10018739 | 10018754 | EMC3 | 0.000434783 |
| 1 | 114495348 | 114495383 | HIPK1 | 0.000437063 |
| 19 | 33406199 | 33406229 | CEP89 | 0.000438819 |
| 14 | 65472874 | 65472892 | MAX(NM_001271069: c.*48_*30del0, | 0.000442092 |
| | | | NM_197957: c.*48_*30del0) | |
| 2 | 48734331 | 48734349 | PPP1R21 | 0.000443095 |
| 9 | 5811331 | 5811349 | ERMP1 | 0.000452489 |
| 3 | 10103903 | 10103946 | FANCD2 | 0.000452489 |
| 1 | 63270823 | 63270838 | ATG4C | 0.000457875 |
| 13 | 43872451 | 43872468 | ENOX1 | 0.000459807 |
| 4 | 25366051 | 25366071 | ZCCHC4 | 0.000461411 |
| 13 | 26151184 | 26151196 | ATP8A2 | 0.0004662 |
| 21 | 33312539 | 33312554 | HUNK | 0.0004662 |
| 7 | 99796751 | 99796765 | STAG3 | 0.0004662 |
| 15 | 82517622 | 82517671 | EFTUD1 | 0.000474158 |
| 2 | 45806939 | 45806979 | SRBD1 | 0.000474158 |
| 7 | 77584159 | 77584171 | PHTF2 | 0.000480031 |
| 1 | 233225957 | 233225969 | PCNXL2 | 0.000480192 |
| 6 | 42611936 | 42611951 | UBR2 | 0.000480769 |
| 6 | 117725383 | 117725399 | ROS1 | 0.000480769 |
| 8 | 104075165 | 104075177 | ATP6V1C1 | 0.000480769 |
| 12 | 28412398 | 28412416 | CCDC91 | 0.00048558 |
| 1 | 79383748 | 79383762 | ELTDI | 0.000489837 |
| 6 | 49815873 | 49815888 | CRISP1 | 0.000494071 |
| 10 | 17204246 | 17204272 | TRDMT1 | 0.000494968 |
| 2 | 160092703 | 160092716 | WDSUB1 | 0.000496032 |
| 14 | 59787325 | 59787338 | DAAM1 | 0.000496032 |
| 6 | 71571688 | 71571707 | SMAP1(NM_001281439: c.*1751_*1770del0, | 0.000496032 |
| | | | NM_021940: c.*1651_*1670del0, | |
| | | | NM_001044305: c.*1651_*1670del0, | |
| | | | NM_001281440: c.*1651_*1670del0) | |
| 17 | 39522910 | 39522922 | KRT33B | 0.000496032 |
| 4 | 17841869 | 17841881 | NCAPG | 0.000503639 |
| 17 | 1265304 | 1265326 | YWHAE | 0.000510204 |
| 10 | 76349020 | 76349033 | ADK | 0.000511467 |
| 2 | 209195472 | 209195485 | PIKFYVE | 0.000512033 |
| 9 | 125655308 | 125655324 | RC3H2 | 0.000512033 |
| 2 | 66670211 | 66670223 | MEIS1 | 0.000512033 |
| 9 | 102730697 | 102730712 | STX17 | 0.000514801 |
| 11 | 58892376 | 58892387 | FAM111B | 0.000514801 |
| 12 | 104714847 | 104714867 | TXNRD1 | 0.000535737 |
| 3 | 121202433 | 121202476 | POLQ | 0.000535737 |
| 6 | 131948816 | 131948831 | MED23 | 0.000539227 |
| 21 | 27136460 | 27136473 | GABPA | 0.000546448 |
| 9 | 7128277 | 7128289 | KDM4C | 0.000546448 |
| 3 | 93754286 | 93754317 | ARL13B | 0.00055571 |
| 4 | 89352469 | 89352487 | HERC6 | 0.00055571 |
| 1 | 215824195 | 215824226 | USH2A | 0.000560224 |
| 18 | 67613932 | 67613959 | CD226 | 0.000560224 |
| 4 | 128814425 | 128814449 | PLK4 | 0.000564972 |
| 19 | 10741681 | 10741697 | SLC44A2 | 0.000568355 |
| 18 | 67614674 | 67614695 | CD226 | 0.000578168 |
| 5 | 118500981 | 118501000 | DMXL1 | 0.000578168 |
| 12 | 45771901 | 45771918 | ANO6 | 0.000580032 |
| 17 | 46190093 | 46190110 | SNX11 | 0.000580032 |
| 2 | 38216766 | 38216779 | RMDN2-AS1 | 0.000580032 |
| 3 | 112253193 | 112253208 | ATG3 | 0.000582798 |
| 17 | 10317164 | 10317176 | MYHAS | 0.000584454 |
| 7 | 107875143 | 107875165 | NRCAM | 0.000584454 |
| 4 | 25315637 | 25315653 | ZCCHC4 | 0.000594177 |
| 2 | 111406968 | 111406984 | BUB1 | 0.000601076 |
| 2 | 169994021 | 169994058 | LRP2 | 0.000604961 |
| 1 | 118502087 | 118502108 | WDR3(NM_006784: c.*17_*38del0) | 0.000604961 |
| 4 | 170658944 | 170658956 | C40rf27 | 0.000604961 |
| 3 | 156411774 | 156411807 | TIPARP | 0.000604961 |
| 12 | 64491002 | 64491019 | SRGAPI | 0.000611154 |
| 13 | 51522081 | 51522094 | RNASEH2B | 0.000611154 |
| 13 | 43491778 | 43491796 | EPSTII | 0.000615214 |
| 13 | 39608335 | 39608360 | PROSERI | 0.000626566 |
| 8 | 67969596 | 67969610 | COPS5 | 0.000626566 |
| X | 133511862 | 133511890 | PHF6 | 0.000626566 |
| 1 | 74901739 | 74901760 | FPGT-TNNI3K, TNNI3K | 0.000629347 |
| 3 | 38257551 | 38257583 | OXSR1 | 0.000635571 |
| 19 | 38282490 | 38282505 | ZNF573(dist = 12260), LOC644554(dist = 25546) | 0.000639386 |
| 16 | 21809188 | 21809208 | RRN3P1 | 0.000649351 |
| 6 | 42626564 | 42626585 | UBR2 | 0.000656111 |
| 1 | 173797451 | 173797462 | DARS2 | 0.000658762 |
| 8 | 107704921 | 107704955 | OXR1 | 0.000672757 |
| 1 | 186330695 | 186330709 | MIR548F1 | 0.000672757 |
| 5 | 147785802 | 147785816 | FBXO38 | 0.000673401 |
| 6 | 88853530 | 88853542 | CNR1(NM_001160259: c.*45_*33del0, | 0.000673401 |
| | | | NM_001160226:c.*45_*33del0, | |
| | | | NM_001160258: c.*45_*33del0, | |
| | | | NM_033181: c.*45_*33del0, | |
| | | | NM_016083: c.*45_*33del0) | |
| 14 | 91365646 | 91365664 | RPS6KA5 | 0.000673401 |
| 5 | 122714134 | 122714153 | CEP120 | 0.000698812 |
| 3 | 113166840 | 113166859 | SPICE1 | 0.000698812 |
| 15 | 80460576 | 80460591 | FAH | 0.000704722 |
| 16 | 56535193 | 56535208 | BBS2 | 0.000708569 |
| 2 | 101885656 | 101885675 | CNOT11 | 0.000721501 |
| 9 | 77550305 | 77550323 | TRPM6(dist = 47295), C9orf40(dist = 11176) | 0.000721501 |
| 17 | 66904031 | 66904045 | ABCA8 | 0.000725241 |
| 5 | 118487582 | 118487593 | DMXL1 | 0.000725689 |
| 4 | 55976947 | 55976961 | KDR | 0.000734536 |
| 12 | 64679694 | 64679708 | C12orf56 | 0.000746095 |
| 9 | 37887570 | 37887587 | SLC25A51 | 0.000746095 |
| 16 | 31284618 | 31284634 | ITGAM | 0.00075188 |
| 7 | 149543201 | 149543232 | ZNF862 | 0.000754148 |
| 13 | 29238622 | 29238633 | POMP | 0.000754148 |
| 4 | 44691795 | 44691810 | GUF1 | 0.00076234 |
| 20 | 55206241 | 55206253 | TFAP2C | 0.000784314 |
| 6 | 111685185 | 111685219 | REV3L | 0.000784314 |
| 9 | 128064242 | 128064260 | GAPVD1 | 0.0008033 |
| 10 | 43199666 | 43199677 | LINC01518(dist = 12459), BMS1(dist = 78277) | 0.000806452 |
| 11 | 122928926 | 122928953 | HSPA8 | 0.000816273 |
| 8 | 80831393 | 80831407 | MRPS28 | 0.000816327 |
| X | 11793084 | 11793105 | MSL3 | 0.000816327 |
| 3 | 172052897 | 172052914 | FNDC3B | 0.000846203 |
| 4 | 128812230 | 128812245 | PLK4 | 0.000853313 |
| 5 | 72185591 | 72185607 | TNPO1 | 0.000853313 |
| 9 | 133498229 | 133498245 | FUBP3 | 0.000853313 |
| 2 | 196738263 | 196738274 | DNAH7 | 0.00087108 |
| 17 | 43587579 | 43587591 | LRRC37A4P | 0.000886525 |
| 12 | 81239721 | 81239737 | LIN7A | 0.000893596 |
| 2 | 179548695 | 179548712 | TTN | 0.000893596 |
| 3 | 19498221 | 19498253 | KCNH8 | 0.000925069 |
| 13 | 115011550 | 115011564 | CDC16 | 0.000936453 |
| 17 | 28773060 | 28773074 | CPD | 0.000936453 |
| 1 | 92108274 | 92108288 | CDC7(dist = 116953), TGFBR3(dist = 37612) | 0.000936453 |
| 16 | 15702117 | 15702141 | KIAA0430 | 0.000938086 |
| 1 | 132179950 | 132179979 | NTM | 0.000945257 |
| 7 | 151921037 | 151921049 | KMT2C | 0.000949367 |
| 8 | 130874614 | 130874639 | FAM49B | 0.000966184 |
| 18 | 77737714 | 77737726 | TXNL4A | 0.000966184 |
| 18 | 21046255 | 21046274 | RIOK3 | 0.000966184 |
| 13 | 23808732 | 23808745 | SGCG | 0.000969921 |
| 10 | 30605169 | 30605182 | MTPAP | 0.00097371 |
| 1 | 86435957 | 86435971 | COL24A1 | 0.000982096 |
| 15 | 73580950 | 73580962 | NEO1 | 0.000982096 |
| 9 | 130127691 | 130127703 | GARNL3 | 0.000982096 |
| 2 | 219350358 | 219350383 | USP37 | 0.000982096 |
| 3 | 12571420 | 12571434 | TSEN2(NM_001145395: c.*9_*23del0) | 0.000999001 |
| 12 | 30887772 | 30887832 | CAPRIN2 | 0.001010101 |
| 3 | 65361369 | 65361402 | MAGI1 | 0.001010101 |
| 1 | 241954005 | 241954024 | WDR64 | 0.001011122 |
| 1 | 220284112 | 220284124 | RNU5F-1 | 0.001012146 |
| 9 | 115060090 | 115060107 | MIR3134 | 0.001012146 |
| 13 | 42772765 | 42772789 | DGKH | 0.00102529 |
| 5 | 141520013 | 141520031 | NDFIP1 | 0.00102529 |
| 1 | 65099701 | 65099716 | CACHD1 | 0.001030752 |
| 11 | 35287000 | 35287014 | SLC1A2 | 0.001030752 |
| 17 | 11697015 | 11697027 | DNAH9 | 0.001030752 |
| 11 | 14539315 | 14539332 | PSMA1 | 0.001036258 |
| 18 | 649879 | 649895 | TYMSOS(NM_001012716: c.*120_*104del0) | 0.001052632 |
| 2 | 99614595 | 99614609 | TSGA10(NM_025244: c.*65_*51del0, | 0.001052632 |
| | | | NM_182911: c.*65_*51del0) | |
| 20 | 13695824 | 13695839 | ESF1 | 0.001052632 |
| 19 | 40587849 | 40587876 | ZNF780A | 0.001057082 |
| 1 | 151134579 | 151134590 | LYSMD1, TNFAIP8L2-SCNM1 | 0.001057082 |
| 11 | 86120270 | 86120294 | CCDC81 | 0.001060391 |
| 1 | 173490547 | 173490571 | SLC9C2 | 0.001081081 |
| X | 110494528 | 110494543 | CAPN6 | 0.001082676 |
| 1 | 220355521 | 220355533 | RAB3GAP2 | 0.001082676 |
| 1 | 117605130 | 117605145 | TTF2 | 0.001086518 |
| 13 | 25911034 | 25911067 | NUPL1 | 0.001094212 |
| 4 | 187627659 | 187627676 | FAT1 | 0.001094691 |
| 18 | 30913142 | 30913153 | CCDC178 | 0.00110742 |
| 1 | 24973106 | 24973116 | SRRM1 | 0.00110742 |
| 1 | 23650293 | 23650315 | HNRNPR | 0.00110742 |
| 11 | 59626477 | 59626491 | TCN1 | 0.00110742 |
| 8 | 35401930 | 35401941 | UNC5D | 0.00110742 |
| 6 | 32147291 | 32147312 | RNF5P1 | 0.0011107 |
| 2 | 230656987 | 230657001 | TRIP12 | 0.001141553 |
| 1 | 155701055 | 155701071 | DAP3 | 0.001141553 |
| 19 | 55044241 | 55044256 | KIR3DX1 | 0.001141553 |
| 12 | 416046 | 416060 | KDM5A | 0.00116144 |
| 2 | 11332238 | 11332250 | ROCK2 | 0.001173709 |
| 3 | 180679235 | 180679250 | FXR1 | 0.001173709 |
| 1 | 155884155 | 155884167 | KIAA0907 | 0.001173709 |
| 11 | 108549186 | 108549200 | DDX10 | 0.001173709 |
| 5 | 55034756 | 55034770 | DDX4 | 0.001185396 |
| 7 | 116556086 | 116556111 | CAPZA2 | 0.001185396 |
| 12 | 91372042 | 91372059 | EPYC | 0.001185771 |
| 1 | 196709736 | 196709747 | CFH | 0.001185995 |
| 7 | 123332378 | 123332391 | WASL | 0.001207243 |
| 12 | 124144572 | 124144584 | GTF2H3 | 0.001219512 |
| 6 | 52400479 | 52400497 | TRAM2 | 0.001221001 |
| 14 | 50760968 | 50760980 | L2HGDH | 0.001221001 |
| 2 | 61462921 | 61462939 | USP34 | 0.001221001 |
| 4 | 42626659 | 42626674 | ATP8A1 | 0.00123354 |
| 10 | 34573008 | 34573020 | PARD3 | 0.001242236 |
| 15 | 63447932 | 63447960 | RPS27L | 0.001242236 |
| 8 | 101253085 | 101253108 | SPAG1 | 0.001242236 |
| 19 | 327260 | 327277 | MIER2 | 0.001260989 |
| 10 | 74874082 | 74874112 | NUDT13 | 0.001260989 |
| 3 | 167045701 | 167045723 | ZBBX | 0.001260989 |
| 1 | 198717210 | 198717235 | PTPRC | 0.001262626 |
| 3 | 158526942 | 158526966 | MFSD1 | 0.001278772 |
| 13 | 111932586 | 111932617 | ARHGEF7 | 0.001278772 |
| 5 | 7896957 | 7896969 | MTRR | 0.001278772 |
| 7 | 8110836 | 8110848 | GLCCI1 | 0.001282051 |
| 2 | 196912236 | 196912247 | DNAH7 | 0.001282051 |
| 13 | 114113737 | 114113748 | DCUN1D2 | 0.001282051 |
| 5 | 94842718 | 94842730 | TTC37 | 0.001297641 |
| 7 | 139097271 | 139097295 | C7orf55-LUC7L2, LUC7L2 | 0.001316945 |
| 8 | 71037103 | 71037115 | NCOA2 | 0.001316945 |
| 7 | 135375909 | 135375932 | SLC13A4 | 0.001316945 |
| 2 | 208606875 | 208606908 | CCNYL1 | 0.001316945 |
| 7 | 72879898 | 72879914 | BAZ1B | 0.001316945 |
| 2 | 62063093 | 62063111 | FAM161A | 0.00132908 |
| 6 | 15651642 | 15651656 | DTNBP1 | 0.00132908 |
| 5 | 158126222 | 158126236 | EBF1 | 0.00132908 |

TABLE 7
Microsatellite Repeat Marker Sequences for Stomach Adenocarcinoma (STAD).
TABLE 7 - STAD

| ch | start_pos | end_pos | | STAD |
|---|---|---|---|---|
| (hg37) | (hg37) | (hg37) | gene | p-value |
| 19 | 14104688 | 14104703 | RFX1(NM_002918: c.-33_-48del0) | 1.28727E−45 |
| 7 | 102727051 | 102727067 | ARMC10 | 9.55899E−44 |
| 7 | 74608740 | 74608754 | GTF2IP1, LOC100093631 | 1.13938E−42 |
| 6 | 64289938 | 64289954 | PTP4A1 | 1.81433E−42 |
| 3 | 10018739 | 10018754 | EMC3 | 4.68392E−42 |
| 22 | 46679850 | 46679864 | TTC38 | 2.43723E−40 |
| 3 | 5249962 | 5249976 | EDEM1 | 2.75634E−40 |
| 12 | 122017894 | 122017916 | KDM2B | 9.66091E−40 |
| 20 | 37146131 | 37146146 | RALGAPB | 1.03207E−39 |
| 13 | 28133956 | 28133972 | LNX2 | 2.02103E−39 |
| 11 | 117152291 | 117152314 | RNF214 | 2.17143E−39 |
| 2 | 120714401 | 120714418 | PTPN4 | 1.11316E−38 |
| 21 | 34117067 | 34117080 | PAXBP1 | 3.95934E−38 |
| 8 | 103287850 | 103287864 | UBR5 | 1.031E−37 |
| 12 | 95488339 | 95488354 | FGD6 | 1.8452E−37 |
| 6 | 157495951 | 157495966 | ARID1B | 2.11186E−37 |
| 1 | 116940481 | 116940497 | ATP1A1-AS1 | 2.29381E−37 |
| 2 | 64112697 | 64112719 | UGP2 | 3.10569E−37 |
| 11 | 118369265 | 118369290 | KMT2A | 1.30605E−36 |
| 6 | 32147291 | 32147312 | RNF5P1 | 1.66973E−36 |
| 16 | 75674252 | 75674271 | KARS | 2.86736E−36 |
| 3 | 172052897 | 172052914 | FNDC3B | 4.26236E−36 |
| 8 | 120856021 | 120856034 | DSCC1 | 5.16378E−36 |
| 12 | 122762763 | 122762779 | CLIP1 | 6.15345E−36 |
| 15 | 41770639 | 41770652 | RTF1 | 6.19935E−36 |
| 19 | 327260 | 327277 | MIER2 | 6.98054E−36 |
| 10 | 70056619 | 70056632 | PBLD | 1.44221E−35 |
| 9 | 97555228 | 97555242 | C9orf3 | 1.63642E−35 |
| 19 | 11565819 | 11565831 | ELAVL3 | 1.90189E−35 |
| 9 | 131456159 | 131456172 | SET | 2.31906E−35 |
| 15 | 42503953 | 42503965 | TMEM87A | 2.94397E−35 |
| 7 | 7572355 | 7572381 | COL28A1 | 3.289E−35 |
| 12 | 125587478 | 125587491 | AACS | 3.89995E−35 |
| 13 | 48664556 | 48664569 | MED4 | 4.07756E−35 |
| 4 | 128814425 | 128814449 | PLK4 | 4.67887E−35 |
| 12 | 51323672 | 51323684 | METTL7A | 4.70829E−35 |
| 10 | 26825151 | 26825168 | APBB1IP | 6.26346E−35 |
| 22 | 35719378 | 35719394 | TOM1 | 6.68163E−35 |
| 13 | 79216356 | 79216381 | RNF219 | 1.1357E−34 |
| 14 | 51348281 | 51348299 | ABHD12B | 1.24803E−34 |
| 22 | 41545024 | 41545039 | EP300 | 1.87092E−34 |
| 7 | 65426054 | 65426069 | GUSB | 2.18258E−34 |
| 4 | 8472785 | 8472805 | TRMT44 | 3.41232E−34 |
| 12 | 99117397 | 99117414 | APAF1 | 3.69336E−34 |
| 2 | 207027356 | 207027368 | EEF1B2 | 4.00635E−34 |
| X | 129267464 | 129267478 | AIFM1 | 4.3021E−34 |
| 10 | 17110577 | 17110588 | CUBN | 4.65827E−34 |
| 18 | 60025458 | 60025474 | TNFRSF11A | 5.11943E−34 |
| 17 | 30500816 | 30500828 | RHOT1 | 6.40288E−34 |
| 2 | 228680153 | 228680168 | CCL20 | 8.57126E−34 |
| 20 | 36431202 | 36431215 | CTNNBL1 | 9.80737E−34 |
| 18 | 649879 | 649895 | TYMSOS(NM_001012716: c.*120_*104del0) | 1.02753E−33 |
| X | 12994867 | 12994886 | TMSB4X | 1.03104E−33 |
| 2 | 175246545 | 175246557 | CIR1 | 1.13971E−33 |
| 13 | 99890849 | 99890862 | MIR548AN | 1.39515E−33 |
| 12 | 132539500 | 132539513 | EP400 | 1.52667E−33 |
| 2 | 48046012 | 48046035 | FBXO11 | 1.59709E−33 |
| 1 | 14057425 | 14057439 | PRDM2 | 2.44452E−33 |
| 2 | 179407763 | 179407779 | MIR548N, TTN-AS1 | 2.98376E−33 |
| 6 | 165809963 | 165809979 | PDE10A | 3.16757E−33 |
| 4 | 128812230 | 128812245 | PLK4 | 3.63777E−33 |
| 3 | 121720514 | 121720530 | ILDR1 | 3.80152E−33 |
| 1 | 222904869 | 222904881 | BROX | 5.06052E−33 |
| 6 | 111329221 | 111329239 | RPF2 | 8.31212E−33 |
| 12 | 48360947 | 48360961 | TMEM106C | 9.87843E−33 |
| 13 | 113965002 | 113965017 | LAMP1 | 1.18782E−32 |
| 7 | 150918586 | 150918602 | ABCF2 | 1.21597E−32 |
| 5 | 33561022 | 33561043 | ADAMTS12 | 1.487E−32 |
| 22 | 30772228 | 30772244 | KIAA1656 | 1.60835E−32 |
| 3 | 113377481 | 113377493 | KIAA2018 | 1.67704E−32 |
| X | 118679315 | 118679331 | CXorf56 | 2.22403E−32 |
| 8 | 95186510 | 95186526 | CDH17 | 2.28161E−32 |
| 8 | 133984814 | 133984834 | TG | 2.38561E−32 |
| 5 | 55034756 | 55034770 | DDX4 | 2.66771E−32 |
| 2 | 61308569 | 61308585 | KIAA1841 | 3.01141E−32 |
| 17 | 73515055 | 73515068 | TSEN54 | 3.03314E−32 |
| 1 | 151384052 | 151384067 | POGZ | 3.0501E−32 |
| 7 | 26883761 | 26883776 | SKAP2 | 3.1717E−32 |
| 4 | 113107829 | 113107845 | C4orf32 | 3.63708E−32 |
| 11 | 8486375 | 8486389 | STK33 | 4.19111E−32 |
| 2 | 228159652 | 228159677 | LOC654841 | 4.33298E−32 |
| 2 | 61577667 | 61577680 | USP34 | 4.72874E−32 |
| 20 | 33338325 | 33338339 | NCOA6 | 5.9106E−32 |
| 8 | 124822507 | 124822519 | FAM91A1 | 6.04363E−32 |
| 17 | 57278908 | 57278920 | PRR11 | 6.5616E−32 |
| 3 | 121515940 | 121515953 | IQCB1 | 7.18008E−32 |
| 15 | 30010328 | 30010349 | TJP1 | 7.28827E−32 |
| 2 | 110922055 | 110922080 | NPHP1 | 7.29612E−32 |
| 8 | 80831393 | 80831407 | MRPS28 | 7.73674E−32 |
| 7 | 139097271 | 139097295 | C7orf55-LUC7L2, LUC7L2 | 8.06811E−32 |
| 12 | 104714847 | 104714867 | TXNRD1 | 8.99373E−32 |
| 14 | 64443258 | 64443278 | MIR548AZ | 1.08002E−31 |
| 6 | 64419024 | 64419039 | PHF3 | 1.16932E−31 |
| 1 | 94964139 | 94964155 | ABCD3 | 1.24921E−31 |
| 7 | 92900502 | 92900527 | CCDC132 | 1.29611E−31 |
| 10 | 101992923 | 101992938 | CWF19L1(NM_018294: c.*61_*46del0) | 1.70679E−31 |
| 20 | 60587772 | 60587788 | TAF4 | 1.72119E−31 |
| 5 | 14485161 | 14485175 | TRIO | 1.7941E−31 |
| 4 | 151850226 | 151850239 | LRBA | 2.54944E−31 |
| 12 | 133375066 | 133375092 | GOLGA3 | 2.84218E−31 |
| 5 | 147785802 | 147785816 | FBXO38 | 2.9737E−31 |
| 7 | 138270744 | 138270757 | TRIM24 | 3.46508E−31 |
| 2 | 32117217 | 32117230 | MEMO1 | 3.69556E−31 |
| 1 | 22030115 | 22030132 | USP48 | 3.94101E−31 |
| 19 | 50911947 | 50911960 | POLD1 | 4.11486E−31 |
| 19 | 9771286 | 9771300 | ZNF562 | 4.58568E−31 |
| 7 | 33217071 | 33217084 | BBS9 | 4.70048E−31 |
| 15 | 80460576 | 80460591 | FAH | 5.92599E−31 |
| 1 | 84956161 | 84956173 | RPF1 | 7.07072E−31 |
| 6 | 13316780 | 13316795 | TBC1D7 | 7.77151E−31 |
| 5 | 78416973 | 78416990 | BHMT | 9.53115E−31 |
| 1 | 44805012 | 44805054 | ERI3 | 1.0275E−30 |
| 1 | 150932953 | 150932989 | SETDB1 | 1.06404E−30 |
| 6 | 56504477 | 56504489 | RNU6-71P | 1.14019E−30 |
| 10 | 103567665 | 103567680 | MGEA5 | 1.22417E−30 |
| 1 | 86435957 | 86435971 | COL24A1 | 1.27042E−30 |
| 6 | 128330423 | 128330435 | PTPRK | 1.46169E−30 |
| 2 | 62063093 | 62063111 | FAM161A | 1.54421E−30 |
| 3 | 180320897 | 180320910 | TTC14 | 1.55368E−30 |
| 8 | 104922508 | 104922534 | RIMS2 | 1.71751E−30 |
| 8 | 30569609 | 30569622 | GSR | 1.72395E−30 |
| 3 | 138193025 | 138193038 | ESYT3 | 1.92985E−30 |
| 2 | 64331815 | 64331833 | PELI1 | 1.93697E−30 |
| 10 | 118891992 | 118892021 | VAX1 | 2.02847E−30 |
| 2 | 234178624 | 234178640 | ATG16L1 | 2.56955E−30 |
| X | 129546260 | 129546274 | RBMX2 | 3.04384E−30 |
| 6 | 15651642 | 15651656 | DTNBP1 | 3.09992E−30 |
| 17 | 66039938 | 66039951 | KPNA2 | 3.3665E−30 |
| 4 | 83970297 | 83970312 | COPS4 | 3.54802E−30 |
| 4 | 119666217 | 119666241 | SEC24D | 3.70716E−30 |
| 9 | 102730697 | 102730712 | STX17 | 4.73147E−30 |
| 9 | 15472611 | 15472623 | PSIP1 | 6.13511E−30 |
| 1 | 153949135 | 153949147 | JTB | 6.16896E−30 |
| 8 | 74204423 | 74204438 | RPL7 | 7.77021E−30 |
| 2 | 10928694 | 10928709 | PDIA6 | 7.83205E−30 |
| 17 | 15973417 | 15973449 | NCOR1 | 8.37048E−30 |
| 8 | 130874614 | 130874639 | FAM49B | 8.91674E−30 |
| 8 | 38940143 | 38940157 | ADAM9 | 9.20495E−30 |
| 8 | 142161666 | 142161681 | DENND3 | 1.12727E−29 |
| 17 | 66038979 | 66038999 | KPNA2 | 1.18635E−29 |
| 20 | 46266543 | 46266558 | NCOA3 | 1.21007E−29 |
| 15 | 55972660 | 55972674 | PRTG | 1.3928E−29 |
| 12 | 98989187 | 98989199 | SLC25A3 | 1.59855E−29 |
| 2 | 120620092 | 120620108 | PTPN4 | 1.60176E−29 |
| 12 | 104461711 | 104461723 | HCFC2 | 1.67358E−29 |
| 15 | 43927623 | 43927636 | CATSPER2 | 1.69383E−29 |
| 8 | 68140383 | 68140394 | ARFGEF1 | 1.81641E−29 |
| 1 | 78432506 | 78432521 | FUBP1 | 1.90834E−29 |
| 1 | 3549854 | 3549874 | WRAP73 | 2.36714E−29 |
| 5 | 70837260 | 70837275 | BDP1 | 2.50137E−29 |
| 5 | 134015495 | 134015508 | SEC24A | 2.52438E−29 |
| 2 | 79314062 | 79314073 | REG1B | 2.58249E−29 |
| 19 | 6026288 | 6026302 | RFX2 | 2.88305E−29 |
| 14 | 104145625 | 104145642 | KLC1 | 2.94657E−29 |
| 11 | 59626477 | 59626491 | TCN1 | 2.95675E−29 |
| 6 | 116914112 | 116914128 | RWDD1 | 3.50117E−29 |
| 7 | 152144203 | 152144216 | FABP5P3(dist = 4103), LINC01003(dist = 16993) | 3.89354E−29 |
| 5 | 94842718 | 94842730 | TTC37 | 4.10289E−29 |
| 1 | 175116046 | 175116071 | TNN | 4.99251E−29 |
| 11 | 47187055 | 47187087 | ARFGAP2 | 5.04417E−29 |
| 9 | 19334957 | 19334970 | DENND4C | 5.1499E−29 |
| 2 | 34907636 | 34907654 | GART | 5.5525E−29 |
| 6 | 163899794 | 163899807 | QKI | 5.6508E−29 |
| 11 | 74053485 | 74053497 | PGM2L1 | 6.42335E−29 |
| 8 | 62550924 | 62550938 | ASPH | 6.49943E−29 |
| 6 | 42611936 | 42611951 | UBR2 | 6.58913E−29 |
| 6 | 36452603 | 36452620 | KCTD20 | 6.60275E−29 |
| 4 | 17841869 | 17841881 | NCAPG | 6.81996E−29 |
| 3 | 19959707 | 19959719 | EFHB | 7.11458E−29 |
| 9 | 131743701 | 131743715 | NUP188 | 7.20219E−29 |
| X | 134025421 | 134025436 | MOSPD1 | 7.59034E−29 |
| 2 | 39240584 | 39240596 | SOS1 | 7.77384E−29 |
| 2 | 111406968 | 111406984 | BUB1 | 8.93529E−29 |
| 17 | 74729588 | 74729600 | METTL23 | 1.03849E−28 |
| 3 | 57850505 | 57850518 | SLMAP | 1.07548E−28 |
| 17 | 8417006 | 8417020 | MYH10 | 1.07706E−28 |
| 12 | 64491002 | 64491019 | SRGAP1 | 1.26622E−28 |
| 16 | 4559351 | 4559367 | HMOX2 | 1.28267E−28 |
| 1 | 41456808 | 41456823 | CTPS1 | 1.30576E−28 |
| 17 | 66041871 | 66041886 | KPNA2 | 1.46969E−28 |
| 13 | 26151184 | 26151196 | ATP8A2 | 1.62391E−28 |
| 1 | 63128836 | 63128850 | DOCK7 | 1.63257E−28 |
| 8 | 37611414 | 37611428 | ERLIN2 | 1.75926E−28 |
| 11 | 124624705 | 124624721 | ESAM | 2.02752E−28 |
| 11 | 124007701 | 124007714 | VWA5A | 2.03767E−28 |
| 8 | 23712066 | 23712089 | STC1(NM_003155: c.-30_-53del0) | 2.05249E−28 |
| 12 | 4645161 | 4645173 | C12orf4 | 2.0984E−28 |
| 6 | 25452453 | 25452466 | LRRC16A | 2.12232E−28 |
| 7 | 92020452 | 92020465 | ANKIB1 | 2.12232E−28 |
| 2 | 70456452 | 70456465 | TIA1 | 2.2463E−28 |
| 7 | 135099044 | 135099059 | CNOT4 | 2.35627E−28 |
| 11 | 73984657 | 73984669 | P4HA3 | 2.43161E−28 |
| 9 | 135773000 | 135773019 | TSC1 | 2.55479E−28 |
| 10 | 32575773 | 32575785 | EPC1 | 2.71151E−28 |
| 10 | 74899343 | 74899356 | ECD | 2.75224E−28 |
| 1 | 89449508 | 89449520 | RBMXL1 | 2.88271E−28 |
| 14 | 88407716 | 88407730 | GALC | 2.98955E−28 |
| 10 | 115380483 | 115380497 | NRAP | 2.99401E−28 |
| 10 | 116605738 | 116605750 | FAM160B1 | 3.08154E−28 |
| X | 135115669 | 135115681 | SLC9A6 | 3.53796E−28 |
| 9 | 163927 | 163943 | CBWD1 | 3.61255E−28 |
| 7 | 94989255 | 94989273 | PON3(NM_000940: c.*30_*12del0) | 3.63573E−28 |
| 3 | 191888452 | 191888477 | FGF12 | 3.64344E−28 |
| 6 | 90366329 | 90366347 | LOC101929057 | 4.1867E−28 |
| 2 | 112854563 | 112854576 | TMEM87B | 4.37261E−28 |
| 8 | 67759411 | 67759438 | C8orf44-SGK3, SGK3 | 4.7387E−28 |
| 11 | 108121410 | 108121426 | ATM | 5.06994E−28 |
| 5 | 93294451 | 93294463 | FAM172A | 5.12501E−28 |
| 12 | 53861636 | 53861650 | PCBP2 | 5.85829E−28 |
| 13 | 39608335 | 39608360 | PROSER1 | 5.87731E−28 |
| 8 | 141293914 | 141293943 | TRAPPC9 | 5.9086E−28 |
| 3 | 62153593 | 62153621 | PTPRG | 6.02936E−28 |
| 19 | 36145409 | 36145425 | COX6B1 | 6.23563E−28 |
| 3 | 48491343 | 48491366 | ATRIP | 6.34319E−28 |
| 8 | 131193153 | 131193166 | ASAP1 | 6.58505E−28 |
| 11 | 125488247 | 125488261 | STT3A | 6.99352E−28 |
| 12 | 104515109 | 104515121 | NFYB | 7.41136E−28 |
| 4 | 25315637 | 25315653 | ZCCHC4 | 7.52201E−28 |
| 5 | 172578549 | 172578567 | BNIP1 | 9.4251E−28 |
| 1 | 145456732 | 145456747 | NBPF10, NBPF20, POLR3GL | 9.85339E−28 |
| 11 | 59368218 | 59368235 | OSBP | 1.11222E−27 |
| 15 | 101550861 | 101550877 | LRRK1 | 1.12118E−27 |
| 5 | 74011541 | 74011556 | HEXB | 1.13187E−27 |
| 7 | 74119460 | 74119488 | LOC101926943 | 1.14187E−27 |
| 13 | 73409508 | 73409522 | PIBF1 | 1.22983E−27 |
| 2 | 64069277 | 64069293 | UGP2(NM_006759: c.-43_-27del-) | 1.35367E−27 |
| X | 100532479 | 100532505 | TAF7L | 1.41147E−27 |
| 12 | 57882764 | 57882777 | MARS | 1.46143E−27 |
| 4 | 108935768 | 108935822 | HADH | 1.84961E−27 |
| 2 | 201534173 | 201534187 | AOX1 | 2.23824E−27 |
| 4 | 114176848 | 114176860 | ANK2 | 2.25481E−27 |
| 12 | 107237602 | 107237626 | RIC8B | 2.2592E−27 |
| 17 | 1326754 | 1326769 | CRK(NM_005206: c.*183_*168del0, | 2.43472E−27 |
| | | | NM_016823: c.*53_*38del0) | |
| 11 | 47701629 | 47701642 | AGBL2 | 2.60331E−27 |
| 16 | 69293926 | 69293945 | SNTB2 | 2.81142E−27 |
| 9 | 17464479 | 17464491 | CNTLN | 2.82293E−27 |
| 8 | 28707622 | 28707636 | INTS9 | 2.93586E−27 |
| 19 | 34945331 | 34945344 | UBA2 | 3.15202E−27 |
| 12 | 123703074 | 123703086 | MPHOSPH9 | 3.20538E−27 |
| 8 | 37704283 | 37704295 | BRF2 | 3.66865E−27 |
| 2 | 74754818 | 74754830 | AUP1 | 3.86606E−27 |
| 20 | 7921106 | 7921120 | HAO1 | 4.20796E−27 |
| 3 | 158407930 | 158407945 | GFM1 | 4.69517E−27 |
| 15 | 25319287 | 25319304 | SNORD116-10 | 4.74832E−27 |
| 17 | 57762402 | 57762414 | CLTC | 4.86717E−27 |
| 17 | 44409890 | 44409927 | LRRC37A | 4.88321E−27 |
| 9 | 80343587 | 80343612 | GNAQ | 4.97372E−27 |
| 21 | 30435646 | 30435668 | CCT8 | 5.32326E−27 |
| 3 | 71008341 | 71008362 | FOXP1(NM_001244816: c.*57_*36del0, | 5.42766E−27 |
| | | | NM_001244812: c.*57_*36del0, | |
| | | | NM_001244813: c.*57_*36del0, | |
| | | | NM_001244808: c.*57_*36del0, | |
| | | | NM_001244815: c.*57_*36del0, | |
| | | | NM_001244814: c.*57_*36del0, | |
| | | | NM_032682: c.*57_*36del0, | |
| | | | NM_001244810: c.*57_*36del0) | |
| 1 | 167871297 | 167871312 | ADCY10 | 5.53076E−27 |
| 16 | 84907499 | 84907520 | CRISPLD2 | 5.84658E−27 |
| 2 | 101885656 | 101885675 | CNOT11 | 5.99198E−27 |
| 5 | 150282674 | 150282694 | ZNF300 | 6.38016E−27 |
| 5 | 140076413 | 140076436 | HARS2 | 6.8234E−27 |
| 16 | 57249981 | 57250000 | RSPRY1 | 7.02244E−27 |
| 3 | 160134195 | 160134221 | SMC4 | 7.09839E−27 |
| 10 | 5788110 | 5788126 | FAM208B | 7.41558E−27 |
| 16 | 19063220 | 19063235 | TMC7 | 7.43645E−27 |
| 5 | 70809269 | 70809285 | BDPI | 7.51021E−27 |
| 1 | 173795803 | 173795821 | DARS2 | 7.77883E−27 |
| 12 | 56500502 | 56500515 | PA2G4 | 7.87435E−27 |
| 13 | 111340025 | 111340053 | CARS2 | 8.22896E−27 |
| 15 | 45725103 | 45725138 | C15orf48 | 9.03216E−27 |
| 15 | 50596341 | 50596355 | GABPB1 | 1.01324E−26 |
| 13 | 50586051 | 50586064 | DLEU2 | 1.06985E−26 |
| 4 | 113568126 | 113568140 | LARP7 | 1.13001E−26 |
| 2 | 211179765 | 211179777 | MYL1 | 1.16606E−26 |
| 11 | 63149670 | 63149682 | SLC22A9 | 1.17544E−26 |
| 5 | 147793666 | 147793680 | FBXO38 | 1.20732E−26 |
| 15 | 72875521 | 72875535 | ARIH1 | 1.22861E−26 |
| 5 | 178030941 | 178030955 | CLK4 | 1.26563E−26 |
| X | 48214185 | 48214200 | SSX3 | 1.40426E−26 |
| 2 | 27463091 | 27463105 | CAD | 1.67084E−26 |
| 3 | 146177594 | 146177608 | PLSCR2 | 1.71727E−26 |
| 19 | 47700438 | 47700453 | SAE1 | 1.74952E−26 |
| 6 | 31832356 | 31832372 | SLC44A4 | 1.8027E−26 |
| 6 | 30619285 | 30619302 | C6orf136 | 1.85971E−26 |
| 2 | 204156968 | 204156980 | CYP20A1 | 1.88602E−26 |
| 2 | 128263363 | 128263377 | IWS1 | 1.902E−26 |
| 5 | 180481216 | 180481233 | BTNL9 | 1.92591E−26 |
| 2 | 47635523 | 47635537 | MSH2 | 2.04143E−26 |
| 5 | 82937251 | 82937262 | HAPLN1(NM_001884: c.*64_*53del0) | 2.23621E−26 |
| 9 | 99408079 | 99408094 | AAED1 | 2.29568E−26 |
| 1 | 150799125 | 150799138 | ARNT | 2.38177E−26 |
| 13 | 45973231 | 45973246 | SLC25A30 | 2.45175E−26 |
| 2 | 175246386 | 175246411 | CIR1 | 2.57125E−26 |
| 17 | 54981571 | 54981588 | TRIM25 | 2.62816E−26 |
| 4 | 52928352 | 52928371 | SPATA18 | 2.69973E−26 |
| 7 | 106523362 | 106523383 | PIK3CG | 2.76304E−26 |
| 19 | 21350520 | 21350533 | ZNF431 | 2.89881E−26 |
| 20 | 34430474 | 34430486 | PHF20 | 2.90299E−26 |
| 17 | 62748076 | 62748093 | LOC146880 | 2.9926E−26 |
| 3 | 185155430 | 185155442 | MAP3K13 | 3.07057E−26 |
| 3 | 33725814 | 33725828 | CLASP2 | 3.50466E−26 |
| 2 | 136527318 | 136527332 | UBXN4 | 3.55504E−26 |
| 15 | 41060070 | 41060085 | DNAJC17(NM_018163: c.*68_*53del0) | 3.5814E−26 |
| 15 | 83808031 | 83808046 | HDGFRP3 | 3.60952E−26 |
| 14 | 91372685 | 91372700 | RPS6KA5 | 3.63751E−26 |
| 12 | 107398862 | 107398876 | CRY1 | 3.68736E−26 |
| 14 | 31191658 | 31191682 | SCFD1 | 3.75564E−26 |
| 1 | 92763091 | 92763128 | GLMN | 3.88999E−26 |
| 1 | 116655183 | 116655197 | MAB21L3(NM_152367: c.-54_-40del-) | 3.89624E−26 |
| 1 | 207753824 | 207753846 | CR1 | 4.64132E−26 |
| 14 | 102476130 | 102476143 | DYNC1H1 | 4.86995E−26 |
| 11 | 118883880 | 118883892 | CCDC84 | 5.13074E−26 |
| 13 | 39435552 | 39435564 | FREM2 | 5.16696E−26 |
| 12 | 90005161 | 90005173 | ATP2B1 | 5.48804E−26 |
| 2 | 120366057 | 120366070 | CFAP221 | 6.27043E−26 |
| 1 | 202736222 | 202736233 | KDM5B | 6.48588E−26 |
| 1 | 155671899 | 155671929 | DAP3 | 6.67349E−26 |
| 12 | 31435608 | 31435620 | FAM60A(NM_001135811: c.*38_*26del0, | 6.74389E−26 |
| | | | NM_001135812: c.*38_*26del0, | |
| | | | NM_021238: c.*38_*26del0) | |
| 16 | 19619602 | 19619617 | C16orf62 | 6.92793E−26 |
| 5 | 139907750 | 139907762 | ANKHD1, ANKHD1-EIF4EBP3 | 7.16687E−26 |
| 2 | 197654578 | 197654593 | GTF3C3 | 7.58006E−26 |
| 13 | 77644856 | 77644877 | MYCBP2 | 7.84163E−26 |
| 11 | 65268479 | 65268492 | MALAT1 | 8.25136E−26 |
| 7 | 102825987 | 102826001 | DPY19L2P2 | 8.58113E−26 |
| 7 | 75651305 | 75651336 | STYXL1 | 8.74332E−26 |
| 3 | 30691871 | 30691882 | TGFBR2 | 9.14943E−26 |
| 14 | 65472874 | 65472892 | MAX(NM_001271069: c.*48_*30del0, | 9.3654E−26 |
| | | | NM_197957: c.*48_*30del0) | |
| 9 | 127287158 | 127287183 | NR6A1 | 1.13275E−25 |
| 11 | 22296094 | 22296106 | ANO5 | 1.20729E−25 |
| 1 | 220284112 | 220284124 | RNU5F-1 | 1.23682E−25 |
| 17 | 57937792 | 57937807 | TUBD1 | 1.34815E−25 |
| 3 | 123332875 | 123332892 | MYLK-AS1 | 1.34868E−25 |
| 2 | 15676703 | 15676717 | NBAS | 1.35141E−25 |
| 15 | 43307839 | 43307852 | UBR1 | 1.3818E−25 |
| 2 | 48032740 | 48032754 | MSH6 | 1.48442E−25 |
| 6 | 55739711 | 55739726 | BMP5(NM_021073: c.-48_-63del0) | 1.65309E−25 |
| 12 | 73012649 | 73012663 | TRHDE | 1.66273E−25 |
| 16 | 3808052 | 3808066 | CREBBP | 1.69752E−25 |
| 8 | 67969596 | 67969610 | COPS5 | 1.71665E−25 |
| 3 | 5024415 | 5024435 | BHLHE40 | 1.72335E−25 |
| 6 | 137325901 | 137325917 | IL20RA | 1.83349E−25 |
| 8 | 127595047 | 127595058 | FAM84B(dist = 24336), PCAT1(dist = 430341) | 1.91057E−25 |
| 7 | 138434066 | 138434081 | ATP6V0A4 | 1.97563E−25 |
| 14 | 20763824 | 20763838 | TTC5 | 2.28252E−25 |
| 1 | 54252939 | 54252952 | NDC1 | 2.46911E−25 |
| 8 | 38839302 | 38839316 | HTRA4 | 2.4758E−25 |
| 11 | 17526254 | 17526267 | USH1C | 2.54098E−25 |
| 4 | 76997157 | 76997173 | ART3 | 2.56062E−25 |
| 19 | 14153179 | 14153202 | IL27RA | 2.61144E−25 |
| 3 | 108224685 | 108224701 | MYH15 | 2.69648E−25 |
| 4 | 128621144 | 128621158 | INTU | 2.698E−25 |
| 9 | 2083313 | 2083338 | SMARCA2 | 3.02695E−25 |
| 20 | 13140617 | 13140637 | SPTLC3 | 3.43592E−25 |
| 2 | 157370018 | 157370033 | GPD2 | 3.5521E−25 |
| 12 | 133237753 | 133237768 | POLE | 3.73314E−25 |
| 4 | 146046123 | 146046135 | ABCE1 | 3.78371E−25 |
| 2 | 66796098 | 66796109 | MEIS1 | 4.00535E−25 |
| 15 | 48826427 | 48826440 | FBN1 | 4.42733E−25 |
| 2 | 99936265 | 99936280 | TXNDC9 | 4.60387E−25 |
| 16 | 70595514 | 70595525 | SF3B3 | 4.61027E−25 |
| 1 | 65138787 | 65138801 | CACHD1 | 4.85138E−25 |
| 5 | 78936847 | 78936860 | PAPD4 | 5.1291E−25 |
| 6 | 32166160 | 32166174 | NOTCH4 | 5.16035E−25 |
| 4 | 89711690 | 89711718 | FAM13A | 5.31308E−25 |
| 10 | 33496676 | 33496688 | NRP1 | 5.47626E−25 |
| 16 | 4873743 | 4873761 | GLYR1 | 5.51924E−25 |
| 3 | 97605460 | 97605472 | CRYBG3 | 5.65216E−25 |
| 4 | 47746602 | 47746616 | CORIN | 5.72865E−25 |
| 16 | 15159022 | 15159039 | PDXDC1, RRN3 | 5.76272E−25 |
| 1 | 165712555 | 165712571 | TMCO1 | 5.82979E−25 |
| 5 | 145647187 | 145647210 | RBM27 | 6.1352E−25 |
| 7 | 95775848 | 95775863 | SLC25A13 | 6.17853E−25 |
| 7 | 100843369 | 100843385 | MOGAT3 | 6.35445E−25 |
| 3 | 100438778 | 100438810 | TFG | 6.56508E−25 |
| 5 | 134015250 | 134015288 | SEC24A | 6.63113E−25 |
| 3 | 113655096 | 113655108 | GRAMD1C | 6.90378E−25 |
| 5 | 118576096 | 118576110 | DMXL1 | 7.21041E−25 |
| 18 | 24039893 | 24039904 | KCTD1 | 8.0175E−25 |
| 1 | 12368705 | 12368721 | VPS13D | 8.11113E−25 |
| 2 | 55883509 | 55883523 | PNPT1 | 8.61191E−25 |
| 17 | 39776817 | 39776829 | KRT17 | 8.64429E−25 |
| 12 | 122017848 | 122017866 | KDM2B | 8.66157E−25 |
| 6 | 99817699 | 99817713 | COQ3 | 8.76368E−25 |
| 19 | 43919911 | 43919926 | TEX101 | 9.24754E−25 |
| 11 | 56216401 | 56216413 | OR5R1(dist = 30693), OR5M9(dist = 13532) | 9.62753E−25 |
| 9 | 134049353 | 134049366 | NUP214 | 9.7287E−25 |
| 11 | 102080325 | 102080341 | YAP1 | 9.74675E−25 |
| 13 | 25899091 | 25899108 | NUPL1 | 1.01972E−24 |
| 6 | 44364261 | 44364277 | CDC5L | 1.1359E−24 |
| 2 | 230656987 | 230657001 | TRIP12 | 1.19674E−24 |
| 1 | 113657018 | 113657033 | LRIG2 | 1.19724E−24 |
| 11 | 57094842 | 57094869 | SSRP1 | 1.20319E−24 |
| 17 | 1584377 | 1584390 | PRPF8 | 1.32252E−24 |
| 9 | 21861902 | 21861914 | MTAP | 1.36108E−24 |
| 3 | 132173004 | 132173026 | DNAJC13 | 1.37153E−24 |
| 1 | 113159364 | 113159384 | ST7L | 1.45017E−24 |
| 4 | 89352469 | 89352487 | HERC6 | 1.45824E−24 |
| 17 | 74286032 | 74286047 | QRICH2 | 1.45824E−24 |
| 2 | 200800572 | 200800585 | TYW5 | 1.47009E−24 |
| X | 19380975 | 19380993 | MAP3K15 | 1.52333E−24 |
| 17 | 33288433 | 33288445 | CCT6B(NM_001193530: c.-21_-33del0, | 1.53997E−24 |
| | | | NM_001193529: c.-21_-33del0, | |
| | | | NM_006584: c.-21_-33del0) | |
| 2 | 27659821 | 27659845 | NRBP1 | 1.53997E−24 |
| 9 | 34343033 | 34343047 | NUDT2 | 1.58732E−24 |
| 12 | 56872770 | 56872791 | GLS2 | 1.62654E−24 |
| 15 | 51017381 | 51017393 | SPPL2A | 1.64404E−24 |
| 3 | 101575877 | 101575895 | NFKBIZ | 1.66704E−24 |
| 10 | 98336613 | 98336626 | TM9SF3 | 1.66776E−24 |
| 7 | 98649755 | 98649771 | SMURF1 | 1.72464E−24 |
| 2 | 27597190 | 27597204 | SNX17 | 1.86401E−24 |
| 10 | 112360315 | 112360333 | SMC3 | 1.89468E−24 |
| 14 | 57101560 | 57101576 | TMEM260 | 1.9325E−24 |
| 7 | 73666736 | 73666750 | RFC2 | 1.94512E−24 |
| 21 | 37706065 | 37706077 | MORC3 | 2.00629E−24 |
| 9 | 27062802 | 27062816 | IFT74(NM_001099223: c.*68_*82del0, | 2.03369E−24 |
| | | | NM_001099222: c.*68_*82del0, | |
| | | | NM_025103: c.*68_*82del0) | |
| 4 | 39233719 | 39233734 | MIR1273H | 2.04583E−24 |
| 11 | 16117684 | 16117698 | SOX6 | 2.05894E−24 |
| 1 | 169586220 | 169586233 | SELP | 2.14713E−24 |
| 8 | 100160282 | 100160300 | VPS13B | 2.32602E−24 |
| 2 | 9558905 | 9558924 | ITGB1BP1 | 2.4205E−24 |
| 19 | 10090461 | 10090482 | COL5A3 | 2.51013E−24 |
| 3 | 130733046 | 130733058 | ASTE1 | 2.53778E−24 |
| 15 | 66794298 | 66794311 | RPL4 | 2.71306E−24 |
| 10 | 25140413 | 25140425 | PRTFDC1 | 2.7448E−24 |
| 6 | 117026217 | 117026229 | KPNA5 | 2.91421E−24 |
| 1 | 231396485 | 231396509 | GNPAT | 3.14115E−24 |
| 4 | 41015553 | 41015566 | APBB2 | 3.23648E−24 |
| 17 | 29325662 | 29325674 | RNF135 | 3.27565E−24 |
| 6 | 126249747 | 126249771 | NCOA7 | 3.36146E−24 |
| 2 | 11332238 | 11332250 | ROCK2 | 3.44599E−24 |
| 12 | 56504143 | 56504159 | PA2G4 | 3.48105E−24 |
| 20 | 46282112 | 46282135 | NCOA3 | 3.51773E−24 |
| 22 | 41551231 | 41551249 | EP300 | 3.62396E−24 |
| 5 | 72185591 | 72185607 | TNPO1 | 3.63236E−24 |
| 20 | 33225781 | 33225808 | PIGU | 3.64795E−24 |
| 8 | 22138812 | 22138831 | PIWIL2 | 3.78516E−24 |
| 7 | 64275470 | 64275486 | ZNF138 | 3.9129E−24 |
| 12 | 102131090 | 102131108 | SYCP3 | 3.95456E−24 |
| 1 | 221875661 | 221875686 | DUSP10(NM_007207: c.*93_*68del0) | 3.95797E−24 |
| 5 | 88018369 | 88018402 | MEF2C(NM_001193350: c.*52_*19del0, | 4.12511E−24 |
| | | | NM_001131005: c.*52_*19del0, | |
| | | | NM_002397: c.*52_*19del0, | |
| | | | NM_001193347: c.*52_*19del0, | |
| | | | NM_001193348: c.*52_*19del0, | |
| | | | NM_001193349: c.*52_*19del0) | |
| 4 | 39511530 | 39511543 | MIR1273H | 4.20591E−24 |
| 9 | 112184972 | 112184987 | PTPN3 | 4.25595E−24 |
| 5 | 52358761 | 52358775 | ITGA2 | 4.3129E−24 |
| 14 | 39594134 | 39594146 | GEMIN2 | 4.36485E−24 |
| 7 | 75185990 | 75186002 | HIP1 | 4.45645E−24 |
| 8 | 42761288 | 42761308 | HOOK3 | 4.84565E−24 |
| 14 | 95571635 | 95571647 | DICER1 | 5.13886E−24 |
| 12 | 91372042 | 91372059 | EPYC | 5.5091E−24 |
| 6 | 152539326 | 152539345 | SYNE1 | 5.55656E−24 |
| 12 | 120741303 | 120741326 | SIRT4 | 5.77498E−24 |
| 3 | 160955882 | 160955898 | NMD3 | 5.96846E−24 |
| 11 | 113697927 | 113697939 | USP28 | 6.04939E−24 |
| 8 | 68139326 | 68139338 | ARFGEF1 | 6.06739E−24 |
| 1 | 112303077 | 112303090 | DDX20 | 6.14144E−24 |
| 20 | 31954819 | 31954834 | CDK5RAP1 | 6.15828E−24 |
| 15 | 37391752 | 37391765 | MEIS2(NM_170677: c.-85_-98del0, | 6.84041E−24 |
| | | | NM_170676: c.-85_-98del0, | |
| | | | NM_170675: c.-85_-98del0, | |
| | | | NM_170674: c.-85_-98del0, | |
| | | | NM_001220482: c.-85_-98del0) | |
| 2 | 21226218 | 21226230 | APOB | 6.85236E−24 |
| 20 | 30818497 | 30818514 | POFUT1 | 7.99199E−24 |
| 8 | 110535444 | 110535460 | PKHD1L1 | 8.01314E−24 |
| 12 | 31820564 | 31820578 | METTL20 | 8.09202E−24 |
| 2 | 99788147 | 99788158 | MITD1 | 8.11916E−24 |
| 10 | 69716602 | 69716614 | HERC4 | 8.59792E−24 |
| X | 48136515 | 48136526 | SSX1(dist = 9636), SSX9(dist = 24459) | 8.89604E−24 |
| 9 | 100997485 | 100997498 | TBC1D2 | 8.95696E−24 |
| 3 | 169992958 | 169992994 | PRKC1 | 9.31808E−24 |
| 7 | 91714092 | 91714106 | AKAP9 | 9.39861E−24 |
| 16 | 47399670 | 47399687 | ITFG1 | 9.40393E−24 |
| 6 | 80910611 | 80910635 | BCKDHB | 9.57803E−24 |
| 14 | 53513439 | 53513452 | DDHD1(NM_001160148: c.*47_*34del0, | 9.92846E−24 |
| | | | NM_001160147: c.*47_*34del0, | |
| | | | NM_030637: c.*47_*34del0) | |
| 4 | 187627659 | 187627676 | FATI | 1.06146E−23 |
| 5 | 67576330 | 67576346 | PIK3R1 | 1.07434E−23 |
| 8 | 109462215 | 109462229 | EMC2 | 1.08462E−23 |
| 8 | 134125650 | 134125662 | TG | 1.09878E−23 |
| 17 | 1383742 | 1383781 | MYO1C | 1.13446E−23 |
| 10 | 70962143 | 70962162 | SUPV3L1 | 1.14778E−23 |
| 21 | 42823193 | 42823206 | MX1 | 1.20656E−23 |
| 2 | 234916771 | 234916787 | TRPM8 | 1.21066E−23 |
| 2 | 64210968 | 64210991 | VPS54 | 1.24098E−23 |
| 19 | 34302673 | 34302689 | KCTD15 | 1.30787E−23 |
| 14 | 73079138 | 73079150 | RGS6(dist = 45900), DPF3(dist = 6854) | 1.37113E−23 |
| 10 | 32327037 | 32327053 | KIF5B | 1.42745E−23 |
| 7 | 100319680 | 100319696 | EPO | 1.50698E−23 |
| 21 | 32597972 | 32597988 | TIAM1 | 1.53271E−23 |
| 19 | 11411997 | 11412010 | TSPAN16 | 1.53859E−23 |
| X | 153994469 | 153994482 | DKC1 | 1.55988E−23 |
| 1 | 79383748 | 79383762 | ELTD1 | 1.58931E−23 |
| 14 | 91639611 | 91639627 | C14orf159 | 1.64087E−23 |
| 21 | 37760118 | 37760134 | CHAF1B | 1.68287E−23 |
| 6 | 62611334 | 62611348 | KHDRBS2 | 1.68424E−23 |
| 15 | 43430749 | 43430765 | TMEM62 | 1.73149E−23 |
| 15 | 102287989 | 102288000 | TARSL2(dist = 23344), OR4F6(dist = 57923) | 1.80266E−23 |
| 16 | 3433714 | 3433726 | ZSCAN32 | 1.8428E−23 |
| 1 | 35885037 | 35885058 | ZMYM4 | 1.84817E−23 |
| 1 | 166962119 | 166962131 | MAEL | 1.89614E−23 |
| 14 | 45587186 | 45587199 | FKBP3 | 1.91118E−23 |
| 11 | 93490699 | 93490715 | C11orf54 | 1.9662E−23 |
| 3 | 3216976 | 3216988 | CRBN | 1.98768E−23 |
| 13 | 23808732 | 23808745 | SGCG | 2.00592E−23 |
| 8 | 30969355 | 30969369 | WRN | 2.04194E−23 |
| 2 | 54146387 | 54146400 | PSME4 | 2.15025E−23 |
| 21 | 46602483 | 46602497 | ADARB1 | 2.16195E−23 |
| 14 | 104465128 | 104465144 | TDRD9 | 2.24287E−23 |
| 3 | 37176656 | 37176670 | LRRFIP2 | 2.29731E−23 |
| 2 | 209195472 | 209195485 | PIKFYVE | 2.3001E−23 |
| 3 | 164905648 | 164905660 | SLITRK3(NM_014926: c.*37_*25del0) | 2.31667E−23 |
| 14 | 81953889 | 81953901 | SEL1L | 2.42696E−23 |
| 1 | 114186351 | 114186362 | MAGI3 | 2.56246E−23 |
| 12 | 51752085 | 51752103 | GALNT6 | 2.5733E−23 |

TABLE 8
Microsatellite Repeat Marker Sequences for Brain Lower Grade Glioma (LGG).
TABLE 8 - LGG

| ch | start_pos | end_pos | | LGG |
|---|---|---|---|---|
| (hg37) | (hg37) | (hg37) | gene | p-value |
| 3 | 5249962 | 5249976 | EDEM1 | 0.003690037 |
| 9 | 127765856 | 127765868 | SCAI | 0.003759398 |
| 1 | 166962119 | 166962131 | MAEL | 0.003891051 |
| 10 | 17110577 | 17110588 | CUBN | 0.00390625 |
| 7 | 137076100 | 137076123 | DGKI | 0.00390625 |
| 1 | 145456732 | 145456747 | NBPF10, NBPF20, POLR3GL | 0.003952569 |
| 3 | 52696310 | 52696322 | PBRM1 | 0.003984064 |
| 1 | 57349140 | 57349151 | C8A | 0.004016064 |
| 2 | 70463334 | 70463348 | TIA1 | 0.004032258 |
| 9 | 130116724 | 130116736 | GARNL3 | 0.004065041 |
| 8 | 30569609 | 30569622 | GSR | 0.004081633 |
| 5 | 128990138 | 128990150 | ADAMTS19 | 0.004081633 |
| 10 | 16824085 | 16824108 | RSU1 | 0.004115226 |
| 14 | 58471378 | 58471390 | C14orf37(NM_001001872: c.*76_*64del0) | 0.004132231 |
| 12 | 54920288 | 54920301 | NCKAP1L | 0.0041841 |
| 1 | 22030115 | 22030132 | USP48 | 0.004219409 |
| 8 | 97245316 | 97245361 | UOCRB | 0.004219409 |
| 9 | 2088640 | 2088651 | SMARCA2 | 0.004255319 |
| 1 | 176012466 | 176012478 | RFWD2 | 0.004291845 |
| 8 | 141293914 | 141293943 | TRAPPC9 | 0.004310345 |
| 7 | 33217071 | 33217084 | BBS9 | 0.004310345 |
| 5 | 132408905 | 132408918 | HSPA4 | 0.004329004 |
| 15 | 65412325 | 65412337 | PDCD7 | 0.004329004 |
| 19 | 7995974 | 7996007 | TIMM44 | 0.004347826 |
| 9 | 125827752 | 125827764 | RABGAP1 | 0.004347826 |
| 12 | 58361014 | 58361034 | XRCC6BP1(dist = 9962), LOC101927653(dist = 598708) | 0.004347826 |
| 9 | 123922400 | 123922412 | CNTRL | 0.004405286 |
| 8 | 54742095 | 54742107 | ATP6V1H | 0.004424779 |
| 15 | 89533168 | 89533181 | MFGE8(dist = 76505), ABHD2(dist = 98200) | 0.004424779 |
| 14 | 76173952 | 76173964 | TTLL5 | 0.004424779 |
| X | 12516777 | 12516790 | FRMPD4 | 0.004444444 |
| 10 | 81667391 | 81667414 | LOC100288974 | 0.004504505 |
| 22 | 29682873 | 29682894 | EWSR1 | 0.004524887 |
| 5 | 80460576 | 80460591 | FAH | 0.004545455 |
| 2 | 220040216 | 220040231 | CNPPD1 | 0.004545455 |
| 14 | 50760968 | 50760980 | L2HGDH | 0.004587156 |
| 2 | 201534173 | 201534187 | AOX1 | 0.004587156 |
| 4 | 17841869 | 17841881 | NCAPG | 0.004587156 |
| 9 | 131721441 | 131721453 | NUP188 | 0.004587156 |
| 2 | 183960133 | 183960155 | DUSP19 | 0.00462963 |
| 7 | 131113756 | 131113779 | MKLN1 | 0.00462963 |
| 21 | 27136460 | 27136473 | GABPA | 0.00462963 |
| 5 | 72185591 | 72185607 | TNPO1 | 0.00462963 |
| 7 | 104707158 | 104707171 | KMT2E | 0.004651163 |
| 6 | 7402813 | 7402824 | RIOK1 | 0.004672897 |
| 9 | 19328009 | 19328021 | DENND4C | 0.004694836 |
| 8 | 1844623 | 1844634 | ARHGEF10 | 0.004694836 |
| 3 | 9730522 | 9730543 | MTMR14 | 0.004694836 |
| 5 | 159841492 | 159841507 | SLU7 | 0.004716981 |
| 2 | 15676703 | 15676717 | NBAS | 0.004739336 |
| 13 | 77644856 | 77644877 | MYCBP2 | 0.004739336 |
| 2 | 114210810 | 114210837 | CBWD2 | 0.004739336 |
| 11 | 60182851 | 60182862 | MS4A14 | 0.004761905 |
| 9 | 19350679 | 19350691 | DENND4C | 0.004784689 |
| 1 | 89614950 | 89614962 | GBP7 | 0.004807692 |
| 11 | 134055425 | 134055436 | NCAPD3 | 0.004807692 |
| 7 | 65426054 | 65426069 | GUSB | 0.004830918 |
| 1 | 71535064 | 71535075 | ZRANB2 | 0.004830918 |
| 20 | 3565277 | 3565289 | ATRN | 0.004854369 |
| 1 | 117633133 | 117633144 | TTF2 | 0.004854369 |
| 4 | 13371622 | 13371645 | RAB28 | 0.004854369 |
| 5 | 145508515 | 145508535 | LARS | 0.004926108 |
| 1 | 235621851 | 235621865 | B3GALNT2 | 0.004950495 |
| 1 | 220355521 | 220355533 | RAB3GAP2 | 0.004950495 |
| 6 | 55739711 | 55739726 | BMP5(NM_021073: c.-48_-63del0) | 0.005 |
| 14 | 39594134 | 39594146 | GEMIN2 | 0.005050505 |
| 6 | 166831580 | 166831594 | RPS6KA2 | 0.005076142 |
| 16 | 71682686 | 71682711 | PHLPP2(NM_015020: c.*107_*82del0, | 0.005102041 |
| | | | NM_001289003: c.*107_*82del0) | |
| 8 | 38940143 | 38940157 | ADAM9 | 0.005128205 |
| 14 | 75508258 | 75508303 | MLH3 | 0.005263158 |
| 4 | 56277766 | 56277777 | TMEM165 | 0.005376344 |
| 20 | 43623620 | 43623658 | STK4 | 0.005464481 |
| 1 | 45976928 | 45976939 | PRDX1(NM_001202431: c.*73_*62del0, | 0.005464481 |
| | | | NM_002574: c.*73_*62del0, | |
| | | | NM_181697: c.*73_*62del0, | |
| | | | NM_181696: c.*73_*62del0) | |
| 11 | 28113094 | 28113107 | KIF18A | 0.005494505 |
| 6 | 137193301 | 137193332 | PEX7 | 0.005494505 |
| 7 | 33192288 | 33192300 | BBS9 | 0.005555556 |
| 15 | 42503953 | 42503965 | TMEM87A | 0.005555556 |
| 6 | 155109168 | 155109189 | SCAF8 | 0.005555556 |
| 12 | 110924566 | 110924578 | FAM216A | 0.005586592 |
| 14 | 73079138 | 73079150 | RGS6(dist = 45900), DPF3(dist = 6854) | 0.005780347 |
| 11 | 58385231 | 58385251 | ZFP91-CNTF | 0.005952381 |
| 5 | 180482303 | 180482317 | BTNL9 | 0.005988024 |
| 1 | 223156384 | 223156419 | DISP1 | 0.005988024 |
| 8 | 124822507 | 124822519 | FAM91A1 | 0.006060606 |
| 3 | 196792356 | 196792367 | DLG1 | 0.006097561 |
| 19 | 16613797 | 16613832 | C19orf44 | 0.00617284 |
| 2 | 209110161 | 209110186 | IDH1 | 0.006289308 |
| 8 | 28989764 | 28989782 | KIF13B | 0.006756757 |
| 19 | 18857939 | 18857956 | CRTC1 | 0.006993007 |
| 7 | 106523362 | 106523383 | PIK3CG | 0.007067138 |
| 2 | 166898938 | 166898948 | SCN1A | 0.007194245 |
| 17 | 29528313 | 29528336 | NF1 | 0.007246377 |
| 2 | 10269544 | 10269555 | RRM2(NM_001165931: c.*31_*42del0, | 0.007407407 |
| | | | NM_001034: c.*31_*42del0) | |
| 12 | 133237753 | 133237768 | POLE | 0.007575758 |
| 12 | 122437594 | 122437605 | WDR66 | 0.007692308 |
| 3 | 50095096 | 50095119 | RBM6 | 0.007751938 |
| 18 | 14218568 | 14218580 | ANKRD20A5P | 0.008097166 |
| 6 | 15651642 | 15651656 | DTNBP1 | 0.008130081 |
| 14 | 62261603 | 62261633 | SNAPC1 | 0.008163265 |
| 2 | 201950159 | 201950178 | NDUFB3 | 0.008264463 |
| 1 | 112303077 | 112303090 | DDX20 | 0.008298755 |
| 8 | 130874614 | 130874639 | FAM49B | 0.008298755 |
| 21 | 37787572 | 37787585 | CHAF1B | 0.008298755 |
| 22 | 38766061 | 38766082 | LOC400927 | 0.008583691 |
| 7 | 134626034 | 134626049 | CALD1 | 0.00862069 |
| 14 | 104145625 | 104145642 | KLC1 | 0.00862069 |
| 11 | 122817187 | 122817199 | C11orf63 | 0.008658009 |
| 8 | 62550924 | 62550938 | ASPH | 0.008658009 |
| 7 | 92883250 | 92883261 | CCDC132 | 0.008695652 |
| 9 | 75420448 | 75420463 | TMC1 | 0.008733624 |
| 2 | 64069277 | 64069293 | UGP2(NM_006759: c.-43_-27del-) | 0.008733624 |
| 18 | 9279678 | 9279703 | ANKRD12 | 0.008733624 |
| 15 | 73418741 | 73418756 | NEO1 | 0.008849558 |
| 7 | 150269192 | 150269204 | GIMAP4 | 0.008849558 |
| 17 | 46248074 | 46248089 | SKAP1 | 0.008849558 |
| 20 | 60861505 | 60861519 | OSBPL2 | 0.008888889 |
| 8 | 38839302 | 38839316 | HTRA4 | 0.00896861 |
| 12 | 25678995 | 25679008 | LMNTD1 | 0.00896861 |
| 7 | 152055766 | 152055780 | KMT2C | 0.00896861 |
| 4 | 123109034 | 123109048 | KIAA1109 | 0.009009009 |
| 1 | 70742532 | 70742545 | ANKRD13C | 0.009009009 |
| 2 | 32427092 | 32427104 | SLC30A6 | 0.009389671 |
| 1 | 78432506 | 78432521 | FUBP1 | 0.009389671 |
| 20 | 47648454 | 47648481 | ARFGEF2 | 0.009478673 |
| 2 | 102785158 | 102785171 | IL1R1 | 0.009478673 |
| 4 | 41015553 | 41015566 | APBB2 | 0.00952381 |
| 2 | 239241445 | 239241460 | TRAF3IP1 | 0.009615385 |
| 12 | 53861636 | 53861650 | PCBP2 | 0.009661836 |
| 5 | 58270446 | 58270485 | PDE4D(NM_001197223: c.*45_*6del0, | 0.009756098 |
| | | | NM_001197221: c.*45_*6del0, | |
| | | | NM_001197220: c.*45_*6del0, | |
| | | | NM_001165899: c.*45_*6del0, | |
| | | | NM_001197218: c.*45_*6del0, | |
| | | | NM_001197222: c.*45_*6del0, | |
| | | | NM_001197219: c.*45_*6del0, | |
| | | | NM_006203: c.*45_*6del0, | |
| | | | NM_001104631: c.*45_*6del0) | |
| 3 | 127820493 | 127820505 | RUVBL1 | 0.009803922 |
| 17 | 49048274 | 49048312 | SPAG9 | 0.009803922 |
| 17 | 47076449 | 47076462 | IGF2BP1 | 0.009803922 |
| 12 | 53470811 | 53470838 | SPRYD3 | 0.00990099 |
| 19 | 47656338 | 47656359 | SAE1 | 0.01 |
| 15 | 41770598 | 41770624 | RTF1 | 0.01010101 |
| 6 | 88853530 | 88853542 | CNR1(NM_001160259: c.*45_*33del0, | 0.010204082 |
| | | | NM_001160226: c.*45_*33del0, | |
| | | | NM_001160258: c.*45_*33del0, | |
| | | | NM_033181: c.*45_*33del0, | |
| | | | NM_016083: c.*45_*33del0) | |
| 11 | 14316128 | 14316148 | RRAS2 | 0.010204082 |
| 19 | 12112120 | 12112142 | ZNF763(dist = 20922), ZNF433(dist = 13390) | 0.010416667 |
| 17 | 74625858 | 74625883 | ST6GALNAC1 | 0.010416667 |
| 6 | 149899943 | 149899954 | GINM1 | 0.010752688 |
| 8 | 194596 | 194607 | ZNF596 | 0.010752688 |
| 10 | 94694025 | 94694037 | EXOC6 | 0.010810811 |
| 5 | 108168451 | 108168468 | FER | 0.010810811 |
| 19 | 34302673 | 34302689 | KCTD15 | 0.010909091 |
| 1 | 149900985 | 149901002 | MTMR11(NM_001145862: c.*36_*19del0) | 0.010948905 |
| 10 | 75576885 | 75576896 | CAMK2G | 0.010989011 |
| 16 | 10851655 | 10851666 | NUBP1 | 0.011173184 |
| 8 | 68140383 | 68140394 | ARFGEF1 | 0.011235955 |
| 12 | 55820958 | 55820971 | OR6C76 | 0.011235955 |
| 6 | 32147291 | 32147312 | RNF5P1 | 0.011428571 |
| 1 | 74575199 | 74575231 | LRRIQ3 | 0.011695906 |
| 12 | 99059316 | 99059327 | APAF1 | 0.012048193 |
| 7 | 73279233 | 73279260 | WBSCR28 | 0.012121212 |
| 6 | 70040500 | 70040513 | BAI3 | 0.012195122 |
| 11 | 74790486 | 74790510 | NEU3(dist = 71743), OR2AT4(dist = 9286) | 0.012269939 |
| 2 | 27708007 | 27708031 | IFT172 | 0.012396694 |
| 10 | 69716602 | 69716614 | HERC4 | 0.012448133 |
| 16 | 67470052 | 67470095 | HSD11B2 | 0.0125 |
| 17 | 10317164 | 10317176 | MYHAS | 0.0125 |
| 19 | 10090461 | 10090482 | COL5A3 | 0.0125 |
| 16 | 19063220 | 19063235 | TMC7 | 0.012658228 |
| 4 | 141448595 | 141448610 | ELMOD2 | 0.012711864 |
| 13 | 73293238 | 73293264 | MZT1 | 0.012765957 |
| 12 | 122966016 | 122966028 | ZCCHC8 | 0.012987013 |
| 3 | 195022926 | 195022944 | ACAP2 | 0.013043478 |
| 6 | 160157277 | 160157288 | WTAP | 0.013215859 |
| 20 | 4850568 | 4850578 | SLC23A2 | 0.013245033 |
| 1 | 14057425 | 14057439 | PRDM2 | 0.013274336 |
| 20 | 10622080 | 10622091 | JAG1 | 0.013513514 |
| 1 | 168200730 | 168200743 | SFT2D2 | 0.013513514 |
| 2 | 179595571 | 179595589 | TTN | 0.013888889 |
| 17 | 57937792 | 57937807 | TUBD1 | 0.013953488 |
| 1 | 180757007 | 180757018 | XPR1 | 0.013953488 |
| 11 | 55656437 | 55656451 | TRIM51 | 0.014218009 |
| 6 | 117753497 | 117753511 | ROS1(dist = 6479), DCBLD1(dist = 50309) | 0.014218009 |
| 17 | 29563068 | 29563089 | NF1 | 0.014285714 |
| 1 | 213180434 | 213180459 | ANGEL2 | 0.014336918 |
| 1 | 19683355 | 19683368 | CAPZB | 0.014354067 |
| 12 | 89995053 | 89995065 | ATP2B1 | 0.014492754 |
| 5 | 36629801 | 36629815 | SLC1A3 | 0.014492754 |
| 3 | 195956726 | 195956749 | SLC51A | 0.014563107 |
| 16 | 75670529 | 75670550 | KARS | 0.014705882 |
| 1 | 193038176 | 193038186 | TROVE2 | 0.014814815 |
| 18 | 56279052 | 56279066 | ALPK2 | 0.015075377 |
| 19 | 37967219 | 37967230 | ZNF570 | 0.015075377 |
| 11 | 108014684 | 108014704 | ACAT1 | 0.015075377 |
| 5 | 96116095 | 96116125 | ERAP1 | 0.015151515 |
| 12 | 21958998 | 21959011 | ABCC9 | 0.015564202 |
| 15 | 55789793 | 55789806 | DYX1C1-CCPG1 | 0.015625 |
| 4 | 47163234 | 47163248 | GABRB1 | 0.015748031 |
| 12 | 16713487 | 16713501 | LMO3 | 0.016129032 |
| 12 | 27841925 | 27841938 | PPFIBP1 | 0.016194332 |
| 4 | 114176848 | 114176860 | ANK2 | 0.016460905 |
| 14 | 69861462 | 69861490 | ERH | 0.016666667 |
| 4 | 156725681 | 156725695 | GUCY1B3 | 0.016666667 |
| 1 | 46726867 | 46726890 | RAD54L | 0.016759777 |
| 7 | 40087320 | 40087333 | CDK13 | 0.016853933 |
| 12 | 53823175 | 53823189 | AMHR2 | 0.016877637 |
| 3 | 77645936 | 77645960 | ROBO2 | 0.016877637 |
| 2 | 167138320 | 167138331 | SCN9A | 0.016949153 |
| 5 | 169020337 | 169020350 | SPDL1 | 0.016949153 |
| 19 | 41188890 | 41188901 | NUMBL | 0.017142857 |
| 15 | 58891938 | 58891962 | ADAM10 | 0.017241379 |
| 16 | 50368677 | 50368692 | BRD7 | 0.017241379 |
| 2 | 158283943 | 158283954 | CYTIP | 0.017241379 |
| 6 | 24412431 | 24412445 | MRS2 | 0.017467249 |
| 1 | 173795803 | 173795821 | DARS2 | 0.017699115 |
| 10 | 12240681 | 12240702 | CDC123 | 0.01793722 |
| 8 | 95507237 | 95507250 | KIAA1429 | 0.018348624 |
| X | 36156447 | 36156461 | CHDC2 | 0.018518519 |
| 15 | 31220709 | 31220744 | FAN1 | 0.019323671 |
| 9 | 135133867 | 135133881 | NTNG2(dist = 15647), SETX(dist = 2946) | 0.019607843 |
| 17 | 16344523 | 16344534 | LRRC75A-AS1 | 0.019736842 |
| 17 | 18708830 | 18708843 | TVP23B | 0.01980198 |
| 5 | 147785802 | 147785816 | FBXO38 | 0.02 |
| 2 | 56102061 | 56102072 | EFEMP1 | 0.020134228 |
| 13 | 42772765 | 42772789 | DGKH | 0.020304569 |
| 12 | 94575162 | 94575177 | PLXNC1 | 0.020547945 |
| 1 | 92765880 | 92765892 | RPAP2 | 0.020833333 |
| 12 | 56500502 | 56500515 | PA2G4 | 0.021052632 |
| 15 | 49097732 | 49097750 | CEP152 | 0.021126761 |
| 7 | 81641570 | 81641584 | LOC101927356 | 0.021551724 |
| 7 | 126086436 | 126086447 | GRM8 | 0.021621622 |
| 3 | 148858987 | 148859001 | HPS3 | 0.02173913 |
| 13 | 111340025 | 111340053 | CARS2 | 0.022058824 |
| 11 | 33374613 | 33374626 | HIPK3 | 0.022222222 |
| 1 | 201754396 | 201754428 | IPO9-AS1, RNU6-79P | 0.02247191 |
| 20 | 50781298 | 50781310 | ZFP64 | 0.022727273 |
| 5 | 67576330 | 67576346 | PIK3R1 | 0.022857143 |
| 4 | 146031236 | 146031253 | ABCE1 | 0.022988506 |
| 17 | 6662948 | 6662977 | XAF1 | 0.023121387 |
| 16 | 15133783 | 15133795 | NTAN1, PDXDC1 | 0.023696682 |
| 4 | 7817686 | 7817710 | AFAP1 | 0.023696682 |
| 6 | 102124470 | 102124483 | GRIK2 | 0.024 |
| 11 | 93454831 | 93454868 | SCARNA9 | 0.024242424 |
| 6 | 49415351 | 49415368 | MUT | 0.025641026 |
| 5 | 137513369 | 137513382 | BRD8 | 0.02617801 |
| 10 | 116735234 | 116735260 | TRUB1(NM_139169: c.*96_*122del0) | 0.026315789 |
| 1 | 70904523 | 70904552 | CTH | 0.026737968 |
| 19 | 37155495 | 37155509 | ZNF461 | 0.027027027 |
| 21 | 30925853 | 30925869 | GRIK1(NM_000830: c.*10_*7del0) | 0.029166667 |
| 14 | 93686568 | 93686587 | UBR7 | 0.029288703 |
| 16 | 4700318 | 4700337 | MGRN1 | 0.03 |
| 4 | 3040279 | 3040293 | GRK4 | 0.03030303 |
| 8 | 75944409 | 75944422 | CRISPLD1 | 0.031674208 |
| 6 | 90366329 | 90366347 | LOC101929057 | 0.03187251 |
| 3 | 122271204 | 122271215 | PARP9 | 0.031914894 |
| 12 | 76763092 | 76763106 | OSBPL8 | 0.033057851 |
| 1 | 236744720 | 236744735 | HEATRI | 0.033613445 |
| | 67165932 | 67165942 | PPPICA(NM_002708: c._*74_*64del0, | 0.034090909 |
| | | | NM_206873: c._*74_*64del0, | |
| | | | NM_001008709: c.*74_*64del0) | |
| 1 | 114092111 | 114092124 | MAGI3 | 0.034188034 |
| 14 | 55429897 | 55429917 | WDHD1 | 0.034482759 |
| 6 | 151859146 | 151859161 | CCDC170 | 0.035175879 |
| 1 | 245021291 | 245021305 | HNRNPU | 0.035353535 |
| 15 | 42111745 | 42111756 | MAPKBP1 | 0.035460993 |
| 6 | 52400479 | 52400497 | TRAM2 | 0.038961039 |
| 4 | 122590883 | 122590899 | ANXA5 | 0.039525692 |
| 2 | 183806893 | 183806904 | NCKAP1 | 0.040983607 |
| 9 | 2097548 | 2097560 | SMARCA2 | 0.042424242 |
| 19 | 41703604 | 41703616 | CYP2S1 | 0.042780749 |
| 12 | 109701959 | 109701975 | ACACB | 0.042857143 |
| 6 | 167453519 | 167453532 | FGFR1OP(NM_007045: c.*53_*66del0, | 0.043478261 |
| | | | NM_194429: c.*53_*66del0) | |
| 13 | 50306823 | 50306836 | KPNA3 | 0.043478261 |
| 10 | 5788110 | 5788126 | FAM208B | 0.04845815 |
| 1 | 67154730 | 67154748 | SGIP1 | 0.048672566 |
| 10 | 71647196 | 71647209 | COL13A1 | 0.049586777 |

TABLE 9
Microsatellite Repeat Marker Sequences for Prostate Adenocarcinoma (PRAD).
TABLE 9 - PRAD

| ch | start_pos | end_pos | | PRAD |
|---|---|---|---|---|
| (hg37) | (hg37) | (hg37) | gene | p-value |
| 1 | 150932953 | 150932989 | SETDB1 | 3.48675E−05 |
| 19 | 14104688 | 14104703 | RFX1(NM_002918: c.-33_-48del0) | 3.6062E−05 |
| 8 | 42761288 | 42761308 | HOOK3 | 3.96825E−05 |
| 3 | 180320897 | 180320910 | TTC14 | 4.07647E−05 |
| 4 | 26417055 | 26417080 | RBPJ | 4.3877E−05 |
| 5 | 94842718 | 94842730 | TTC37 | 4.64511E−05 |
| 2 | 66796098 | 66796109 | MEIS1 | 4.69021E−05 |
| 1 | 202736222 | 202736233 | KDM5B | 4.69021E−05 |
| 1 | 151380720 | 151380737 | POGZ | 4.69021E−05 |
| 3 | 164905648 | 164905660 | SLITRK3(NM_014926: c.*37_*25del0) | 4.97512E−05 |
| 22 | 32482127 | 32482141 | SLC5A1 | 5.02513E−05 |
| 10 | 115380483 | 115380497 | NRAP | 5.12742E−05 |
| 22 | 31942815 | 31942840 | SFI1 | 5.45375E−05 |
| 10 | 27037487 | 27037499 | ABI1 | 6.20732E−05 |
| 10 | 122630877 | 122630889 | MIR5694 | 6.348E−05 |
| 2 | 8998775 | 8998788 | MBOAT2(NM_138799: c.*34_*21del0) | 6.56814E−05 |
| 1 | 161829973 | 161829987 | ATF6 | 6.64408E−05 |
| 6 | 32147291 | 32147312 | RNF5P1 | 6.96136E−05 |
| 20 | 54823684 | 54823699 | MC3R | 7.21449E−05 |
| 13 | 37596114 | 37596125 | SUPT20H | 7.57404E−05 |
| 11 | 117152291 | 117152314 | RNF214 | 8.2713E−05 |
| 5 | 178140630 | 178140648 | ZNF354A | 9.3188E−05 |
| 11 | 32610726 | 32610740 | EIF3M | 0.000106372 |
| 22 | 35719378 | 35719394 | TOM1 | 0.000111957 |
| 8 | 95186510 | 95186526 | CDH17 | 0.000111957 |
| 6 | 111329221 | 111329239 | RPF2 | 0.000114916 |
| 14 | 73079138 | 73079150 | RGS6(dist = 45900), DPF3(dist = 6854) | 0.00011566 |
| 6 | 13316780 | 13316795 | TBC1D7 | 0.000120115 |
| 8 | 101933023 | 101933037 | YWHAZ | 0.000123031 |
| 20 | 2474136 | 2474149 | ZNF343 | 0.000123406 |
| 18 | 649879 | 649895 | TYMSOS(NM_001012716: c.*120_*104del0) | 0.000126834 |
| 2 | 60988862 | 60988872 | PAPOLG | 0.000129032 |
| 20 | 30818497 | 30818514 | POFUT1 | 0.000129199 |
| 14 | 58471378 | 58471390 | C14orf37(NM_001001872: c.*76_*64del0) | 0.000130406 |
| 1 | 84956161 | 84956173 | RPF1 | 0.000140706 |
| 21 | 37706065 | 37706077 | MORC3 | 0.000143472 |
| 2 | 86998651 | 86998676 | RMND5A | 0.000147776 |
| 13 | 111340025 | 111340053 | CARS2 | 0.000155255 |
| 12 | 48360947 | 48360961 | TMEM106C | 0.000192604 |
| 12 | 31820564 | 31820578 | METTL20 | 0.000194805 |
| 4 | 47163234 | 47163248 | GABRB1 | 0.000205754 |
| 15 | 83808031 | 83808046 | HDGFRP3 | 0.000210963 |
| 15 | 42503953 | 42503965 | TMEM87A | 0.000216435 |
| 5 | 67576330 | 67576346 | PIK3R1 | 0.000227221 |
| 15 | 41770639 | 41770652 | RTF1 | 0.000242395 |
| 8 | 141293914 | 141293943 | TRAPPC9 | 0.000242395 |
| 1 | 151384052 | 151384067 | POGZ | 0.000258398 |
| 6 | 31832356 | 31832372 | SLC44A4 | 0.000268264 |
| 2 | 201534173 | 201534187 | AOX1 | 0.000278707 |
| 9 | 163927 | 163943 | CBWD1 | 0.000279564 |
| 13 | 28133956 | 28133972 | LNX2 | 0.000295552 |
| 12 | 8692327 | 8692338 | CLEC4E | 0.000320496 |
| 7 | 94989255 | 94989273 | PON3(NM_000940: c.*30_*12del0) | 0.000323834 |
| 2 | 183841691 | 183841704 | NCKAP1 | 0.000333 |
| 17 | 8417006 | 8417020 | MYH10 | 0.000345006 |
| 20 | 55206241 | 55206253 | TFAP2C | 0.000432869 |
| 16 | 4873743 | 4873761 | GLYR1 | 0.000448954 |
| 5 | 78416973 | 78416990 | BHMT | 0.00049373 |
| 9 | 77550305 | 77550323 | TRPM6(dist = 47295), C9orf40(dist = 11176) | 0.000589971 |
| 8 | 23712066 | 23712089 | STC1(NM_003155: c.-30_-53del0) | 0.000606061 |
| 11 | 65268479 | 65268492 | MALAT1 | 0.000613874 |
| 14 | 74534274 | 74534284 | ALDH6A1 | 0.000657895 |
| 8 | 17422434 | 17422446 | SLC7A2 | 0.000686342 |
| 17 | 80443296 | 80443308 | NARF | 0.000910692 |
| 7 | 92129161 | 92129183 | PEX1 | 0.001057865 |
| 1 | 67154730 | 67154748 | SGIP1 | 0.001326072 |
| 5 | 70809269 | 70809285 | BDP1 | 0.001407035 |
| 16 | 15133783 | 15133795 | NTAN1, PDXDC1 | 0.001559454 |
| 4 | 52928352 | 52928371 | SPATA18 | 0.002125357 |
| 10 | 94366347 | 94366370 | KIF11 | 0.003623188 |
| 8 | 100287517 | 100287536 | VPS13B | 0.003921569 |
| 7 | 114303617 | 114303633 | FOXP2 | 0.004101162 |
| 1 | 24857651 | 24857670 | RCAN3 | 0.004219409 |
| 12 | 21958998 | 21959011 | ABCC9 | 0.004310345 |
| 14 | 21702046 | 21702071 | HNRNPC | 0.004310345 |
| 3 | 52696310 | 52696322 | PBRM1 | 0.004347826 |
| 8 | 95777578 | 95777591 | DPY19L4 | 0.004347826 |
| 21 | 30433900 | 30433919 | CCT8 | 0.004347826 |
| 10 | 26998490 | 26998502 | PDSS1 | 0.004347826 |
| 19 | 6026288 | 6026302 | RFX2 | 0.004464286 |
| 3 | 113655096 | 113655108 | GRAMD1C | 0.004464286 |
| 12 | 133237753 | 133237768 | POLE | 0.004484305 |
| 8 | 104054513 | 104054532 | ATP6V1C1 | 0.004484305 |
| 2 | 157370018 | 157370033 | GPD2 | 0.004504505 |
| 4 | 47886322 | 47886342 | LOC101927179 | 0.004524887 |
| 19 | 4292947 | 4292975 | TMIGD2 | 0.004524887 |
| 6 | 46636556 | 46636581 | SLC25A27 | 0.004545455 |
| 9 | 98703711 | 98703723 | ERCC6L2 | 0.004545455 |
| 3 | 172052897 | 172052914 | FNDC3B | 0.00456621 |
| 1 | 173795803 | 173795821 | DARS2 | 0.00456621 |
| 17 | 1584377 | 1584390 | PRPF8 | 0.00456621 |
| 2 | 32117217 | 32117230 | MEMO1 | 0.004587156 |
| 2 | 192011485 | 192011497 | STAT4 | 0.004608295 |
| 11 | 107904512 | 107904524 | CUL5 | 0.004608295 |
| 8 | 71037103 | 71037115 | NCOA2 | 0.004672897 |
| 7 | 131113756 | 131113779 | MKLN1 | 0.004672897 |
| X | 18183097 | 18183113 | BEND2(NM_153346: c.*32_*16del0) | 0.004672897 |
| X | 153994469 | 153994482 | DKC1 | 0.004694836 |
| 2 | 48688258 | 48688273 | PPP1R21 | 0.004694836 |
| 22 | 26899774 | 26899785 | TFIP11 | 0.004694836 |
| X | 113965679 | 113965691 | HTR2C | 0.004694836 |
| 17 | 39776817 | 39776829 | KRT17 | 0.004761905 |
| 4 | 57897508 | 57897530 | IGFBP7 | 0.004784689 |
| 6 | 42559866 | 42559879 | UBR2 | 0.004830918 |
| 13 | 103301601 | 103301612 | TPP2 | 0.004830918 |
| 10 | 112649255 | 112649287 | PDCD4 | 0.004854369 |
| 3 | 97605460 | 97605472 | CRYBG3 | 0.004901961 |
| 4 | 89352469 | 89352487 | HERC6 | 0.004950495 |
| 22 | 29879265 | 29879302 | NEFH | 0.004975124 |
| 7 | 5239305 | 5239336 | WIPI2 | 0.005 |
| 3 | 113169372 | 113169386 | SPICE1 | 0.005025126 |
| 7 | 131130509 | 131130538 | MKLN1 | 0.005025126 |
| 13 | 99890849 | 99890862 | MIR548AN | 0.005076142 |
| 6 | 117026217 | 117026229 | KPNA5 | 0.005076142 |
| 17 | 18785823 | 18785842 | PRPSAP2 | 0.005076142 |
| 6 | 128330423 | 128330435 | PTPRK | 0.005128205 |
| 8 | 74495112 | 74495130 | STAU2 | 0.005128205 |
| 14 | 103915242 | 103915254 | MARK3 | 0.005181347 |
| 10 | 88834244 | 88834265 | GLUD1 | 0.005181347 |
| 3 | 100992404 | 100992415 | IMPG2 | 0.005181347 |
| 3 | 100438778 | 100438810 | TFG | 0.005208333 |
| 20 | 4839890 | 4839911 | SLC23A2 | 0.005263158 |
| 6 | 163235158 | 163235170 | PACRG | 0.005263158 |
| 21 | 33068865 | 33068877 | SCAF4 | 0.005263158 |
| 2 | 54146387 | 54146400 | PSME4 | 0.005291005 |
| 17 | 4880231 | 4880257 | CAMTA2 | 0.005291005 |
| 6 | 96971185 | 96971208 | UFL1 | 0.005319149 |
| 15 | 55841195 | 55841209 | PYGO1 | 0.005347594 |
| 5 | 129070812 | 129070838 | ADAMTS19 | 0.005347594 |
| 7 | 74608740 | 74608754 | GTF2IP1, LOC100093631 | 0.005376344 |
| 9 | 97555228 | 97555242 | C9orf3 | 0.005376344 |
| 9 | 2083313 | 2083338 | SMARCA2 | 0.005376344 |
| 11 | 59836986 | 59836998 | MS4A3 | 0.005405405 |
| 16 | 15138162 | 15138182 | NTAN1, PDXDC1 | 0.005405405 |
| 5 | 137763868 | 137763912 | KDM3B | 0.005405405 |
| 11 | 73753268 | 73753288 | C2CD3 | 0.005434783 |
| 3 | 170715672 | 170715694 | SLC2A2 | 0.005464481 |
| 9 | 33886999 | 33887011 | UBE2R2 | 0.005524862 |
| 3 | 24270412 | 24270424 | THRB | 0.005524862 |
| 6 | 166831580 | 166831594 | RPS6KA2 | 0.005524862 |
| 10 | 75576885 | 75576896 | CAMK2G | 0.005555556 |
| 4 | 56770490 | 56770501 | EXOC1 | 0.005586592 |
| 15 | 59961570 | 59961607 | BNIP2 | 0.005617978 |
| 2 | 32427092 | 32427104 | SLC30A6 | 0.005649718 |
| 6 | 163899794 | 163899807 | QKI | 0.005681818 |
| 20 | 46266543 | 46266558 | NCOA3 | 0.005714286 |
| 21 | 42838093 | 42838104 | TMPRSS2 | 0.005714286 |
| 2 | 222290623 | 222290635 | EPHA4(NM_004438: c.*125_*113del0) | 0.005747126 |
| 5 | 37326073 | 37326084 | NUP 155 | 0.005747126 |
| 3 | 44815871 | 44815883 | KIF15 | 0.005780347 |
| 11 | 110306734 | 110306760 | FDX1 | 0.005813953 |
| 18 | 31523144 | 31523156 | NOL4 | 0.005813953 |
| 1 | 115276971 | 115276990 | CSDE1 | 0.005844535 |
| 2 | 238632159 | 238632170 | LRRFIP1 | 0.005847953 |
| 17 | 26653806 | 26653817 | TMEM97 | 0.005847953 |
| 19 | 7670033 | 7670064 | CAMSAP3 | 0.005847953 |
| 20 | 60711773 | 60711796 | PSMA7(NM_002792: c.*130_*117del0) | 0.005882353 |
| 2 | 175246545 | 175246557 | CIR1 | 0.005952381 |
| 15 | 57545438 | 57545449 | TCF12 | 0.006060606 |
| 16 | 5129689 | 5129703 | ALG1 | 0.006097561 |
| 6 | 49459994 | 49460005 | CENPQ(NM_018132: c.*6_*17del0) | 0.006097561 |
| 15 | 70961809 | 70961821 | UACA | 0.006097561 |
| 14 | 73465027 | 73465043 | ZFYVE1 | 0.006097561 |
| 2 | 61622374 | 61622387 | USP34 | 0.006134969 |
| 3 | 47604262 | 47604281 | CSPG5 | 0.006134969 |
| 5 | 150856066 | 150856091 | SLC36A1 | 0.00617284 |
| 1 | 99164246 | 99164260 | SNX7 | 0.00625 |
| 6 | 20758808 | 20758822 | CDKAL1 | 0.006289308 |
| 15 | 35149313 | 35149327 | AQR | 0.006289308 |
| 15 | 41364265 | 41364305 | INO80 | 0.006329114 |
| 1 | 225607366 | 225607387 | LBR | 0.006329114 |
| 3 | 141622616 | 141622641 | ATP1B3 | 0.006329114 |
| 3 | 52728303 | 52728323 | GNL3(NM_014366: c.*12_*32del0, | 0.006369427 |
| | | | NM_206825: c.*12_*32del0, | |
| | | | NM_206826: c.*12_*32del0) | |
| 2 | 165365365 | 165365376 | GRB14 | 0.006410256 |
| 6 | 80837242 | 80837253 | BCKDHB | 0.006410256 |
| 10 | 121678931 | 121678952 | SEC23IP | 0.006410256 |
| 2 | 103281540 | 103281552 | SLC9A2 | 0.006451613 |
| 8 | 68140383 | 68140394 | ARFGEF1 | 0.006493506 |
| 4 | 71693494 | 71693508 | GRSF1 | 0.006493506 |
| 19 | 9528423 | 9528438 | ZNF266 | 0.006493506 |
| 7 | 23823176 | 23823194 | STK31 | 0.006535948 |
| 2 | 64210968 | 64210991 | VPS54 | 0.006578947 |
| 11 | 89502007 | 89502036 | TRIM77(dist = 50967), TRIM49(dist = 28787) | 0.006578947 |
| 12 | 104515109 | 104515121 | NFYB | 0.006666667 |
| 13 | 39603371 | 39603407 | PROSER1 | 0.006666667 |
| 17 | 35979746 | 35979761 | DDX52 | 0.006711409 |
| 1 | 41456808 | 41456823 | CTPS1 | 0.006802721 |
| 17 | 7306338 | 7306349 | TMEM256-PLSCR3 | 0.006802721 |
| 3 | 154007618 | 154007629 | DHX36 | 0.006802721 |
| 4 | 184567431 | 184567444 | RWDD4 | 0.006802721 |
| 20 | 3928832 | 3928855 | RNF24 | 0.006896552 |
| X | 135328309 | 135328320 | MAP7D3 | 0.007092199 |
| 16 | 29110397 | 29110417 | RRN3P2 | 0.007092199 |
| 1 | 1688823 | 1688845 | NADK | 0.007142857 |
| 12 | 69107631 | 69107642 | NUP107 | 0.007194245 |
| 5 | 72168575 | 72168596 | TNPO1 | 0.007352941 |
| 14 | 64496810 | 64496822 | MIR548AZ | 0.007352941 |
| 14 | 92562967 | 92562986 | ATXN3 | 0.007352941 |
| 18 | 30913142 | 30913153 | CCDC178 | 0.007407407 |
| 17 | 73515055 | 73515068 | TSEN54 | 0.007462687 |
| 7 | 44663890 | 44663910 | OGDH | 0.007462687 |
| 18 | 63430040 | 63430078 | CDH7(NM_033646: c.-39_-1del-, | 0.007462687 |
| | | | NM_004361: c.-39_-1del-) | |
| 5 | 134007593 | 134007616 | SEC24A | 0.007462687 |
| 5 | 143853686 | 143853711 | KCTD16(NM_020768: c.*9_*34del0) | 0.007518797 |
| 17 | 7700419 | 7700440 | DNAH2 | 0.007633588 |
| 16 | 31202698 | 31202716 | FUS | 0.007843137 |
| 17 | 5324558 | 5324611 | RPAIN | 0.007874016 |
| 15 | 59009930 | 59009941 | ADAM10 | 0.007874016 |
| 14 | 21960425 | 21960451 | TOX4 | 0.007936508 |
| 2 | 215821543 | 215821563 | LOC101928103 | 0.007936508 |
| 2 | 111406968 | 111406984 | BUB1 | 0.008032129 |
| 17 | 30190540 | 30190551 | UTP6 | 0.008064516 |
| 15 | 86768264 | 86768280 | AGBL1 | 0.008130081 |
| 4 | 89711690 | 89711718 | FAM13A | 0.008196721 |
| 17 | 5044640 | 5044653 | USP6 | 0.008196721 |
| 8 | 27637666 | 27637677 | ESCO2 | 0.008196721 |
| 17 | 38028508 | 38028519 | ZPBP2 | 0.008196721 |
| 22 | 36650908 | 36650919 | APOL1 | 0.008333333 |
| 4 | 76997157 | 76997173 | ART3 | 0.008368201 |
| 17 | 1582787 | 1582825 | PRPF8 | 0.008368201 |
| 3 | 50095096 | 50095119 | RBM6 | 0.008368201 |
| 2 | 27824348 | 27824360 | ZNF512 | 0.008368201 |
| 9 | 52625 | 52641 | FAM138C(dist = 16761), PGM5P3-AS1(dist = 20049) | 0.008403361 |
| 19 | 34302673 | 34302689 | KCTD15 | 0.008403361 |
| 7 | 31132235 | 31132249 | ADCYAP1R1 | 0.008403361 |
| 22 | 36872638 | 36872662 | TXN2 | 0.008403361 |
| 2 | 206997835 | 206997847 | NDUFS1 | 0.008403361 |
| 4 | 141448595 | 141448610 | ELMOD2 | 0.008438819 |
| 9 | 36109839 | 36109853 | RECK | 0.008438819 |
| 12 | 54646020 | 54646054 | CBX5 | 0.008474576 |
| 5 | 98224945 | 98224957 | CHD1 | 0.008474576 |
| 3 | 57850505 | 57850518 | SLMAP | 0.008510638 |
| 14 | 57101560 | 57101576 | TMEM260 | 0.008510638 |
| 16 | 69293926 | 69293945 | SNTB2 | 0.008547009 |
| 1 | 220284112 | 220284124 | RNU5F-1 | 0.008547009 |
| 12 | 59307848 | 59307866 | LRIG3 | 0.008547009 |
| 1 | 54252939 | 54252952 | NDC1 | 0.008583691 |
| 1 | 222904869 | 222904881 | BROX | 0.00862069 |
| 1 | 169845236 | 169845247 | SCYL3 | 0.00862069 |
| 16 | 31196218 | 31196237 | FUS | 0.008658009 |
| 9 | 73164596 | 73164610 | TRPM3 | 0.008658009 |
| 3 | 137892531 | 137892543 | DBR1 | 0.008658009 |
| 5 | 78373251 | 78373265 | BHMT2 | 0.008658009 |
| 14 | 61446607 | 61446626 | TRMT5 | 0.008658009 |
| 12 | 4645161 | 4645173 | C12orf4 | 0.008695652 |
| 1 | 12368705 | 12368721 | VPS13D | 0.008695652 |
| 8 | 134125650 | 134125662 | TG | 0.008733624 |
| 17 | 29508819 | 29508836 | NF1 | 0.00877193 |
| 2 | 9558905 | 9558924 | ITGB1BP1 | 0.00877193 |
| 10 | 26825151 | 26825168 | APBB1IP | 0.008810573 |
| 8 | 133984814 | 133984834 | TG | 0.008849558 |
| 7 | 138951165 | 138951177 | UBN2 | 0.008849558 |
| 6 | 166578165 | 166578177 | T | 0.008849558 |
| 10 | 16824085 | 16824108 | RSU1 | 0.008888889 |
| 5 | 179032860 | 179032871 | RUFY1 | 0.008888889 |
| 11 | 108188266 | 108188280 | ATM | 0.008888889 |
| 17 | 10317164 | 10317176 | MYHAS | 0.008928571 |
| 7 | 7572355 | 7572381 | COL28A1 | 0.008928571 |
| 10 | 112044559 | 112044572 | MXI1 | 0.008928571 |
| 9 | 5811331 | 5811349 | ERMP1 | 0.00896861 |
| 1 | 173490547 | 173490571 | SLC9C2 | 0.00896861 |
| 1 | 221875661 | 221875686 | DUSP10(NM_007207: c.*93_*68del0) | 0.00896861 |
| 9 | 19334957 | 19334970 | DENND4C | 0.009009009 |
| 3 | 160960468 | 160960478 | NMD3 | 0.009009009 |
| 15 | 52903303 | 52903313 | FAM214A | 0.009009009 |
| 10 | 112360315 | 112360333 | SMC3 | 0.009049774 |
| 11 | 122817187 | 122817199 | C11orf63 | 0.009049774 |
| 9 | 131743701 | 131743715 | NUP188 | 0.009090909 |
| 9 | 79820192 | 79820224 | VPS13A | 0.009090909 |
| 12 | 56994331 | 56994349 | BAZ2A | 0.009090909 |
| 1 | 112958898 | 112958910 | CTTNBP2NL | 0.009090909 |
| 20 | 10273694 | 10273715 | SNAP25 | 0.00913242 |
| 11 | 47187055 | 47187087 | ARFGAP2 | 0.00913242 |
| 9 | 80343587 | 80343612 | GNAQ | 0.00913242 |
| 5 | 31435975 | 31435987 | DROSHA | 0.00913242 |
| 1 | 155747369 | 155747381 | GON4L | 0.00913242 |
| 1 | 115226990 | 115227003 | AMPD1 | 0.009174312 |
| 10 | 101510104 | 101510123 | CUTC | 0.009174312 |
| 3 | 19498221 | 19498253 | KCNH8 | 0.00921659 |
| 1 | 3549854 | 3549874 | WRAP73 | 0.00921659 |
| 3 | 67058329 | 67058357 | KBTBD8 | 0.00921659 |
| 15 | 66794298 | 66794311 | RPL4 | 0.009259259 |
| 19 | 11411997 | 11412010 | TSPAN16 | 0.009259259 |
| 7 | 137076100 | 137076123 | DGKI | 0.009345794 |
| 2 | 179407763 | 179407779 | MIR548N, TTN-AS1 | 0.009345794 |
| 12 | 56380950 | 56380964 | RAB5B | 0.009345794 |
| 20 | 42265908 | 42265920 | IFT52 | 0.009345794 |
| 6 | 111701462 | 111701475 | REV3L | 0.009345794 |
| X | 107169290 | 107169321 | LOC101928335 | 0.009345794 |
| 6 | 80910611 | 80910635 | BCKDHB | 0.009389671 |
| 19 | 10090461 | 10090482 | COL5A3 | 0.009389671 |
| 10 | 12162338 | 12162351 | DHTKD1 | 0.009389671 |
| 1 | 70654981 | 70655003 | LRRC40 | 0.009433962 |
| 2 | 27463091 | 27463105 | CAD | 0.009433962 |
| 12 | 9220823 | 9220847 | A2M | 0.009433962 |
| 19 | 53050992 | 53051013 | ZNF808 | 0.009433962 |
| 3 | 33725814 | 33725828 | CLASP2 | 0.009478673 |
| 5 | 6623405 | 6623418 | NSUN2 | 0.00952381 |
| 5 | 94903575 | 94903589 | ARSK | 0.00952381 |
| 3 | 42602535 | 42602546 | SEC22C | 0.00952381 |
| 3 | 15613279 | 15613304 | HACL1 | 0.009569378 |
| 8 | 107704921 | 107704955 | OXR1 | 0.009615385 |
| 14 | 50760968 | 50760980 | L2HGDH | 0.009615385 |
| 9 | 114341239 | 114341255 | PTGR1 | 0.009615385 |
| 12 | 107080617 | 107080640 | LOC100287944 | 0.009615385 |
| 19 | 37967219 | 37967230 | ZNF570 | 0.009615385 |
| 16 | 21138881 | 21138894 | DNAH3 | 0.009615385 |
| 19 | 50951721 | 50951743 | MYBPC2 | 0.009661836 |
| 4 | 151850226 | 151850239 | LRBA | 0.009708738 |
| 2 | 37487341 | 37487353 | PRKD3 | 0.009708738 |
| 6 | 133093231 | 133093247 | SLC18B1 | 0.009708738 |
| 2 | 197531518 | 197531530 | CCDC150 | 0.009708738 |
| 3 | 142241692 | 142241705 | ATR | 0.009708738 |
| 7 | 6485740 | 6485753 | DAGLB | 0.009708738 |
| 2 | 37464877 | 37464888 | NDUFAF7 | 0.009708738 |
| 3 | 65456181 | 65456211 | MAGI1 | 0.009756098 |
| 3 | 62153593 | 62153621 | PTPRG | 0.009803922 |
| 3 | 146167163 | 146167186 | PLSCR2 | 0.009803922 |
| 9 | 78638564 | 78638576 | PCSK5 | 0.009803922 |
| 2 | 217329413 | 217329447 | SMARCAL1 | 0.009803922 |
| 4 | 83970297 | 83970312 | COPS4 | 0.009803922 |
| 13 | 39435552 | 39435564 | FREM2 | 0.009803922 |
| 12 | 42768646 | 42768660 | PPHLN1 | 0.009803922 |
| 6 | 42611936 | 42611951 | UBR2 | 0.009852217 |
| 14 | 59787325 | 59787338 | DAAM1 | 0.009852217 |
| 2 | 79314062 | 79314073 | REG1B | 0.009852217 |
| 9 | 33280749 | 33280775 | CHMP5 | 0.009852217 |
| 11 | 28113094 | 28113107 | KIF18A | 0.00990099 |
| 11 | 134188851 | 134188867 | GLB1L3(NM_001080407: c.*15_*31del0) | 0.00990099 |
| 15 | 48717850 | 48717884 | FBN1 | 0.009950249 |
| 7 | 76883792 | 76883819 | CCDC146 | 0.009950249 |
| 1 | 204494748 | 204494783 | MDM4 | 0.009950249 |
| 3 | 113442784 | 113442795 | NAA50 | 0.01 |
| 7 | 135099044 | 135099059 | CNOT4 | 0.01 |
| 5 | 179150788 | 179150800 | CANX | 0.01 |
| 17 | 58013510 | 58013529 | RPS6KB1 | 0.01 |
| 13 | 50134014 | 50134026 | RCBTB1 | 0.01 |
| 5 | 137451460 | 137451472 | NME5 | 0.010050251 |
| 22 | 44589636 | 44589647 | PARVG | 0.010050251 |
| 15 | 45725103 | 45725138 | C15orf48 | 0.01010101 |
| 1 | 11134470 | 11134485 | EXOSC10 | 0.01010101 |
| 17 | 39976721 | 39976731 | FKBP10 | 0.01010101 |
| 11 | 134118382 | 134118401 | THYN1 | 0.01010101 |
| 1 | 24180806 | 24180833 | FUCA1 | 0.01010101 |
| 3 | 51504906 | 51504917 | VPRBP | 0.01010101 |
| 16 | 3808052 | 3808066 | CREBBP | 0.010152284 |
| 7 | 123332378 | 123332391 | WASL | 0.010152284 |
| 2 | 102979238 | 102979250 | IL18R1 | 0.010152284 |
| 5 | 34945797 | 34945810 | DNAJC21 | 0.010152284 |
| 2 | 197654578 | 197654593 | GTF3C3 | 0.010204082 |
| 6 | 90452808 | 90452835 | MDN1 | 0.010204082 |
| 2 | 207027356 | 207027368 | EEF1B2 | 0.010204082 |
| 6 | 25452453 | 25452466 | LRRC16A | 0.010204082 |
| 1 | 153949135 | 153949147 | JTB | 0.01025641 |
| 11 | 55656437 | 55656451 | TRIM51 | 0.01025641 |
| 4 | 169612452 | 169612484 | PALLD | 0.01025641 |
| 16 | 48333493 | 48333512 | MIR548AE2 | 0.01025641 |
| 20 | 13140617 | 13140637 | SPTLC3 | 0.010309278 |
| 12 | 81239721 | 81239737 | LIN7A | 0.010309278 |
| 1 | 158298679 | 158298691 | CD1B | 0.010309278 |
| 12 | 31435608 | 31435620 | FAM60A(NM_001135811: c.*38_*26del0, | 0.010362694 |
| | | | NM_001135812: c.*38_*26del0, | |
| | | | NM_021238: c.*38_*26del0) | |
| 21 | 30435646 | 30435668 | CCT8 | 0.010416667 |
| 15 | 48826427 | 48826440 | FBN1 | 0.010416667 |
| 9 | 98230924 | 98230947 | LOC100507346 | 0.010416667 |
| 2 | 152424933 | 152424962 | NEB | 0.010416667 |
| 4 | 47746602 | 47746616 | CORIN | 0.010471204 |
| 7 | 75185990 | 75186002 | HIP1 | 0.010471204 |
| 5 | 122343337 | 122343374 | SNX24 | 0.010471204 |
| 2 | 61319728 | 61319740 | KIAA1841 | 0.010471204 |
| 7 | 99672649 | 99672660 | ZNF3 | 0.010471204 |
| 17 | 74286032 | 74286047 | QRICH2 | 0.010526316 |
| 14 | 81953889 | 81953901 | SEL1L | 0.010526316 |
| 6 | 76618182 | 76618195 | MY06 | 0.010526316 |
| 20 | 34430474 | 34430486 | PHF20 | 0.010582011 |
| 6 | 137325901 | 137325917 | IL20RA | 0.010582011 |
| 1 | 114186351 | 114186362 | MAGI3 | 0.010582011 |
| 1 | 155679543 | 155679557 | DAP3 | 0.010582011 |
| 17 | 29482977 | 29482998 | NF1 | 0.010582011 |
| 6 | 70459209 | 70459222 | LMBRD1 | 0.010638298 |
| 1 | 186330695 | 186330709 | MIR548F1 | 0.010638298 |
| 8 | 80831393 | 80831407 | MRPS28 | 0.010638298 |
| 3 | 186302391 | 186302439 | DNAJB11 | 0.010638298 |
| 2 | 172187261 | 172187296 | METTL8 | 0.010638298 |
| 7 | 6771663 | 6771684 | ZNF12(dist = 25097), PMS2CL(dist = 3252) | 0.010638298 |
| 14 | 92549604 | 92549623 | ATXN3 | 0.010695187 |
| 1 | 19683355 | 19683368 | CAPZB | 0.010695187 |
| 8 | 94817177 | 94817188 | TMEM67 | 0.010752688 |
| 1 | 150799125 | 150799138 | ARNT | 0.010810811 |
| 14 | 31762500 | 31762511 | HEATR5A | 0.010810811 |
| 12 | 51323672 | 51323684 | METTL7A | 0.010869565 |
| 4 | 74010555 | 74010571 | ANKRD17 | 0.010869565 |
| 9 | 75420448 | 75420463 | TMC1 | 0.010928962 |
| 16 | 67854644 | 67854670 | TSNAXIP1 | 0.010928962 |
| 9 | 19299209 | 19299224 | DENND4C | 0.010989011 |
| 11 | 119145463 | 119145476 | CBL | 0.010989011 |
| 7 | 63673449 | 63673467 | ZNF735 | 0.010989011 |
| 12 | 93149720 | 93149731 | PLEKHG7 | 0.010989011 |
| X | 52654536 | 52654550 | SSX8 | 0.011049724 |
| 1 | 22030891 | 22030902 | USP48 | 0.011049724 |
| 1 | 202407189 | 202407200 | PPP1R12B | 0.011111111 |
| 10 | 21901247 | 21901268 | MLLT10 | 0.011111111 |
| 9 | 98009613 | 98009626 | FANCC | 0.011173184 |
| 3 | 160134195 | 160134221 | SMC4 | 0.011173184 |
| 6 | 136429995 | 136430013 | PDE7B | 0.011173184 |
| 13 | 25416104 | 25416116 | RNF17 | 0.011173184 |
| 2 | 71645619 | 71645662 | ZNF638 | 0.011173184 |
| 11 | 3381798 | 3381814 | ZNF195 | 0.011173184 |
| 16 | 20749284 | 20749296 | THUMPD1 | 0.011235955 |
| 2 | 11913727 | 11913739 | LPIN1 | 0.011235955 |
| 5 | 87494728 | 87494767 | TMEM161B | 0.011235955 |
| 13 | 48664556 | 48664569 | MED4 | 0.011299435 |
| 2 | 21226218 | 21226230 | APOB | 0.011299435 |
| 5 | 138760853 | 138760870 | DNAJC18 | 0.011299435 |
| 3 | 122081881 | 122081893 | CCDC58 | 0.011363636 |
| 5 | 180482303 | 180482317 | BTNL9 | 0.011428571 |
| 7 | 121699789 | 121699802 | PTPRZ1 | 0.011494253 |
| 20 | 37126168 | 37126180 | RALGAPB | 0.011494253 |
| 1 | 210411266 | 210411281 | SERTAD4 | 0.011494253 |
| 11 | 134064516 | 134064540 | NCAPD3 | 0.011494253 |
| 7 | 23239063 | 23239074 | NUPL2 | 0.011494253 |
| 1 | 89449508 | 89449520 | RBMXL1 | 0.011560694 |
| 12 | 3935279 | 3935290 | PARP11 | 0.011627907 |
| 12 | 53573482 | 53573510 | CSAD | 0.011627907 |
| 18 | 214605 | 214622 | THOC1(NM_005131: c.*21_*4del0) | 0.011627907 |
| 13 | 48828102 | 48828118 | ITM2B | 0.011695906 |
| 6 | 24589046 | 24589057 | KIAA0319 | 0.011695906 |
| 16 | 4163683 | 4163694 | ADCY9 | 0.011695906 |
| 1 | 52912109 | 52912134 | ZCCHC11 | 0.011764706 |
| 11 | 134078847 | 134078858 | NCAPD3 | 0.011764706 |
| 6 | 112937974 | 112937995 | RFPL4B(dist = 265476), LOC101927686(dist = 1006742) | 0.011764706 |
| 4 | 128814425 | 128814449 | PLK4 | 0.01183432 |
| 9 | 34343033 | 34343047 | NUDT2 | 0.01183432 |
| 14 | 76211938 | 76211949 | TTLL5 | 0.01183432 |
| 5 | 150282674 | 150282694 | ZNF300 | 0.011904762 |
| 16 | 58585178 | 58585191 | CNOT1 | 0.011904762 |
| 17 | 1558921 | 1558934 | PRPF8 | 0.011904762 |
| 19 | 41188890 | 41188901 | NUMBL | 0.011976048 |
| 6 | 17669303 | 17669328 | NUP153 | 0.012048193 |
| 21 | 34907636 | 34907654 | GART | 0.012096774 |
| 21 | 42823193 | 42823206 | MX1 | 0.012121212 |
| 10 | 93841262 | 93841273 | CPEB3 | 0.012121212 |
| 10 | 49632498 | 49632510 | MAPK8 | 0.012121212 |
| 2 | 167138320 | 167138331 | SCN9A | 0.012269939 |
| 22 | 16920145 | 16920157 | OR11H1(dist = 470341), CCT8L2(dist = 151491) | 0.012269939 |
| 7 | 87056260 | 87056297 | ABCB4 | 0.012269939 |
| 12 | 111926224 | 111926237 | ATXN2 | 0.012269939 |
| 8 | 74585483 | 74585494 | STAU2 | 0.012269939 |
| 5 | 36123065 | 36123135 | LMBRD2 | 0.012345679 |
| 6 | 36839451 | 36839462 | PPIL1 | 0.012345679 |
| 17 | 40856565 | 40856586 | EZH1 | 0.012345679 |
| 11 | 59420570 | 59420588 | PATL1 | 0.012345679 |
| 12 | 54676165 | 54676176 | HNRNPA1 | 0.012345679 |
| 5 | 36629801 | 36629815 | SLC1A3 | 0.01242236 |
| 11 | 93490699 | 93490715 | C11orf54 | 0.01242236 |
| 12 | 31289662 | 31289674 | DDX11(dist = 31937), FAM60A(dist = 143846) | 0.01242236 |
| 20 | 41306488 | 41306500 | PTPRT | 0.0125 |
| 1 | 47581121 | 47581151 | CYP4Z1 | 0.0125 |
| 12 | 56677664 | 56677675 | CS | 0.0125 |
| 10 | 134628521 | 134628532 | CFAP46 | 0.012578616 |
| 4 | 120423686 | 120423697 | PDE5A | 0.012658228 |
| 4 | 141481203 | 141481225 | UCP1 | 0.012658228 |
| 13 | 25911034 | 25911067 | NUPL1 | 0.012658228 |
| 11 | 124007701 | 124007714 | VWA5A | 0.012658228 |
| 5 | 134086670 | 134086684 | CAMLG(NM_001745: c.*30_*44del0) | 0.012658228 |
| 17 | 4712563 | 4712580 | PLD2 | 0.012658228 |
| 12 | 122700993 | 122701012 | DIABLO | 0.012658228 |
| 4 | 146031236 | 146031253 | ABCE1 | 0.012738854 |
| 14 | 39594134 | 39594146 | GEMIN2 | 0.012738854 |
| 19 | 54264352 | 54264364 | MIR516A2 | 0.012738854 |
| 12 | 120741303 | 120741326 | SIRT4 | 0.012820513 |
| 5 | 14678762 | 14678776 | OTULIN | 0.012820513 |
| 1 | 226027067 | 226027104 | EPHX1 | 0.012903226 |
| 5 | 153174310 | 153174322 | GRIA1 | 0.012987013 |
| 9 | 94118279 | 94118310 | AUH | 0.012987013 |
| 17 | 57762402 | 57762414 | CLTC | 0.013071895 |
| 3 | 196532148 | 196532160 | PAK2 | 0.013157895 |
| 14 | 53522638 | 53522650 | DDHD1 | 0.013157895 |
| 12 | 66531936 | 66531947 | TMBIM4 | 0.013333333 |
| 15 | 59094494 | 59094508 | FAM63B | 0.013333333 |
| 1 | 212217944 | 212217961 | DTL | 0.013333333 |
| X | 153714027 | 153714038 | UBL4A | 0.013333333 |
| 1 | 183498191 | 183498202 | SMG7 | 0.013333333 |
| 4 | 114176848 | 114176860 | ANK2 | 0.013392857 |
| 4 | 120192436 | 120192448 | USP53 | 0.013422819 |
| 2 | 141272360 | 141272372 | LRP1B | 0.013422819 |
| 22 | 36901817 | 36901832 | FOXRED2 | 0.013422819 |
| 14 | 60587879 | 60587900 | PCNXL4 | 0.013452915 |
| 5 | 115167564 | 115167575 | ATG12 | 0.013513514 |
| 20 | 60861505 | 60861519 | OSBPL2 | 0.013574661 |
| 13 | 113818805 | 113818818 | PROZ | 0.013605442 |
| 21 | 47821449 | 47821460 | PCNT | 0.013605442 |
| X | 18599975 | 18599996 | CDKL5 | 0.013793103 |
| 6 | 102130410 | 102130422 | GRIK2 | 0.013888889 |
| 11 | 59368218 | 59368235 | OSBP | 0.013888889 |
| 16 | 70595514 | 70595525 | SF3B3 | 0.013888889 |
| 17 | 61278039 | 61278050 | TANC2 | 0.013888889 |
| 8 | 124440326 | 124440339 | WDYHV1 | 0.013888889 |
| 5 | 137464887 | 137464899 | NME5 | 0.013986014 |
| 1 | 236721452 | 236721466 | HEATR1 | 0.013986014 |
| 16 | 64294574 | 64294585 | NONE(dist = NONE), CDH11(dist = 686098) | 0.014084507 |

TABLE 10
Microsatellite Repeat Marker Sequences for Cervical Squamous Cell Carcinoma (CESC).
TABLE 10 - CESC

| ch | start_pos | end_pos | | CESC |
|---|---|---|---|---|
| (hg37) | (hg37) | (hg37) | gene | p-value |
| 12 | 64491002 | 64491019 | SRGAP1 | 2.65648E−07 |
| 10 | 112360315 | 112360333 | SMC3 | 1.1309E−06 |
| 7 | 100319680 | 100319696 | EPO | 4.50826E−06 |
| 10 | 18280054 | 18280071 | SLC39A12 | 4.89822E−06 |
| 5 | 36629801 | 36629815 | SLC1A3 | 8.23147E−06 |
| 15 | 52561891 | 52561907 | MYO5C | 9.43441E−06 |
| 22 | 16404838 | 16404857 | POTEH(dist = 116901), OR11H1(dist = 43967) | 9.68917E−06 |
| 11 | 16117684 | 16117698 | SOX6 | 9.77135E−06 |
| 5 | 41909830 | 41909846 | C5orf51 | 1.01245E−05 |
| 4 | 166388811 | 166388838 | CPE | 1.04949E−05 |
| 1 | 35885037 | 35885058 | ZMYM4 | 1.08836E−05 |
| 9 | 97555228 | 97555242 | C9orf3 | 1.36705E−05 |
| 2 | 87122096 | 87122121 | ANAPC1P1 | 1.36705E−05 |
| 6 | 80910611 | 80910635 | BCKDHB | 1.42248E−05 |
| 3 | 160144007 | 160144039 | SMC4 | 1.48093E−05 |
| 3 | 78695353 | 78695364 | ROBO1 | 1.54264E−05 |
| 1 | 153617510 | 153617526 | CHTOP | 1.57816E−05 |
| 9 | 130243534 | 130243551 | LRSAM1 | 2.40024E−05 |
| 21 | 34117067 | 34117080 | PAXBP1 | 2.71297E−05 |
| 1 | 33087567 | 33087584 | ZBTB8OS | 2.79955E−05 |
| 2 | 230653669 | 230653682 | TRIP12 | 2.79955E−05 |
| 7 | 128413997 | 128414041 | OPNISW | 2.79955E−05 |
| 14 | 76638065 | 76638087 | GPATCH2L | 3.08247E−05 |
| 10 | 48980563 | 48980577 | GLUD1P7 | 3.08247E−05 |
| 8 | 22138812 | 22138831 | PIWIL2 | 3.41763E−05 |
| 16 | 29110397 | 29110417 | RRN3P2 | 3.8117E−05 |
| X | 2343183 | 2343213 | DHRSX | 4.04981E−05 |
| 12 | 2997058 | 2997074 | RHNO1 | 4.19798E−05 |
| 7 | 74608740 | 74608754 | GTF2IP1, LOC100093631 | 4.35346E−05 |
| 9 | 15472611 | 15472623 | PSIP1 | 4.35346E−05 |
| X | 19024426 | 19024440 | GPR64 | 5.2579E−05 |
| 17 | 40986454 | 40986487 | PSME3 | 6.17055E−05 |
| 16 | 68155860 | 68155874 | NFATC3 | 6.70691E−05 |
| 2 | 189859434 | 189859447 | COL3A1 | 6.99851E−05 |
| 16 | 75674252 | 75674271 | KARS | 7.04722E−05 |
| 2 | 27463091 | 27463105 | CAD | 7.30727E−05 |
| 8 | 108315599 | 108315615 | ANGPT1 | 7.46024E−05 |
| 6 | 89638988 | 89639004 | RNGTT | 8.72094E−05 |
| 3 | 195010023 | 195010035 | ACAP2 | 8.74126E−05 |
| 10 | 71647196 | 71647209 | COL13A1 | 9.43441E−05 |
| 17 | 18708830 | 18708843 | TVP23B | 0.000100728 |
| 2 | 66796098 | 66796109 | MEIS1 | 0.000100728 |
| 2 | 239241445 | 239241460 | TRAF3IP1 | 0.000101215 |
| 11 | 74053485 | 74053497 | PGM2L1 | 0.000105764 |
| 18 | 21644075 | 21644099 | TTC39C | 0.000116891 |
| 7 | 92855628 | 92855641 | HEPACAM2 | 0.000126456 |
| 9 | 163927 | 163943 | CBWD1 | 0.000129634 |
| 13 | 28133956 | 28133972 | LNX2 | 0.000135019 |
| 19 | 34951356 | 34951371 | UBA2 | 0.000135019 |
| 3 | 58296209 | 58296232 | RPP14 | 0.000136705 |
| 13 | 21370398 | 21370412 | XPO4 | 0.000136705 |
| 16 | 12145683 | 12145699 | SNX29 | 0.000186706 |
| 10 | 8111345 | 8111359 | GATA3 | 0.000190862 |
| 6 | 136429995 | 136430013 | PDE7B | 0.000214777 |
| 14 | 90735723 | 90735735 | PSMC1 | 0.000218531 |
| 12 | 107237602 | 107237626 | RIC8B | 0.000219298 |
| 8 | 22175821 | 22175848 | PIWIL2 | 0.000223964 |
| 12 | 109510086 | 109510099 | USP30 | 0.000234481 |
| 4 | 146031236 | 146031253 | ABCE1 | 0.000238892 |
| 8 | 134125650 | 134125662 | TG | 0.0002442 |
| 19 | 54264352 | 54264364 | MIR516A2 | 0.0002442 |
| 17 | 1383742 | 1383781 | MYO1C | 0.0002442 |
| 20 | 35695535 | 35695562 | RBL1 | 0.000255363 |
| 6 | 76618182 | 76618195 | MYO6 | 0.000261233 |
| 10 | 27333121 | 27333134 | ANKRD26 | 0.00026536 |
| 8 | 33356825 | 33356839 | MAK16(NM_032509: c.*678_*692del0) | 0.000273598 |
| 2 | 166758447 | 166758460 | TTC21B | 0.000273598 |
| 3 | 138193025 | 138193038 | ESYT3 | 0.000273598 |
| 11 | 68338491 | 68338520 | PPP6R3 | 0.000280112 |
| 1 | 169845236 | 169845247 | SCYL3 | 0.000280112 |
| 2 | 54146387 | 54146400 | PSME4 | 0.000286862 |
| 19 | 34945331 | 34945344 | UBA2 | 0.000286862 |
| 1 | 197059221 | 197059244 | ASPM | 0.000286862 |
| 11 | 18231788 | 18231799 | LOC494141 | 0.000286862 |
| 19 | 22836873 | 22836886 | ZNF492 | 0.000293858 |
| 17 | 27816599 | 27816619 | TAOK1 | 0.000293858 |
| 3 | 191097928 | 191097944 | CCDC50 | 0.000296187 |
| 9 | 19299209 | 19299224 | DENND4C | 0.000301114 |
| 3 | 100438778 | 100438810 | TFG | 0.000301114 |
| 16 | 31196218 | 31196237 | FUS | 0.000301114 |
| 4 | 47886322 | 47886342 | LOC101927179 | 0.000301114 |
| 14 | 39602843 | 39602863 | GEMIN2 | 0.000301114 |
| 2 | 201778717 | 201778733 | ORC2 | 0.000308642 |
| 6 | 18258299 | 18258311 | DEK | 0.000308642 |
| 2 | 55883509 | 55883523 | PNPT1 | 0.000316456 |
| 3 | 93768420 | 93768439 | ARL13B | 0.000316456 |
| 2 | 61308569 | 61308585 | KIAA1841 | 0.00032457 |
| 5 | 118500981 | 118501000 | DMXL1 | 0.00032457 |
| 11 | 83180169 | 83180185 | DLG2 | 0.00032457 |
| 2 | 69553369 | 69553389 | GFPT1 | 0.00032457 |
| 8 | 117861112 | 117861124 | RAD21 | 0.00032457 |
| 14 | 96800846 | 96800858 | ATG2B | 0.000341763 |
| 8 | 17504626 | 17504637 | MTUS1 | 0.000350877 |
| 1 | 168205770 | 168205781 | SFT2D2 | 0.00036036 |
| 2 | 215812345 | 215812356 | LOC101928103 | 0.00036036 |
| 17 | 40850552 | 40850587 | CNTNAP1 | 0.00036075 |
| 6 | 55739711 | 55739726 | BMP5(NM_021073: c.-48_-63del0) | 0.000370233 |
| 5 | 145508515 | 145508535 | LARS | 0.000370233 |
| 4 | 74010555 | 74010571 | ANKRD17 | 0.000380518 |
| 1 | 115226990 | 115227003 | AMPD1 | 0.000391236 |
| 6 | 107420417 | 107420451 | BEND3 | 0.000397875 |
| 20 | 49354351 | 49354368 | PARD6B | 0.000402414 |
| 3 | 10301805 | 10301816 | TATDN2 | 0.000402414 |
| 16 | 31202698 | 31202716 | FUS | 0.000413223 |
| 7 | 98639904 | 98639917 | SMURF1 | 0.000414079 |
| 8 | 124382376 | 124382403 | ATAD2 | 0.000414079 |
| X | 48053437 | 48053450 | SSX5 | 0.000414079 |
| 2 | 141625630 | 141625642 | LRP1B | 0.000427183 |
| X | 129546260 | 129546274 | RBMX2 | 0.000437685 |
| 7 | 7572355 | 7572381 | COL28A1 | 0.000438982 |
| 20 | 16025192 | 16025206 | MACROD2 | 0.000438982 |
| 7 | 20826270 | 20826284 | SP8 | 0.000438982 |
| 11 | 49056627 | 49056644 | TRIM49B | 0.000445931 |
| 6 | 100901513 | 100901552 | SIM1 | 0.000445931 |
| 13 | 76134860 | 76134871 | UCHL3 | 0.000452284 |
| 4 | 55131001 | 55131019 | PDGFRA | 0.00045534 |
| 20 | 58587783 | 58587794 | CDH26 | 0.0004662 |
| 2 | 203817257 | 203817270 | CARF | 0.0004662 |
| 16 | 20803249 | 20803265 | ACSM3, ERI2 | 0.0004662 |
| 2 | 211527815 | 211527840 | CPS1 | 0.000480769 |
| 17 | 8417006 | 8417020 | MYH10 | 0.00049584 |
| 1 | 144918011 | 144918022 | NBPF20, NBPF9, PDE4DIP | 0.000496032 |
| 2 | 88875981 | 88875993 | EIF2AK3 | 0.000496032 |
| 8 | 121502680 | 121502691 | MTBP | 0.000496032 |
| 6 | 26501598 | 26501613 | BTN1A1 | 0.000496032 |
| 12 | 54920288 | 54920301 | NCKAP1L | 0.000528821 |
| 13 | 113527868 | 113527879 | ATP11A | 0.000528821 |
| X | 106092450 | 106092473 | TBC1D8B | 0.000539084 |
| 8 | 54742095 | 54742107 | ATP6V1H | 0.000546448 |
| 1 | 64022708 | 64022725 | EFCAB7 | 0.000549451 |
| 7 | 36465606 | 36465622 | ANLN | 0.000560119 |
| 3 | 183857809 | 183857826 | EIF2B5 | 0.000560119 |
| 3 | 146234962 | 146234973 | PLSCR1 | 0.000564972 |
| 19 | 39412756 | 39412767 | SARS2 | 0.000580032 |
| X | 48631645 | 48631660 | GLOD5 | 0.000582411 |
| 7 | 92900502 | 92900527 | CCDC132 | 0.000584454 |
| 1 | 16248728 | 16248740 | SPEN | 0.000584454 |
| 9 | 86395372 | 86395398 | GKAP1 | 0.000594059 |
| 1 | 155686739 | 155686757 | DAP3 | 0.000594059 |
| 1 | 176012466 | 176012478 | RFWD2 | 0.000604961 |
| X | 24516919 | 24516938 | PDK3 | 0.000604961 |
| 14 | 55612006 | 55612020 | LGALS3(NM_002306: c.*17_*31del0) | 0.000606061 |
| 14 | 19571455 | 19571478 | POTEG | 0.000611154 |
| 14 | 102550069 | 102550088 | HSP90AA1 | 0.000618429 |
| 17 | 1582787 | 1582825 | PRPF8 | 0.000618429 |
| 18 | 649879 | 649895 | TYMSOS(NM_001012716: c.*120_*104del0) | 0.000618429 |
| 16 | 70176311 | 70176336 | PDPR | 0.00063118 |
| X | 153357614 | 153357628 | MECP2 | 0.00064433 |
| 17 | 45105631 | 45105644 | RPRML(dist = 49017), ARL17A(dist = 3308) | 0.000649351 |
| 5 | 108203451 | 108203465 | FER | 0.000656111 |
| 4 | 141448595 | 141448610 | ELMOD2 | 0.000657895 |
| 5 | 67584512 | 67584525 | PIK3R1(NM_181524: c.-52_-39del-) | 0.000657895 |
| 2 | 37487341 | 37487353 | PRKD3 | 0.000671892 |
| 3 | 58351660 | 58351690 | PXK | 0.000686342 |
| 1 | 220284112 | 220284124 | RNU5F-1 | 0.000686342 |
| 6 | 116914112 | 116914128 | RWDD1 | 0.000686342 |
| 20 | 33225781 | 33225808 | PIGU | 0.000686342 |
| 2 | 238277142 | 238277163 | COL6A3 | 0.000698812 |
| 12 | 63964640 | 63964653 | DPY19L2 | 0.000701262 |
| 4 | 113568126 | 113568140 | LARP7 | 0.000716675 |
| 14 | 58690427 | 58690451 | ACTR10 | 0.000716675 |
| 7 | 131113756 | 131113779 | MKLN1 | 0.000732601 |
| 7 | 26883761 | 26883776 | SKAP2 | 0.000732601 |
| 3 | 58792080 | 58792092 | C3orf67 | 0.000732601 |
| X | 40588605 | 40588618 | MED14 | 0.000749064 |
| 3 | 52696310 | 52696322 | PBRM1 | 0.000766088 |
| 1 | 39802338 | 39802351 | MACF1 | 0.000766088 |
| 17 | 66039938 | 66039951 | KPNA2 | 0.000783699 |
| 10 | 101992923 | 101992938 | CWF19L1(NM_018294: c.*61_*46del0) | 0.000801925 |
| 14 | 59790749 | 59790763 | DAAM1 | 0.000801925 |
| 2 | 219614190 | 219614206 | TTLL4 | 0.000801925 |
| 3 | 3194099 | 3194118 | CRBN | 0.000801925 |
| 2 | 44445604 | 44445617 | PPM1B | 0.000816327 |
| 21 | 17135361 | 17135372 | USP25 | 0.000816327 |
| 1 | 173795803 | 173795821 | DARS2 | 0.000820793 |
| 6 | 131211616 | 131211641 | EPB41L2 | 0.000820793 |
| X | 106396617 | 106396641 | NUP62CL | 0.000840336 |
| 3 | 44815871 | 44815883 | KIF15 | 0.000840336 |
| 1 | 230798864 | 230798876 | COG2 | 0.000840336 |
| 1 | 161829973 | 161829987 | ATF6 | 0.00085034 |
| 10 | 64022582 | 64022593 | RTKN2 | 0.000860585 |
| 16 | 21559162 | 21559174 | SLC7A5P2(dist = 27397), LOC101927814(dist = 41845) | 0.000860585 |
| X | 52640310 | 52640321 | XAGE1E(dist = 94113), SSX8(dist = 11664) | 0.000860585 |
| 18 | 66367726 | 66367742 | TMX3 | 0.000881575 |
| 17 | 57937792 | 57937807 | TUBD1 | 0.000881575 |
| 4 | 39233719 | 39233734 | MIR1273H | 0.000903342 |
| 3 | 178937630 | 178937642 | PIK3CA | 0.000903342 |
| 21 | 17236804 | 17236816 | USP25 | 0.000903342 |
| 17 | 30293149 | 30293164 | SUZ12 | 0.000925926 |
| 15 | 45725103 | 45725138 | C15orf48 | 0.000925926 |
| 6 | 117243181 | 117243196 | RFX6 | 0.000925926 |
| 5 | 147785802 | 147785816 | FBXO38 | 0.000949367 |
| 1 | 200080464 | 200080490 | NR5A2 | 0.000965251 |
| 6 | 36452603 | 36452620 | KCTD20 | 0.00097371 |
| 2 | 64112697 | 64112719 | UGP2 | 0.00097371 |
| 20 | 47734948 | 47734972 | STAU1 | 0.00097371 |
| X | 83403148 | 83403160 | RPS6KA6 | 0.00097371 |
| 10 | 15113934 | 15113946 | OLAH | 0.00097371 |
| 10 | 28908452 | 28908478 | WAC | 0.00097371 |
| 2 | 38545608 | 38545626 | ATL2 | 0.00097371 |
| 5 | 137507104 | 137507121 | BRD8 | 0.000979792 |
| 20 | 13090733 | 13090745 | SPTLC3 | 0.000982096 |
| 3 | 132169705 | 132169718 | DNAJC13 | 0.000999001 |
| 16 | 19063220 | 19063235 | TMC7 | 0.000999001 |
| 3 | 5024415 | 5024435 | BHLHE40 | 0.000999001 |
| 6 | 13597295 | 13597307 | SIRT5 | 0.000999001 |
| 15 | 86768264 | 86768280 | AGBL1 | 0.001010101 |
| 11 | 118454476 | 118454495 | ARCN1 | 0.001010101 |
| 15 | 82517622 | 82517671 | EFTUD1 | 0.00102529 |
| 18 | 44424651 | 44424667 | PIAS2 | 0.00102529 |
| 7 | 106888946 | 106888972 | COG5 | 0.00102529 |
| 3 | 130660392 | 130660405 | ATP2C1 | 0.00102529 |
| 9 | 99408079 | 99408094 | AAED1 | 0.001038422 |
| 5 | 74011541 | 74011556 | HEXB | 0.001052632 |
| 5 | 32057910 | 32057923 | PDZD2 | 0.001052632 |
| 6 | 49459994 | 49460005 | CENPO(NM_018132: c.*6_*17del0) | 0.001052632 |
| 3 | 67058329 | 67058357 | KBTBD8 | 0.001081081 |
| 5 | 81601142 | 81601155 | ATP6AP1L(NM_001017971: c.-79_-66del-) | 0.001081081 |
| 2 | 236877066 | 236877084 | AGAP1 | 0.001098901 |
| 18 | 19110456 | 19110477 | ESCO1 | 0.00110742 |
| 7 | 95775848 | 95775863 | SLC25A13 | 0.0011107 |
| X | 79991590 | 79991604 | BRWD3 | 0.0011107 |
| 6 | 111329221 | 111329239 | RPF2 | 0.001113119 |
| 11 | 107518175 | 107518187 | ELMOD1 | 0.001141553 |
| 10 | 95549836 | 95549852 | LGI1 | 0.001142204 |
| 1 | 24978892 | 24978909 | SRRM1 | 0.001150599 |
| 2 | 201534173 | 201534187 | AOX1 | 0.00116144 |
| 3 | 132173004 | 132173026 | DNAJC13 | 0.001173709 |
| 7 | 75513199 | 75513231 | RHBDD2 | 0.001173709 |
| 2 | 179442292 | 179442314 | MIR548N, TTN-AS1 | 0.001173709 |
| 14 | 53513439 | 53513452 | DDHD1(NM_001160148: c.*47_*34del0, | 0.001207243 |
| | | | NM_001160147: c.*47_*34del0, | |
| | | | NM_030637: c.*47_*34del0) | |
| 2 | 98907089 | 98907103 | VWA3B | 0.001207243 |
| 2 | 170413815 | 170413834 | FASTKD1 | 0.001207243 |
| 3 | 138248009 | 138248026 | CEP70 | 0.001242236 |
| 3 | 186295417 | 186295431 | DNAJB11 | 0.001242236 |
| 2 | 178364328 | 178364342 | AGPS | 0.001242236 |
| 3 | 10018739 | 10018754 | EMC3 | 0.001262361 |
| 3 | 132166148 | 132166162 | DNAJC13 | 0.001278772 |
| 6 | 131948469 | 131948501 | MED23 | 0.001315789 |
| 2 | 158390343 | 158390363 | ACVR1C(NM_001111032: c.*87_*67del0, | 0.001315789 |
| | | | NM_001111031: c.*87_*67del0, | |
| | | | NM_001111033: c.*87_*67del0, | |
| | | | NM_145259: c.*87_*67del0) | |
| 7 | 23226646 | 23226667 | NUPL2 | 0.001316945 |
| 4 | 25160732 | 25160748 | SEPSECS | 0.001316945 |
| 6 | 158049351 | 158049365 | ZDHHC14 | 0.001316945 |
| 1 | 156203572 | 156203588 | PMF1, PMF1-BGLAP | 0.001343785 |
| 9 | 137803188 | 137803208 | FCN1 | 0.001349528 |
| 6 | 52768392 | 52768408 | GSTA3 | 0.001356852 |
| 2 | 234916771 | 234916787 | TRPM8 | 0.001372684 |
| 8 | 38839302 | 38839316 | HTRA4 | 0.001372684 |
| 12 | 50867692 | 50867704 | LARP4 | 0.001398601 |
| 19 | 43382540 | 43382581 | PSG1 | 0.001398601 |
| 7 | 5087800 | 5087822 | RBAK, RBAK-RBAKDN | 0.001402525 |
| 5 | 128990138 | 128990150 | ADAMTS19 | 0.001402525 |
| 20 | 30818497 | 30818514 | POFUT1 | 0.001402525 |
| 1 | 22030115 | 22030132 | USP48 | 0.001433349 |
| 17 | 66033401 | 66033412 | KPNA2 | 0.001442308 |
| 14 | 104145625 | 104145642 | KLC1 | 0.001442308 |
| 13 | 41814298 | 41814313 | MTRF1 | 0.001442308 |
| 3 | 100016726 | 100016752 | TBC1D23 | 0.001442308 |
| 11 | 64715330 | 64715349 | C11orf85 | 0.001465201 |
| 8 | 103287850 | 103287864 | UBR5 | 0.001488095 |
| 4 | 57897508 | 57897530 | IGFBP7 | 0.001488095 |
| 10 | 81667391 | 81667414 | LOC100288974 | 0.001498127 |
| 15 | 40509618 | 40509629 | PAK6(NM_001128629: c.-47358_-47358delins-, | 0.001501502 |
| | | | NM_001128628: c.-47358_-47358delins-) | |
| 12 | 124242458 | 124242472 | ATP6V0A2 | 0.001532176 |
| 2 | 27597190 | 27597204 | SNX17 | 0.001532176 |
| 1 | 197898395 | 197898415 | LHX9(NM_001014434: c.*6_*26del0, | 0.001536098 |
| | | | NM_020204: c.*6_*26del0) | |
| 12 | 56744020 | 56744045 | STAT2 | 0.001536098 |
| 12 | 95488339 | 95488354 | FGD6 | 0.001547836 |
| X | 23693084 | 23693098 | PRDX4 | 0.001567398 |
| 3 | 138400782 | 138400796 | PIK3CB | 0.001567398 |
| 11 | 108141955 | 108141971 | ATM | 0.001576945 |
| 11 | 49196555 | 49196567 | FOLH1 | 0.001586462 |
| 15 | 48634311 | 48634341 | DUT(NM_001025249: c.*36_*66del0, | 0.001586462 |
| | | | NM_001025248: c.*36_*66del0, | |
| | | | NM_001948: c.*36_*66del0) | |
| 2 | 109371849 | 109371865 | RANBP2 | 0.001603849 |
| 7 | 72159820 | 72159832 | TYW1B | 0.001603849 |
| 12 | 21791410 | 21791426 | LDHB | 0.001639344 |
| 5 | 56546749 | 56546777 | GPBPI | 0.001641587 |
| 6 | 34735652 | 34735670 | SNRPC | 0.001669376 |
| 8 | 101933023 | 101933037 | YWHAZ | 0.001680672 |
| 5 | 70809269 | 70809285 | BDP1 | 0.001680672 |
| 2 | | | | |
| 17 | 36477914 | 36477929 | MRPL45 | 0.001680672 |
| 18 | 22017875 | 22017901 | IMPACT | 0.001694915 |
| 5 | 14287248 | 14287259 | TRIO | 0.001694915 |
| 21 | 34635068 | 34635095 | IFNAR2 | 0.001694915 |
| 12 | 43964612 | 43964623 | ADAMTS20(dist = 18888), PUS7L(dist = 157787) | 0.001753361 |
| 3 | 185155430 | 185155442 | MAP3K13 | 0.001753361 |
| 18 | 61064220 | 61064237 | VPS4B | 0.001753361 |
| 9 | 88287588 | 88287615 | AGTPBP1 | 0.001806685 |
| 4 | 166215662 | 166215692 | KLHL2 | 0.001806685 |
| 11 | 27414071 | 27414102 | LGR4 | 0.001806685 |
| 2 | 152293320 | 152293334 | RIF1 | 0.001806685 |
| 1 | 8385485 | 8385496 | SLC45A1 | 0.001806685 |
| X | 48214185 | 48214200 | SSX3 | 0.001851852 |
| 21 | 38092247 | 38092259 | SIM2 | 0.001851852 |
| X | 52654536 | 52654550 | SSX8 | 0.001879699 |
| 6 | 136710669 | 136710685 | MAP7 | 0.001893939 |
| 12 | 104077096 | 104077126 | STAB2 | 0.001898734 |
| 2 | 37487307 | 37487330 | PRKD3 | 0.001898734 |
| 7 | 54819993 | 54820005 | SEC61G(NM_014302: c.*111_*99del0, | 0.001919425 |
| | | | NM_001012456: c.*111_*99del0) | |
| 8 | 39114668 | 39114704 | ADAM32 | 0.00194742 |
| 4 | 187627659 | 187627676 | FAT1 | 0.00194742 |
| X | 129483202 | 129483222 | SLC25A14 | 0.00194742 |
| 11 | 73984657 | 73984669 | P4HA3 | 0.001948052 |
| 12 | 108140266 | 108140281 | LOC101929162 | 0.001959686 |
| 1 | 229662901 | 229662916 | ABCB10 | 0.001998002 |
| 2 | 118854094 | 118854108 | INSIG2(NM_016133: c.-39_-25del-) | 0.002016129 |
| X | 3539376 | 3539390 | PRKX | 0.002020202 |
| 17 | 62855003 | 62855017 | LRRC37A3 | 0.002050581 |
| 19 | 6026288 | 6026302 | RFX2 | 0.002088374 |
| 3 | 19959707 | 19959719 | EFHB | 0.002096436 |
| 11 | 68318688 | 68318706 | PPP6R3 | 0.002096436 |
| 12 | 31289662 | 31289674 | DDX11(dist = 31937), FAM60A(dist = 143846) | 0.002162162 |
| 1 | 173797451 | 173797462 | DARS2 | 0.002177068 |
| 7 | 74172385 | 74172400 | GTF2I | 0.002177068 |
| 9 | 133498229 | 133498245 | FUBP3 | 0.002181339 |
| 5 | 89977095 | 89977106 | GPR98 | 0.002221399 |
| X | 150573322 | 150573335 | VMA21 | 0.002230167 |
| 5 | 94903575 | 94903589 | ARSK | 0.002230167 |
| 4 | 39347146 | 39347159 | MIR1273H | 0.002262443 |
| X | 77393375 | 77393394 | TAF9B | 0.002262443 |
| 20 | 55206241 | 55206253 | TFAP2C | 0.002280652 |
| 1 | 186330695 | 186330709 | MIR548F1 | 0.002280652 |
| 8 | 42829373 | 42829384 | HOOK3 | 0.002283105 |
| 11 | 102080325 | 102080341 | YAP1 | 0.002298851 |
| 20 | 37146131 | 37146146 | RALGAPB | 0.002332872 |
| 17 | 43226461 | 43226506 | HEXIM1(NM_006460: c.-97_-52del-) | 0.002347418 |
| 3 | 52676065 | 52676079 | PBRM1 | 0.002352941 |
| 18 | 30275526 | 30275539 | KLHL14 | 0.002352941 |
| 15 | 41663884 | 41663900 | NUSAP1 | 0.002386905 |
| 17 | 39659874 | 39659890 | KRT13 | 0.002414487 |
| 5 | 96116095 | 96116125 | ERAP1 | 0.002414487 |
| 22 | 46679850 | 46679864 | TTC38 | 0.002442838 |
| 2 | 91887905 | 91887919 | LOC654342(dist = 39930), GGT8P(dist = 75449) | 0.00244898 |
| 13 | 102235764 | 102235780 | ITGBL1 | 0.00244898 |
| 21 | 46602483 | 46602497 | ADARB1 | 0.002500759 |
| 1 | 113159364 | 113159384 | ST7L | 0.002557545 |
| 5 | 180482303 | 180482317 | BTNL9 | 0.002645503 |
| 5 | 147793666 | 147793680 | FBXO38 | 0.002645503 |
| 1 | 158584103 | 158584116 | SPTA1 | 0.002687444 |
| 21 | 33068865 | 33068877 | SCAF4 | 0.002687444 |
| 3 | 150282051 | 150282075 | EIF2A | 0.002713704 |
| 6 | 7595241 | 7595255 | SNRNP48 | 0.002713704 |
| 15 | 54586266 | 54586278 | UNC13C | 0.002754337 |
| 1 | 212969842 | 212969856 | TATDN3 | 0.002884615 |
| X | 135115669 | 135115681 | SLC9A6 | 0.002895838 |
| 7 | 116556086 | 116556111 | CAPZA2 | 0.002895838 |
| 1 | 99164246 | 99164260 | SNX7 | 0.002898551 |
| 12 | 89853387 | 89853405 | POC1B | 0.002970297 |
| 22 | 30051705 | 30051722 | NF2 | 0.002970713 |
| 5 | 133707247 | 133707259 | UBE2B(NM_003337: c.-40_-28del-) | 0.002970713 |
| 15 | 30033645 | 30033656 | TJP1 | 0.003030303 |
| 12 | 76780051 | 76780066 | OSBPL8 | 0.00304853 |
| 17 | 35550269 | 35550290 | ACACA | 0.00304853 |
| 2 | 15536509 | 15536532 | NBAS | 0.003172924 |
| 13 | 113818805 | 113818818 | PROZ | 0.003278689 |
| 1 | 237965133 | 237965146 | RYR2 | 0.003301246 |
| 11 | 118247264 | 118247277 | UBE4A | 0.003301246 |
| 8 | 59518439 | 59518456 | NSMAF | 0.003381378 |
| 10 | 90362299 | 90362324 | LIPJ | 0.003381378 |
| 1 | 155330237 | 155330253 | ASH1L | 0.003392501 |
| 17 | 27829588 | 27829602 | TAOK1 | 0.003417635 |
| 19 | 37721404 | 37721419 | ZNF383 | 0.003484321 |
| 20 | 33033065 | 33033083 | ITCH | 0.003586889 |
| 6 | 42611936 | 42611951 | UBR2 | 0.003734483 |
| 16 | 69965396 | 69965409 | WWP2 | 0.00381172 |
| 3 | 146177594 | 146177608 | PLSCR2 | 0.003891377 |
| 3 | 142241692 | 142241705 | ATR | 0.003891377 |
| 12 | 81239721 | 81239737 | LIN7A | 0.003891377 |
| 19 | 23845789 | 23845807 | ZNF675 | 0.003910927 |
| 1 | 3549854 | 3549874 | WRAP73 | 0.003910927 |
| 2 | 39046321 | 39046333 | DHX57 | 0.003952569 |
| 17 | 66038979 | 66038999 | KPNA2 | 0.004140787 |
| 12 | 8813345 | 8813362 | MFAP5 | 0.004145924 |
| 10 | 93752074 | 93752105 | BTAF1 | 0.004145924 |
| 6 | 133093231 | 133093247 | SLC18B1 | 0.004145924 |
| 12 | 72067966 | 72067980 | THAP2 | 0.004152098 |
| 15 | 50581627 | 50581647 | GABPB1 | 0.004152098 |
| 4 | 110433282 | 110433303 | SEC24B | 0.004236343 |
| 2 | 136527318 | 136527332 | UBXN4 | 0.004236343 |
| 13 | 111076981 | 111076999 | COL4A2 | 0.004236343 |
| 1 | 28374836 | 28374848 | EYA3 | 0.004236343 |
| 5 | 172578549 | 172578567 | BNIP1 | 0.004262575 |
| 1 | 155679543 | 155679557 | DAP3 | 0.004426285 |
| 1 | 94964139 | 94964155 | ABCD3 | 0.004736072 |
| 2 | 32427092 | 32427104 | SLC30A6 | 0.004761905 |
| 8 | 35401930 | 35401941 | UNC5D | 0.00484659 |
| 16 | 24185815 | 24185831 | PRKCB | 0.005079552 |
| X | 114871127 | 114871141 | PLS3 | 0.005102041 |
| 19 | 4297855 | 4297872 | TMIGD2 | 0.005202381 |
| 1 | 10357206 | 10357224 | KIF1B | 0.005461791 |
| 5 | 68564968 | 68564998 | CDK7 | 0.005461791 |
| 1 | 245025697 | 245025717 | HNRNPU | 0.005552794 |
| 11 | 88068256 | 88068270 | CTSC | 0.005598832 |
| 2 | 70066571 | 70066582 | GMCL1 | 0.005598832 |
| 16 | 10783088 | 10783102 | TEKT5 | 0.005649718 |
| 17 | 28811834 | 28811848 | GOSR1 | 0.005888848 |
| 1 | 154227292 | 154227307 | UBAP2L | 0.005888848 |
| 18 | 25727760 | 25727775 | CDH2 | 0.006048387 |
| 19 | 10741681 | 10741697 | SLC44A2 | 0.006056781 |
| 17 | 66041871 | 66041886 | KPNA2 | 0.006171026 |
| 6 | 24412431 | 24412445 | MRS2 | 0.006540787 |
| 20 | 47705763 | 47705776 | CSE1L | 0.006584723 |
| 4 | 15826415 | 15826426 | CD38 | 0.006720689 |
| 6 | 30154350 | 30154364 | TRIM26 | 0.006896552 |
| 6 | 10926514 | 10926528 | SYCP2L | 0.007103491 |
| 14 | 96770942 | 96770967 | ATG2B | 0.007217929 |
| 1 | 113196179 | 113196217 | CAPZA1 | 0.007519945 |
| 3 | 172046695 | 172046709 | FNDC3B | 0.007544507 |
| 16 | 4873743 | 4873761 | GLYR1 | 0.007611458 |
| 15 | 89533168 | 89533181 | MFGE8(dist = 76505), ABHD2(dist = 98200) | 0.007832463 |
| 5 | 413428 | 413452 | AHRR | 0.007974115 |
| 5 | 72795055 | 72795066 | BTF3 | 0.007974115 |
| 4 | 55976947 | 55976961 | KDR | 0.007998358 |
| 11 | 116629734 | 116629748 | BUD13 | 0.008097166 |
| 14 | 104219578 | 104219590 | PPP1R13B | 0.008196721 |
| 5 | 127522153 | 127522180 | SLC12A2 | 0.008395953 |
| 13 | 58299427 | 58299446 | PCDH17 | 0.008395953 |
| 6 | 151859146 | 151859161 | CCDC170 | 0.008807588 |
| 3 | 73440108 | 73440119 | PDZRN3 | 0.008949965 |
| 1 | 65138787 | 65138801 | CACHD1 | 0.009250243 |
| 4 | 156725681 | 156725695 | GUCY1B3 | 0.009250243 |
| 8 | 95861778 | 95861797 | INTS8 | 0.009727115 |
| 8 | 42323207 | 42323223 | SLC20A2 | 0.009803922 |
| 9 | 69127139 | 69127155 | PGM5P2 | 0.009992739 |
| 2 | 203079025 | 203079039 | SUMO1 | 0.010278746 |
| 17 | 13399486 | 13399503 | HS3ST3A1(NM_006042: c.*28_*11del0) | 0.01048218 |
| X | 44949951 | 44949963 | KDM6A | 0.010526316 |
| 20 | 33665775 | 33665786 | TRPC4AP | 0.010526316 |
| 18 | 180211 | 180228 | USP14 | 0.010638298 |
| 6 | 97000341 | 97000356 | UFL1 | 0.010695187 |
| X | 35944077 | 35944088 | CXorf22 | 0.010737856 |
| 12 | 56380950 | 56380964 | RAB5B | 0.010752688 |
| 10 | 70520707 | 70520747 | CCARI | 0.010752688 |
| 20 | 10273694 | 10273715 | SNAP25 | 0.010869565 |
| 14 | 31424778 | 31424804 | STRN3 | 0.010869565 |
| 17 | 62748076 | 62748093 | LOC146880 | 0.010885341 |
| 7 | 47979920 | 47979933 | PKD1L1 | 0.010989011 |
| 17 | 60374173 | 60374189 | TBC1D3P2(dist = 21157), EFCAB3(dist = 73390) | 0.011004818 |
| 2 | 211179765 | 211179777 | MYL1 | 0.011139093 |
| 12 | 69214081 | 69214094 | MDM2 | 0.011235955 |
| 3 | 57850505 | 57850518 | SLMAP | 0.011235955 |
| X | 24760276 | 24760289 | POLA1 | 0.011267606 |
| X | 20204548 | 20204563 | RPS6KA3 | 0.011363636 |
| 2 | 192011485 | 192011497 | STAT4 | 0.011363636 |
| 14 | 104465128 | 104465144 | TDRD9 | 0.011363636 |
| 6 | 129905035 | 129905050 | ARHGAP18 | 0.011363636 |
| 12 | 15776223 | 15776244 | EPS8 | 0.011494253 |
| 13 | 39435552 | 39435564 | FREM2 | 0.011627907 |
| 3 | 180679235 | 180679250 | FXR1 | 0.011627907 |
| 1 | 28929961 | 28929973 | TAF12(NM_001135218: c.*90_*78del0, | 0.011627907 |
| | | | NM_005644: c.*90_*78del0) | |
| 11 | 47726034 | 47726053 | AGBL2 | 0.011627907 |
| 15 | 50754453 | 50754465 | USP8 | 0.011627907 |
| 19 | 47700438 | 47700453 | SAE1 | 0.011764706 |
| 15 | 33218472 | 33218487 | FMN1 | 0.011764706 |
| 11 | 57094842 | 57094869 | SSRP1 | 0.011904762 |
| 2 | 173861105 | 173861120 | RAPGEF4 | 0.011904762 |
| 7 | 44715476 | 44715493 | OGDH | 0.011904762 |
| 2 | 219341717 | 219341730 | USP37 | 0.012048193 |
| 16 | 28842257 | 28842275 | ATXN2L | 0.012048193 |
| 11 | 47701629 | 47701642 | AGBL2 | 0.012048193 |
| 17 | 10543281 | 10543296 | MYH3 | 0.012096774 |
| 3 | 108175734 | 108175745 | MYH15 | 0.012195122 |
| 1 | 155671899 | 155671929 | DAP3 | 0.012195122 |
| 6 | 31765848 | 31765875 | LSM2 | 0.012195122 |
| 3 | 37360696 | 37360708 | GOLGA4 | 0.012345679 |
| 3 | 108081175 | 108081187 | HHLA2 | 0.012345679 |
| X | 111090289 | 111090311 | TRPC5 | 0.012345679 |
| 2 | 228356364 | 228356398 | AGFG1 | 0.012406001 |
| 2 | 47635523 | 47635537 | MSH2 | 0.0125 |
| 3 | 30691871 | 30691882 | TGFBR2 | 0.0125 |
| 3 | 137907220 | 137907259 | ARMC8(NM_213654: c.-127_-111delins-) | 0.0125 |
| 3 | 5249962 | 5249976 | EDEM1 | 0.012658228 |
| 2 | 120567392 | 120567406 | PTPN4 | 0.012658228 |
| 17 | 67178764 | 67178794 | ABCA10 | 0.012658228 |
| 3 | 51504906 | 51504917 | VPRBP | 0.012658228 |
| 20 | 57611828 | 57611841 | SLMO2-ATP5E | 0.012658228 |
| 6 | 44216544 | 44216556 | HSP90AB1 | 0.012704174 |
| 7 | 646958 | 646969 | LOC101926963 | 0.012720908 |
| 3 | 108224685 | 108224701 | MYH15 | 0.012820513 |
| 1 | 185120927 | 185120940 | TRMT1L | 0.012820513 |
| 1 | 65129233 | 65129268 | CACHD1 | 0.012820513 |
| 14 | 23870220 | 23870234 | MYH6 | 0.012820513 |
| 1 | 39751172 | 39751188 | MACF1 | 0.012903226 |
| 19 | 36145409 | 36145425 | COX6B1 | 0.012987013 |
| 4 | 17841869 | 17841881 | NCAPG | 0.012987013 |
| 2 | 33500751 | 33500763 | LTBP1 | 0.012987013 |
| 14 | 39619587 | 39619599 | TRAPPC6B | 0.012987013 |
| 20 | 23473522 | 23473535 | CST8 | 0.013157895 |
| 7 | 63673449 | 63673467 | ZNF735 | 0.013157895 |
| 14 | 39591613 | 39591627 | GEMIN2 | 0.013186884 |
| 2 | 207027356 | 207027368 | EEF1B2 | 0.013333333 |
| 5 | 54552380 | 54552391 | DHX29 | 0.013333333 |
| 8 | 120800692 | 120800703 | TAF2 | 0.013333333 |
| 14 | 53180739 | 53180777 | PSMC6 | 0.013333333 |
| 3 | 33339136 | 33339148 | FBXL2 | 0.013333333 |
| 6 | 71009951 | 71009963 | COL9A1 | 0.013333333 |
| 5 | 115628120 | 115628134 | COMMD10 | 0.013420066 |
| 12 | 22215194 | 22215212 | CMAS | 0.013513514 |
| 21 | 30339205 | 30339217 | LTN1 | 0.013513514 |
| 4 | 183600752 | 183600764 | TENM3 | 0.01355087 |

TABLE 11
Microsatellite Repeat Marker Sequences for Lymphoid Neoplasm Diffuse
Large B-cell Lymphoma (DLBC).
TABLE 11 - DLBC

| ch | start_pos | end_pos | | DLBC |
|---|---|---|---|---|
| (hg37) | (hg37) | (hg37) | gene | p-value |
| 7 | 66463195 | 66463206 | TYW1 | 0.000112931 |
| X | 22244528 | 22244549 | PHEX | 0.000326797 |
| 1 | 75614380 | 75614410 | LHX8 | 0.000564653 |
| 9 | 98230924 | 98230947 | LOC100507346 | 0.000877193 |
| 4 | 65180308 | 65180357 | TECRL | 0.000877193 |
| 1 | 153907278 | 153907322 | DENND4B | 0.000877193 |
| 6 | 51910735 | 51910747 | PKHD1 | 0.000999001 |
| 4 | 70280786 | 70280798 | UGT2B28(dist = 120018), UGT2B4(dist = 65085) | 0.00122549 |
| 4 | 73933671 | 73933706 | COX18 | 0.00122549 |
| 12 | 117768089 | 117768134 | NOS1 | 0.00128999 |
| 9 | 15458080 | 15458092 | SNAPC3 | 0.001470588 |
| 2 | 233271682 | 233271728 | ALPPL2 | 0.001470588 |
| 3 | 189690816 | 189690828 | LEPREL1 | 0.001470588 |
| 3 | 113285185 | 113285212 | SIDT1 | 0.001470588 |
| 1 | 155631307 | 155631342 | YY1AP1 | 0.001538462 |
| 12 | 122097043 | 122097074 | MORN3 | 0.001633987 |
| 17 | 39021015 | 39021026 | KRT12 | 0.001785714 |
| 14 | 92588155 | 92588170 | CPSF2, NDUFB1 | 0.001785714 |
| 12 | 123103008 | 123103016 | KNTC1 | 0.001976285 |
| 17 | 13399486 | 13399503 | HS3ST3A1(NM_006042: c.*28_*11del0) | 0.001998002 |
| 1 | 32119347 | 32119376 | COL16A1 | 0.002020202 |
| 4 | 143326312 | 143326333 | INPP4B | 0.002020202 |
| 21 | 19653342 | 19653355 | TMPRSS15 | 0.002050581 |
| 1 | 75679569 | 75679580 | SLC44A5 | 0.00210084 |
| 2 | 192225477 | 192225514 | MYO1B | 0.002258611 |
| 1 | 155737425 | 155737454 | GON4L | 0.002258611 |
| 1 | 43664319 | 43664330 | CFAP57 | 0.002597403 |
| 17 | 27614746 | 27614770 | NUFIP2 | 0.002597403 |
| 3 | 148885779 | 148885790 | HPS3 | 0.002747253 |
| 3 | 146177594 | 146177608 | PLSCR2 | 0.003007519 |
| 4 | 66286113 | 66286124 | EPHA5 | 0.003007519 |
| 15 | 33915964 | 33915975 | RYR3 | 0.003007519 |
| 12 | 1042252 | 1042272 | RAD52 | 0.003030303 |
| 12 | 85285920 | 85285938 | SLC6A15(NM_182767: c.-21_-39del0, | 0.003095975 |
| | | | NM_018057: c.-21_-39del0) | |
| X | 52706249 | 52706291 | SSX7(dist = 22299), SSX2B(dist = 19655) | 0.003095975 |
| 12 | 43964612 | 43964623 | ADAMTS20(dist = 18888), PUS7L(dist = 157787) | 0.003496503 |
| 2 | 113310197 | 113310210 | POLR1B | 0.003496503 |
| 20 | 23349607 | 23349618 | GZF1 | 0.003952569 |
| 2 | 70066571 | 70066582 | GMCL1 | 0.004127967 |
| 13 | 39603371 | 39603407 | PROSER1 | 0.004127967 |
| 4 | 10027509 | 10027520 | SLC2A9 | 0.004127967 |
| 11 | 88068256 | 88068270 | CTSC | 0.004329004 |
| 1 | 92944314 | 92944351 | GFI1 | 0.004449388 |
| 1 | 38483296 | 38483310 | UTP11L | 0.004761905 |
| 3 | 113376110 | 113376147 | KIAA2018 | 0.004901961 |
| 19 | 52132524 | 52132559 | SIGLEC5 | 0.005263158 |
| 12 | 66771108 | 66771126 | GRIP1 | 0.005263158 |
| 16 | 84121078 | 84121113 | MBTPS1 | 0.005847953 |
| 6 | 107420417 | 107420451 | BEND3 | 0.005847953 |
| 17 | 17039561 | 17039595 | MPRIP | 0.005882353 |
| 16 | 70693872 | 70693884 | IL34 | 0.005882353 |
| 5 | 83432985 | 83433037 | EDIL3 | 0.005882353 |
| 5 | 180663941 | 180663952 | GNB2L1(NM_006098: c.*36_*25del0) | 0.005882353 |
| 17 | 10411322 | 10411354 | MYHAS | 0.005882353 |
| 2 | 112725684 | 112725699 | MERTK | 0.005882353 |
| 17 | 10543281 | 10543296 | MYH3 | 0.006060606 |
| 19 | 46815969 | 46815994 | HIF3A | 0.006060606 |
| 3 | 10076842 | 10076853 | FANCD2 | 0.006105006 |
| 16 | 22579717 | 22579776 | LOC653786 | 0.006105951 |
| 11 | 26692594 | 26692629 | SLC5A12(NM_178498: c.*55_*20del0) | 0.006302521 |
| 1 | 198717210 | 198717235 | PTPRC | 0.006493506 |
| 16 | 21991811 | 21991822 | UQCRC2 | 0.006493506 |
| 2 | 215876589 | 215876603 | ABCA12 | 0.006535948 |
| 2 | 61187478 | 61187496 | PUS10 | 0.006535948 |
| 1 | 68952564 | 68952605 | DEPDC1 | 0.006535948 |
| 17 | 28811658 | 28811669 | GOSR1 | 0.006993007 |
| 15 | 50570999 | 50571024 | GABPB1 | 0.007142857 |
| 19 | 22836873 | 22836886 | ZNF492 | 0.007352941 |
| 1 | 114224782 | 114224799 | MAGI3 | 0.007352941 |
| 1 | 54692854 | 54692865 | SSBP3 | 0.007352941 |
| 3 | 179103328 | 179103352 | MFN1 | 0.007352941 |
| 12 | 14949720 | 14949731 | WBP11 | 0.007352941 |
| 7 | 134925241 | 134925252 | STRA8 | 0.007518797 |
| 11 | 114398627 | 114398638 | NXPE1 | 0.007703081 |
| 1 | 115276971 | 115276990 | CSDE1 | 0.008333333 |
| 20 | 47615074 | 47615089 | ARFGEF2 | 0.008333333 |
| 4 | 88231324 | 88231350 | HSD17B13 | 0.008598572 |
| 14 | 50605267 | 50605280 | SOS2 | 0.00862069 |
| 4 | 119674051 | 119674062 | SEC24D | 0.00877193 |
| 14 | 88651822 | 88651847 | KCNK10(NM_138317: c.*57_*32del0, | 0.00877193 |
| | | | NM_138318: c.*57_*32del0, | |
| | | | NM_021161: c.*57_*32del0) | |
| 10 | 21177138 | 21177164 | NEBL | 0.00952381 |
| 1 | 236703817 | 236703841 | LGALS8 | 0.00952381 |
| 4 | 77065476 | 77065492 | NUP54 | 0.00952381 |
| 7 | 14028245 | 14028266 | ETV1 | 0.010319917 |
| 2 | 227967830 | 227967842 | COL4A4 | 0.010989011 |
| 12 | 22215194 | 22215212 | CMAS | 0.010989011 |
| 5 | 148207659 | 148207671 | ADRB2(NM_000024: c.*23_*35del0) | 0.010989011 |
| 7 | 146805220 | 146805232 | CNTNAP2 | 0.010989011 |
| 3 | 42251577 | 42251608 | TRAK1 | 0.010989011 |
| 2 | 271998 | 272015 | ACP1 | 0.011857708 |
| 16 | 87935958 | 87935971 | CA5A | 0.011857708 |
| 3 | 183493743 | 183493771 | YEATS2 | 0.011904762 |
| 10 | 26802633 | 26802648 | APBB1IP | 0.012820513 |
| 1 | 202990110 | 202990128 | TMEM183A, TMEM183B | 0.012820513 |
| 5 | 157180930 | 157180952 | LSM11 | 0.012987013 |
| 5 | 122359502 | 122359533 | PPIC(NM_000943: c.*68_*37del0) | 0.013931889 |
| 4 | 146297387 | 146297396 | OTUD4(dist = 196555), SMAD1(dist = 105555) | 0.013986014 |
| 6 | 51930886 | 51930901 | PKHD1 | 0.014285714 |
| 3 | 43756577 | 43756612 | ABHD5 | 0.014285714 |
| 12 | 3018675 | 3018690 | TULP3 | 0.014705882 |
| 17 | 18708830 | 18708843 | TVP23B | 0.015037594 |
| 10 | 115904180 | 115904217 | CCDC186 | 0.015151515 |
| 4 | 155469959 | 155469971 | PLRG1 | 0.015151515 |
| 4 | 110606384 | 110606404 | CCDC109B | 0.015151515 |
| 12 | 76461257 | 76461268 | NAP1L1 | 0.015151515 |
| 13 | 25073399 | 25073415 | PARP4 | 0.015217391 |
| 3 | 47143087 | 47143124 | SETD2 | 0.015217391 |
| 3 | 154010515 | 154010550 | DHX36 | 0.015737874 |
| 7 | 149589490 | 149589511 | ATP6V0E2(dist = 11689), ACTR3C(dist = 354790) | 0.015789474 |
| 22 | 16404838 | 16404857 | POTEH(dist = 116901), OR11H1(dist = 43967) | 0.016205534 |
| 3 | 66434357 | 66434397 | LRIG1 | 0.017094017 |
| 2 | 139308383 | 139308420 | SPOPL | 0.017266486 |
| 2 | 202494630 | 202494642 | TMEM237 | 0.01754386 |
| 3 | 186295417 | 186295431 | DNAJB11 | 0.01754386 |
| 11 | 14539315 | 14539332 | PSMA1 | 0.01754386 |
| 1 | 111059812 | 111059831 | KCNA10 | 0.01754386 |
| 11 | 89914885 | 89914911 | NAALAD2 | 0.01754386 |
| 2 | 58453843 | 58453856 | FANCL | 0.017857143 |
| 3 | 154002357 | 154002370 | DHX36 | 0.017857143 |
| 12 | 107080617 | 107080640 | LOC100287944 | 0.018181818 |
| 1 | 220789355 | 220789373 | MARK1 | 0.018181818 |
| 7 | 114303491 | 114303502 | FOXP2 | 0.018181818 |
| 6 | 87796161 | 87796171 | CGA | 0.018181818 |
| 2 | 74756111 | 74756122 | AUP1 | 0.018181818 |
| 1 | 118623681 | 118623714 | SPAG17 | 0.018351214 |
| 11 | 60778650 | 60778677 | CD6 | 0.018461538 |
| 1 | 245025697 | 245025717 | HNRNPU | 0.019607843 |
| 1 | 175087683 | 175087694 | TNN | 0.019607843 |
| 3 | 125651602 | 125651614 | ALG1L | 0.019607843 |
| 12 | 8807982 | 8807993 | MFAP5 | 0.019607843 |
| 17 | 53124546 | 53124579 | STXBP4 | 0.019607843 |
| 10 | 52610342 | 52610369 | A1CF | 0.019607843 |
| 14 | 96770942 | 96770967 | ATG2B | 0.019762846 |
| 15 | 78839102 | 78839127 | PSMA4 | 0.02 |
| 9 | 85597720 | 85597749 | RASEF | 0.021978022 |
| 2 | 15319070 | 15319084 | NBAS | 0.021978022 |
| 5 | 96342077 | 96342093 | LNPEP | 0.022058824 |
| 17 | 40986454 | 40986487 | PSME3 | 0.022058824 |
| 1 | 94544805 | 94544830 | ABCA4 | 0.022058824 |
| 20 | 52188386 | 52188410 | ZNF217 | 0.022058824 |
| X | 70594925 | 70594942 | BCYRN1 | 0.022222222 |
| 2 | 182399497 | 182399529 | ITGA4 | 0.022268908 |
| 9 | 34017005 | 34017043 | UBAP2 | 0.023715415 |
| 11 | 116629734 | 116629748 | BUD13 | 0.023715415 |
| 17 | 15643372 | 15643392 | TBC1D26 | 0.024242424 |
| 9 | 97555228 | 97555242 | C9orf3 | 0.024630542 |
| 6 | 153292551 | 153292568 | FBXO5 | 0.025 |
| 1 | 9770690 | 9770741 | PIK3CD | 0.025974026 |
| 4 | 88532182 | 88532215 | DSPP | 0.025974026 |
| 3 | 170857167 | 170857204 | TNIK | 0.025974026 |
| 20 | 29449657 | 29449688 | NONE(dist = NONE), FRG1B(dist = 162191) | 0.025974026 |
| 8 | 121293133 | 121293158 | COL14A1 | 0.026315789 |
| 4 | 148457005 | 148457019 | EDNRA | 0.027568922 |
| 12 | 105260159 | 105260194 | SLC41A2 | 0.027777778 |
| 4 | 46994972 | 46994992 | GABRA4 | 0.028571429 |
| 2 | 152359276 | 152359297 | NEB | 0.028571429 |
| 10 | 88458996 | 88459025 | LDB3 | 0.028571429 |
| 7 | 64004813 | 64004850 | ZNF680 | 0.028571429 |
| 1 | 153954763 | 153954831 | RAB13 | 0.028571429 |
| 10 | 106974123 | 106974140 | SORCS3 | 0.028571429 |
| 3 | 47103861 | 47103876 | SETD2 | 0.028571429 |
| 20 | 49551780 | 49551793 | ADNP-AS1 | 0.028571429 |
| 1 | 50956053 | 50956192 | FAF1 | 0.030701754 |
| 13 | 110859316 | 110859336 | COL4A1 | 0.031578947 |
| 17 | 31324330 | 31324379 | SPACA3 | 0.031578947 |
| 15 | 33256195 | 33256250 | FMN1 | 0.031620553 |
| 12 | 9021018 | 9021072 | A2ML1 | 0.031991744 |
| 11 | 35287000 | 35287014 | SLC1A2 | 0.032967033 |
| 4 | 15826415 | 15826426 | CD38 | 0.032967033 |
| 7 | 77221481 | 77221508 | PTPN12 | 0.032967033 |
| 19 | 21558015 | 21558037 | ZNF738 | 0.032967033 |
| 19 | 38282490 | 38282505 | ZNF573(dist = 12260), LOC644554(dist = 25546) | 0.033333333 |
| 4 | 110268615 | 110268631 | COL25A1(dist = 44816), SEC24B-AS1(dist = 82488) | 0.033333333 |
| 1 | 202107422 | 202107460 | ARL8A | 0.034443817 |
| 3 | 159976432 | 159976443 | IFT80 | 0.035087719 |
| 21 | 47647618 | 47647632 | LSS | 0.035714286 |
| 3 | 21606027 | 21606040 | ZNF385D-AS1 | 0.035714286 |
| 19 | 55438923 | 55438944 | NLRP7 | 0.038461538 |
| 10 | 34985197 | 34985210 | PARD3 | 0.038461538 |
| 13 | 31722620 | 31722638 | HSPH1 | 0.039215686 |
| 21 | 38522344 | 38522368 | TTC3 | 0.039215686 |
| 8 | 73983976 | 73984004 | SBSPON | 0.039215686 |
| 9 | 79897218 | 79897232 | VPS13A | 0.039215686 |
| 12 | 21639385 | 21639398 | RECQL | 0.039525692 |
| 4 | 96035796 | 96035838 | BMPR1B | 0.041353383 |
| 9 | 117803175 | 117803204 | TNC | 0.042735043 |
| 9 | 74349917 | 74349938 | TMEM2 | 0.043290043 |
| 3 | 48226193 | 48226204 | CDC25A | 0.043478261 |
| 12 | 111701486 | 111701497 | CUX2 | 0.044117647 |
| 1 | 24973106 | 24973116 | SRRM1 | 0.044117647 |
| 3 | 75466006 | 75466019 | MIR4444-1(dist = 202307), FAM86DP(dist = 4684) | 0.044117647 |
| 15 | 31220709 | 31220744 | FAN1 | 0.045454545 |
| 5 | 70805272 | 70805288 | BDP1 | 0.045454545 |
| 14 | 81574687 | 81574712 | TSHR | 0.045454545 |
| 19 | 41798468 | 41798491 | HNRNPUL1 | 0.045454545 |
| 2 | 128608271 | 128608284 | POLR2D | 0.045796309 |
| 7 | 135375909 | 135375932 | SLC13A4 | 0.046153846 |
| 4 | 84519775 | 84519790 | AGPAT9 | 0.047385621 |
| 9 | 32986030 | 32986069 | APTX | 0.047619048 |
| 5 | 130535204 | 130535217 | LYRM7 | 0.047619048 |
| 17 | 80443296 | 80443308 | NARF | 0.047619048 |
| 5 | 162945387 | 162945400 | MAT2B(NM_182796: c.*18_*31del0, | 0.047619048 |
| | | | NM_013283: c.*18_*31del0) | |
| 3 | 5022981 | 5022992 | BHLHE40 | 0.047619048 |
| 17 | 4906146 | 4906166 | KIF1C | 0.047619048 |
| 20 | 44512744 | 44512783 | ZSWIM1(NM_080603: c.*55_*94del0) | 0.047619048 |
| 7 | 102825987 | 102826001 | DPY19L2P2 | 0.05 |
| 3 | 108102580 | 108102602 | MYH15 | 0.05 |
| 18 | 29101215 | 29101231 | DSG2 | 0.05 |
| 12 | 59307848 | 59307866 | LRIG3 | 0.05 |
| 1 | 39900058 | 39900076 | MACF1 | 0.05 |
| 1 | 114372332 | 114372354 | AP4B1-AS1 | 0.05 |
| 7 | 882808 | 882841 | SUN1 | 0.05 |

TABLE 12
Microsatellite Repeat Marker Sequences for Endometrial Carcinoma (UCEC).
TABLE 12 - UCEC

| ch | start_pos | end_pos | | UCEC |
|---|---|---|---|---|
| (hg37) | (hg37) | (hg37) | gene | p-value |
| 7 | 100319680 | 100319696 | EPO | 5.87473E−48 |
| 8 | 103287850 | 103287864 | UBR5 | 3.55758E−45 |
| X | 48214185 | 48214200 | SSX3 | 1.25198E−42 |
| 10 | 74874082 | 74874112 | NUDT13 | 3.88283E−42 |
| 2 | 99936265 | 99936280 | TXNDC9 | 1.95768E−41 |
| 3 | 10018739 | 10018754 | EMC3 | 7.08584E−40 |
| 20 | 37146131 | 37146146 | RALGAPB | 3.04311E−39 |
| 5 | 14485161 | 14485175 | TRIO | 4.20049E−38 |
| 7 | 102727051 | 102727067 | ARMC10 | 7.75873E−38 |
| 2 | 201778717 | 201778733 | ORC2 | 8.73497E−38 |
| 4 | 115586972 | 115586988 | UGT8 | 9.83442E−38 |
| 18 | 180211 | 180228 | USP14 | 3.60196E−37 |
| 6 | 111329221 | 111329239 | RPF2 | 4.32986E−37 |
| X | 48631645 | 48631660 | GLOD5 | 5.92777E−37 |
| 10 | 18280054 | 18280071 | SLC39A12 | 6.63708E−37 |
| X | 20204548 | 20204563 | RPS6KA3 | 1.25803E−36 |
| 5 | 36123065 | 36123135 | LMBRD2 | 1.78204E−36 |
| 1 | 78432506 | 78432521 | FUBP1 | 2.28578E−36 |
| 18 | 649879 | 649895 | TYMSOS(NM_001012716: c.*120_*104del0) | 2.63159E−36 |
| 9 | 104306988 | 104307005 | RNF20 | 1.42702E−35 |
| X | 106092450 | 106092473 | TBC1D8B | 2.05775E−35 |
| 19 | 34951356 | 34951371 | UBA2 | 2.56362E−35 |
| 3 | 129168854 | 129168881 | IFT122 | 3.31279E−35 |
| 5 | 172578549 | 172578567 | BNIP1 | 3.32569E−35 |
| 12 | 124242458 | 124242472 | ATP6V0A2 | 5.614E−35 |
| 2 | 120714401 | 120714418 | PTPN4 | 9.28995E−35 |
| 4 | 146031236 | 146031253 | ABCE1 | 1.39297E−34 |
| 2 | 44445604 | 44445617 | PPM1B | 2.68401E−34 |
| 9 | 125655308 | 125655324 | RC3H2 | 3.79755E−34 |
| 5 | 41909830 | 41909846 | C5orf51 | 4.10845E−34 |
| 8 | 95186510 | 95186526 | CDH17 | 4.12325E−34 |
| 14 | 53513439 | 53513452 | DDHD1(NM_001160148: c.*47_*34del0, | 4.19123E−34 |
| | | | NM_001160147: c.*47_*34del0, | |
| | | | NM_030637: c.*47_*34del0) | |
| 1 | 39751172 | 39751188 | MACF1 | 4.3787E−34 |
| X | 23693084 | 23693098 | PRDX4 | 5.16618E−34 |
| 7 | 98639904 | 98639917 | SMURF1 | 8.36556E−34 |
| 16 | 74372596 | 74372618 | LOC283922 | 8.56745E−34 |
| 7 | 74608740 | 74608754 | GTF2IP1, LOC100093631 | 1.43276E−33 |
| 1 | 39929162 | 39929178 | MACF1 | 1.55105E−33 |
| X | 129267464 | 129267478 | AIFM1 | 1.69459E−33 |
| X | 52654536 | 52654550 | SSX8 | 1.87368E−33 |
| 1 | 41456808 | 41456823 | CTPS1 | 3.92856E−33 |
| 10 | 95549836 | 95549852 | LGI1 | 1.17421E−32 |
| 10 | 90362299 | 90362324 | LIPJ | 1.33214E−32 |
| 6 | 34735652 | 34735670 | SNRPC | 1.36955E−32 |
| 8 | 80831393 | 80831407 | MRPS28 | 1.42129E−32 |
| 6 | 15651642 | 15651656 | DTNBP1 | 1.64398E−32 |
| 11 | 49056627 | 49056644 | TRIM49B | 1.70778E−32 |
| 8 | 100160282 | 100160300 | VPS13B | 5.77233E−32 |
| 11 | 88068256 | 88068270 | CTSC | 5.88968E−32 |
| 3 | 5249962 | 5249976 | EDEM1 | 6.36915E−32 |
| 13 | 79216356 | 79216381 | RNF219 | 8.48926E−32 |
| 5 | 55034756 | 55034770 | DDX4 | 8.71302E−32 |
| 2 | 55883509 | 55883523 | PNPT1 | 9.02468E−32 |
| 4 | 44691795 | 44691810 | GUF1 | 1.50215E−31 |
| 6 | 146256315 | 146256344 | SHPRH | 2.34454E−31 |
| 20 | 33225781 | 33225808 | PIGU | 2.75604E−31 |
| X | 79991590 | 79991604 | BRWD3 | 2.8027E−31 |
| 6 | 89638988 | 89639004 | RNGTT | 3.1474E−31 |
| 9 | 19299209 | 19299224 | DENND4C | 3.83185E−31 |
| 9 | 114341239 | 114341255 | PTGR1 | 6.34751E−31 |
| 15 | 101550861 | 101550877 | LRRK1 | 7.08612E−31 |
| 3 | 52696310 | 52696322 | PBRM1 | 9.24234E−31 |
| X | 129546260 | 129546274 | RBMX2 | 9.63292E−31 |
| 12 | 104714847 | 104714867 | TXNRD1 | 9.65902E−31 |
| 16 | 19063220 | 19063235 | TMC7 | 1.37058E−30 |
| 9 | 102730697 | 102730712 | STX17 | 1.58617E−30 |
| 7 | 114303617 | 114303633 | FOXP2 | 1.6595E−30 |
| 17 | 30293149 | 30293164 | SUZ12 | 2.84185E−30 |
| 3 | 196088809 | 196088826 | UBXN7 | 4.16839E−30 |
| 1 | 173795803 | 173795821 | DARS2 | 4.18459E−30 |
| 5 | 94842718 | 94842730 | TTC37 | 4.70941E−30 |
| X | 110494528 | 110494543 | CAPN6 | 5.20825E−30 |
| 12 | 48360947 | 48360961 | TMEM106C | 5.62633E−30 |
| 19 | 36145409 | 36145425 | COX6B1 | 5.66865E−30 |
| 1 | 113159364 | 113159384 | ST7L | 5.71755E−30 |
| 3 | 20189528 | 20189540 | KAT2B | 6.34461E−30 |
| 2 | 48032740 | 48032754 | MSH6 | 6.84671E−30 |
| X | 19606999 | 19607014 | SH3KBP1 | 6.95349E−30 |
| 1 | 33087567 | 33087584 | ZBTB8OS | 7.24192E−30 |
| 13 | 73409508 | 73409522 | PIBF1 | 8.89678E−30 |
| 4 | 76528928 | 76528945 | CDKL2 | 1.12498E−29 |
| 2 | 64112697 | 64112719 | UGP2 | 1.24428E−29 |
| 1 | 172577864 | 172577879 | SUCO | 1.27534E−29 |
| 2 | 179407763 | 179407779 | MIR548N, TTN-AS1 | 1.47356E−29 |
| 2 | 178364328 | 178364342 | AGPS | 1.62863E−29 |
| 12 | 95488339 | 95488354 | FGD6 | 1.71018E−29 |
| 6 | 152728079 | 152728093 | SYNE1 | 1.73993E−29 |
| 5 | 52358761 | 52358775 | ITGA2 | 2.19395E−29 |
| 6 | 152740681 | 152740695 | SYNE1 | 2.68788E−29 |
| 22 | 29682873 | 29682894 | EWSR1 | 2.84429E−29 |
| 10 | 74906122 | 74906136 | ECD | 2.8723E−29 |
| 5 | 132408905 | 132408918 | HSPA4 | 3.38894E−29 |
| 7 | 135099044 | 135099059 | CNOT4 | 4.04666E−29 |
| 20 | 58497481 | 58497497 | SYCP2(NM_014258: c.-13_-29del0) | 4.2019E−29 |
| 13 | 21370398 | 21370412 | XPO4 | 4.95193E−29 |
| 16 | 70176311 | 70176336 | PDPR | 5.44704E−29 |
| 2 | 207027356 | 207027368 | EEF1B2 | 5.7094E−29 |
| 1 | 114092111 | 114092124 | MAGI3 | 6.3653E−29 |
| 1 | 176105710 | 176105723 | RFWD2 | 8.23522E−29 |
| 6 | 97000341 | 97000356 | UFL1 | 8.40277E−29 |
| X | 44949951 | 44949963 | KDM6A | 8.70466E−29 |
| X | 148582573 | 148582585 | IDS | 8.83709E−29 |
| 5 | 172421760 | 172421776 | ATP6V0E1 | 9.22535E−29 |
| 2 | 203417391 | 203417403 | BMPR2 | 9.23405E−29 |
| 14 | 76638065 | 76638087 | GPATCH2L | 9.31556E−29 |
| X | 19380975 | 19380993 | MAP3K15 | 9.58034E−29 |
| 6 | 80910611 | 80910635 | BCKDHB | 1.0545E−28 |
| 6 | 17669262 | 17669277 | NUP153 | 2.26794E−28 |
| 6 | 116912033 | 116912049 | RWDD1 | 2.47805E−28 |
| 2 | 54146387 | 54146400 | PSME4 | 2.54564E−28 |
| 1 | 150297334 | 150297351 | PRPF3 | 2.77807E−28 |
| 1 | 32381592 | 32381607 | PTP4A2 | 2.79089E−28 |
| 3 | 172052897 | 172052914 | FNDC3B | 2.89631E−28 |
| 4 | 141448595 | 141448610 | ELMOD2 | 3.24235E−28 |
| 2 | 197654578 | 197654593 | GTF3C3 | 3.28075E−28 |
| 6 | 70040500 | 70040513 | BAI3 | 3.4673E−28 |
| 14 | 91372685 | 91372700 | RPS6KA5 | 4.00986E−28 |
| 22 | 46679850 | 46679864 | TTC38 | 4.18236E−28 |
| 7 | 138434066 | 138434081 | ATP6V0A4 | 4.89056E−28 |
| 19 | 6908675 | 6908689 | EMR1 | 5.27457E−28 |
| 12 | 96704958 | 96704972 | CDK17 | 5.76102E−28 |
| 15 | 64496611 | 64496630 | CSNK1G1 | 5.81415E−28 |
| 17 | 27829588 | 27829602 | TAOK1 | 5.93681E−28 |
| 2 | 179548695 | 179548712 | TTN | 6.16285E−28 |
| 11 | 59626477 | 59626491 | TCN1 | 6.80027E−28 |
| 2 | 157370018 | 157370033 | GPD2 | 6.93556E−28 |
| 19 | 38282490 | 38282505 | ZNF573(dist = 12260), LOC644554(dist = 25546) | 7.07268E−28 |
| 7 | 36465606 | 36465622 | ANLN | 7.07308E−28 |
| 22 | 41551231 | 41551249 | EP300 | 7.39952E−28 |
| 10 | 112360315 | 112360333 | SMC3 | 7.66214E−28 |
| 12 | 81239721 | 81239737 | LIN7A | 8.09633E−28 |
| 12 | 129360344 | 129360381 | GLT1D1 | 8.64142E−28 |
| 2 | 15536509 | 15536532 | NBAS | 1.30742E−27 |
| 5 | 147785802 | 147785816 | FBXO38 | 1.3383E−27 |
| 6 | 42611936 | 42611951 | UBR2 | 1.5148E−27 |
| 11 | 8486375 | 8486389 | STK33 | 1.55137E−27 |
| 4 | 146046123 | 146046135 | ABCE1 | 1.62305E−27 |
| 9 | 34017005 | 34017043 | UBAP2 | 1.76307E−27 |
| 17 | 54981571 | 54981588 | TRIM25 | 1.76981E−27 |
| 8 | 54742095 | 54742107 | ATP6V1H | 1.77626E−27 |
| 14 | 64460536 | 64460551 | SYNE2 | 1.8423E−27 |
| 15 | 37391752 | 37391765 | MEIS2(NM_170677: c.-85_-98del0, | 1.90794E−27 |
| | | | NM_170676: c.-85_-98del0, | |
| | | | NM_170675: c.-85_-98del0, | |
| | | | NM_170674: c.-85_-98del0, | |
| | | | NM_001220482: c.-85_-98del0) | |
| 12 | 57006059 | 57006072 | BAZ2A | 2.01711E−27 |
| 2 | 66796098 | 66796109 | MEIS1 | 2.05516E−27 |
| 1 | 115226990 | 115227003 | AMPD1 | 2.19661E−27 |
| 1 | 158260878 | 158260892 | CD1C | 2.63234E−27 |
| 8 | 39775377 | 39775390 | IDO1 | 2.71157E−27 |
| 19 | 34945331 | 34945344 | UBA2 | 2.75847E−27 |
| 2 | 87122096 | 87122121 | ANAPC1P1 | 3.20613E−27 |
| 3 | 108224685 | 108224701 | MYH15 | 3.61334E−27 |
| 6 | 88221097 | 88221109 | SLC35A1 | 3.73678E−27 |
| 4 | 39233719 | 39233734 | MIR1273H | 3.79264E−27 |
| 2 | 219341717 | 219341730 | USP37 | 3.94686E−27 |
| 2 | 64323751 | 64323763 | PELI1 | 3.95947E−27 |
| 20 | 34435246 | 34435270 | PHF20 | 4.01226E−27 |
| 15 | 22848150 | 22848166 | TUBGCP5 | 4.46315E−27 |
| 12 | 42835328 | 42835365 | PPHLN1(NM_201439: c.*17_*54del0, | 5.73096E−27 |
| | | | NM_201440: c.*17_*54del0, | |
| | | | NM_001143788: c.*17_*54del0, | |
| | | | NM_001143789: c.*17_*54del0, | |
| | | | NM_201515: c.*17_*54del0) | |
| 4 | 39511530 | 39511543 | MIR1273H | 5.81468E−27 |
| 7 | 107336262 | 107336274 | SLC26A4 | 5.8531E−27 |
| 1 | 222904869 | 222904881 | BROX | 6.00763E−27 |
| 5 | 14678762 | 14678776 | OTULIN | 6.00959E−27 |
| 8 | 30933816 | 30933829 | WRN | 6.78308E−27 |
| 19 | 21350520 | 21350533 | ZNF431 | 7.6725E−27 |
| 1 | 112305406 | 112305423 | DDX20 | 7.75246E−27 |
| 8 | 23712066 | 23712089 | STC1(NM_003155: c.-30_-53del0) | 7.89933E−27 |
| X | 135115669 | 135115681 | SLC9A6 | 9.02664E−27 |
| 7 | 138453892 | 138453909 | ATP6V0A4 | 9.30126E−27 |
| 6 | 108076870 | 108076886 | SCML4 | 1.00496E−26 |
| 11 | 125763610 | 125763624 | PUS3(NM_001271985: c.*70_*56del0, | 1.01141E−26 |
| | | | NM_031307: c.*70_*56del0) | |
| 12 | 64491002 | 64491019 | SRGAP1 | 1.11012E−26 |
| 3 | 16636093 | 16636108 | DAZL | 1.13442E−26 |
| 6 | 71571688 | 71571707 | SMAP1(NM_001281439: c.*1751_*1770del0, | 1.45305E−26 |
| | | | NM_021940: c.*1651_*1670del0, | |
| | | | NM_001044305: c.*1651_*1670del0, | |
| | | | NM_001281440: c.*1651_*1670del0) | |
| 1 | 165712555 | 165712571 | TMCO1 | 1.70207E−26 |
| 22 | 30051705 | 30051722 | NF2 | 1.71632E−26 |
| 16 | 67384060 | 67384075 | LRRC36 | 1.7743E−26 |
| 13 | 115011550 | 115011564 | CDC16 | 1.78567E−26 |
| 2 | 236817336 | 236817351 | AGAP1 | 1.83794E−26 |
| 14 | 75199555 | 75199568 | FCF1 | 1.88459E−26 |
| 2 | 64331815 | 64331833 | PELI1 | 2.07837E−26 |
| 1 | 117605130 | 117605145 | TTF2 | 2.08662E−26 |
| 12 | 72067966 | 72067980 | THAP2 | 2.30261E−26 |
| 9 | 133498229 | 133498245 | FUBP3 | 2.56464E−26 |
| 2 | 228680153 | 228680168 | CCL20 | 2.64191E−26 |
| 9 | 131456159 | 131456172 | SET | 2.85063E−26 |
| 7 | 64275470 | 64275486 | ZNF138 | 2.8571E−26 |
| 20 | 35695535 | 35695562 | RBL1 | 3.4436E−26 |
| 14 | 104465128 | 104465144 | TDRD9 | 4.04883E−26 |
| 9 | 15472611 | 15472623 | PSIP1 | 4.17961E−26 |
| 16 | 19619602 | 19619617 | C16orf62 | 4.57527E−26 |
| 3 | 71008341 | 71008362 | FOXP1(NM_001244816: c.*57_*36del0, | 4.64001E−26 |
| | | | NM_001244812: c*57_*36del0, | |
| | | | NM_001244813: c.*57_*36del0, | |
| | | | NM_001244808: c.*57_*36del0, | |
| | | | NM_001244815: c.*57_*36del0, | |
| | | | NM_001244814: c.*57_*36del0, | |
| | | | NM_032682: c.*57_*36del0, | |
| | | | NM_001244810: c.*57_*36del0) | |
| 2 | 239241445 | 239241460 | TRAF3IP1 | 4.6996E−26 |
| 6 | 116914112 | 116914128 | RWDD1 | 4.92428E−26 |
| 2 | 183832100 | 183832113 | NCKAP1 | 5.55817E−26 |
| 1 | 220369745 | 220369758 | RAB3GAP2 | 5.65277E−26 |
| 2 | 203079025 | 203079039 | SUMO1 | 7.1382E−26 |
| 7 | 75155143 | 75155157 | PMS2P3 | 7.40022E−26 |
| 12 | 22215194 | 22215212 | CMAS | 7.86679E−26 |
| 1 | 94964139 | 94964155 | ABCD3 | 8.27646E−26 |
| 6 | 117243181 | 117243196 | RFX6 | 8.37787E−26 |
| 13 | 77644856 | 77644877 | MYCBP2 | 8.42077E−26 |
| 4 | 103505784 | 103505799 | NFKB1 | 9.16828E−26 |
| 17 | 40881033 | 40881048 | EZH1 | 9.23728E−26 |
| 2 | 61145498 | 61145512 | REL | 9.32516E−26 |
| 2 | 70456452 | 70456465 | TIA1 | 9.68104E−26 |
| 3 | 180679235 | 180679250 | FXR1 | 9.8396E−26 |
| X | 44386435 | 44386460 | FUNDC1 | 1.03952E−25 |
| 2 | 141294271 | 141294299 | LRP1B | 1.04396E−25 |
| 4 | 71888332 | 71888348 | DCK | 1.06332E−25 |
| 20 | 7921106 | 7921120 | HAO1 | 1.08762E−25 |
| 1 | 86435957 | 86435971 | COL24A1 | 1.10853E−25 |
| 3 | 10128965 | 10128985 | FANCD2, FANCD2OS | 1.10945E−25 |
| 17 | 57278908 | 57278920 | PRR11 | 1.19296E−25 |
| 17 | 74736999 | 74737016 | MFSD11 | 1.19457E−25 |
| 15 | 65273147 | 65273161 | SPG21 | 1.38181E−25 |
| 16 | 24574691 | 24574710 | RBBP6 | 1.38528E−25 |
| 6 | 20758808 | 20758822 | CDKAL1 | 1.43421E−25 |
| 17 | 8417006 | 8417020 | MYH10 | 1.53755E−25 |
| 3 | 172046695 | 172046709 | FNDC3B | 1.59251E−25 |
| 6 | 64289938 | 64289954 | PTP4A1 | 1.69401E−25 |
| 9 | 86354663 | 86354679 | GKAP1 | 1.96947E−25 |
| 12 | 107237602 | 107237626 | RIC8B | 2.04862E−25 |
| 6 | 129955151 | 129955165 | ARHGAP18 | 2.062E−25 |
| 13 | 28133956 | 28133972 | LNX2 | 2.12464E−25 |
| 1 | 179089233 | 179089251 | ABL2 | 2.17746E−25 |
| 1 | 229662901 | 229662916 | ABCB10 | 2.20616E−25 |
| 2 | 61308569 | 61308585 | KIAA1841 | 2.36012E−25 |
| 12 | 8813345 | 8813362 | MFAP5 | 2.41616E−25 |
| 12 | 31435608 | 31435620 | FAM60A(NM_001135811: c.*38_*26del0, | 2.57286E−25 |
| | | | NM_001135812: c.*38_*26del0, | |
| | | | NM_021238: c.*38_*26del0) | |
| 3 | 176751870 | 176751887 | TBL1XR1 | 2.59409E−25 |
| 5 | 96342077 | 96342093 | LNPEP | 2.61713E−25 |
| 3 | 160955882 | 160955898 | NMD3 | 2.62196E−25 |
| 3 | 71739332 | 71739345 | EIF4E3 | 2.79307E−25 |
| X | 117530912 | 117530925 | WDR44 | 2.83955E−25 |
| 22 | 30772228 | 30772244 | KIAA1656 | 3.03216E−25 |
| 1 | 99164246 | 99164260 | SNX7 | 3.22713E−25 |
| 10 | 27333121 | 27333134 | ANKRD26 | 3.24697E−25 |
| 15 | 40005793 | 40005806 | FSIP1 | 3.24723E−25 |
| 1 | 46739974 | 46739989 | RAD54L | 3.55395E−25 |
| 7 | 7572355 | 7572381 | COL28A1 | 3.75805E−25 |
| 18 | 43445730 | 43445748 | EPG5 | 3.78319E−25 |
| 3 | 113222099 | 113222114 | SPICE1 | 3.88128E−25 |
| 13 | 98634737 | 98634749 | IPO5 | 3.93558E−25 |
| 1 | 155701055 | 155701071 | DAP3 | 4.07066E−25 |
| 18 | 33058318 | 33058335 | INO80C | 4.12759E−25 |
| 6 | 64419024 | 64419039 | PHF3 | 4.2205E−25 |
| 4 | 164440461 | 164440474 | TMA16 | 4.71879E−25 |
| 10 | 32327037 | 32327053 | KIF5B | 4.73759E−25 |
| 6 | 100403812 | 100403827 | MCHR2 | 4.84698E−25 |
| 10 | 70056619 | 70056632 | PBLD | 5.23991E−25 |
| 11 | 108141955 | 108141971 | ATM | 5.49788E−25 |
| 2 | 135887514 | 135887528 | RAB3GAP1 | 5.65482E−25 |
| X | 24760276 | 24760289 | POLA1 | 6.02405E−25 |
| 5 | 6623405 | 6623418 | NSUN2 | 6.04656E−25 |
| 18 | 43487885 | 43487905 | EPG5 | 6.28651E−25 |
| 5 | 180481216 | 180481233 | BTNL9 | 6.99221E−25 |
| 18 | 6873646 | 6873663 | ARHGAP28 | 7.55919E−25 |
| 4 | 39501722 | 39501754 | MIR1273H | 7.62153E−25 |
| 4 | 25315637 | 25315653 | ZCCHC4 | 7.88524E−25 |
| 2 | 160092703 | 160092716 | WDSUB1 | 7.88627E−25 |
| 8 | 96069973 | 96069988 | NDUFAF6 | 7.94326E−25 |
| 2 | 74089266 | 74089281 | STAMBP | 8.17545E−25 |
| 15 | 42503953 | 42503965 | TMEM87A | 8.18018E−25 |
| 7 | 152144203 | 152144216 | FABP5P3(dist = 4103), LINC01003(dist = 16993) | 8.39608E−25 |
| 1 | 225707271 | 225707295 | ENAH | 8.51443E−25 |
| 18 | 25727760 | 25727775 | CDH2 | 9.77418E−25 |
| 11 | 47701629 | 47701642 | AGBL2 | 1.05709E−24 |
| 14 | 35497274 | 35497286 | SRP54 | 1.12815E−24 |
| X | 138864932 | 138864946 | ATP11C | 1.19716E−24 |
| 6 | 53138203 | 53138233 | ELOVL5 | 1.24256E−24 |
| 7 | 40234742 | 40234756 | SUGCT | 1.27685E−24 |
| 1 | 47123898 | 47123914 | ATPAF1 | 1.29229E−24 |
| 6 | 13316780 | 13316795 | TBC1D7 | 1.33818E−24 |
| 4 | 83376098 | 83376111 | ENOPH1 | 1.37417E−24 |
| 8 | 67969596 | 67969610 | COPS5 | 1.38893E−24 |
| 7 | 6064470 | 6064488 | EIF2AK1 | 1.43907E−24 |
| 2 | 209195472 | 209195485 | PIKFYVE | 1.5602E−24 |
| 1 | 167871297 | 167871312 | ADCY10 | 1.56745E−24 |
| 6 | 149846372 | 149846387 | PPIL4 | 1.5972E−24 |
| 3 | 33626219 | 33626236 | CLASP2 | 1.59992E−24 |
| 20 | 47649496 | 47649525 | ARFGEF2 | 1.70335E−24 |
| 2 | 47635523 | 47635537 | MSH2 | 1.70781E−24 |
| 15 | 52561891 | 52561907 | MYO5C | 1.78761E−24 |
| 1 | 247729206 | 247729223 | GCSAML | 1.79977E−24 |
| 5 | 78416973 | 78416990 | BHMT | 2.03856E−24 |
| 10 | 24784014 | 24784030 | KIAA1217 | 2.15454E−24 |
| 2 | 120567392 | 120567406 | PTPN4 | 2.26889E−24 |
| 7 | 63454255 | 63454269 | MIR4283-2(dist = 372708), LINC01005(dist = 30527) | 2.27302E−24 |
| 10 | 16824085 | 16824108 | RSU1 | 2.28695E−24 |
| 1 | 9633387 | 9633400 | SLC25A33 | 2.38094E−24 |
| 1 | 100542702 | 100542718 | HIAT1 | 2.38209E−24 |
| 7 | 75185990 | 75186002 | HIP1 | 2.48176E−24 |
| 8 | 33356825 | 33356839 | MAK16(NM_032509: c.*678_*692del0) | 2.51773E−24 |
| 12 | 132539500 | 132539513 | EP400 | 2.52104E−24 |
| 2 | 192011485 | 192011497 | STAT4 | 2.85475E−24 |
| 20 | 33033065 | 33033083 | ITCH | 2.89318E−24 |
| 14 | 39591613 | 39591627 | GEMIN2 | 3.01178E−24 |
| 7 | 97939975 | 97939988 | BAIAP2L1 | 3.03704E−24 |
| 1 | 212241691 | 212241730 | DTL | 3.12062E−24 |
| 10 | 21461410 | 21461435 | NEBL | 3.21502E−24 |
| 9 | 19334957 | 19334970 | DENND4C | 3.35705E−24 |
| 15 | 30010328 | 30010349 | TJP1 | 3.41309E−24 |
| X | 19037666 | 19037686 | GPR64 | 3.55721E−24 |
| 6 | 126249747 | 126249771 | NCOA7 | 3.56802E−24 |
| 3 | 37550027 | 37550040 | ITGA9 | 3.67318E−24 |
| 20 | 31954819 | 31954834 | CDK5RAP1 | 3.77175E−24 |
| 1 | 95615737 | 95615761 | TMEM56, TMEM56-RWDD3 | 3.83925E−24 |
| 10 | 94266330 | 94266346 | IDE | 3.84635E−24 |
| 3 | 58351660 | 58351690 | PXK | 4.06465E−24 |
| 15 | 63571279 | 63571293 | APH1B | 4.06645E−24 |
| 11 | 110128925 | 110128941 | RDX | 4.37666E−24 |
| 13 | 50586051 | 50586064 | DLEU2 | 4.50972E−24 |
| 7 | 92900502 | 92900527 | CCDC132 | 4.54812E−24 |
| 1 | 153617510 | 153617526 | CHTOP | 4.7715E−24 |
| 6 | 70459209 | 70459222 | LMBRD1 | 4.86036E−24 |
| 6 | 100988266 | 100988280 | ASCC3 | 4.94482E−24 |
| 7 | 33217071 | 33217084 | BBS9 | 5.49301E−24 |
| 5 | 36629801 | 36629815 | SLC1A3 | 5.55088E−24 |
| 9 | 131715001 | 131715017 | NUP188 | 5.93468E−24 |
| 5 | 122940980 | 122941019 | CSNK1G3 | 6.37687E−24 |
| 15 | 58974349 | 58974361 | ADAM10 | 6.53338E−24 |
| 7 | 26883761 | 26883776 | SKAP2 | 6.76383E−24 |
| 17 | 30500816 | 30500828 | RHOT1 | 7.03669E−24 |
| 2 | 120620092 | 120620108 | PTPN4 | 7.11287E−24 |
| 22 | 44083327 | 44083345 | EFCAB6 | 7.12959E−24 |
| X | 52674318 | 52674335 | SSX7 | 7.22303E−24 |
| 2 | 33586464 | 33586479 | LTBP1 | 7.27653E−24 |
| X | 83403148 | 83403160 | RPS6KA6 | 8.4411E−24 |
| 8 | 130874614 | 130874639 | FAM49B | 8.44845E−24 |
| 19 | 47700438 | 47700453 | SAE1 | 8.49465E−24 |
| 2 | 55559651 | 55559667 | CCDC88A | 8.63493E−24 |
| 19 | 10090461 | 10090482 | COL5A3 | 8.7274E−24 |
| 16 | 3808052 | 3808066 | CREBBP | 9.42602E−24 |
| 3 | 121515940 | 121515953 | IQCB1 | 1.08938E−23 |
| 2 | 32117217 | 32117230 | MEMO1 | 1.10789E−23 |
| 9 | 73164596 | 73164610 | TRPM3 | 1.11048E−23 |
| 9 | 97555228 | 97555242 | C9orf3 | 1.12839E−23 |
| X | 151996539 | 151996552 | CETN2 | 1.15342E−23 |
| 14 | 68582259 | 68582279 | RAD51B | 1.16257E−23 |
| 21 | 30356973 | 30356986 | LTN1 | 1.2089E−23 |
| 21 | 27136460 | 27136473 | GABPA | 1.35269E−23 |
| 7 | 139097271 | 139097295 | C7orf55-LUC7L2, LUC7L2 | 1.38188E−23 |
| 3 | 132166148 | 132166162 | DNAJC13 | 1.39138E−23 |
| 14 | 60616914 | 60616937 | DHRS7 | 1.43147E−23 |
| 6 | 128330423 | 128330435 | PTPRK | 1.50497E−23 |
| 14 | 103192774 | 103192791 | RCOR1 | 1.53703E−23 |
| 7 | 95775848 | 95775863 | SLC25A13 | 1.54628E−23 |
| 8 | 96047806 | 96047820 | NDUFAF6 | 1.56854E−23 |
| 9 | 21861902 | 21861914 | MTAP | 1.57606E−23 |
| 22 | 39079920 | 39079936 | TOMM22(NM_020243: c.*74_*90del0) | 1.58221E−23 |
| 7 | 107875143 | 107875165 | NRCAM | 1.6402E−23 |
| 14 | 23870220 | 23870234 | MYH6 | 1.68664E−23 |
| 2 | 48734331 | 48734349 | PPP1R21 | 1.69891E−23 |
| 8 | 30569609 | 30569622 | GSR | 1.78358E−23 |
| 13 | 115002097 | 115002111 | CDC16 | 1.81724E−23 |
| 13 | 102235764 | 102235780 | ITGBL1 | 1.94129E−23 |
| 8 | 38138934 | 38138975 | WHSC1L1 | 1.94582E−23 |
| 5 | 134015495 | 134015508 | SEC24A | 2.00715E−23 |
| 1 | 173490547 | 173490571 | SLC9C2 | 2.07462E−23 |
| 8 | 110535444 | 110535460 | PKHD1L1 | 2.20731E−23 |
| 4 | 57897508 | 57897530 | IGFBP7 | 2.25137E−23 |
| 22 | 16404838 | 16404857 | POTEH(dist = 116901), OR11H1(dist = 43967) | 2.26113E−23 |
| X | 19024426 | 19024440 | GPR64 | 2.27979E−23 |
| X | 134033226 | 134033239 | MOSPD1 | 2.32895E−23 |
| 14 | 35899480 | 35899493 | NFKBIA(dist = 25520), INSM2(dist = 103755) | 2.41338E−23 |
| 1 | 22030115 | 22030132 | USP48 | 2.52505E−23 |
| 2 | 162081128 | 162081140 | TANK | 2.59828E−23 |
| 8 | 38940143 | 38940157 | ADAM9 | 2.84384E−23 |
| 21 | 33312539 | 33312554 | HUNK | 3.05261E−23 |
| 1 | 233295992 | 233296007 | PCNXL2 | 3.2603E−23 |
| 3 | 178937630 | 178937642 | PIK3CA | 3.66385E−23 |
| 6 | 52400479 | 52400497 | TRAM2 | 3.67028E−23 |
| 1 | 63044645 | 63044661 | DOCK7 | 3.6725E−23 |
| 12 | 104461711 | 104461723 | HCFC2 | 3.7452E−23 |
| 1 | 53267474 | 53267488 | ZYG11B | 3.76262E−23 |
| 12 | 97051927 | 97051940 | CDK17(dist = 257561), NEDD1(dist = 249061) | 3.95815E−23 |
| 8 | 96260054 | 96260070 | C8orf37 | 4.05657E−23 |
| 4 | 25160732 | 25160748 | SEPSECS | 5.03112E−23 |
| 11 | 18379446 | 18379459 | GTF2H1 | 5.13142E−23 |
| 2 | 9558905 | 9558924 | ITGB1BP1 | 5.37084E−23 |
| 3 | 124397249 | 124397262 | KALRN | 5.383E−23 |
| 5 | 134015250 | 134015288 | SEC24A | 5.54528E−23 |
| 3 | 160233406 | 160233421 | KPNA4 | 5.78467E−23 |
| 7 | 122757719 | 122757733 | SLC13A1 | 5.82457E−23 |
| 15 | 76205523 | 76205536 | FBXO22 | 6.00755E−23 |
| X | 101409243 | 101409271 | BEX5(NM_001159560: c.-6_-34del0, | 6.0101E−23 |
| | | | NM_001012978: c.-6_-34del0) | |
| 2 | 230653669 | 230653682 | TRIP12 | 6.0464E−23 |
| 10 | 103567665 | 103567680 | MGEA5 | 6.16804E−23 |
| 6 | 129905035 | 129905050 | ARHGAP18 | 6.4384E−23 |
| 12 | 4645161 | 4645173 | C12orf4 | 6.63486E−23 |
| X | 15332451 | 15332469 | ASB11 | 6.64968E−23 |
| 19 | 9771286 | 9771300 | ZNF562 | 6.75884E−23 |
| 10 | 121675238 | 121675251 | SEC23IP | 6.85653E−23 |
| 7 | 29771546 | 29771561 | DPY19L2P3(dist = 43687), WIPF3(dist = 74609) | 6.91336E−23 |
| 2 | 48033875 | 48033909 | MSH6 | 7.30708E−23 |
| 14 | 36334905 | 36334921 | BRMS1L | 7.31319E−23 |
| 19 | 327260 | 327277 | MIER2 | 7.59677E−23 |
| 18 | 21120381 | 21120398 | NPC1 | 7.88708E−23 |
| 16 | 75674252 | 75674271 | KARS | 7.93746E−23 |
| 2 | 120831654 | 120831667 | EPB41L5 | 8.13895E−23 |
| 7 | 16676082 | 16676097 | ANKMY2 | 8.78762E−23 |
| 1 | 231094050 | 231094067 | TTC13 | 9.21226E−23 |
| 18 | 44424651 | 44424667 | PIAS2 | 1.01973E−22 |
| 2 | 211521213 | 211521243 | CPS1 | 1.03248E−22 |
| 5 | 169020337 | 169020350 | SPDL1 | 1.04823E−22 |
| 12 | 2997058 | 2997074 | RHNO1 | 1.0679E−22 |
| 6 | 136710669 | 136710685 | MAP7 | 1.12811E−22 |
| 14 | 58690427 | 58690451 | ACTR10 | 1.16142E−22 |
| 17 | 34151245 | 34151260 | TAF15 | 1.18761E−22 |
| X | 114871127 | 114871141 | PLS3 | 1.25087E−22 |
| 5 | 115167564 | 115167575 | ATG12 | 1.27832E−22 |
| 11 | 83180169 | 83180185 | DLG2 | 1.28716E−22 |
| 15 | 82517622 | 82517671 | EFTUD1 | 1.30736E−22 |
| 12 | 111160513 | 111160526 | PPP1CC | 1.33503E−22 |
| 3 | 169992958 | 169992994 | PRKCI | 1.34012E−22 |
| 3 | 32571823 | 32571841 | DYNC1LI1 | 1.4043E−22 |
| 8 | 89128708 | 89128722 | MMP16 | 1.46501E−22 |
| 5 | 41862862 | 41862875 | OXCT1 | 1.6183E−22 |
| 6 | 36452603 | 36452620 | KCTD20 | 1.65928E−22 |
| 12 | 130883554 | 130883568 | RIMBP2(NM_015347: c.*126_*112del0) | 1.67958E−22 |
| 7 | 66747794 | 66747810 | PMS2P4 | 1.81482E−22 |
| 1 | 11134470 | 11134485 | EXOSC10 | 1.99162E−22 |
| 1 | 113657018 | 113657033 | LRIG2 | 2.00756E−22 |
| 4 | 40123997 | 40124010 | N4BP2 | 2.01059E−22 |
| 4 | 88986745 | 88986761 | PKD2 | 2.10571E−22 |
| 5 | 118576096 | 118576110 | DMXL1 | 2.14366E−22 |
| 1 | 207753824 | 207753846 | CR1 | 2.26732E−22 |
| 15 | 80460576 | 80460591 | FAH | 2.27205E−22 |
| 16 | 22271671 | 22271685 | EEF2K | 2.32896E−22 |
| X | 135095655 | 135095677 | SLC9A6 | 2.50078E−22 |
| 20 | 30142484 | 30142500 | HM13 | 2.55811E−22 |
| 9 | 112184972 | 112184987 | PTPN3 | 2.59524E−22 |
| 20 | 50292756 | 50292777 | ATP9A | 2.61241E−22 |
| 2 | 27597190 | 27597204 | SNX17 | 2.69262E−22 |
| X | 36156447 | 36156461 | CHDC2 | 2.69443E−22 |
| 14 | 96916232 | 96916246 | AK7 | 2.76663E−22 |
| 2 | 234916771 | 234916787 | TRPM8 | 2.80786E−22 |
| 12 | 76780051 | 76780066 | OSBPL8 | 2.87725E−22 |
| 1 | 200080464 | 200080490 | NR5A2 | 2.90477E−22 |
| 17 | 73515055 | 73515068 | TSEN54 | 2.96355E−22 |
| 17 | 40850552 | 40850587 | CNTNAP1 | 3.04468E−22 |
| 12 | 63964640 | 63964653 | DPY19L2 | 3.05617E−22 |
| 4 | 151835502 | 151835539 | LRBA | 3.27142E−22 |
| 13 | 30854363 | 30854376 | KATNAL1 | 3.28141E−22 |
| 3 | 138248009 | 138248026 | CEP70 | 3.70122E−22 |
| 5 | 86679495 | 86679514 | RASA1 | 3.80988E−22 |
| 21 | 34117067 | 34117080 | PAXBP1 | 4.10987E−22 |
| 20 | 13140617 | 13140637 | SPTLC3 | 4.21262E−22 |
| 12 | 110006724 | 110006738 | MMAB | 4.21923E−22 |
| 2 | 61577667 | 61577680 | USP34 | 4.39221E−22 |
| 2 | 101885656 | 101885675 | CNOT11 | 4.55281E−22 |
| 8 | 30969355 | 30969369 | WRN | 4.60908E−22 |
| 5 | 178030941 | 178030955 | CLK4 | 4.85908E−22 |
| 6 | 70840124 | 70840137 | COL19A1 | 4.90706E−22 |
| 2 | 207013834 | 207013846 | NDUFS1 | 5.06749E−22 |
| 21 | 33982072 | 33982086 | C21orf59 | 5.17312E−22 |
| 15 | 51768755 | 51768768 | DMXL2 | 5.19323E−22 |
| X | 44918221 | 44918236 | KDM6A | 5.54968E−22 |
| 14 | 20763824 | 20763838 | TTC5 | 5.69605E−22 |
| 17 | 1326754 | 1326769 | CRK(NM_005206: c.*183_*168del0, | 6.30936E−22 |
| | | | NM_016823: c.*53_*38del0) | |
| 2 | 170062201 | 170062216 | LRP2 | 6.43151E−22 |
| 18 | 59942586 | 59942600 | KIAA1468 | 6.44631E−22 |
| 15 | 55972660 | 55972674 | PRTG | 6.47028E−22 |
| X | 153994469 | 153994482 | DKC1 | 6.8533E−22 |
| 3 | 132169705 | 132169718 | DNAJC13 | 7.40264E−22 |
| 12 | 45269439 | 45269452 | NELL2(NM_001145109: c.103_98delins-) | 8.60994E−22 |
| 15 | 64231569 | 64231591 | DAPK2 | 8.9625E−22 |
| 12 | 107398862 | 107398876 | CRY1 | 9.27778E−22 |
| 15 | 52185961 | 52185975 | TMOD3 | 9.41472E−22 |
| 7 | 35189891 | 35189905 | DPY19L2P1 | 9.46007E−22 |
| 5 | 50122645 | 50122660 | PARP8 | 9.59727E−22 |
| 15 | 54586266 | 54586278 | UNC13C | 9.74458E−22 |
| 4 | 114176848 | 114176860 | ANK2 | 9.80011E−22 |
| 10 | 115380483 | 115380497 | NRAP | 9.84952E−22 |
| 11 | 121367557 | 121367571 | SORL1 | 9.87851E−22 |
| X | 154133316 | 154133329 | F8 | 1.0125E−21 |
| 4 | 42592935 | 42592965 | ATP8A1 | 1.0357E−21 |
| 1 | 54395673 | 54395692 | HSPB11 | 1.16117E−21 |
| 14 | 31191658 | 31191682 | SCFD1 | 1.16405E−21 |
| 4 | 178357514 | 178357527 | AGA | 1.17267E−21 |
| 19 | 9361740 | 9361752 | OR7E24 | 1.23798E−21 |
| 18 | 19119813 | 19119828 | ESCO1 | 1.28329E−21 |
| 9 | 27062802 | 27062816 | IFT74(NM_001099223: c.*68_*82del0, | 1.31233E−21 |
| | | | NM_001099222: c.*68_*82del0, | |
| | | | NM_025103: c.*68_*82del0) | |
| 12 | 21639528 | 21639542 | RECOL | 1.32292E−21 |
| 3 | 113169372 | 113169386 | SPICE1 | 1.34831E−21 |
| X | 118679315 | 118679331 | CXorf56 | 1.37496E−21 |
| 8 | 74204423 | 74204438 | RPL7 | 1.40497E−21 |
| 1 | 155679543 | 155679557 | DAP3 | 1.4121E−21 |
| 8 | 87111362 | 87111382 | ATP6V0D2 | 1.44112E−21 |
| 8 | 86180906 | 86180924 | CA13 | 1.46089E−21 |
| X | 154578865 | 154578881 | LOC101927830, TMLHE-AS1 | 1.50205E−21 |
| 9 | 134049353 | 134049366 | NUP214 | 1.50207E−21 |
| 6 | 76728583 | 76728598 | IMPG1 | 1.52647E−21 |
| 5 | 137451460 | 137451472 | NME5 | 1.5494E−21 |
| 1 | 84956161 | 84956173 | RPF1 | 1.67924E−21 |
| X | 135481956 | 135481970 | GPR112 | 1.68262E−21 |
| 2 | 95766553 | 95766567 | MRPS5 | 1.6861E−21 |
| 20 | 46270911 | 46270923 | NCOA3 | 1.89093E−21 |
| 14 | 96848565 | 96848580 | GSKIP | 1.90392E−21 |
| 1 | 182812410 | 182812425 | DHX9 | 2.02987E−21 |
| 20 | 35672436 | 35672456 | RBL1 | 2.0729E−21 |

TABLE 13
Microsatellite Repeat Marker Sequences for Kidney Renal Clear Cell Carcinoma (KIRC).
TABLE 13 - KIRC

| ch | start_pos | end_pos | | KIRC |
|---|---|---|---|---|
| (hg37) | (hg37) | (hg37) | gene | p-value |
| 11 | 28113094 | 28113107 | KIF18A | 0.000123031 |
| 4 | 46994972 | 46994992 | GABRA4 | 0.000124984 |
| 5 | 50122645 | 50122660 | PARP8 | 0.000126984 |
| 6 | 102130410 | 102130422 | GRIK2 | 0.0001638 |
| 4 | 170989708 | 170989719 | AADAT | 0.000219298 |
| 2 | 234178624 | 234178640 | ATG16L1 | 0.000232919 |
| 7 | 103206001 | 103206013 | RELN | 0.000322027 |
| 15 | 40005793 | 40005806 | FSIP1 | 0.000341763 |
| 7 | 139097271 | 139097295 | C7orf55-LUC7L2, LUC7L2 | 0.000363372 |
| 15 | 59693969 | 59693985 | MYO1E(dist = 28898), FAM81A(dist = 36387) | 0.000380952 |
| 20 | 25057191 | 25057229 | VSX1 | 0.000391236 |
| 11 | 59626477 | 59626491 | TCN1 | 0.000434594 |
| 17 | 39871551 | 39871566 | GAST | 0.000480769 |
| 1 | 65138787 | 65138801 | CACHD1 | 0.000571102 |
| 12 | 970078 | 970128 | WNK1 | 0.000615214 |
| 1 | 235826382 | 235826396 | LYST | 0.000644053 |
| 1 | 36888302 | 36888316 | OSCP1 | 0.000653595 |
| 9 | 112184972 | 112184987 | PTPN3 | 0.000673325 |
| 17 | 39723415 | 39723439 | KRT9 | 0.000701262 |
| 9 | 133498229 | 133498245 | FUBP3 | 0.000715564 |
| 11 | 134054705 | 134054746 | NCAPD3 | 0.000715564 |
| 2 | 198257652 | 198257684 | SF3B1 | 0.00079968 |
| 1 | 75172756 | 75172771 | CRYZ | 0.000826446 |
| 12 | 97303682 | 97303713 | NEDD1 | 0.000846323 |
| 7 | 63454255 | 63454269 | MIR4283-2(dist = 372708), LINC01005(dist = 30527) | 0.00089955 |
| 1 | 211280724 | 211280748 | KCNH1 | 0.000915332 |
| 1 | 245021291 | 245021305 | HNRNPU | 0.000931532 |
| 3 | 122259738 | 122259756 | PARP9 | 0.000949367 |
| 6 | 168348504 | 168348521 | MLLT4 | 0.001052632 |
| 1 | 236590675 | 236590687 | EDARADD | 0.001084394 |
| 4 | 42626659 | 42626674 | ATP8A1 | 0.001139212 |
| 13 | 40298620 | 40298639 | COG6 | 0.001174398 |
| 5 | 68524006 | 68524019 | MRPS36 | 0.001249844 |
| 16 | 69492952 | 69492989 | CYB5B | 0.0013328 |
| 14 | 95923421 | 95923440 | SYNE3 | 0.001343785 |
| 3 | 191097928 | 191097944 | CCDC50 | 0.00135483 |
| 11 | 67165932 | 67165942 | PPP1CA(NM_002708: c.*74_*64del0, | 0.001413708 |
| | | | NM_206873: c.*74_*64del0, | |
| | | | NM_001008709: c.*74_*64del0) | |
| 3 | 10018739 | 10018754 | EMC3 | 0.001439101 |
| 14 | 55636060 | 55636079 | DLGAP5 | 0.001444788 |
| 2 | 179616770 | 179616786 | TTN | 0.001488095 |
| 2 | 189859434 | 189859447 | COL3A1 | 0.001596227 |
| 15 | 50570999 | 50571024 | GABPB1 | 0.001668057 |
| 7 | 138453892 | 138453909 | ATP6V0A4 | 0.00172117 |
| 12 | 16713487 | 16713501 | LMO3 | 0.001780898 |
| 4 | 25315637 | 25315653 | ZCCHC4 | 0.001832075 |
| 4 | 5630470 | 5630482 | EVC2 | 0.001851852 |
| 21 | 30910210 | 30910231 | GRIK1 | 0.001893939 |
| 10 | 18280054 | 18280071 | SLC39A12 | 0.00212073 |
| 3 | 172634021 | 172634039 | SPATA16 | 0.002172968 |
| 12 | 53823175 | 53823189 | AMHR2 | 0.002192982 |
| 17 | 17697093 | 17697133 | RAI1 | 0.00255102 |
| 6 | 17669262 | 17669277 | NUP153 | 0.002709677 |
| 10 | 94369023 | 94369100 | KIF11 | 0.00308289 |
| 17 | 47109368 | 47109378 | IGF2BP1 | 0.003092146 |
| 17 | 17152363 | 17152377 | COPS3 | 0.003521914 |
| 5 | 172421760 | 172421776 | ATP6V0E1 | 0.003583373 |
| 11 | 700526 | 700541 | TMEM80 | 0.00381971 |
| 2 | 227967830 | 227967842 | COL4A4 | 0.004056207 |
| 7 | 99711461 | 99711472 | TAF6 | 0.004158416 |
| 3 | 131186893 | 131186917 | MRPL3 | 0.004197901 |
| 1 | 220369745 | 220369758 | RAB3GAP2 | 0.004201681 |
| 11 | 1905146 | 1905158 | LSP1 | 0.004302926 |
| 2 | 215890346 | 215890382 | ABCA12 | 0.005438464 |
| 2 | 236817336 | 236817351 | AGAP1 | 0.005597933 |
| 6 | 41876911 | 41876925 | MED20 | 0.005597933 |
| 2 | 183066377 | 183066403 | PDE1A | 0.005681818 |
| 11 | 3424760 | 3424773 | TSSC2 | 0.006060606 |
| 8 | 42761230 | 42761249 | HOOK3 | 0.00617284 |
| 20 | 48562797 | 48562813 | RNF114 | 0.00621118 |
| 6 | 146256315 | 146256344 | SHPRH | 0.006289308 |
| 7 | 72966422 | 72966438 | BCL7B | 0.006329114 |
| 15 | 44024081 | 44024112 | CKMT1A(dist = 32661), CATSPER2P1(dist = 4034) | 0.006493506 |
| 1 | 165712555 | 165712571 | TMCO1 | 0.006578947 |
| 21 | 35094849 | 35094878 | ITSN1 | 0.006578947 |
| 5 | 37212582 | 37212612 | C5orf42 | 0.006578947 |
| 2 | 54146387 | 54146400 | PSME4 | 0.006622517 |
| 14 | 93712634 | 93712660 | BTBD7 | 0.006622517 |
| 11 | 86120270 | 86120294 | CCDC81 | 0.006666667 |
| 17 | 12920526 | 12920549 | ELAC2 | 0.006711409 |
| 17 | 35979746 | 35979761 | DDX52 | 0.006711409 |
| 12 | 2997058 | 2997074 | RHNO1 | 0.006756757 |
| 11 | 118885668 | 118885701 | CCDC84 | 0.006802721 |
| X | 20204548 | 20204563 | RPS6KA3 | 0.006802721 |
| 1 | 176105710 | 176105723 | RFWD2 | 0.006849315 |
| 14 | 59787325 | 59787338 | DAAM1 | 0.006849315 |
| 7 | 64275470 | 64275486 | ZNF138 | 0.006849315 |
| 1 | 100671860 | 100671880 | DBT | 0.006849315 |
| 10 | 35314130 | 35314161 | CUL2 | 0.006896552 |
| 3 | 176751870 | 176751887 | TBL1XR1 | 0.006896552 |
| 4 | 40123997 | 40124010 | N4BP2 | 0.006896552 |
| 19 | 37721404 | 37721419 | ZNF383 | 0.006896552 |
| 1 | 155671942 | 155671962 | DAP3 | 0.006993007 |
| 17 | 465993 | 466012 | VPS53 | 0.006993007 |
| 6 | 150123316 | 150123329 | PCMT1 | 0.007063882 |
| 10 | 32327037 | 32327053 | KIF5B | 0.007092199 |
| 5 | 78416973 | 78416990 | BHMT | 0.007092199 |
| 12 | 42835328 | 42835365 | PPHLN1(NM_201439: c.*17_*54del0, | 0.007092199 |
| | | | NM_201440: c.*17_*54del0, | |
| | | | NM_001143788: c.*17_*54del0, | |
| | | | NM_001143789: c.*17_*54del0, | |
| | | | NM_201515: c.*17_*54del0) | |
| 10 | 94266330 | 94266346 | IDE | 0.007092199 |
| 3 | 172052897 | 172052914 | FNDC3B | 0.007142857 |
| 20 | 47649496 | 47649525 | ARFGEF2 | 0.007142857 |
| 8 | 48732074 | 48732096 | PRKDC | 0.007142857 |
| 4 | 94693160 | 94693173 | GRID2 | 0.007194245 |
| 4 | 38942697 | 38942726 | FAM114A1 | 0.007246377 |
| 6 | 18148155 | 18148167 | TPMT | 0.00729927 |
| 17 | 17039561 | 17039595 | MPRIP | 0.007368421 |
| 1 | 118623681 | 118623714 | SPAG17 | 0.007406576 |
| 13 | 50098426 | 50098438 | PHF11 | 0.007407407 |
| X | 19086800 | 19086823 | GPR64 | 0.007462687 |
| 10 | 89720622 | 89720649 | PTEN | 0.007462687 |
| X | 119676896 | 119676908 | CUL4B | 0.007462687 |
| 14 | 94745068 | 94745095 | PPP4R4(NM_058237: c.*88_*115del0) | 0.007462687 |
| 2 | 136529897 | 136529910 | UBXN4 | 0.007462687 |
| 1 | 149900985 | 149901002 | MTMR11(NM_001145862: c.*36_*19del0) | 0.007518797 |
| 12 | 72067966 | 72067980 | THAP2 | 0.007518797 |
| 11 | 117222647 | 117222659 | CEP164 | 0.007518797 |
| 4 | 119950975 | 119950989 | SYNPO2 | 0.007567568 |
| 14 | 75199555 | 75199568 | FCF1 | 0.007575758 |
| 7 | 105122891 | 105122924 | PUS7 | 0.007575758 |
| 4 | 41015553 | 41015566 | APBB2 | 0.007633588 |
| 6 | 42611936 | 42611951 | UBR2 | 0.007633588 |
| 17 | 16344523 | 16344534 | LRRC75A-AS1 | 0.007680492 |
| 19 | 7995974 | 7996007 | TIMM44 | 0.007692308 |
| 11 | 47187055 | 47187087 | ARFGAP2 | 0.007692308 |
| 1 | 169819526 | 169819538 | C1orf112 | 0.007692308 |
| 5 | 36123065 | 36123135 | LMBRD2 | 0.007751938 |
| 8 | 124817605 | 124817620 | FAM91A1 | 0.007751938 |
| 17 | 8166113 | 8166126 | PFAS | 0.0078125 |
| 1 | 36019913 | 36019925 | KIAA0319L | 0.0078125 |
| 5 | 112903585 | 112903598 | YTHDC2 | 0.007874016 |
| 2 | 48688258 | 48688273 | PPP1R21 | 0.007874016 |
| 10 | 74874082 | 74874112 | NUDT13 | 0.007874016 |
| 2 | 85589223 | 85589237 | ELMOD3 | 0.007874016 |
| 2 | 239241445 | 239241460 | TRAF3IP1 | 0.007936508 |
| 14 | 69861462 | 69861490 | ERH | 0.007936508 |
| 12 | 28412398 | 28412416 | CCDC91 | 0.007936508 |
| 12 | 62972198 | 62972223 | MON2 | 0.007936508 |
| 18 | 18588229 | 18588257 | ROCK1 | 0.007936508 |
| 1 | 153949135 | 153949147 | JTB | 0.008 |
| 2 | 109123970 | 109124000 | GCC2 | 0.008 |
| 20 | 50713983 | 50714011 | ZFP64 | 0.008 |
| 20 | 47648454 | 47648481 | ARFGEF2 | 0.008064516 |
| 1 | 36479764 | 36479778 | AGO3 | 0.008064516 |
| 3 | 180679235 | 180679250 | FXR1 | 0.008130081 |
| 6 | 121412191 | 121412203 | TBC1D32 | 0.008196721 |
| 9 | 33056971 | 33056996 | SMU1 | 0.008196721 |
| 8 | 38138934 | 38138975 | WHSC1L1 | 0.008264463 |
| 1 | 242264075 | 242264108 | PLD5 | 0.008264463 |
| 17 | 11697015 | 11697027 | DNAH9 | 0.008333333 |
| 2 | 9593027 | 9593038 | CPSF3 | 0.008333333 |
| 6 | 88126360 | 88126373 | C6orf165 | 0.008333333 |
| 13 | 50306915 | 50306932 | KPNA3 | 0.008333333 |
| 1 | 154144673 | 154144694 | TPM3 | 0.008403361 |
| 8 | 120856021 | 120856034 | DSCC1 | 0.008403361 |
| 7 | 2403894 | 2403908 | EIF3B | 0.008403361 |
| 12 | 97306603 | 97306631 | NEDD1 | 0.008474576 |
| 5 | 38978758 | 38978770 | RICTOR | 0.008474576 |
| 7 | 151046112 | 151046124 | NUB1 | 0.008474576 |
| 7 | 26247984 | 26247997 | CBX3 | 0.008547009 |
| 2 | 61577667 | 61577680 | USP34 | 0.008547009 |
| 6 | 43021976 | 43021989 | MRPL2(NM_015950: c.*36_*23del0, | 0.008598078 |
| | | | NM_001300848: c.*202_*189del0) | |
| 5 | 67584512 | 67584525 | PIK3R1(NM_181524: c.-52_-39del-) | 0.00862069 |
| 4 | 143181722 | 143181748 | INPP4B | 0.00862069 |
| 20 | 42265908 | 42265920 | IFT52 | 0.00862069 |
| 4 | 39511530 | 39511543 | MIR1273H | 0.00877193 |
| 3 | 146167163 | 146167186 | PLSCR2 | 0.00877193 |
| 16 | 31284618 | 31284634 | ITGAM | 0.00877193 |
| 12 | 22065994 | 22066028 | ABCC9 | 0.00877193 |
| 1 | 161201297 | 161201309 | NR1I3 | 0.00877193 |
| 20 | 37187110 | 37187134 | RALGAPB | 0.00877193 |
| 17 | 66039938 | 66039951 | KPNA2 | 0.008849558 |
| 4 | 93225752 | 93225765 | GRID2(NM_001286838: c.-56_-43del-, | 0.008849558 |
| | | | NM_001510: c.-56_-43del-) | |
| 12 | 76453670 | 76453694 | NAP1L1 | 0.008849558 |
| 2 | 37487307 | 37487330 | PRKD3 | 0.008928571 |
| 10 | 76349020 | 76349033 | ADK | 0.009009009 |
| 4 | 47746602 | 47746616 | CORIN | 0.009090909 |
| 12 | 57006059 | 57006072 | BAZ2A | 0.009090909 |
| 10 | 90034667 | 90034679 | RNLS(NM_018363: c.*51_*39del0) | 0.009090909 |
| 3 | 45776875 | 45776914 | SACM1L | 0.009090909 |
| 6 | 105405900 | 105405913 | LIN28B | 0.009174312 |
| 16 | 71718538 | 71718563 | PHLPP2 | 0.009174312 |
| 8 | 101933023 | 101933037 | YWHAZ | 0.009259259 |
| 15 | 50920509 | 50920523 | TRPM7 | 0.009259259 |
| 6 | 167416662 | 167416676 | FGFR1OP | 0.009259259 |
| 2 | 179325203 | 179325214 | MIR548N | 0.009259259 |
| 6 | 39580887 | 39580898 | KIF6 | 0.009259259 |
| 10 | 134628521 | 134628532 | CFAP46 | 0.009345794 |
| 19 | 34945331 | 34945344 | UBA2 | 0.009345794 |
| 5 | 59901692 | 59901704 | DEPDC1B | 0.009433962 |
| 1 | 22030891 | 22030902 | USP48 | 0.009615385 |
| 5 | 150282674 | 150282694 | ZNF300 | 0.009615385 |
| 7 | 74172385 | 74172400 | GTF2I | 0.009615385 |
| 8 | 130874614 | 130874639 | FAM49B | 0.009708738 |
| 7 | 150918586 | 150918602 | ABCF2 | 0.009708738 |
| 17 | 4646533 | 4646547 | ZMYND15 | 0.009708738 |
| X | 129265852 | 129265866 | AIFM1 | 0.009708738 |
| 2 | 158406601 | 158406612 | ACVR1C | 0.009708738 |
| 15 | 34654339 | 34654358 | LPCAT4 | 0.009727115 |
| 2 | 111881263 | 111881275 | BCL2L11 | 0.009803922 |
| 15 | 73580950 | 73580962 | NEO1 | 0.009803922 |
| 1 | 197009613 | 197009644 | F13B | 0.01 |
| 2 | 200800572 | 200800585 | TYW5 | 0.01 |
| 3 | 185641778 | 185641789 | TRA2B | 0.01 |
| 13 | 45556282 | 45556303 | NUFIP1 | 0.01 |
| 2 | 120831654 | 120831667 | EPB41L5 | 0.01010101 |
| 11 | 113688357 | 113688368 | USP28 | 0.010204082 |
| 1 | 32692168 | 32692186 | EIF3I | 0.010309278 |
| 2 | 203676382 | 203676403 | ICA1L | 0.010471204 |
| 12 | 112308870 | 112308881 | MAPKAPK5 | 0.010638298 |
| 11 | 128354631 | 128354658 | ETS1 | 0.010638298 |
| 12 | 14947675 | 14947706 | WBP11 | 0.010989011 |
| 19 | 45540541 | 45540557 | RELB | 0.010989011 |
| 2 | 47600881 | 47600892 | EPCAM | 0.011111111 |
| 8 | 103664685 | 103664719 | KLF10 | 0.011173184 |
| 10 | 96108988 | 96109000 | NOC3L | 0.011235955 |
| 6 | 44081957 | 44081968 | MRPL14 | 0.011363636 |
| 6 | 13620380 | 13620391 | NOL7 | 0.011363636 |
| 3 | 19498221 | 19498253 | KCNH8 | 0.011494253 |
| 1 | 197059221 | 197059244 | ASPM | 0.011494253 |
| 16 | 7743418 | 7743437 | RBFOX1 | 0.011560694 |
| 17 | 29508819 | 29508836 | NF1 | 0.011560694 |
| 14 | 39591613 | 39591627 | GEMIN2 | 0.011597333 |
| 2 | 70456452 | 70456465 | TIA1 | 0.011627907 |
| 10 | 122630877 | 122630889 | MIR5694 | 0.011627907 |
| 14 | 104040415 | 104040450 | APOPT1 | 0.011627907 |
| 16 | 67409030 | 67409054 | LRRC36 | 0.011764706 |
| 10 | 112649255 | 112649287 | PDCD4 | 0.011764706 |
| 20 | 480602 | 480627 | CSNK2A1 | 0.01183432 |
| 7 | 94181585 | 94181596 | CASD1 | 0.011904762 |
| 16 | 31202698 | 31202716 | FUS | 0.011976048 |
| 2 | 45806939 | 45806979 | SRBD1 | 0.012048193 |
| 1 | 183481943 | 183481964 | SMG7 | 0.012048193 |
| 13 | 73547700 | 73547726 | PIBF1 | 0.012048193 |
| 20 | 48494613 | 48494625 | SLC9A8 | 0.012048193 |
| 16 | 4700318 | 4700337 | MGRN1 | 0.012195122 |
| 12 | 110834030 | 110834049 | ANAPC7 | 0.012195122 |
| 14 | 56104386 | 56104405 | KTN1 | 0.012195122 |
| 1 | 155884155 | 155884167 | KIAA0907 | 0.012269939 |
| 18 | 19110456 | 19110477 | ESCO1 | 0.012303486 |
| 17 | 30500816 | 30500828 | RHOT1 | 0.012345679 |
| 7 | 100319680 | 100319696 | EPO | 0.012345679 |
| X | 135767763 | 135767792 | ARHGEF6 | 0.012345679 |
| 16 | 28883087 | 28883097 | SH2B1 | 0.012345679 |
| 15 | 44778956 | 44778975 | CTDSPL2 | 0.01242236 |
| 2 | 186689258 | 186689269 | FSIP2 | 0.0125 |
| 2 | 98866980 | 98866995 | VWA3B | 0.0125 |
| 3 | 10128965 | 10128985 | FANCD2, FANCD2OS | 0.012578616 |
| 14 | 103192774 | 103192791 | RCOR1 | 0.012578616 |
| 19 | 1825786 | 1825805 | REXO1 | 0.012658228 |
| 4 | 102839351 | 102839362 | BANK1 | 0.012658228 |
| 2 | 131883287 | 131883301 | PLEKHB2 | 0.012738854 |
| 13 | 48828102 | 48828118 | ITM2B | 0.012738854 |
| 5 | 95224700 | 95224719 | ELL2 | 0.012738854 |
| 1 | 89449508 | 89449520 | RBMXL1 | 0.012820513 |
| 15 | 45725103 | 45725138 | C15orf48 | 0.012820513 |
| 13 | 25911034 | 25911067 | NUPL1 | 0.012820513 |
| 4 | 88986745 | 88986761 | PKD2 | 0.012820513 |
| 10 | 93752074 | 93752105 | BTAF1 | 0.012820513 |
| 11 | 77672189 | 77672229 | INTS4 | 0.012820513 |
| 12 | 122017894 | 122017916 | KDM2B | 0.012903226 |
| 18 | 59942586 | 59942600 | KIAA1468 | 0.012987013 |
| 12 | 45771901 | 45771918 | ANO6 | 0.012987013 |
| 22 | 41551231 | 41551249 | EP300 | 0.012987013 |
| 1 | 95615737 | 95615761 | TMEM56, TMEM56-RWDD3 | 0.012987013 |
| 2 | 214794938 | 214794951 | SPAG16 | 0.012987013 |
| 2 | 15676456 | 15676468 | NBAS | 0.012987013 |
| 7 | 150269192 | 150269204 | GIMAP4 | 0.013071895 |
| 15 | 101019693 | 101019705 | CERS3 | 0.013071895 |
| 2 | 111406968 | 111406984 | BUB1 | 0.013071895 |
| 9 | 20346363 | 20346406 | MLLT3(NM_004529: c.*78_*35del0, | 0.013071895 |
| | | | NM_001286691: c.*78_*35del0) | |
| 10 | 62544391 | 62544411 | CDK1 | 0.013071895 |
| 18 | 6975847 | 6975857 | LAMA1 | 0.013157895 |
| 16 | 72107664 | 72107726 | HPR | 0.013157895 |
| 5 | 161494990 | 161495003 | GABRG2(NM_198903: c.-16_-3del-, | 0.013157895 |
| | | | NM_198904: c.-16_-3del-, | |
| | | | NM_000816: c.-16_-3del-) | |
| 9 | 114295951 | 114295963 | ZNF483 | 0.013157895 |
| 19 | 37581821 | 37581861 | ZNF420 | 0.013245033 |
| 10 | 69961537 | 69961564 | MYPN | 0.013245033 |
| 2 | 108999483 | 108999515 | SULT1C4 | 0.013245033 |
| 18 | 21120381 | 21120398 | NPC1 | 0.013333333 |
| 9 | 116039089 | 116039115 | PRPF4 | 0.013333333 |
| 20 | 61299715 | 61299725 | SLCO4A1 | 0.013333333 |
| 1 | 113159364 | 113159384 | ST7L | 0.013422819 |
| 21 | 40181937 | 40181956 | ETS2 | 0.013422819 |
| 14 | 21964623 | 21964635 | TOX4 | 0.013422819 |
| 18 | 9279678 | 9279703 | ANKRD12 | 0.013513514 |
| 2 | 207013834 | 207013846 | NDUFS1 | 0.013513514 |
| 14 | 68582259 | 68582279 | RAD51B | 0.013513514 |
| 14 | 103449863 | 103449880 | CDC42BPB | 0.013513514 |
| 1 | 148594259 | 148594272 | NBPF25P | 0.013513514 |
| 20 | 3565277 | 3565289 | ATRN | 0.013605442 |
| 5 | 6623405 | 6623418 | NSUN2 | 0.013605442 |
| 12 | 129360344 | 129360381 | GLT1D1 | 0.013605442 |
| X | 138864932 | 138864946 | ATP11C | 0.013605442 |
| 1 | 185951573 | 185951589 | HMCN1 | 0.013605442 |
| 13 | 77750765 | 77750791 | MYCBP2 | 0.013605442 |
| 2 | 211179765 | 211179777 | MYL1 | 0.01369863 |
| 11 | 63149670 | 63149682 | SLC22A9 | 0.01369863 |
| 14 | 59790749 | 59790763 | DAAM1 | 0.01369863 |
| 1 | 47581121 | 47581151 | CYP4Z1 | 0.01369863 |
| 5 | 150846858 | 150846882 | SLC36A1 | 0.01369863 |
| 20 | 47734948 | 47734972 | STAU1 | 0.01369863 |
| 4 | 178274919 | 178274938 | NEIL3 | 0.01369863 |
| 11 | 57505520 | 57505534 | TMX2-CTNND1 | 0.01369863 |
| 8 | 103236408 | 103236428 | RRM2B | 0.01369863 |
| 16 | 74372596 | 74372618 | LOC283922 | 0.013793103 |
| 7 | 5780521 | 5780558 | RNF216 | 0.013793103 |
| 21 | 27136460 | 27136473 | GABPA | 0.013888889 |
| 9 | 86354663 | 86354679 | GKAP1 | 0.013888889 |
| 7 | 95818864 | 95818897 | SLC25A13 | 0.013888889 |
| 11 | 102575512 | 102575536 | MMP27 | 0.013888889 |
| 4 | 41263718 | 41263729 | UCHL1 | 0.013888889 |
| 9 | 13193320 | 13193332 | MPDZ | 0.013888889 |
| 2 | 217280250 | 217280286 | SMARCAL1 | 0.013888889 |
| 20 | 4839890 | 4839911 | SLC23A2 | 0.013986014 |
| 15 | 78839102 | 78839127 | PSMA4 | 0.013986014 |
| 18 | 59942746 | 59942760 | KIAA1468 | 0.013986014 |
| 1 | 169560727 | 169560740 | SELP | 0.013986014 |
| 20 | 4850568 | 4850578 | SLC23A2 | 0.014084507 |
| 14 | 96848565 | 96848580 | GSKIP | 0.014084507 |
| 6 | 17292448 | 17292460 | RBM24(NM_001143942: c.*98_*110del0, | 0.014084507 |
| | | | NM_001143941: c.*98_*110del0, | |
| | | | NM_153020: c.*98_*110del0) | |
| 2 | 120712718 | 120712731 | PTPN4 | 0.014084507 |
| 1 | 10177680 | 10177693 | UBE4B | 0.014084507 |
| 12 | 62785259 | 62785270 | USP15 | 0.014084507 |
| 2 | 162877041 | 162877054 | DPP4 | 0.014084507 |
| 18 | 12817106 | 12817118 | PTPN2 | 0.014084507 |
| 15 | 44002670 | 44002699 | CKMT1A(dist = 11250), | 0.014084507 |
| | | | CATSPER2P1(dist = 25447) | |
| 18 | 21047278 | 21047290 | RIOK3 | 0.014084507 |
| 6 | 49415351 | 49415368 | MUT | 0.014184397 |
| 3 | 160141137 | 160141173 | SMC4 | 0.014184397 |
| 16 | 48333493 | 48333512 | MIR548AE2 | 0.014184397 |
| 2 | 196762491 | 196762509 | DNAH7 | 0.014184397 |
| 20 | 37191391 | 37191408 | RALGAPB | 0.014184397 |
| 6 | 149846372 | 149846387 | PPIL4 | 0.014184397 |
| 15 | 50596341 | 50596355 | GABPB1 | 0.014184397 |
| 18 | 30275526 | 30275539 | KLHL14 | 0.014184397 |
| 11 | 65268479 | 65268492 | MALAT1 | 0.014285714 |
| 1 | 215914883 | 215914896 | USH2A | 0.014285714 |
| 8 | 53543090 | 53543106 | RB1CC1 | 0.014285714 |
| 2 | 160092703 | 160092716 | WDSUB1 | 0.014285714 |
| 1 | 231396485 | 231396509 | GNPAT | 0.014285714 |
| 12 | 108140266 | 108140281 | LOC101929162 | 0.014285714 |
| 7 | 6064470 | 6064488 | EIF2AK1 | 0.014285714 |
| 1 | 42776699 | 42776711 | FOXJ3 | 0.014285714 |
| 12 | 77421978 | 77421991 | E2F7 | 0.014285714 |
| 16 | 4861103 | 4861130 | GLYR1 | 0.014285714 |
| 8 | 80831393 | 80831407 | MRPS28 | 0.014388489 |
| 5 | 134305852 | 134305866 | CATSPER3 | 0.014388489 |
| 3 | 50112589 | 50112605 | RBM6 | 0.014388489 |
| 1 | 210414859 | 210414894 | SERTAD4 | 0.014388489 |
| 15 | 42503953 | 42503965 | TMEM87A | 0.014492754 |
| 17 | 30190540 | 30190551 | UTP6 | 0.014492754 |
| 2 | 32095023 | 32095056 | MEMO1 | 0.014492754 |
| 3 | 108102580 | 108102602 | MYH15 | 0.014492754 |
| 10 | 70951558 | 70951580 | SUPV3L1 | 0.014492754 |
| 11 | 43419087 | 43419112 | TTC17 | 0.014492754 |
| 3 | 50091941 | 50091957 | RBM6 | 0.014492754 |
| 3 | 100378512 | 100378523 | GPR128 | 0.014492754 |
| 11 | 119144791 | 119144809 | CBL | 0.014492754 |
| 2 | 197762941 | 197762956 | PGAP1 | 0.014492754 |
| 9 | 75420448 | 75420463 | TMC1 | 0.01459854 |
| 4 | 146031236 | 146031253 | ABCE1 | 0.01459854 |
| 10 | 118891992 | 118892021 | VAX1 | 0.01459854 |
| 7 | 75651305 | 75651336 | STYXL1 | 0.01459854 |
| 6 | 138192324 | 138192336 | TNFAIP3 | 0.01459854 |
| 8 | 39114668 | 39114704 | ADAM32 | 0.01459854 |
| 5 | 139907713 | 139907738 | ANKHD1, ANKHD1-EIF4EBP3 | 0.01459854 |
| 3 | 108182168 | 108182180 | MYH15 | 0.01459854 |
| 6 | 144179190 | 144179202 | LTV1 | 0.014705882 |
| 20 | 37395979 | 37396016 | ACTR5 | 0.014705882 |
| 7 | 75113378 | 75113398 | POM121C | 0.014705882 |
| 12 | 26807065 | 26807090 | ITPR2 | 0.014705882 |
| 10 | 120867694 | 120867705 | FAM45B | 0.014705882 |
| 1 | 200080464 | 200080490 | NR5A2 | 0.014814815 |
| 3 | 37176656 | 37176670 | LRRFIP2 | 0.014814815 |
| 4 | 128812230 | 128812245 | PLK4 | 0.014814815 |
| 5 | 74011541 | 74011556 | HEXB | 0.014814815 |
| 2 | 202494630 | 202494642 | TMEM237 | 0.014814815 |
| 9 | 135526541 | 135526560 | DDX31 | 0.014814815 |
| 3 | 127820493 | 127820505 | RUVBL1 | 0.014925373 |
| 3 | 186522293 | 186522308 | RFC4 | 0.014925373 |
| 6 | 20548763 | 20548778 | CDKAL1 | 0.014925373 |
| 7 | 122757719 | 122757733 | SLC13A1 | 0.014925373 |
| 2 | 201470233 | 201470249 | AOX1 | 0.014925373 |
| 11 | 59368218 | 59368235 | OSBP | 0.015037594 |
| 15 | 52239617 | 52239629 | LEO1 | 0.015037594 |
| 12 | 122017848 | 122017866 | KDM2B | 0.015037594 |
| 18 | 29101215 | 29101231 | DSG2 | 0.015037594 |
| 10 | 17646048 | 17646060 | PTPLA | 0.015037594 |
| 11 | 46568608 | 46568621 | AMBRA1 | 0.015037594 |
| 9 | 103212778 | 103212790 | MSANTD3, MSANTD3-TMEFF1 | 0.015037594 |
| 6 | 7575506 | 7575518 | DSP | 0.015037594 |
| 2 | 143913281 | 143913292 | ARHGAP15 | 0.015037594 |
| 3 | 179298584 | 179298597 | ACTL6A | 0.015151515 |
| 1 | 233295992 | 233296007 | PCNXL2 | 0.015151515 |
| 11 | 68338491 | 68338520 | PPP6R3 | 0.015151515 |
| 6 | 117859721 | 117859732 | DCBLD1 | 0.015151515 |
| 16 | 57494395 | 57494422 | COQ9 | 0.015151515 |
| 12 | 54646020 | 54646054 | CBX5 | 0.015151515 |
| 6 | 163235158 | 163235170 | PACRG | 0.015267176 |
| 11 | 118369265 | 118369290 | KMT2A | 0.015267176 |
| 22 | 26769464 | 26769477 | SEZ6L | 0.015267176 |
| 12 | 123103115 | 123103129 | KNTC1 | 0.015267176 |
| 7 | 92855628 | 92855641 | HEPACAM2 | 0.015267176 |
| X | 106396617 | 106396641 | NUP62CL | 0.015267176 |
| 3 | 194159705 | 194159736 | ATP13A3 | 0.015267176 |
| 15 | 75909933 | 75909947 | SNUPN | 0.015267176 |
| 2 | 95816017 | 95816037 | ZNF514 | 0.015267176 |
| 4 | 52928352 | 52928371 | SPATA18 | 0.015348501 |
| 8 | 62550924 | 62550938 | ASPH | 0.015384615 |
| 17 | 2323953 | 2323979 | METTL16 | 0.015384615 |
| 6 | 90452808 | 90452835 | MDN1 | 0.015384615 |
| 19 | 55044241 | 55044256 | KIR3DX1 | 0.015384615 |
| 3 | 160138723 | 160138736 | SMC4 | 0.015384615 |
| 1 | 28233572 | 28233599 | RPA2 | 0.015384615 |
| 4 | 155180652 | 155180681 | DCHS2 | 0.015384615 |
| 5 | 180482303 | 180482317 | BTNL9 | 0.015503876 |
| 3 | 67058329 | 67058357 | KBTBD8 | 0.015503876 |
| 14 | 67848479 | 67848493 | EIF2S1 | 0.015503876 |
| 1 | 92108274 | 92108288 | CDC7(dist = 116953), TGFBR3(dist = 37612) | 0.015503876 |
| 2 | 191155233 | 191155245 | HIBCH | 0.015503876 |
| 2 | 152424933 | 152424962 | NEB | 0.015625 |
| 6 | 89638988 | 89639004 | RNGTT | 0.015625 |
| 1 | 16248728 | 16248740 | SPEN | 0.015625 |
| 17 | 66033401 | 66033412 | KPNA2 | 0.015625 |
| X | 48054294 | 48054306 | SSX5 | 0.015625 |
| 8 | 38961268 | 38961280 | ADAM9(NM_003816: c.*49_*61del0) | 0.015625 |
| 17 | 57128711 | 57128722 | TRIM37 | 0.015625 |
| 13 | 21373161 | 21373193 | XPO4 | 0.015625 |
| 12 | 105538478 | 105538491 | KIAA1033 | 0.015625 |
| 15 | 83805209 | 83805222 | TM6SF1 | 0.015748031 |
| 6 | 149842148 | 149842176 | PPIL4 | 0.015748031 |
| 6 | 51930886 | 51930901 | PKHD1 | 0.015748031 |
| 12 | 122437594 | 122437605 | WDR66 | 0.015873016 |
| 11 | 119145463 | 119145476 | CBL | 0.015873016 |
| 6 | 18258299 | 18258311 | DEK | 0.015873016 |
| 20 | 16359356 | 16359369 | KIF16B | 0.015873016 |
| 5 | 54701447 | 54701491 | SKIV2L2 | 0.015873016 |
| 8 | 96069973 | 96069988 | NDUFAF6 | 0.015873016 |
| 3 | 17418001 | 17418012 | TBC1D5 | 0.015873016 |
| 16 | 53721713 | 53721724 | RPGRIP1L | 0.015873016 |
| 7 | 105108955 | 105108983 | PUS7 | 0.015873016 |
| 2 | 120567392 | 120567406 | PTPN4 | 0.016 |
| 1 | 179089233 | 179089251 | ABL2 | 0.016 |
| 11 | 86868352 | 86868374 | TMEM135 | 0.016 |
| 15 | 54586266 | 54586278 | UNC13C | 0.016129032 |
| X | 21534510 | 21534521 | CNKSR2 | 0.016129032 |
| 2 | 141625834 | 141625854 | LRP1B | 0.016129032 |
| 17 | 74729588 | 74729600 | METTL23 | 0.016129032 |
| 18 | 12352952 | 12352964 | AFG3L2 | 0.016129032 |
| 8 | 59337246 | 59337258 | UBXN2B | 0.016129032 |
| 10 | 70506892 | 70506914 | CCAR1 | 0.016129032 |
| 2 | 233392012 | 233392045 | CHRND | 0.016129032 |
| X | 49030763 | 49030811 | PLP2 | 0.016129032 |
| 15 | 55972660 | 55972674 | PRTG | 0.016260163 |
| 8 | 67759411 | 67759438 | C8orf44-SGK3, SGK3 | 0.016260163 |
| 2 | 152359276 | 152359297 | NEB | 0.016260163 |
| 1 | 197150249 | 197150268 | ZBTB41 | 0.016260163 |
| 13 | 115007533 | 115007545 | CDC16 | 0.016260163 |
| 16 | 4790059 | 4790097 | C16orf71 | 0.016260163 |
| 2 | 21226218 | 21226230 | APOB | 0.016393443 |
| 6 | 90347394 | 90347406 | LYRM2 | 0.016393443 |
| 2 | 61622374 | 61622387 | USP34 | 0.016393443 |
| 2 | 169994021 | 169994058 | LRP2 | 0.016393443 |
| 7 | 98649755 | 98649771 | SMURF1 | 0.016393443 |
| 2 | 44934524 | 44934535 | CAMKMT | 0.016393443 |
| 13 | 72063066 | 72063077 | DACH1 | 0.016393443 |
| 12 | 120295568 | 120295581 | CIT | 0.016393443 |
| 10 | 102716310 | 102716330 | FAM178A | 0.016528926 |
| 1 | 160302354 | 160302365 | COPA | 0.016528926 |
| 9 | 79835312 | 79835353 | VPS13A | 0.016528926 |
| 1 | 161326722 | 161326735 | SDHC | 0.016528926 |
| 6 | 8417604 | 8417616 | SLC35B3 | 0.016528926 |
| 7 | 151884279 | 151884292 | KMT2C | 0.016528926 |
| 12 | 30822226 | 30822240 | IPO8 | 0.016528926 |
| 1 | 160268834 | 160268846 | COPA | 0.016528926 |
| 9 | 33311294 | 33311306 | NFX1 | 0.016528926 |
| 2 | 209113048 | 209113059 | IDH1 | 0.016528926 |
| X | 135095655 | 135095677 | SLC9A6 | 0.016666667 |
| 11 | 108332126 | 108332138 | C11orf65 | 0.016666667 |
| 15 | 82517622 | 82517671 | EFTUD1 | 0.016666667 |
| 7 | 24329103 | 24329114 | NPY | 0.016666667 |
| 7 | 106888946 | 106888972 | COG5 | 0.016806723 |
| 17 | 57278908 | 57278920 | PRR11 | 0.016806723 |
| 5 | 141511525 | 141511538 | NDFIP1 | 0.016806723 |
| 11 | 75316787 | 75316823 | MAP6(NM_207577: c.*62_*26del0) | 0.016806723 |
| 6 | 28876873 | 28876904 | TRIM27 | 0.016949153 |
| 1 | 161829973 | 161829987 | ATF6 | 0.016949153 |
| 9 | 88287588 | 88287615 | AGTPBP1 | 0.016949153 |
| 3 | 4669375 | 4669386 | ITPR1 | 0.016949153 |
| 13 | 41382557 | 41382568 | TPTE2P5 | 0.016949153 |
| 11 | 65270090 | 65270116 | MALAT1 | 0.016949153 |
| 12 | 8692327 | 8692338 | CLEC4E | 0.017094017 |
| 14 | 35497274 | 35497286 | SRP54 | 0.017241379 |
| 14 | 74875955 | 74875968 | SYNDIG1L | 0.017241379 |
| 1 | 155701055 | 155701071 | DAP3 | 0.017241379 |
| 8 | 24348425 | 24348437 | LOC101929294, LOC101929315 | 0.017391304 |
| 2 | 183832100 | 183832113 | NCKAP1 | 0.017391304 |
| 20 | 16025192 | 16025206 | MACROD2 | 0.017391304 |
| 2 | 28521140 | 28521152 | BRE | 0.017391304 |
| 15 | 73418741 | 73418756 | NEO1 | 0.01754386 |
| 12 | 113645152 | 113645163 | IQCD | 0.01754386 |
| 1 | 46650191 | 46650201 | TSPAN1 | 0.01754386 |
| 17 | 5324558 | 5324611 | RPAIN | 0.01754386 |
| 5 | 151180439 | 151180475 | G3BP1 | 0.01754386 |
| 10 | 71055448 | 71055460 | HK1 | 0.01754386 |
| 4 | 99802300 | 99802325 | EIF4E | 0.01754386 |
| 7 | 137782593 | 137782609 | AKR1D1 | 0.01754386 |
| 21 | 30400154 | 30400165 | USP16 | 0.01754386 |
| 10 | 91483908 | 91483928 | KIF20B | 0.01754386 |

TABLE 14A
Microsatellite Repeat Marker Sequences for Breast Invasive Carcinoma (BRCA).
TABLE 14A - BRCA

| ch | start_pos | end_pos | | BRCA |
|---|---|---|---|---|
| (hg37) | (hg37) | (hg37) | gene | p-value |
| 5 | 141520013 | 141520031 | NDFIP1 | 7.39469E−13 |
| 17 | 54981571 | 54981588 | TRIM25 | 1.13604E−10 |
| 22 | 41550984 | 41550996 | EP300 | 2.74259E−10 |
| X | 52654536 | 52654550 | SSX8 | 1.56045E−09 |
| 22 | 46679850 | 46679864 | TTC38 | 2.77117E−09 |
| 20 | 37146131 | 37146146 | RALGAPB | 4.42782E−09 |
| 5 | 137498739 | 137498760 | BRD8 | 4.76868E−09 |
| 6 | 17602786 | 17602812 | FAM8A1 | 5.7796E−09 |
| 11 | 59626477 | 59626491 | TCN1 | 1.1164E−08 |
| 12 | 64679694 | 64679708 | C12orf56 | 1.79237E−08 |
| 13 | 28133956 | 28133972 | LNX2 | 1.82274E−08 |
| 2 | 109371849 | 109371865 | RANBP2 | 2.62379E−08 |
| 5 | 137507104 | 137507121 | BRD8 | 2.85486E−08 |
| 9 | 19299209 | 19299224 | DENND4C | 3.94966E−08 |
| 1 | 98325237 | 98325262 | TM9SF3 | 3.9937E−08 |
| 12 | 107398862 | 107398876 | CRY1 | 4.03538E−08 |
| 15 | 65874368 | 65874380 | VWA9 | 4.37731E−08 |
| 16 | 74920075 | 74920089 | WDR59 | 5.49102E−08 |
| 2 | 15536509 | 15536532 | NBAS | 6.0039E−08 |
| 17 | 74286032 | 74286047 | ORICH2 | 6.11255E−08 |
| 3 | 108224685 | 108224701 | MYH15 | 7.07079E−08 |
| 14 | 91365646 | 91365664 | RPS6KA5 | 9.75311E−08 |
| 6 | 15651642 | 15651656 | DTNBP1 | 1.11363E−07 |
| 2 | 70456452 | 70456465 | TLA1 | 1.3204E−07 |
| 2 | 120620092 | 120620108 | PTPN4 | 1.40601E−07 |
| 18 | 649879 | 649895 | TYMSOS(NM_001012716: c.*120_*104del0) | 1.50586E−07 |
| 4 | 153268227 | 153268252 | FBXW7 | 1.60586E−07 |
| 12 | 124242458 | 124242472 | ATP6V0A2 | 1.74295E−07 |
| 3 | 38257551 | 38257583 | OXSR1 | 1.84433E−07 |
| 7 | 73666736 | 73666750 | RFC2 | 1.89725E−07 |
| 11 | 700526 | 700541 | TMEM80 | 2.19644E−07 |
| 6 | 117026217 | 117026229 | KPNA5 | 2.24588E−07 |
| 11 | 110128925 | 110128941 | RDX | 2.59053E−07 |
| 12 | 95488339 | 95488354 | FGD6 | 2.62525E−07 |
| 2 | 85589223 | 85589237 | ELMOD3 | 2.65083E−07 |
| 6 | 102130410 | 102130422 | GRIK2 | 2.8621E−07 |
| 18 | 70532185 | 70532210 | NETO1 | 2.89676E−07 |
| 14 | 56116576 | 56116587 | KTN1 | 3.48417E−07 |
| 1 | 200080464 | 200080490 | NR5A2 | 3.55386E−07 |
| 12 | 40258687 | 40258699 | SLC2A13 | 3.68811E−07 |
| 6 | 131999103 | 131999115 | ENPP3 | 3.96019E−07 |
| X | 44386435 | 44386460 | FUNDC1 | 3.9669E−07 |
| 8 | 89128708 | 89128722 | MMP16 | 4.23715E−07 |
| 7 | 2406737 | 2406755 | EIF3B | 4.50223E−07 |
| 2 | 101627343 | 101627360 | RPL31, TBC1D8 | 4.72142E−07 |
| 5 | 172421760 | 172421776 | ATP6V0E1 | 4.81253E−07 |
| X | 117035914 | 117035926 | KLHL13 | 4.88511E−07 |
| 9 | 133498017 | 133498047 | FUBP3 | 4.92464E−07 |
| 15 | 72864384 | 72864397 | ARIH1 | 5.00396E−07 |
| 20 | 34435246 | 34435270 | PHF20 | 5.51837E−07 |
| 9 | 131743701 | 131743715 | NUP188 | 5.66832E−07 |
| 2 | 160092703 | 160092716 | WDSUB1 | 5.68459E−07 |
| 14 | 39602843 | 39602863 | GEMIN2 | 5.68887E−07 |
| 6 | 64419024 | 64419039 | PHF3 | 5.69496E−07 |
| 6 | 131997990 | 131998015 | ENPP3 | 6.23312E−07 |
| 9 | 135523692 | 135523704 | DDX31 | 7.45582E−07 |
| 20 | 30142484 | 30142500 | HM13 | 8.56195E−07 |
| 19 | 51919434 | 51919447 | LOC100129083, SIGLEC10 | 8.76743E−07 |
| 4 | 55131001 | 55131019 | PDGFRA | 8.78839E−07 |
| X | 107827760 | 107827773 | COL4A5 | 8.94998E−07 |
| 7 | 107336262 | 107336274 | SLC26A4 | 9.33065E−07 |
| 7 | 23226646 | 23226667 | NUPL2 | 9.65402E−07 |
| 9 | 100700505 | 100700528 | HEMGN(NM_197978: c.-87_-110del0) | 9.71155E−07 |
| 8 | 42798394 | 42798409 | HOOK3 | 9.85408E−07 |
| 14 | 21964623 | 21964635 | TOX4 | 9.9999E−07 |
| 12 | 62261077 | 62261088 | FAM19A2 | 1.03774E−06 |
| X | 52706249 | 52706291 | SSX7(dist = 22299), SSX2B(dist = 19655) | 1.04282E−06 |
| 21 | 37619920 | 37619943 | DOPEY2 | 1.11088E−06 |
| 6 | 36452603 | 36452620 | KCTD20 | 1.18027E−06 |
| 9 | 36109839 | 36109853 | RECK | 1.25128E−06 |
| 1 | 22848150 | 22848166 | TUBGCP5 | 1.25244E−06 |
| 8 | 80831393 | 80831407 | MRPS28 | 1.26108E−06 |
| 5 | 55034756 | 55034770 | DDX4 | 1.28081E−06 |
| 1 | 231502230 | 231502245 | EGLN1 | 1.28156E−06 |
| 21 | 30435646 | 30435668 | CCT8 | 1.31987E−06 |
| 9 | 5811331 | 5811349 | ERMP1 | 1.32712E−06 |
| 11 | 8494831 | 8494856 | STK33 | 1.36887E−06 |
| X | 2326742 | 2326763 | DHRSX | 1.38274E−06 |
| 5 | 81601142 | 81601155 | ATP6AP1L(NM_001017971: c.-79_-66del-) | 1.46347E−06 |
| 8 | 62557195 | 62557207 | ASPH | 1.4896E−06 |
| 9 | 125827752 | 125827764 | RABGAP1 | 1.62925E−06 |
| X | 154133316 | 154133329 | F8 | 1.71999E−06 |
| 2 | 207013834 | 207013846 | NDUFS1 | 1.94239E−06 |
| 16 | 74372596 | 74372618 | LOC283922 | 1.95895E−06 |
| 1 | 179271948 | 179271961 | SOAT1 | 2.00939E−06 |
| 17 | 57278908 | 57278920 | PRR11 | 2.09166E−06 |
| 12 | 53470811 | 53470838 | SPRYD3 | 2.13088E−06 |
| 6 | 52768392 | 52768408 | GSTA3 | 2.14768E−06 |
| 8 | 74204423 | 74204438 | RPL7 | 2.25564E−06 |
| 3 | 160233406 | 160233421 | KPNA4 | 2.39065E−06 |
| 19 | 47700438 | 47700453 | SAE1 | 2.393E−06 |
| 2 | 55559651 | 55559667 | CCDC88A | 2.40511E−06 |
| 15 | 43307839 | 43307852 | UBR1 | 2.41375E−06 |
| 17 | 7811876 | 7811889 | CHD3 | 2.44862E−06 |
| X | 48214185 | 48214200 | SSX3 | 2.50868E−06 |
| 4 | 113506885 | 113506900 | ZGRF1 | 2.72291E−06 |
| 22 | 16920145 | 16920157 | OR11H1(dist = 470341), CCT8L2(dist = 151491) | 2.76507E−06 |
| 19 | 36145409 | 36145425 | COX6B1 | 2.94377E−06 |
| 3 | 154002357 | 154002370 | DHX36 | 3.00052E−06 |
| 10 | 13633928 | 13633944 | PRPF18 | 3.0722E−06 |
| 8 | 33356825 | 33356839 | MAK16(NM_032509: c.*678_*692del0) | 3.09035E−06 |
| 14 | 68944343 | 68944358 | RAD51B | 3.11938E−06 |
| 6 | 26373465 | 26373491 | BTN3A2 | 3.1902E−06 |
| 7 | 92020452 | 92020465 | ANKIB1 | 3.20526E−06 |
| 1 | 172577864 | 172577879 | SUCO | 3.21437E−06 |
| 2 | 212495336 | 212495354 | ERBB4 | 3.21515E−06 |
| 10 | 102019924 | 102019955 | CWF19L1 | 3.25878E−06 |
| 7 | 50444181 | 50444192 | IKZF1 | 3.275E−06 |
| 14 | 88454869 | 88454880 | GALC | 3.71214E−06 |
| 4 | 128812230 | 128812245 | PLK4 | 4.08579E−06 |
| 17 | 1584377 | 1584390 | PRPF8 | 4.11035E−06 |
| 5 | 86679495 | 86679514 | RASA1 | 4.26896E−06 |
| 3 | 19959707 | 19959719 | EFHB | 4.33968E−06 |
| 4 | 128817905 | 128817919 | PLK4 | 4.56348E−06 |
| 1 | 99762456 | 99762467 | LPPR4 | 4.59205E−06 |
| 17 | 16041578 | 16041592 | NCOR1 | 4.82547E−06 |
| 5 | 77745712 | 77745752 | SCAMP1 | 4.99553E−06 |
| 10 | 15113934 | 15113946 | OLAH | 5.10614E−06 |
| 3 | 52696310 | 52696322 | PBRM1 | 5.20749E−06 |
| 10 | 75214137 | 75214149 | PPP3CB | 5.33266E−06 |
| 1 | 161326722 | 161326735 | SDHC | 5.40943E−06 |
| 11 | 117222647 | 117222659 | CEP164 | 5.41437E−06 |
| 2 | 183960133 | 183960155 | DUSP19 | 5.55667E−06 |
| IC | 16911550 | 16911567 | CUBN | 5.60648E−06 |
| 13 | 79216356 | 79216381 | RNF219 | 5.675E−06 |
| X | 79991590 | 79991604 | BRWD3 | 5.6906E−06 |
| 5 | 96116095 | 96116125 | ERAP1 | 5.70625E−06 |
| 3 | 167045701 | 167045723 | ZBBX | 5.77137E−06 |
| 2 | 30356973 | 30356986 | LTN1 | 5.92892E−06 |
| 2 | 99936265 | 99936280 | TXNDC9 | 5.94906E−06 |
| 4 | 85556378 | 85556414 | CDS1 | 6.08622E−06 |
| 12 | 4645161 | 4645173 | C12orf4 | 6.10107E−06 |
| 1 | 28929961 | 28929973 | TAF12(NM_001135218: c.*90_*78del0, | 6.30636E−06 |
| | | | NM_005644: c.*90_*78del0) | |
| 15 | 51017381 | 51017393 | SPPL2A | 6.30656E−06 |
| 15 | 63571279 | 63571293 | APH1B | 6.63087E−06 |
| 17 | 686296 | 686309 | RNMTL1 | 6.71783E−06 |
| 16 | 69385403 | 69385421 | TMED6 | 6.83438E−06 |
| 2 | 183033020 | 183033033 | PDELA | 6.95836E−06 |
| 2 | 203079025 | 203079039 | SUMO1 | 6.97934E−06 |
| 1 | 25679278 | 25679290 | TMEM50A | 7.18956E−06 |
| 8 | 42295130 | 42295156 | SLC20A2 | 7.26099E−06 |
| 2 | 170062201 | 170062216 | LRP2 | 7.41014E−06 |
| 2 | 47649496 | 47649525 | ARFGEF2 | 7.53143E−06 |
| 19 | 22836873 | 22836886 | ZNF492 | 7.57428E−06 |
| 15 | 59974525 | 59974539 | BNIP2 | 7.6175E−06 |
| 18 | 18619416 | 18619428 | ROCK1 | 7.70041E−06 |
| 15 | 80460576 | 80460591 | FAH | 7.738E−06 |
| 2 | 200800572 | 200800585 | TYW5 | 8.08649E−06 |
| 15 | 64717845 | 64717857 | TRIP4 | 8.10818E−06 |
| 11 | 74053485 | 74053497 | PGM2L1 | 8.18019E−06 |
| 1 | 93159821 | 93159835 | EVI5 | 8.33371E−06 |
| 15 | 58974349 | 58974361 | ADAM10 | 8.36316E−06 |
| 17 | 44833088 | 44833123 | NSF | 8.44987E−06 |
| 12 | 6858889 | 6858905 | MLF2 | 8.57753E−06 |
| 12 | 81239721 | 81239737 | LIN7A | 8.61207E−06 |
| 6 | 149842148 | 149842176 | PPIL4 | 8.61927E−06 |
| 14 | 60616914 | 60616937 | DHRS7 | 8.79386E−06 |
| 20 | 4839890 | 4839911 | SLC23A2 | 8.92537E−06 |
| 10 | 11047207 | 11047257 | CELF2 | 9.06402E−06 |
| 14 | 64632236 | 64632249 | MIR548AZ | 9.21319E−06 |
| 18 | 57684454 | 57684466 | PMAIP1(dist = 112916), MC4R(dist = 354098) | 9.22399E−06 |
| 7 | 7572355 | 7572381 | COL28A1 | 9.30353E−06 |
| 12 | 104461711 | 104461723 | HCFC2 | 9.38876E−06 |
| 14 | 36218039 | 36218056 | RALGAPA1P | 9.44793E−06 |
| 1 | 154227292 | 154227307 | UBAP2L | 1.00433E−05 |
| 15 | 45725103 | 45725138 | C15orf48 | 1.02325E−05 |
| 8 | 3611416 | 3611429 | CSMD1 | 1.02542E−05 |
| 20 | 13140617 | 13140637 | SPTLC3 | 1.06428E−05 |
| 11 | 8486375 | 8486389 | STK33 | 1.08438E−05 |
| 9 | 27062802 | 27062816 | IFT74(NM_001099223: c.*68_*82del0, | 1.11177E−05 |
| | | | NM_001099222: c.*68_*82del0, | |
| | | | NM_025103: c.*68_*82del0) | |
| 2 | 64069277 | 64069293 | UGP2(NM_006759: c.-43-27del-) | 1.12033E−05 |
| 6 | 146256315 | 146256344 | SHPRH | 1.12897E−05 |
| 15 | 83808031 | 83808046 | HDGFRP3 | 1.17034E−05 |
| 5 | 94842718 | 94842730 | TTC37 | 1.18964E−05 |
| 11 | 47701629 | 47701642 | AGBL2 | 1.21899E−05 |
| 9 | 134049353 | 134049366 | NUP214 | 1.26095E−05 |
| 4 | 146046123 | 146046135 | ABCE1 | 1.31256E−05 |
| 5 | 16474764 | 16474795 | FAM134B(NM_001034850: c.*86_*55del0, | 1.35483E−05 |
| | | | NM_019000: c.*86_*55del0) | |
| 22 | 30772228 | 30772244 | KIAA1656 | 1.41669E−05 |
| 11 | 88068256 | 88068270 | CTSC | 1.43837E−05 |
| 10 | 74874082 | 74874112 | NUDT13 | 1.45304E−05 |
| 11 | 113697927 | 113697939 | USP28 | 1.46898E−05 |
| 16 | 5129689 | 5129703 | ALG1 | 1.49148E−05 |
| 15 | 50596341 | 50596355 | GABPB1 | 1.49173E−05 |
| 5 | 180482303 | 180482317 | BTNL9 | 1.4988E−05 |
| 7 | 74119460 | 74119488 | LOC101926943 | 1.51261E−05 |
| 2 | 37487307 | 37487330 | PRKD3 | 1.5167E−05 |
| 7 | 5239305 | 5239336 | WIPI2 | 1.5343E−05 |
| 15 | 55841195 | 55841209 | PYGO1 | 1.54703E−05 |
| X | 106028279 | 106028291 | RNF128 | 1.54895E−05 |
| 12 | 53861636 | 53861650 | PCBP2 | 1.63997E−05 |
| 5 | 72168575 | 72168596 | TNPO1 | 1.65647E−05 |
| 1 | 202990110 | 202990128 | TMEM183A, TMEM183B | 1.6701E−05 |
| 13 | 103257289 | 103257311 | TPP2 | 1.68273E−05 |
| 12 | 98989187 | 98989199 | SLC25A3 | 1.70421E−05 |
| 9 | 19334957 | 19334970 | DENND4C | 1.74754E−05 |
| 7 | 65426054 | 65426069 | GUSB | 1.83556E−05 |
| 2 | 196738263 | 196738274 | DNAH7 | 1.88079E−05 |
| 4 | 22444251 | 22444267 | GPR125 | 1.93115E−05 |
| 11 | 63149670 | 63149682 | SLC22A9 | 1.9384E−05 |
| 2 | 234916771 | 234916787 | TRPM8 | 1.95462E−05 |
| 11 | 117152291 | 117152314 | RNF214 | 1.95726E−05 |
| 1 | 215914883 | 215914896 | USH2A | 1.96468E−05 |
| 7 | 40234742 | 40234756 | SUGCT | 2.02964E−05 |
| 3 | 130653386 | 130653399 | ATP2C1 | 2.0348E−05 |
| 4 | 151850226 | 151850239 | LRBA | 2.05149E−05 |
| 8 | 67710676 | 67710692 | C8orf44-SGK3, SGK3 | 2.07821E−05 |
| 1 | 205129466 | 205129482 | DSTYK | 2.08628E−05 |
| 13 | 77792099 | 77792113 | MYCBP2 | 2.11048E−05 |
| 3 | 122081881 | 122081893 | CCDC58 | 2.14008E−05 |
| 8 | 38940143 | 38940157 | ADAM9 | 2.20437E−05 |
| 1 | 117633133 | 117633144 | TTF2 | 2.2361E−05 |
| 4 | 1701673 | 1701686 | SLBP | 2.24738E−05 |
| 4 | 123109034 | 123109048 | KIAA1109 | 2.25353E−05 |
| X | 40588605 | 40588618 | MED14 | 2.28258E−05 |
| 1 | 63836738 | 63836761 | ALG6 | 2.32945E−05 |
| 3 | 186302391 | 186302439 | DNAJB11 | 2.32978E−05 |
| 8 | 30569609 | 30569622 | GSR | 2.33037E−05 |
| 8 | 133854714 | 133854728 | PHF20L1 | 2.33072E−05 |
| 4 | 159590922 | 159590934 | C4orf46 | 2.37701E−05 |
| 1 | 109350198 | 109350216 | STXBP3 | 2.39252E−05 |
| 14 | 58825825 | 58825847 | ARID4A | 2.39775E−05 |
| 22 | 40696618 | 40696645 | TNRC6B | 2.39847E−05 |
| 14 | 59787325 | 59787338 | DAAM1 | 2.43646E−05 |
| 22 | 46739288 | 46739300 | TRMU | 2.43653E−05 |
| 7 | 98649755 | 98649771 | SMURF1 | 2.47958E−05 |
| 12 | 42835328 | 42835365 | PPHLN1(NM_201439: c.*17_*54del0, | 2.55894E−05 |
| | | | NM_201440: c.*17_*54del0, | |
| | | | NM_001143788: c.*17_*54del0, | |
| | | | NM_001143789: c.*17_*54del0, | |
| | | | NM_201515: c.*17_*54del0) | |
| 7 | 140125524 | 140125548 | RAB19 | 2.55904E−05 |
| X | 52674318 | 52674335 | SSX7 | 2.5792E−05 |
| 15 | 73409213 | 73409225 | NEO1 | 2.60029E−05 |
| 14 | 76638065 | 76638087 | GPATCH2L | 2.61507E−05 |
| 7 | 131130509 | 131130538 | MKLN1 | 2.65076E−05 |
| 1 | 38301478 | 38301491 | MTF1 | 2.66706E−05 |
| 14 | 35242702 | 35242726 | BAZ1A | 2.66842E−05 |
| 2 | 61145498 | 61145512 | REL | 2.68108E−05 |
| 13 | 100913132 | 100913161 | PCCA | 2.69009E−05 |
| 13 | 77818089 | 77818117 | MYCBP2 | 2.72538E−05 |
| 13 | 114240056 | 114240102 | TFDP1(NM_007111: c.-64_-51delins-) | 2.79044E−05 |
| 20 | 7921106 | 7921120 | HAO1 | 2.89797E−05 |
| 17 | 37119364 | 37119376 | FBXO47 | 2.91622E−05 |
| 20 | 3245219 | 3245232 | C20orf194 | 2.99609E−05 |
| 11 | 32610726 | 32610740 | EIF3M | 3.00843E−05 |
| 14 | 58690427 | 58690451 | ACTR10 | 3.03792E−05 |
| X | 148582573 | 148582585 | IDS | 3.04166E−05 |
| 20 | 48467300 | 48467311 | SLC9A8 | 3.08203E−05 |
| 1 | 41456808 | 41456823 | CTPS1 | 3.11072E−05 |
| 1 | 36884530 | 36884544 | OSCP1 | 3.15591E−05 |
| 3 | 185641778 | 185641789 | TRA2B | 3.20123E−05 |
| 21 | 40650627 | 40650652 | BRWD1 | 3.29558E−05 |
| 12 | 123103115 | 123103129 | KNTC1 | 3.32684E−05 |
| 4 | 186188373 | 186188388 | SNX25 | 3.38557E−05 |
| 3 | 69112658 | 69112688 | UBA3 | 3.40289E−05 |
| 22 | 30367073 | 30367092 | MTMR3 | 3.4236E−05 |
| 2 | 162849944 | 162849956 | DPP4 | 3.61128E−05 |
| 6 | 89638988 | 89639004 | RNGTT | 3.63608E−05 |
| 18 | 21124907 | 21124917 | NPC1 | 3.72203E−05 |
| 6 | 100403812 | 100403827 | MCHR2 | 3.73882E−05 |
| 2 | 27597190 | 27597204 | SNX17 | 3.7498E−05 |
| 12 | 93195359 | 93195370 | EEA1 | 3.75154E−05 |
| X | 48054294 | 48054306 | SSX5 | 3.78256E−05 |
| 6 | 88853530 | 88853542 | CNR1(NM_001160259: c.*45_*33del0, | 3.8126E−05 |
| | | | NM_001160226: c*45_*33del0, | |
| | | | NM_001160258: c.*45_*33del0, | |
| | | | NM_033181: c.*45_*33del0, | |
| | | | NM_016083: c.*45_*33del0) | |
| 16 | 21809188 | 21809208 | RRN3P1 | 4.03224E−05 |
| 1 | 59942586 | 59942600 | KIAA1468 | 4.03953E−05 |
| 19 | 39909218 | 39909231 | PLEKHG2 | 4.04858E−05 |
| 2 | 211521213 | 211521243 | CPS1 | 4.07345E−05 |
| 9 | 118164375 | 118164388 | DEC1 | 4.13609E−05 |
| 15 | 102287989 | 102288000 | TARSL2(dist = 23344), OR4F6(dist = 57923) | 4.14236E−05 |
| 1 | 90229786 | 90229798 | PEX11A | 4.21992E−05 |
| 11 | 16339918 | 16339934 | SOX6 | 4.22764E−05 |
| 2 | 54146387 | 54146400 | PSME4 | 4.28541E−05 |
| 2 | 32706267 | 32706279 | BIRC6 | 4.32867E−05 |
| 1 | 64022708 | 64022725 | EFCAB7 | 4.42689E−05 |
| 17 | 48202125 | 48202172 | SAMD14 | 4.48308E−05 |
| 11 | 112832276 | 112832287 | LOC101928847 | 4.50464E−05 |
| 7 | 107875143 | 107875165 | NRCAM | 4.52821E−05 |
| 8 | 95186510 | 95186526 | CDH17 | 4.66014E−05 |
| 5 | 96093391 | 96093408 | CAST | 4.69355E−05 |
| 4 | 74010555 | 74010571 | ANKRD17 | 4.7575E−05 |
| 2 | 87049358 | 87049371 | CD8B | 4.76077E−05 |
| 2 | 55863360 | 55863372 | PNPT1 | 4.79939E−05 |
| 15 | 41060070 | 41060085 | DNAJC17(NM_018163: c.*68_*53del0) | 4.80818E−05 |
| 19 | 37155495 | 37155509 | ZNF461 | 4.88873E−05 |
| 7 | 16704933 | 16704945 | BZW2 | 4.96515E−05 |
| 11 | 16117684 | 16117698 | SOX6 | 4.96882E−05 |
| 5 | 60170512 | 60170537 | ERCC8 | 4.97431E−05 |
| 15 | 60748719 | 60748828 | ICE2 | 5.02163E−05 |
| 3 | 129168854 | 129168881 | IFT122 | 5.07038E−05 |
| 5 | 134086670 | 134086684 | CAMLG(NM_001745: c.*30_*44del0) | 5.07339E−05 |
| 3 | 71008341 | 71008362 | FOXP1(NM_001244816: c.*57_*36del0, | 5.0789E−05 |
| | | | NM_001244812: c.*57_*36del0, | |
| | | | NM_001244813: c.*57_*36del0, | |
| | | | NM_001244808: c.*57_*36del0, | |
| | | | NM_001244815: c.*57_*36del0, | |
| | | | NM_001244814: c.*57_*36del0, | |
| | | | NM_032682: c.*57_*36del0, | |
| | | | NM_001244810: c.*57_*36del0) | |
| 19 | 21350520 | 21350533 | ZNF431 | 5.09785E−05 |
| 12 | 108140266 | 108140281 | LOC101929162 | 5.09936E−05 |
| 12 | 72067966 | 72067980 | THAP2 | 5.21438E−05 |
| 6 | 34735652 | 34735670 | SNRPC | 5.24827E−05 |
| 6 | 52400479 | 52400497 | TRAM2 | 5.3806E−05 |
| 15 | 59806473 | 59806486 | FAM81A | 5.5757E−05 |
| 6 | 116914112 | 116914128 | RWDD1 | 5.59729E−05 |
| X | 20204548 | 20204563 | RPS6KA3 | 5.73558E−05 |
| 1 | 94212930 | 94212942 | MRE11A | 5.73564E−05 |
| 1 | 52242432 | 52242447 | OSBPL9 | 5.76012E−05 |
| 3 | 10018739 | 10018754 | EMC3 | 5.89287E−05 |
| 10 | 94366347 | 94366370 | KIF11 | 5.95986E−05 |
| X | 15373247 | 15373259 | PIR-FIGF | 6.00975E−05 |
| 4 | 147628705 | 147628716 | TTC29 | 6.01661E−05 |
| 14 | 45587186 | 45587199 | FKBP3 | 6.0548E−05 |
| 2 | 74754818 | 74754830 | AUP1 | 6.09097E−05 |
| 12 | 28459652 | 28459664 | CCDC91 | 6.22686E−05 |
| 4 | 48529683 | 48529699 | FRYL | 6.29885E−05 |
| 14 | 96788613 | 96788623 | ATG2B | 6.34898E−05 |
| 2 | 55883509 | 55883523 | PNPT1 | 6.3674E−05 |
| 8 | 121455551 | 121455586 | MRPL13 | 6.47651E−05 |
| 17 | 58013510 | 58013529 | RPS6KB1 | 6.57242E−05 |
| X | 3539376 | 3539390 | PRKX | 6.63723E−05 |
| 8 | 131812577 | 131812590 | ADCY8 | 6.6825E−05 |
| X | 24084241 | 24084254 | EIF2S3 | 6.7347E−05 |
| 7 | 100843369 | 100843385 | MOGAT3 | 6.78625E−05 |
| 17 | 19744865 | 19744877 | ULK2 | 6.79165E−05 |
| 2 | 29004734 | 29004745 | PPP1CB | 6.8567E−05 |
| 2 | 11716433 | 11716447 | GREB1 | 6.89306E−05 |
| 15 | 43990706 | 43990741 | CKMT1A | 6.8996E−05 |
| 3 | 180320897 | 180320910 | TTC14 | 6.99007E−05 |
| 8 | 28989764 | 28989782 | KIF13B | 6.99888E−05 |
| 1 | 39751172 | 39751188 | MACF1 | 7.03684E−05 |
| 15 | 63447932 | 63447960 | RPS27L | 7.04191E−05 |
| 18 | 6873646 | 6873663 | ARHGAP28 | 7.08486E−05 |
| 15 | 36910962 | 36910982 | C15orf41 | 7.1382E−05 |
| 16 | 19063220 | 19063235 | TMC7 | 7.15085E−05 |
| 5 | 14485161 | 14485175 | TRIO | 7.15971E−05 |
| 8 | 71037103 | 71037115 | NCOA2 | 7.20881E−05 |
| 20 | 29633958 | 29633977 | FRG1B | 7.2897E−05 |
| 8 | 130874614 | 130874639 | FAM49B | 7.29254E−05 |
| 22 | 42415224 | 42415240 | WBP2NL | 7.34744E−05 |
| 5 | 34945797 | 34945810 | DNAJC21 | 7.3858E−05 |
| 3 | 112719791 | 112719808 | GTPBP8(NM_014170: c.*25_*42del0, | 7.42669E−05 |
| | | | NM_138485: c.*25_*42del0) | |
| 2 | 203417391 | 203417403 | BMPR2 | 7.47128E−05 |
| 1 | 220284112 | 220284124 | RNU5F-1 | 7.71697E−05 |
| 6 | 17675386 | 17675402 | NUP153 | 7.78483E−05 |
| 8 | 23712066 | 23712089 | STC1(NM_003155: c.-30_-53del0) | 7.84037E−05 |
| 2 | 230653669 | 230653682 | TRIP12 | 7.98255E−05 |
| 14 | 20822853 | 20822873 | PARP2 | 7.9997E−05 |
| 3 | 185155430 | 185155442 | MAP3K13 | 8.02043E−05 |
| 6 | 80910611 | 80910635 | BCKDHB | 8.08064E−05 |
| 14 | 104465128 | 104465144 | TDRD9 | 8.13188E−05 |
| 2 | 33586464 | 33586479 | LTBP1 | 8.13887E−05 |
| 2 | 64139831 | 64139844 | VPS54 | 8.15116E−05 |
| 13 | 113527868 | 113527879 | ATP11A | 8.32646E−05 |
| X | 1464126 | 1464139 | IL3RA | 8.41898E−05 |
| 1 | 110551590 | 110551602 | AHCYL1 | 8.5073E−05 |
| 11 | 108141955 | 108141971 | ATM | 8.56614E−05 |
| 1 | 93584864 | 93584885 | MTF2 | 8.62756E−05 |
| 2 | 64323751 | 64323763 | PELI1 | 8.75648E−05 |
| X | 3238003 | 3238028 | MXRA5 | 8.99918E−05 |
| 2 | 24345196 | 24345211 | FAM228B, PFN4 | 9.07112E−05 |
| 1 | 25571794 | 25571805 | RSRP1 | 9.10558E−05 |
| 5 | 153174310 | 153174322 | GRIA1 | 9.17151E−05 |
| 3 | 130660392 | 130660405 | ATP2C1 | 9.26142E−05 |
| 19 | 11411997 | 11412010 | TSPAN16 | 9.35861E−05 |
| 13 | 48664556 | 48664569 | MED4 | 9.53509E−05 |
| 11 | 102575512 | 102575536 | MMP27 | 9.58789E−05 |
| 20 | 47648454 | 47648481 | ARFGEF2 | 9.68143E−05 |
| 8 | 79629738 | 79629753 | ZC2HC1A(NM_016010: c.*10_*25del0) | 9.70187E−05 |
| 19 | 55044241 | 55044256 | KIR3DX1 | 9.72074E−05 |
| 13 | 33349228 | 33349251 | PDS5B(NM_015032: c.*38_*61del0) | 9.79287E−05 |
| 3 | 176751870 | 176751887 | TBL1XR1 | 9.79673E−05 |
| 1 | 118369265 | 118369290 | KMT2A | 9.86801E−05 |
| 18 | 29238076 | 29238091 | B4GALT6 | 9.98031E−05 |
| 9 | 20346363 | 20346406 | MLLT3(NM_004529: c.*78_*35del0, | 0.000100456 |
| | | | NM_001286691: c.*78_*35del0) | |
| 9 | 133498229 | 133498245 | FUBP3 | 0.000100818 |
| 17 | 19749952 | 19749969 | ULK2 | 0.000101112 |
| 12 | 97306603 | 97306631 | NEDD1 | 0.000101316 |
| 1 | 65099701 | 65099716 | CACHD1 | 0.000101484 |
| 5 | 122359456 | 122359480 | PPIC(NM_000943: c.*114_*90del0) | 0.00010254 |
| 15 | 66794298 | 66794311 | RPL4 | 0.000102816 |
| 3 | 3216976 | 3216988 | CRBN | 0.000102985 |
| 12 | 100496478 | 100496498 | UHRF1BP1L | 0.00010353 |
| 10 | 96317838 | 96317849 | HELLS | 0.000103543 |
| 10 | 32575773 | 32575785 | EPC1 | 0.000103972 |
| 6 | 116912033 | 116912049 | RWDD1 | 0.000106324 |
| 7 | 33192288 | 33192300 | BBS9 | 0.000106487 |
| 7 | 16705132 | 16705155 | BZW2 | 0.000107395 |
| 2 | 201778717 | 201778733 | ORC2 | 0.000108986 |
| 13 | 26151184 | 26151196 | ATP8A2 | 0.000109325 |
| 8 | 131859768 | 131859803 | ADCY8 | 0.000110123 |
| 10 | 93784489 | 93784504 | BTAF1 | 0.000110624 |
| 2 | 206997835 | 206997847 | NDUFS1 | 0.000111763 |
| 3 | 169992958 | 169992994 | PRKCI | 0.000112151 |
| 6 | 70840124 | 70840137 | COL19A1 | 0.000112286 |
| 3 | 146167163 | 146167186 | PLSCR2 | 0.00011286 |
| 1 | 38052867 | 38052907 | GNL2 | 0.000113072 |
| 2 | 165365365 | 165365376 | GRB14 | 0.000113965 |
| 1 | 53255587 | 53255601 | ZYG11B | 0.000113969 |
| 1 | 49196555 | 49196567 | FOLH1 | 0.000114053 |
| 1 | 156203572 | 156203588 | PMF1, PMF1-BGLAP | 0.000115527 |
| 10 | 70056619 | 70056632 | PBLD | 0.000115905 |
| X | 117530912 | 117530925 | WDR44 | 0.000116052 |
| 14 | 55629650 | 55629664 | DLGAP5 | 0.000116241 |
| X | 54042926 | 54042942 | PHF8 | 0.000116241 |
| 10 | 101992923 | 101992938 | CWF19L1(NM_018294: c.*61_*46del0) | 0.000116687 |
| 8 | 74495112 | 74495130 | STAU2 | 0.000116687 |
| 1 | 115280193 | 115280207 | CSDE1 | 0.000119521 |
| 2 | 179478746 | 179478757 | MIR548N, TTN-AS1 | 0.000121052 |
| 1 | 23636874 | 23636886 | HNRNPR(NM_001102397: c.*73_*61del0, | 0.000121082 |
| | | | NM_001102398: c.*73_*61del0, | |
| | | | NM_001102399: c.*73_*61del0, | |
| | | | NM_001297621: c.*73_*61del0, | |
| | | | NM_005826: c.*73_*61del0, | |
| | | | NM_001297622: c.*73_*61del0, | |
| | | | NM_001297620: c.*73_*61del0) | |
| 17 | 57937792 | 57937807 | TUBD1 | 0.000121406 |
| 8 | 98817552 | 98817571 | LAPTM4B | 0.000122918 |
| 15 | 65273147 | 65273161 | SPG21 | 0.000123656 |
| 12 | 9706558 | 9706571 | DDX12P(dist = 105790), KLRB1(dist = 41299) | 0.000124364 |
| 8 | 68140383 | 68140394 | ARFGEF1 | 0.000124626 |
| 11 | 46666872 | 46666883 | ATG13 | 0.000125707 |
| 7 | 21646233 | 21646254 | DNAH11 | 0.000125754 |
| 8 | 134125650 | 134125662 | TG | 0.000126143 |
| 12 | 118463653 | 118463672 | RFC5 | 0.000128595 |
| 13 | 25467010 | 25467036 | CENPJ | 0.000129675 |
| 7 | 95775848 | 95775863 | SLC25A13 | 0.000129763 |
| 12 | 54963479 | 54963504 | PDE1B | 0.000130574 |
| 8 | 30933816 | 30933829 | WRN | 0.000130739 |
| 9 | 3937244 | 3937266 | GLIS3 | 0.000131271 |
| 7 | 131113756 | 131113779 | MKLN1 | 0.000131353 |
| 2 | 102959416 | 102959427 | IL1RL1 | 0.000137854 |
| 2 | 48046012 | 48046035 | FBXO11 | 0.000138729 |
| 6 | 44216544 | 44216556 | HSP90AB1 | 0.000138791 |
| 2 | 61456592 | 61456645 | USP34 | 0.000140362 |
| 3 | 52676065 | 52676079 | PBRM1 | 0.000141221 |
| 11 | 86120270 | 86120294 | CCDC81 | 0.000141469 |
| 16 | 69293926 | 69293945 | SNTB2 | 0.000141826 |
| 5 | 76707435 | 76707447 | PDE8B | 0.000141841 |
| 15 | 101019693 | 101019705 | CERS3 | 0.000142232 |
| 1 | 241954005 | 241954024 | WDR64 | 0.000142972 |
| 4 | 39511530 | 39511543 | MIR1273H | 0.000143658 |
| 5 | 170818290 | 170818301 | NPM1 | 0.000144503 |
| 10 | 52502803 | 52502820 | ASAH2B | 0.000144537 |
| 17 | 33288433 | 33288445 | CCT6B(NM_001193530: c.-21_-33del0, | 0.00014505 |
| | | | NM_001193529: c.-21_-33del0, | |
| | | | NM_006584: c.-21_-33del0) | |
| 14 | 72941317 | 72941328 | RGS6 | 0.000147191 |
| 6 | 17669262 | 17669277 | NUP153 | 0.000147437 |
| 13 | 24410498 | 24410511 | MIPEP | 0.000147674 |
| 2 | 60988862 | 60988872 | PAPOLG | 0.000150282 |
| 1 | 153516162 | 153516173 | S100A4(NM_019554: c.*73_*62del0, | 0.000150567 |
| | | | NM_002961: c.*73_*62del0) | |
| 14 | 39594134 | 39594146 | GEMIN2 | 0.000151984 |
| 2 | 158275111 | 158275135 | CYTIP | 0.000153201 |
| 7 | 77006696 | 77006740 | GSAP | 0.000153239 |
| 11 | 119145463 | 119145476 | CBL | 0.000153271 |
| 6 | 100006295 | 100006309 | CCNC | 0.000153773 |
| 19 | 42213456 | 42213487 | CEACAM5 | 0.000154108 |
| 6 | 8417604 | 8417616 | SLC35B3 | 0.00015465 |
| 5 | 32057910 | 32057923 | PDZD2 | 0.000155854 |
| 8 | 103287850 | 103287864 | UBR5 | 0.000156041 |
| 4 | 40123997 | 40124010 | N4BP2 | 0.000156489 |
| 7 | 64004813 | 64004850 | ZNF680 | 0.000158124 |
| 16 | 3558262 | 3558286 | CLUAP1 | 0.00015942 |
| 15 | 50782416 | 50782446 | USP8 | 0.000159535 |
| 5 | 145888687 | 145888714 | TCERG1 | 0.000160126 |
| X | 48156078 | 48156097 | SSX1(dist = 29199), SSX9(dist = 4888) | 0.000160216 |
| 11 | 104869775 | 104869786 | CASP5 | 0.000160312 |
| 5 | 41862862 | 41862875 | OXCT1 | 0.000160345 |
| 17 | 65919172 | 65919190 | BPTF | 0.00016091 |
| 17 | 29192700 | 29192715 | ATAD5 | 0.000161778 |
| 5 | 137506862 | 137506874 | BRD8 | 0.000164042 |
| 1 | 28218773 | 28218784 | RPA2 | 0.000164065 |
| 17 | 16137244 | 16137257 | PIGL | 0.000164626 |
| 16 | 31284618 | 31284634 | ITGAM | 0.000164629 |
| 2 | 100035414 | 100035425 | REV1 | 0.000164645 |
| 6 | 56504477 | 56504489 | RNU6-71P | 0.000165004 |
| 7 | 44663890 | 44663910 | OGDH | 0.000165068 |
| 14 | 50050396 | 50050408 | RPS29 | 0.000165132 |
| 20 | 43623620 | 43623658 | STK4 | 0.000167071 |
| 15 | 65847112 | 65847125 | PTPLAD1 | 0.000168471 |
| 17 | 2323953 | 2323979 | METTL16 | 0.000169004 |
| 12 | 31820564 | 31820578 | METTL20 | 0.000169079 |
| 17 | 62855003 | 62855017 | LRRC37A3 | 0.000169397 |
| 11 | 5687320 | 5687338 | TRIM5 | 0.000171126 |
| 7 | 54819993 | 54820005 | SEC61G(NM_014302: c.*111_*99del0, | 0.000171145 |
| | | | NM_001012456: c.*111_*99del0) | |
| 5 | 150856066 | 150856091 | SLC36A1 | 0.000176203 |
| 7 | 135098181 | 135098192 | CNOT4 | 0.000176239 |
| 5 | 72875091 | 72875102 | UTP15 | 0.000179126 |
| 19 | 16591361 | 16591374 | CALR3 | 0.000179261 |
| 17 | 10543281 | 10543296 | MYH3 | 0.000180269 |
| 10 | 74653468 | 74653479 | OIT3(NM_152635: c.-89_-78del-) | 0.000182549 |
| 6 | 138192324 | 138192336 | TNFAIP3 | 0.000185237 |
| 2 | 119748135 | 119748152 | MARCO | 0.000188321 |
| 1 | 207753824 | 207753846 | CR1 | 0.000190019 |
| 14 | 92549604 | 92549623 | ATXN3 | 0.000191433 |
| 3 | 137981439 | 137981456 | ARMC8, NME9 | 0.000192654 |
| 5 | 112346416 | 112346447 | DCP2 | 0.000192739 |
| 1 | 176050430 | 176050447 | RFWD2 | 0.000193515 |
| 18 | 9954233 | 9954247 | VAPA(NM_003574: c.*25_*39del0, | 0.000193536 |
| | | | NM_194434: c.*25_*39del0) | |
| 5 | 177676986 | 177677011 | COL23A1 | 0.000193663 |
| 12 | 46758392 | 46758417 | SLC38A2 | 0.000194061 |
| 15 | 41364265 | 41364305 | INO80 | 0.000196303 |
| 17 | 27816599 | 27816619 | TAOK1 | 0.000196406 |
| 21 | 34655334 | 34655346 | IL10RB | 0.000196457 |
| 12 | 110006724 | 110006738 | MMAB | 0.000197329 |
| 4 | 8176744 | 8176762 | ABLIM2(dist = 16185), SH3TC1(dist = 24298) | 0.000198718 |
| 6 | 84649715 | 84649747 | CYB5R4 | 0.000199255 |
| 5 | 180481216 | 180481233 | BTNL9 | 0.000201144 |
| 3 | 178937630 | 178937642 | PIK3CA | 0.000201385 |
| 4 | 39233719 | 39233734 | MIR1273H | 0.000201789 |
| 4 | 76528928 | 76528945 | CDKL2 | 0.000204475 |
| 20 | 46266543 | 46266558 | NCOA3 | 0.000206036 |
| 6 | 138628598 | 138628611 | KIAA1244 | 0.00020697 |
| 1 | 22030115 | 22030132 | USP48 | 0.000209782 |
| 2 | 55874657 | 55874673 | PNPT1 | 0.000210064 |
| 11 | 102272460 | 102272474 | TMEM123 | 0.000211224 |

TABLE 14B
Microsatellite Repeat Marker Sequences for Breast Invasive Carcinoma (BRCA),
top 501-800.
TABLE 14B - BRCA (top 501-800)

| ch | start_pos | end_pos | | BRCA |
|---|---|---|---|---|
| (hg37) | (hg37) | (hg37) | gene | p-value |
| 7 | 6064470 | 6064488 | EIF2AK1 | 0.000215139 |
| 1 | 109525421 | 109525434 | WDR47 | 0.00021611 |
| 3 | 191888452 | 191888477 | FGF12 | 0.000216675 |
| 1 | 144923886 | 144923908 | NBPF20, NBPF9, PDE4DIP | 0.000217108 |
| 11 | 73753268 | 73753288 | C2CD3 | 0.000218485 |
| 6 | 30573637 | 30573656 | PPP1R10 | 0.000220796 |
| 21 | 32597972 | 32597988 | TIAM1 | 0.000221172 |
| 1 | 197150249 | 197150268 | ZBTB41 | 0.00022148 |
| 7 | 63454255 | 63454269 | MIR4283-2(dist = 372708), | 0.000223592 |
| | | | LINC01005(dist = 30527) | |
| 13 | 73293238 | 73293264 | MZT1 | 0.00022874 |
| 13 | 98634737 | 98634749 | IPO5 | 0.000230862 |
| 12 | 112308870 | 112308881 | MAPKAPK5 | 0.000231363 |
| 9 | 98009613 | 98009626 | FANCC | 0.000231592 |
| 10 | 7747034 | 7747047 | ITIH2 | 0.000234104 |
| 3 | 108081175 | 108081187 | HHLA2 | 0.000234278 |
| 18 | 180211 | 180228 | USP14 | 0.000236119 |
| 7 | 77221481 | 77221508 | PTPN12 | 0.000236394 |
| 7 | 87516629 | 87516640 | DBF4 | 0.000236508 |
| 10 | 99197102 | 99197124 | EXOSC1 | 0.000238254 |
| 2 | 206630073 | 206630104 | NRP2 | 0.000238549 |
| 7 | 104707158 | 104707171 | KMT2E | 0.000238915 |
| 7 | 89982116 | 89982127 | GTPBP10 | 0.000240015 |
| 8 | 101725020 | 101725049 | PABPC1 | 0.00024033 |
| 9 | 78638564 | 78638576 | PCSK5 | 0.000240635 |
| 9 | 71538318 | 71538330 | PIP5K1B | 0.000240872 |
| 19 | 56329186 | 56329204 | NLRP11 | 0.000241089 |
| 1 | 86435957 | 86435971 | COL24A1 | 0.000241824 |
| 14 | 75497428 | 75497440 | MLH3 | 0.000243816 |
| 12 | 19410474 | 19410488 | PLEKHA5 | 0.000244258 |
| 5 | 36629801 | 36629815 | SLC1A3 | 0.000245173 |
| 12 | 48360947 | 48360961 | TMEM106C | 0.000245589 |
| 2 | 189926219 | 189926240 | COL5A2 | 0.000245777 |
| 5 | 65088487 | 65088503 | NLN | 0.000246233 |
| 6 | 121412191 | 121412203 | TBC1D32 | 0.000246816 |
| 6 | 148761410 | 148761421 | SASH1 | 0.000248157 |
| 1 | 245021291 | 245021305 | HNRNPU | 0.000248446 |
| X | 1467519 | 1467589 | IL3RA | 0.000249364 |
| 5 | 118451836 | 118451847 | DMXL1 | 0.000253336 |
| 19 | 34951356 | 34951371 | UBA2 | 0.000255632 |
| 14 | 45716018 | 45716030 | MIS18BP1 | 0.000255938 |
| 7 | 74608740 | 74608754 | GTF2IP1, LOC100093631 | 0.000256607 |
| 11 | 73429947 | 73429972 | RAB6A | 0.000259703 |
| 16 | 31435918 | 31435935 | ITGAD | 0.00026029 |
| 5 | 134305852 | 134305866 | CATSPER3 | 0.000261255 |
| 4 | 146031236 | 146031253 | ABCE1 | 0.000263404 |
| 6 | 7410735 | 7410747 | RIOK1 | 0.000266156 |
| 4 | 44691795 | 44691810 | GUF1 | 0.000266198 |
| 11 | 107518175 | 107518187 | ELMOD1 | 0.000266907 |
| 2 | 114691775 | 114691788 | ACTR3 | 0.000267567 |
| 1 | 100624000 | 100624012 | LRRC39 | 0.000268684 |
| 12 | 77421978 | 77421991 | E2F7 | 0.000270478 |
| 12 | 31289662 | 31289674 | DDX11(dist = 31937), | 0.00027167 |
| | | | FAM60A(dist = 143846) | |
| 2 | 112625555 | 112625566 | ANAPC1 | 0.000273396 |
| 19 | 10288056 | 10288083 | DNMT1 | 0.0002743 |
| 3 | 33725814 | 33725828 | CLASP2 | 0.000277579 |
| 1 | 70904523 | 70904552 | CTH | 0.000278482 |
| 7 | 23239063 | 23239074 | NUPL2 | 0.000285563 |
| 3 | 179481971 | 179482004 | USP13 | 0.000285752 |
| 19 | 12543765 | 12543777 | ZNF443 | 0.00028884 |
| 5 | 33561022 | 33561043 | ADAMTS12 | 0.000290387 |
| 19 | 33406199 | 33406229 | CEP89 | 0.000293182 |
| 3 | 180679235 | 180679250 | FXR1 | 0.000294248 |
| 7 | 95864044 | 95864057 | SLC25A13 | 0.000295824 |
| 19 | 18453807 | 18453828 | PGPEP1 | 0.000296643 |
| 6 | 37328387 | 37328414 | RNF8 | 0.000300188 |
| 3 | 138248009 | 138248026 | CEP70 | 0.000300509 |
| 19 | 21116763 | 21116804 | ZNF85 | 0.000301771 |
| 5 | 98224945 | 98224957 | CHD1 | 0.000304934 |
| 9 | 73479430 | 73479465 | TRPM3 | 0.000305947 |
| 12 | 89995053 | 89995065 | ATP2B1 | 0.000306087 |
| 2 | 238005446 | 238005457 | COPS8 | 0.000307607 |
| 4 | 107037269 | 107037282 | TBCK | 0.000308459 |
| 1 | 117605130 | 117605145 | TTF2 | 0.000310064 |
| 10 | 26825151 | 26825168 | APBB1IP | 0.000310141 |
| 8 | 63948170 | 63948202 | GGH | 0.000310478 |
| 14 | 71517327 | 71517341 | PCNX | 0.000310486 |
| 3 | 121515940 | 121515953 | IQCB1 | 0.00031238 |
| X | 67741418 | 67741434 | YIPF6 | 0.000321373 |
| 2 | 186689258 | 186689269 | FSIP2 | 0.000322176 |
| 3 | 93768420 | 93768439 | ARL13B | 0.000323021 |
| 6 | 90503754 | 90503779 | MDN1 | 0.000324941 |
| 15 | 76205523 | 76205536 | FBXO22 | 0.000325112 |
| 2 | 118860645 | 118860670 | INSIG2 | 0.000325658 |
| 6 | 135286438 | 135286451 | HBS1L | 0.000326898 |
| 19 | 7119636 | 7119690 | INSR | 0.000331431 |
| 2 | 111721142 | 111721186 | ACOXL | 0.000332494 |
| 12 | 105540322 | 105540335 | KIAA1033 | 0.000333725 |
| 6 | 139598061 | 139598085 | TXLNB | 0.000334997 |
| 14 | 20857306 | 20857321 | TEP1 | 0.000335308 |
| 2 | 228159652 | 228159677 | LOC654841 | 0.000337791 |
| 3 | 100992404 | 100992415 | IMPG2 | 0.00033855 |
| 21 | 33979904 | 33979922 | C21orf59 | 0.000339462 |
| 14 | 35899480 | 35899493 | NFKBIA(dist = 25520), | 0.000340538 |
| | | | INSM2(dist = 103755) | |
| 5 | 139907750 | 139907762 | ANKHD1, ANKHD1-EIF4EBP3 | 0.000340919 |
| 19 | 22585521 | 22585535 | ZNF98 | 0.000341515 |
| 14 | 81574687 | 81574712 | TSHR | 0.000341771 |
| X | 110494528 | 110494543 | CAPN6 | 0.000343324 |
| 13 | 49684436 | 49684450 | FNDC3A | 0.000343963 |
| 4 | 123374811 | 123374832 | IL2 | 0.000345147 |
| 1 | 154178185 | 154178198 | C1orf189 | 0.000345147 |
| 9 | 77457028 | 77457044 | TRPM6 | 0.000347826 |
| 20 | 48562797 | 48562813 | RNF114 | 0.000349748 |
| 2 | 62063093 | 62063111 | FAM161A | 0.000350662 |
| 2 | 66796098 | 66796109 | MEIS1 | 0.000353069 |
| 2 | 28464369 | 28464382 | BRE | 0.00035345 |
| 19 | 10270725 | 10270758 | DNMT1 | 0.000353765 |
| 9 | 86354663 | 86354679 | GKAP1 | 0.00035517 |
| 6 | 11192516 | 11192554 | NEDD9 | 0.000355793 |
| 12 | 21639528 | 21639542 | RECQL | 0.000357453 |
| 5 | 137451460 | 137451472 | NME5 | 0.000358028 |
| 21 | 33068865 | 33068877 | SCAF4 | 0.000360163 |
| 20 | 47770443 | 47770478 | STAU1 | 0.000360337 |
| 20 | 37126168 | 37126180 | RALGAPB | 0.000361914 |
| 1 | 181714466 | 181714481 | CACNA1E | 0.000363372 |
| 3 | 111873902 | 111873917 | SLC9C1 | 0.000368608 |
| 7 | 87056260 | 87056297 | ABCB4 | 0.000369293 |
| 15 | 73567127 | 73567163 | NEO1 | 0.000369773 |
| 1 | 14057425 | 14057439 | PRDM2 | 0.000370053 |
| 6 | 10989873 | 10989887 | ELOVL2 | 0.00037325 |
| 15 | 49064607 | 49064620 | CEP152 | 0.000373471 |
| 8 | 22138812 | 22138831 | PIWIL2 | 0.000374497 |
| 14 | 75476004 | 75476015 | EIF2B2 | 0.00037452 |
| 8 | 67759411 | 67759438 | C8orf44-SGK3, SGK3 | 0.000375342 |
| 12 | 101598350 | 101598362 | SLC5A8 | 0.000378825 |
| 3 | 132166148 | 132166162 | DNAJC13 | 0.000382559 |
| 12 | 132539500 | 132539513 | EP400 | 0.000383983 |
| 1 | 94230106 | 94230131 | ANKRD49 | 0.000385032 |
| 15 | 70963344 | 70963368 | UACA | 0.000385269 |
| 3 | 171455451 | 171455462 | PLD1 | 0.000385719 |
| 19 | 36572251 | 36572263 | WDR62 | 0.000386586 |
| 2 | 192011485 | 192011497 | STAT4 | 0.00038737 |
| 3 | 119348345 | 119348362 | PLA1A | 0.000387842 |
| X | 153357614 | 153357628 | MECP2 | 0.000393618 |
| 16 | 19619602 | 19619617 | C16orf62 | 0.00039685 |
| 22 | 29682873 | 29682894 | EWSR1 | 0.000397816 |
| 3 | 65361369 | 65361402 | MAGI1 | 0.000399074 |
| 6 | 32123361 | 32123410 | PPT2-EGFL8 | 0.000402145 |
| 3 | 124998098 | 124998118 | ZNF148 | 0.000403189 |
| 1 | 109856836 | 109856854 | SORT1 | 0.000407145 |
| 15 | 51768755 | 51768768 | DMXL2 | 0.000407952 |
| 2 | 190321967 | 190321989 | WDR75 | 0.000412862 |
| 1 | 225607366 | 225607387 | LBR | 0.000413342 |
| 13 | 38171301 | 38171312 | POSTN | 0.000413642 |
| 19 | 7995974 | 7996007 | TIMM44 | 0.000414721 |
| 10 | 12226870 | 12226882 | NUDT5 | 0.000415483 |
| 6 | 109480681 | 109480693 | CEP57L1 | 0.000416475 |
| X | 135328309 | 135328320 | MAP7D3 | 0.000417679 |
| 5 | 137513369 | 137513382 | BRD8 | 0.000417833 |
| 3 | 33893929 | 33893949 | PDCD6IP | 0.000423268 |
| 1 | 237060945 | 237060970 | MTR | 0.000427426 |
| 3 | 10429902 | 10429913 | ATP2B2 | 0.000428436 |
| 1 | 92944314 | 92944351 | GFI1 | 0.000430436 |
| 6 | 131948469 | 131948501 | MED23 | 0.000431417 |
| 3 | 32933344 | 32933365 | TRIM71 | 0.000431546 |
| 14 | 64460536 | 64460551 | SYNE2 | 0.000432367 |
| 7 | 73279233 | 73279260 | WBSCR28 | 0.000432896 |
| 2 | 191941054 | 191941078 | STAT4 | 0.000433708 |
| 4 | 151835502 | 151835539 | LRBA | 0.000440614 |
| 3 | 194159705 | 194159736 | ATP13A3 | 0.000442086 |
| 5 | 60982634 | 60982645 | LOC101928651 | 0.000444366 |
| 1 | 207679180 | 207679198 | CR1 | 0.00044455 |
| 12 | 21639385 | 21639398 | RECQL | 0.000446918 |
| 16 | 20803249 | 20803265 | ACSM3, ERI2 | 0.000450863 |
| 5 | 115628120 | 115628134 | COMMD10 | 0.00045249 |
| 1 | 33087567 | 33087584 | ZBTB8OS | 0.000455645 |
| 11 | 65647789 | 65647810 | CTSW | 0.000455868 |
| 15 | 52239617 | 52239629 | LEO1 | 0.000459931 |
| 7 | 36465606 | 36465622 | ANLN | 0.000460705 |
| 15 | 41770639 | 41770652 | RTF1 | 0.000460705 |
| 16 | 90141288 | 90141302 | PRDM7 | 0.000465942 |
| 7 | 150918586 | 150918602 | ABCF2 | 0.00046677 |
| 5 | 126866119 | 126866130 | PRRC1 | 0.000467188 |
| 14 | 53513439 | 53513452 | DDHD1 | 0.000478649 |
| 14 | 93712634 | 93712660 | BTBD7 | 0.000479866 |
| 10 | 69716602 | 69716614 | HERC4 | 0.000481752 |
| 8 | 21846488 | 21846505 | XPO7 | 0.000484079 |
| 20 | 35632021 | 35632042 | RBL1 | 0.000488807 |
| 11 | 118883880 | 118883892 | CCDC84 | 0.000490191 |
| 17 | 27829588 | 27829602 | TAOK1 | 0.000493431 |
| 19 | 54264352 | 54264364 | MIR516A2 | 0.000495727 |
| 18 | 31523144 | 31523156 | NOL4 | 0.000496542 |
| 4 | 10027509 | 10027520 | SLC2A9 | 0.000497979 |
| 1 | 215345245 | 215345313 | KCNK2 | 0.000499439 |
| 6 | 144758647 | 144758660 | UTRN | 0.000499476 |
| 4 | 57686807 | 57686822 | SPINK2 | 0.000503914 |
| 2 | 102785158 | 102785171 | IL1R1 | 0.000511534 |
| 2 | 32427092 | 32427104 | SLC30A6 | 0.000513291 |
| 4 | 169190129 | 169190143 | DDX60 | 0.000514018 |
| X | 135053121 | 135053154 | MMGT1 | 0.000515531 |
| 10 | 123310733 | 123310744 | FGFR2 | 0.000518238 |
| 6 | 38905667 | 38905680 | LOC100131047 | 0.000519154 |
| 18 | 43445730 | 43445748 | EPG5 | 0.000521147 |
| 2 | 47635523 | 47635537 | MSH2 | 0.000521166 |
| 1 | 33799658 | 33799669 | PHC2 | 0.000522835 |
| 7 | 92883250 | 92883261 | CCDC132 | 0.000524287 |
| 6 | 47567210 | 47567222 | CD2AP | 0.000524605 |
| 12 | 10205420 | 10205435 | CLEC9A | 0.000526676 |
| X | 44949951 | 44949963 | KDM6A | 0.000529903 |
| 15 | 30406088 | 30406103 | ULK4P3 | 0.000533847 |
| 1 | 150676674 | 150676692 | HORMAD1 | 0.000535678 |
| 1 | 74575199 | 74575231 | LRRIQ3 | 0.000536674 |
| 10 | 114154662 | 114154673 | ACSL5 | 0.000540155 |
| X | 83403148 | 83403160 | RPS6KA6 | 0.00054065 |
| 5 | 140812775 | 140812788 | PCDHGA12 | 0.000540991 |
| 19 | 55444906 | 55444921 | NLRP7 | 0.00054188 |
| 10 | 12240681 | 12240702 | CDC123 | 0.000541893 |
| 21 | 38498341 | 38498356 | TTC3 | 0.000541948 |
| 15 | 63040516 | 63040533 | TLN2 | 0.000545158 |
| 1 | 210411266 | 210411281 | SERTAD4 | 0.000545366 |
| 9 | 102730697 | 102730712 | STX17 | 0.00055051 |
| 7 | 106888946 | 106888972 | COG5 | 0.000550939 |
| 3 | 113377481 | 113377493 | KIAA2018 | 0.000553112 |
| 7 | 135099044 | 135099059 | CNOT4 | 0.000553432 |
| 1 | 109773427 | 109773451 | SARS | 0.000553805 |
| 9 | 130413851 | 130413880 | STXBP1 | 0.00055571 |
| 11 | 35287000 | 35287014 | SLC1A2 | 0.000560024 |
| 2 | 228680153 | 228680168 | CCL20 | 0.000564239 |
| 1 | 154112163 | 154112180 | NUP210L | 0.00056524 |
| 6 | 110426583 | 110426595 | WASF1 | 0.000567544 |
| 11 | 124624705 | 124624721 | ESAM | 0.000571783 |
| 15 | 86279430 | 86279457 | AKAP13 | 0.000573655 |
| 6 | 123545159 | 123545186 | TRDN | 0.000585494 |
| 12 | 30822226 | 30822240 | IPO8 | 0.000587147 |
| 15 | 23333801 | 23333815 | HERC2P2 | 0.000588027 |
| 1 | 32631598 | 32631676 | KPNA6 | 0.000589911 |
| 11 | 22296094 | 22296106 | ANO5 | 0.000591354 |
| 9 | 84267021 | 84267054 | TLE1 | 0.000592855 |
| 6 | 119501609 | 119501620 | MAN1A1 | 0.000594016 |
| 11 | 47840864 | 47840879 | NUP160 | 0.000594717 |
| 4 | 167020771 | 167020795 | TLL1 | 0.000597441 |
| 12 | 13364426 | 13364437 | EMP1 | 0.000597661 |
| 12 | 56744020 | 56744045 | STAT2 | 0.000599413 |
| X | 24745018 | 24745047 | POLA1 | 0.000600192 |
| 6 | 24412431 | 24412445 | MRS2 | 0.000608675 |
| 3 | 186522293 | 186522308 | RFC4 | 0.000609401 |
| 2 | 120712718 | 120712731 | PTPN4 | 0.000609401 |
| 12 | 27841925 | 27841938 | PPFIBP1 | 0.000609509 |
| 12 | 27877126 | 27877148 | MRPS35 | 0.000610049 |
| 2 | 91887905 | 91887919 | LOC654342(dist = 39930), | 0.000610207 |
| | | | GGT8P(dist = 75449) | |
| 12 | 99059316 | 99059327 | APAF1 | 0.000610207 |
| 12 | 45771901 | 45771918 | ANO6 | 0.000610833 |
| X | 22244528 | 22244549 | PHEX | 0.000615633 |
| 20 | 23473522 | 23473535 | CST8 | 0.000622379 |
| 2 | 61187478 | 61187496 | PUS10 | 0.000635196 |
| 19 | 12128923 | 12128937 | ZNF433 | 0.000635849 |
| 7 | 151884279 | 151884292 | KMT2C | 0.000636802 |
| 22 | 30051705 | 30051722 | NF2 | 0.000637223 |
| 15 | 73418741 | 73418756 | NEO1 | 0.000638666 |
| 6 | 90366329 | 90366347 | LOC101929057 | 0.000645585 |
| 5 | 70848879 | 70848890 | BDP1 | 0.000648316 |
| 1 | 155884155 | 155884167 | KIAA0907 | 0.000648654 |
| 3 | 107910327 | 107910342 | IFT57 | 0.000649415 |
| 8 | 17422434 | 17422446 | SLC7A2 | 0.000651271 |
| 1 | 98011812 | 98011842 | DPYD | 0.000662468 |
| 2 | 61577667 | 61577680 | USP34 | 0.000663347 |
| 4 | 119666217 | 119666241 | SEC24D | 0.000663806 |
| 2 | 48033875 | 48033909 | MSH6 | 0.000668448 |
| 2 | 169994021 | 169994058 | LRP2 | 0.000671582 |
| 1 | 234565078 | 234565090 | TARBP1 | 0.00067177 |
| 22 | 32548036 | 32548047 | C22orf42 | 0.000671841 |
| 2 | 179442292 | 179442314 | MIR548N, TTN-AS1 | 0.000671892 |
| 2 | 152589615 | 152589626 | NEB | 0.000675485 |
| 16 | 3828847 | 3828870 | CREBBP | 0.000679186 |
| 12 | 56380950 | 56380964 | RAB5B | 0.000681492 |
| X | 19086800 | 19086823 | GPR64 | 0.000681492 |
| 2 | 157370018 | 157370033 | GPD2 | 0.000681993 |
| 12 | 131476665 | 131476692 | GPR133 | 0.00068284 |
| 6 | 18258299 | 18258311 | DEK | 0.000693505 |
| 1 | 144916415 | 144916426 | NBPF20, NBPF9, PDE4DIP | 0.000695925 |
| 4 | 25160732 | 25160748 | SEPSECS | 0.000697507 |
| 15 | 50152754 | 50152776 | ATP8B4 | 0.000698812 |
| 2 | 101581294 | 101581308 | NPAS2 | 0.000704355 |
| 1 | 52403174 | 52403187 | RAB3B | 0.000704639 |
| 6 | 158049351 | 158049365 | ZDHHC14 | 0.000707336 |
| 4 | 41015553 | 41015566 | APBB2 | 0.000708852 |
| 7 | 6771663 | 6771684 | ZNF12(dist = 25097), | 0.00071122 |
| | | | PMS2CL(dist = 3252) | |
| 14 | 76173952 | 76173964 | TTLL5 | 0.000712406 |
| 11 | 125763610 | 125763624 | PUS3 | 0.000716302 |
| 1 | 231094050 | 231094067 | TTC13 | 0.000718986 |
| 3 | 190338028 | 190338060 | IL1RAP | 0.000720894 |
| 2 | 191155233 | 191155245 | HIBCH | 0.000730721 |
| 3 | 143235916 | 143235929 | SLC9A9 | 0.000732601 |
| 7 | 77797468 | 77797490 | MAGI2 | 0.000732859 |
| 1 | 183498191 | 183498202 | SMG7 | 0.000735791 |
| 19 | 21428062 | 21428087 | ZNF431(dist = 59257), | 0.000736402 |
| | | | ZNF708(dist = 45875) | |
| 17 | 7726648 | 7726698 | DNAH2 | 0.000744655 |
| 1 | 9192336 | 9192365 | DENND5A | 0.00075596 |
| 2 | 68772281 | 68772292 | APLF | 0.000757479 |
| 6 | 88221097 | 88221109 | SLC35A1 | 0.000761518 |
| 16 | 15162143 | 15162165 | PDXDC1, RRN3 | 0.000766289 |
| 21 | 37706087 | 37706100 | MORC3 | 0.000768534 |
| 5 | 108516618 | 108516640 | FER | 0.000772105 |
| 17 | 29563068 | 29563089 | NF1 | 0.0007733 |
| 18 | 33058318 | 33058335 | INO80C | 0.000776871 |
| 11 | 132179950 | 132179979 | NTM | 0.00077799 |
| 17 | 35988718 | 35988739 | DDX52 | 0.000780376 |
| 13 | 37596114 | 37596125 | SUPT20H | 0.000781111 |
| 8 | 124244005 | 124244016 | C8orf76, ZHX1-C8orf76 | 0.000784314 |
| 11 | 121367557 | 121367571 | SORL1 | 0.000787353 |
| X | 15364158 | 15364186 | PIR-FIGF | 0.000787738 |

TABLE 15
Microsatellite Repeat Marker Sequences for Ovarian Serous Cystadenocarcinoma (OV).
TABLE 15 - OV

| ch | start_pos | end_pos | | |
|---|---|---|---|---|
| (hg37) | (hg37) | (hg37) | gene | OV p-value |
| 7 | 23761159 | 23761171 | STK31 | 0.000138358 |
| 3 | 9102112 | 9102129 | SRGAP3 | 0.000154596 |
| 17 | 52991026 | 52991047 | TOM1L1 | 0.000164836 |
| X | 135115669 | 135115681 | SLC9A6 | 0.000306057 |
| 8 | 100287517 | 100287536 | VPS13B | 0.000388149 |
| 1 | 74818936 | 74818947 | FPGT-TNNI3K, TNNI3K | 0.000510827 |
| 12 | 64891393 | 64891418 | TBK1 | 0.000580495 |
| 10 | 88458996 | 88459025 | LDB3 | 0.00079249 |
| 7 | 104309164 | 104309177 | LHFPL3 | 0.001077249 |
| 1 | 206566852 | 206566872 | SRGAP2D | 0.00112585 |
| X | 131231430 | 131231468 | FRMD7 | 0.001292105 |
| 15 | 65273147 | 65273161 | SPG21 | 0.0013328 |
| 12 | 124116817 | 124116847 | EIF2B1 | 0.001966955 |
| 1 | 158224559 | 158224624 | CD1A | 0.002282105 |
| 15 | 34654339 | 34654358 | LPCAT4 | 0.002298213 |
| 20 | 42199704 | 42199743 | SGK2 | 0.002363852 |
| 1 | 176105710 | 176105723 | RFWD2 | 0.002407981 |
| 6 | 49415351 | 49415368 | MUT | 0.002510486 |
| 11 | 14063262 | 14063275 | SPON1 | 0.002583661 |
| 18 | 6975847 | 6975857 | LAMA1 | 0.002633889 |
| 1 | 79383748 | 79383762 | ELTD1 | 0.002877698 |
| 8 | 7191539 | 7191558 | FAM66B | 0.002912056 |
| 9 | 128064242 | 128064260 | GAPVD1 | 0.002939735 |
| 12 | 109701959 | 109701975 | ACACB | 0.003011318 |
| 12 | 49091972 | 49092009 | CCNT1 | 0.00329608 |
| 1 | 46650191 | 46650201 | TSPAN1 | 0.003361142 |
| 17 | 18708830 | 18708843 | TVP23B | 0.003661465 |
| 17 | 61898129 | 61898142 | FTSJ3 | 0.003830439 |
| 6 | 111702448 | 111702460 | REV3L | 0.004010111 |
| 2 | 131888361 | 131888376 | PLEKHB2 | 0.004016064 |
| 10 | 64022582 | 64022593 | RTKN2 | 0.004262575 |
| 8 | 65528842 | 65528854 | CYP7B1 | 0.004516712 |
| 1 | 113657018 | 113657033 | LRIG2 | 0.004645161 |
| 6 | 7413094 | 7413107 | RIOK1 | 0.004856512 |
| 7 | 91509436 | 91509457 | MTERF1 | 0.004897959 |
| 1 | 173795803 | 173795821 | DARS2 | 0.005042017 |
| 1 | 207753824 | 207753846 | CR1 | 0.005128205 |
| 12 | 16052767 | 16052780 | STRAP | 0.005366726 |
| 12 | 107398862 | 107398876 | CRY1 | 0.005367724 |
| 5 | 94842718 | 94842730 | TTC37 | 0.005589194 |
| 1 | 220369745 | 220369758 | RAB3GAP2 | 0.005664965 |
| 1 | 201832479 | 201832492 | IPO9 | 0.005734543 |
| X | 21534510 | 21534521 | CNKSR2 | 0.005797101 |
| 3 | 107910327 | 107910342 | IFT57 | 0.005865422 |
| 2 | 87049358 | 87049371 | CD8B | 0.005961252 |
| 15 | 35083508 | 35083555 | LOC101928174 | 0.006058371 |
| X | 122598635 | 122598648 | GRIA3 | 0.006143242 |
| 2 | 21226218 | 21226230 | APOB | 0.006265664 |
| 2 | 136690414 | 136690443 | DARS | 0.006303735 |
| 7 | 33075634 | 33075645 | NT5C3A | 0.006342495 |
| 12 | 55354876 | 55354889 | TESPA1 | 0.006518905 |
| 16 | 15501658 | 15501670 | MPV17L | 0.00669203 |
| 1 | 58380192 | 58380205 | ZFP91-CNTF | 0.006714213 |
| 16 | 81051115 | 81051128 | CENPN | 0.006858499 |
| 1 | 211280724 | 211280748 | KCNH1 | 0.007211241 |
| 14 | 24571860 | 24571874 | PCK2 | 0.007510446 |
| 2 | 236706612 | 236706623 | AGAP1 | 0.007519945 |
| 19 | 22585521 | 22585535 | ZNF98 | 0.007611165 |
| X | 21544904 | 21544935 | CNKSR2 | 0.007692308 |
| 1 | 175087683 | 175087694 | TNN | 0.007747087 |
| 22 | 42415224 | 42415240 | WBP2NL | 0.007784946 |
| 11 | 59368218 | 59368235 | OSBP | 0.00784744 |
| 4 | 17964528 | 17964539 | LCORL | 0.008097166 |
| 5 | 68413042 | 68413072 | SLC30A5 | 0.008206581 |
| 5 | 68396764 | 68396793 | SLC30A5 | 0.008764684 |
| 7 | 33192288 | 33192300 | BBS9 | 0.008766803 |
| 19 | 6026288 | 6026302 | RFX2 | 0.008885851 |
| X | 1467519 | 1467589 | IL3RA | 0.009114066 |
| 11 | 58892376 | 58892387 | FAM111B | 0.009324009 |
| 13 | 20325367 | 20325378 | PSPC1 | 0.009398496 |
| 2 | 103281540 | 103281552 | SLC9A2 | 0.009484187 |
| 19 | 56329186 | 56329204 | NLRP11 | 0.00966544 |
| 6 | 84649715 | 84649747 | CYB5R4 | 0.00970483 |
| 14 | 103192774 | 103192791 | RCOR1 | 0.009886234 |
| 14 | 51477220 | 51477231 | TRIM9 | 0.01 |
| 17 | 35567303 | 35567314 | ACACA | 0.010486231 |
| 17 | 54981571 | 54981588 | TRIM25 | 0.010512345 |
| 5 | 50122645 | 50122660 | PARP8 | 0.01072434 |
| 20 | 18505555 | 18505576 | SEC23B | 0.01072434 |
| 17 | 53014442 | 53014457 | TOM1L1 | 0.010755482 |
| 14 | 81574687 | 81574712 | TSHR | 0.010977266 |
| 13 | 50586051 | 50586064 | DLEU2 | 0.011144436 |
| 15 | 86188984 | 86189019 | AKAP13 | 0.011177158 |
| 14 | 52981552 | 52981563 | TXNDC16 | 0.011310323 |
| 12 | 94818770 | 94818802 | CEP83 | 0.011378114 |
| X | 44949951 | 44949963 | KDM6A | 0.011471034 |
| 10 | 45984725 | 45984740 | MARCH8 | 0.011555078 |
| 8 | 28707622 | 28707636 | INTS9 | 0.011645202 |
| 14 | 21936762 | 21936776 | RAB2B | 0.011656661 |
| 6 | 116914112 | 116914128 | RWDD1 | 0.011724301 |
| X | 2343183 | 2343213 | DHRSX | 0.011904684 |
| 15 | 41350749 | 41350793 | INO80 | 0.01209145 |
| 6 | 152631788 | 152631821 | SYNE1 | 0.012200061 |
| 4 | 113107829 | 113107845 | C4orf32 | 0.012294372 |
| 5 | 61745737 | 61745750 | IPO11 | 0.012337984 |
| 2 | 63182611 | 63182622 | EHBP1 | 0.012479102 |
| 2 | 9552383 | 9552396 | ITGB1BP1 | 0.012575687 |
| 2 | 56611386 | 56611398 | CCDC85A | 0.012663953 |
| 16 | 4873743 | 4873761 | GLYR1 | 0.012691424 |
| 2 | 136511898 | 136511910 | UBXN4 | 0.012720908 |
| 14 | 50605267 | 50605280 | SOS2 | 0.013151028 |
| 12 | 95488339 | 95488354 | FGD6 | 0.013547775 |
| 6 | 105405900 | 105405913 | LIN28B | 0.013888889 |
| 2 | 211457713 | 211457724 | CPS1 | 0.013888889 |
| 9 | 86920104 | 86920135 | SLC28A3 | 0.014739765 |
| 8 | 95507237 | 95507250 | KIAA1429 | 0.014787155 |
| 7 | 106888946 | 106888972 | COG5 | 0.01509952 |
| 7 | 102825987 | 102826001 | DPY19L2P2 | 0.015242366 |
| 1 | 29476535 | 29476555 | SRSF4 | 0.015625 |
| 12 | 56744020 | 56744045 | STAT2 | 0.015961596 |
| 7 | 152144203 | 152144216 | FABP5P3(dist = 4103), LINC01003(dist = 16993) | 0.016069586 |
| 15 | 50581627 | 50581647 | GABPB1 | 0.016129032 |
| 2 | 119739127 | 119739142 | MARCO | 0.016278948 |
| 2 | 43778857 | 43778869 | THADA | 0.016574113 |
| 6 | 80837242 | 80837253 | BCKDHB | 0.016579523 |
| 5 | 41909830 | 41909846 | 5orf5 | 0.016637417 |
| 5 | 33546113 | 33546143 | ADAMTS12 | 0.016947811 |
| 13 | 77792099 | 77792113 | MYCBP2 | 0.017281606 |
| 9 | 85597720 | 85597749 | RASEF | 0.017337643 |
| 21 | 17238572 | 17238582 | USP25 | 0.017500648 |
| 1 | 38052867 | 38052907 | GNL2 | 0.0178959 |
| 20 | 10622080 | 10622091 | JAG1 | 0.018087462 |
| 7 | 18688019 | 18688056 | HDAC9 | 0.018103117 |
| 3 | 130660392 | 130660405 | ATP2C1 | 0.018365415 |
| 1 | 248524799 | 248524818 | OR2T4 | 0.018731926 |
| 17 | 71223424 | 71223438 | FAM104A | 0.018918799 |
| X | 110494528 | 110494543 | CAPN6 | 0.019645318 |
| 10 | 26825151 | 26825168 | APBB1IP | 0.019718823 |
| 1 | 158584103 | 158584116 | SPTA1 | 0.019822501 |
| 12 | 88926290 | 88926302 | KITLG | 0.019961408 |
| 9 | 117783551 | 117783586 | TNC | 0.020082879 |
| 1 | 36888302 | 36888316 | OSCP1 | 0.020260267 |
| 17 | 4646533 | 4646547 | ZMYND15 | 0.020449216 |
| 7 | 20180507 | 20180526 | MACC1(NM_182762: c.*62_43del0) | 0.020578912 |
| 1 | 47325299 | 47325313 | CYP4Z2P | 0.020804132 |
| 12 | 22063251 | 22063266 | ABCC9 | 0.020909254 |
| 6 | 51910735 | 51910747 | PKHD1 | 0.021012212 |
| 6 | 36483295 | 36483334 | STK38 | 0.021307895 |
| X | 133511862 | 133511890 | PHF6 | 0.02158136 |
| 1 | 169797067 | 169797078 | C1orf112 | 0.02173913 |
| 3 | 124998098 | 124998118 | ZNF148 | 0.022213678 |
| 19 | 20317771 | 20317824 | ZNF486(dist = 6472), MIR1270(dist = 192257) | 0.022222222 |
| 10 | 90362299 | 90362324 | LIPJ | 0.022555223 |
| 15 | 98513086 | 98513103 | ARRDC4 | 0.023165075 |
| 16 | 57249981 | 57250000 | RSPRY1 | 0.023343584 |
| X | 77264514 | 77264533 | ATP7A | 0.023724164 |
| 18 | 56245965 | 56246014 | ALPK2 | 0.023834984 |
| 17 | 31266427 | 31266441 | TMEM98 | 0.024691358 |
| 14 | 93686568 | 93686587 | UBR7 | 0.025385559 |
| 17 | 10317164 | 10317176 | MYHAS | 0.02540136 |
| 5 | 142601811 | 142601862 | ARHGAP26 | 0.025725356 |
| 5 | 83550060 | 83550071 | EDIL3 | 0.0258581 |
| 20 | 480602 | 480627 | CSNK2A1 | 0.026556271 |
| X | 123204978 | 123204993 | STAG2 | 0.02673838 |
| 6 | 33408890 | 33408917 | SYNGAP1 | 0.026771921 |
| 16 | 89576879 | 89576895 | SPG7 | 0.026923077 |
| 11 | 61977858 | 61977881 | SCGB2A1 | 0.027878415 |
| 10 | 17279211 | 17279223 | VIM | 0.028169014 |
| 7 | 102038153 | 102038199 | LOC100630923 | 0.0282742 |
| 19 | 44662274 | 44662288 | ZNF234(NM_006630: c.*2_*16del0, | 0.028565004 |
| | | | NM_001144824: c.*2_*16del0) | |
| 8 | 131193153 | 131193166 | ASAP1 | 0.028604681 |
| 13 | 42772765 | 42772789 | DGKH | 0.029332546 |
| 2 | 215876589 | 215876603 | ABCA12 | 0.029371838 |
| 10 | 134628521 | 134628532 | CFAP46 | 0.029411765 |
| 15 | 44216559 | 44216573 | FRMD5 | 0.029913113 |
| 17 | 33318204 | 33318216 | LIG3 | 0.03030303 |
| 11 | 89395303 | 89395315 | FOLH1B | 0.030808872 |
| 17 | 30293149 | 30293164 | SUZ12 | 0.03125 |
| 1 | 200559220 | 200559231 | KIF14 | 0.03125 |
| 3 | 62153593 | 62153621 | PTPRG | 0.031265558 |
| 9 | 99408079 | 99408094 | AAED1 | 0.031298797 |
| 8 | 24253166 | 24253199 | LOC101929294 | 0.031464833 |
| 1 | 29016705 | 29016737 | GMEB1 | 0.031496063 |
| 6 | 148761410 | 148761421 | SASH1 | 0.031736765 |
| 11 | 125488247 | 125488261 | STT3A | 0.031746032 |
| 1 | 3117272 | 3117311 | IL32 | 0.032126697 |
| 3 | 160141137 | 160141173 | SMC4 | 0.032258065 |
| 2 | 234178624 | 234178640 | ATG16L1 | 0.032487998 |
| 14 | 20666587 | 20666598 | OR11G2 | 0.03249381 |
| 6 | 144814625 | 144814664 | UTRN | 0.032967033 |
| 2 | 47702411 | 47702471 | MSH2 | 0.033057851 |
| 12 | 104515109 | 104515121 | NFYB | 0.033333333 |
| 10 | 32099547 | 32099559 | ARHGAP12 | 0.033421118 |
| 12 | 131476665 | 131476692 | GPR133 | 0.033464212 |
| 5 | 135496820 | 135496834 | SMAD5 | 0.033740307 |
| 1 | 16354441 | 16354448 | CLCNKA | 0.034002981 |
| 2 | 225035745 | 225035758 | SERPINE2(dist = 131709), FAM124B(dist = 207657) | 0.034782609 |
| 2 | 139308383 | 139308420 | SPOPL | 0.034836366 |
| 13 | 32907535 | 32907547 | BRCA2 | 0.034965035 |
| 16 | 47399670 | 47399687 | ITFG1 | 0.035255751 |
| 7 | 92900502 | 92900527 | CCDC132 | 0.035294118 |
| 11 | 14539315 | 14539332 | PSMA1 | 0.03536144 |
| 10 | 43671378 | 43671392 | CSGALNACT2 | 0.03536144 |
| 1 | 66829000 | 66829011 | PDE4B | 0.035714286 |
| 14 | 51062236 | 51062290 | ATL1 | 0.036136806 |
| 8 | 110334770 | 110334782 | NUDCD1 | 0.036363636 |
| 19 | 9771286 | 9771300 | ZNF562 | 0.036641489 |
| 17 | 45992566 | 45992604 | SP2-AS1 | 0.037003666 |
| 17 | 57278908 | 57278920 | PRR11 | 0.037037037 |
| 4 | 143181722 | 143181748 | INPP4B | 0.037037037 |
| 11 | 101362164 | 101362178 | TRPC6 | 0.037096174 |
| 16 | 69385403 | 69385421 | TMED6 | 0.037313433 |
| 4 | 54327211 | 54327223 | LNX1 | 0.037405047 |
| 7 | 26247984 | 26247997 | CBX3 | 0.0375 |
| 6 | 134210497 | 134210516 | TCF21(NM_198392: c.-39_-20del-, | 0.037553034 |
| | | | NM_003206: c.-39_-20del-) | |
| 3 | 38137959 | 38137970 | DLEC1 | 0.037878788 |
| 2 | 39240584 | 39240596 | SOS1 | 0.038167939 |
| 12 | 114837219 | 114837235 | TBX5 | 0.038228009 |
| 1 | 74901739 | 74901760 | FPGT-TNNI3K, TNNI3K | 0.038250334 |
| 2 | 69420574 | 69420634 | ANTXR1 | 0.038461538 |
| 20 | 9360821 | 9360845 | PLCB4 | 0.038461538 |
| 13 | 50098426 | 50098438 | PHF11 | 0.038461538 |
| 9 | 104306988 | 104307005 | RNF20 | 0.038834951 |
| 14 | 60587879 | 60587900 | PCNXL4 | 0.038944472 |
| 3 | 67058329 | 67058357 | KBTBD8 | 0.039215686 |
| 10 | 25273311 | 25273328 | ENKUR | 0.039370079 |
| 12 | 110834030 | 110834049 | ANAPC7 | 0.03968254 |
| 1 | 218578726 | 218578745 | TGFB2 | 0.03979798 |
| 8 | 90933269 | 90933296 | OSGIN2 | 0.04 |
| 1 | 184692999 | 184693020 | EDEM3 | 0.040197897 |
| X | 129265852 | 129265866 | AIFM1 | 0.040268456 |
| 1 | 155631307 | 155631342 | YY1AP1 | 0.040342941 |
| 6 | 117026217 | 117026229 | KPNA5 | 0.04040404 |
| 14 | 45696752 | 45696769 | MIS18BP1 | 0.040677344 |
| 9 | 123945492 | 123945515 | RAB14 | 0.040677344 |
| 19 | 45781081 | 45781156 | MARK4 | 0.040873436 |
| 19 | 55452749 | 55452775 | NLRP7 | 0.041237113 |
| 10 | 6273170 | 6273182 | PFKFB3 | 0.04131008 |
| 22 | 26769464 | 26769477 | SEZ6L | 0.041411937 |
| 2 | 80101515 | 80101528 | CTNNA2 | 0.041428455 |
| 6 | 154489255 | 154489267 | IPCEF1, OPRM1 | 0.041648418 |
| 9 | 30689085 | 30689104 | LINC01242(dist = 280633), ACO1(dist = 1695497) | 0.041666667 |
| 10 | 116605738 | 116605750 | FAM160B1 | 0.041666667 |
| 1 | 28785729 | 28785740 | PHACTR4 | 0.041666667 |
| 5 | 67576330 | 67576346 | PIK3R1 | 0.041666667 |
| 15 | 34553225 | 34553236 | SLC12A6 | 0.041666667 |
| 21 | 44513110 | 44513140 | U2AF1(NM_001025204: c.*102_*72del0, | 0.042016807 |
| | | | NM_006758: c.*102_*72del0, | |
| | | | NM_001025203: c.*102_*72del0) | |
| 12 | 27877126 | 27877148 | MRPS35 | 0.042253521 |
| 1 | 186089102 | 186089113 | MIR548F1 | 0.042253521 |
| 20 | 36431202 | 36431215 | CTNNBL1 | 0.042253521 |
| 7 | 20180425 | 20180476 | MACC1(NM_182762: c.*144_*93del0) | 0.042263434 |
| 16 | 19619602 | 19619617 | C16orf62 | 0.042403642 |
| 5 | 16764255 | 16764277 | MYO10 | 0.042421256 |
| 2 | 64139831 | 64139844 | VPS54 | 0.042553191 |
| 12 | 64491002 | 64491019 | SRGAP1 | 0.042735043 |
| 13 | 73636910 | 73636929 | KLF5 | 0.042778962 |
| 9 | 33062183 | 33062195 | SMU1 | 0.042855001 |
| 4 | 7780663 | 7780674 | AFAP1 | 0.042857143 |
| 8 | 82583104 | 82583133 | IMPA1 | 0.042857143 |
| 4 | 141484371 | 141484383 | UCP1 | 0.042933022 |
| 2 | 170413815 | 170413834 | FASTKD1 | 0.042944785 |
| 10 | 105139888 | 105139903 | TAF5 | 0.043165468 |
| 13 | 43491778 | 43491796 | EPSTI1 | 0.043315982 |
| 12 | 132539500 | 132539513 | EP400 | 0.043478261 |
| 5 | 112903585 | 112903598 | YTHDC2 | 0.043478261 |
| 2 | 84668258 | 84668269 | SUCLG1 | 0.043478261 |
| 9 | 19334957 | 19334970 | DENND4C | 0.044025157 |
| 13 | 21729952 | 21729979 | SKA3 | 0.044040367 |
| 14 | 73079138 | 73079150 | RGS6(dist = 45900), DPF3(dist = 6854) | 0.044247788 |
| 20 | 47268978 | 47268996 | PREX1 | 0.044247788 |
| 15 | 43720155 | 43720172 | RNU6-28P | 0.044405603 |
| 17 | 73515055 | 73515068 | TSEN54 | 0.044585987 |
| X | 119675354 | 119675368 | CUL4B | 0.044585987 |
| 11 | 47187055 | 47187087 | ARFGAP2 | 0.044871795 |
| 1 | 11134470 | 11134485 | EXOSC10 | 0.044871795 |
| 11 | 57472141 | 57472167 | MED19 | 0.045045045 |
| 10 | 93695545 | 93695556 | BTAF1 | 0.045454545 |
| 16 | 31196218 | 31196237 | FUS | 0.045454545 |
| 7 | 95775848 | 95775863 | SLC25A13 | 0.045454545 |
| 15 | 53815527 | 53815550 | WDR72 | 0.045612905 |
| 8 | 89128708 | 89128722 | MMP16 | 0.045893255 |
| 2 | 192225477 | 192225514 | MYO1B | 0.045961358 |
| 1 | 214825289 | 214825312 | CENPF | 0.046052632 |
| 9 | 69127139 | 69127155 | PGM5P2 | 0.046073819 |
| 15 | 55972660 | 55972674 | PRTG | 0.046113395 |
| 20 | 4839890 | 4839911 | SLC23A2 | 0.046357616 |
| 7 | 142139624 | 142139663 | TRY2P(dist = 167556), MTRNR2L6(dist = 234468) | 0.046357616 |
| 7 | 77006092 | 77006113 | GSAP | 0.046511628 |
| 16 | 28842257 | 28842275 | ATXN2L | 0.047169811 |
| 17 | 72285659 | 72285679 | DNAI2 | 0.047463312 |
| 20 | 33225781 | 33225808 | PIGU | 0.047619048 |
| 15 | 81187286 | 81187297 | CEMIP | 0.047619048 |
| 5 | 54701447 | 54701491 | SKIV2L2 | 0.047619048 |
| 5 | 74097328 | 74097361 | FAM169A | 0.047619048 |
| 12 | 6657402 | 6657417 | IFFO1 | 0.047619048 |
| 2 | 157370018 | 157370033 | GPD2 | 0.047904192 |
| 7 | 16704933 | 16704945 | BZW2 | 0.048076923 |
| 13 | 99890849 | 99890862 | MIR548AN | 0.048192771 |
| 18 | 319944 | 319956 | COLEC12(NM_130386: c.*101_*89del0) | 0.048275862 |
| X | 36156447 | 36156461 | CHDC2 | 0.048354085 |
| 11 | 32852019 | 32852043 | PRRG4 | 0.048387097 |
| 1 | 62740780 | 62740792 | KANK4 | 0.048611111 |
| 4 | 115586972 | 115586988 | UGT8 | 0.048780488 |
| 13 | 110833751 | 110833764 | COL4A1 | 0.048780488 |
| 4 | 183600752 | 183600764 | TENM3 | 0.048803828 |
| 18 | 12955439 | 12955452 | SEH1L | 0.048979213 |
| 3 | 71739332 | 71739345 | EIF4E3 | 0.049079755 |
| X | 54042926 | 54042942 | PHF8 | 0.049180328 |
| 1 | 28218773 | 28218784 | RPA2 | 0.049180328 |
| 6 | 90503754 | 90503779 | MDN1 | 0.049180328 |
| 6 | 42611936 | 42611951 | UBR2 | 0.049295775 |
| X | 22244528 | 22244549 | PHEX | 0.049484117 |
| 3 | 7494245 | 7494274 | GRM7 | 0.049558489 |
| 9 | 134049353 | 134049366 | NUP214 | 0.049586777 |
| 2 | 131883287 | 131883301 | PLEKHB2 | 0.049586777 |
| 14 | 67848479 | 67848493 | EIF2S1 | 0.049586777 |
| 20 | 33665775 | 33665786 | TRPC4AP | 0.04964539 |
| 16 | 7743418 | 7743437 | RBFOX1 | 0.049723757 |
| 11 | 16117460 | 16117478 | SOX6 | 0.05 |
| 14 | 75508258 | 75508303 | MLH3 | 0.05 |
| X | 117530912 | 117530925 | WDR44 | 0.05 |
| 14 | 75476004 | 75476015 | EIF2B2(NM_014239: c.*113_*124del0) | 0.05 |

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

An “agent” can be any chemical, entity or moiety, including without limitation synthetic and naturally-occurring proteinaceous and non-proteinaceous entities. In some embodiments, an agent is a nucleic acid, nucleic acid analog, protein, antibody, peptide, aptamer, oligomer of nucleic acids, amino acids, or carbohydrates including without limitation a protein, oligonucleotide, ribozyme, DNAzyme, glycoprotein, siRNAs, lipoprotein and/or a modification or combinations thereof etc. In certain embodiments, agents are small molecule chemical moieties. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

An agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertions and other variants.

The term “therapeutically effective amount” refers to an amount of a therapeutic as described herein, that is effective to treat a disease or disorder as the terms “treat” or “treatment” are defined herein. Amounts will vary depending on the specific disease or disorder, its state of progression, age, weight and gender of a subject, among other variables. Thus, it is not possible to specify an exact “effective amount”. However, for any given case, an appropriate “effective amount” can be determined by one of ordinary skill in the art using only routine experimentation.

As used herein, the term “checkpoint inhibitor immunotherapy” refers to any agent, small molecule, antibody, or the like that can reduce or inhibit the level or activity of an immune checkpoint molecule. Inhibition of an immune checkpoint molecule can promote an immune response, e.g., against cancer or a tumor which otherwise evades such response. Immune checkpoint molecules can include but are not limited to PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7), and those described, e.g., Pardoll et al., Nature Reviews Cancer 12, 252-264 (2012), which is incorporated herein by reference in its entirety. Non-limiting examples of checkpoint inhibitors include pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®)

As used herein, the term “non-checkpoint inhibitor cancer therapy” refers to any therapy or agent useful in treating cancer other than checkpoint inhibitor immunotherapy. One of skill in the art can readily identify a non-checkpoint inhibitor chemotherapeutic agent, e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; Baltzer L, Berkery R (eds): Oncology Pocket Guide to Chemotherapy, 2nd ed. St. Louis, Mosby-Year Book, 1995; Fischer D S (ed): The Cancer Chemotherapy Handbook, 4th ed. St. Louis, Mosby-Year Book, 2003)).

As used herein, the term “cancer” refers to a hyperproliferation of cells that exhibit a loss of normal cellular control that results in unregulated growth, lack of differentiation, local tissue invasion, and metastasis. Non-limiting examples of cancer types include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, heavy chain disease. In some embodiments of any of the aspects, the cancer type is selected from the group consisting of: BLCA, Bladder Urothelial Carcinoma; BRCA, Breast invasive carcinoma; CESC, Cervical squamous cell carcinoma and endocervical adenocarcinoma; CHOL, Cholangiocarcinoma; COAD, Colon adenocarcinoma; DLBC, Lymphoid Neoplasm Diffuse Large B-cell Lymphoma; ESCA, Esophageal carcinoma; GBM, Glioblastoma multiforme; HNSC, Head and Neck squamous cell carcinoma; KICH, Kidney Chromophobe; KIRC, Kidney renal clear cell carcinoma; KIRP, Kidney renal papillary cell carcinoma; LAML, Acute Myeloid Leukemia; LGG, Brain Lower Grade Glioma; LIHC, Liver hepatocellular carcinoma; LUAD, Lung adenocarcinoma; LUSC, Lung squamous cell carcinoma; MESO, Mesothelioma; OV, Ovarian serous cystadenocarcinoma; PAAD, Pancreatic adenocarcinoma; PCPG, Pheochromocytoma and Paraganglioma; PRAD, Prostate adenocarcinoma; READ, Rectum adenocarcinoma; SARC, Sarcoma; SKCM, Skin Cutaneous Melanoma; STAD, Stomach adenocarcinoma; TGCT, Testicular Germ Cell Tumors; THCA, Thyroid carcinoma; THYM, Thymoma; UCEC, Uterine Corpus Endometrial Carcinoma; UCS, Uterine Carcinosarcoma; and UVM, Uveal Melanoma.

As used herein the term “microsatellite instability” or “MSI” refers to a molecular tumor phenotype that is indicative of genomic hypermutability, marked by spontaneous gains or losses of nucleotides from repetitive DNA tracts, resulting in new alleles of differing length.

As used herein, the term “microsatellite repeat sequence” refers to a repetitive nucleotide sequence of about 1-6 base pairs or more in length. The repeat sequences can vary in number of repeats, generally ranging from about 5 to about 60 repeats. The methods provided herein are based, in part, on microsatellite mutations relative to microsatellite repeat sequences in the reference human genome, GRCh37/hg19. A microsatellite repeat sequence can be identified, for example, using tools such as the microsatellite identification tool (MISA), found on the world wide web at <webblast.ipk-gatersleben.de/misa/>.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total). The term “treatment” of a disease also includes providing relief from the symptoms or side-effects of the disease (including palliative treatment).

The terms “decrease”, “reduce”, “reduction”, or “inhibit” are all used herein to mean a decrease by a statistically significant amount. In some embodiments, “reduce,” “reduction”, “decrease” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a “subject” is a human or a non-human animal. Usually the non-human animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases including diseases and disorders involving inappropriate immunosuppression. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at risk of developing that condition. In one embodiment, a subject in need either has the condition or has been diagnosed as having the condition.

As used herein, a “reference level” refers to the level of a given, e.g., biomarker or parameter useful as a gauge for an experimental or diagnostic measurement. In one embodiment, a reference level is the level of such marker or parameter in a normal, otherwise unaffected cell population or tissue (e.g., a biological sample obtained from a healthy subject, or a level of the marker or parameter from a sample obtained from the subject at a prior time point, e.g., a biological sample obtained from a patient prior to being diagnosed with a disease or disorder, or a biological sample from an individual that has not been contacted with a therapeutic composition). It is contemplated that one can also use a biomarker or parameter from an individual whose cancer does respond to checkpoint inhibitor immunotherapy as a reference. Microsatellites as described herein are compared to those in reference human genome build GRCh37/hg19.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of predicting whether a subject's cancer will respond to checkpoint inhibitor immunotherapy, the method comprising:
   (a) receiving microsatellite instability data for a defined set of microsatellite repeat marker sequences in cells of the subject's cancer; and
   (b) processing the microsatellite instability data to output a categorical measure of microsatellite instability high (MSI-H) or microsatellite stable (MSS);
   wherein when the subject's cancer is determined to exhibit MSI-H, it is predicted that the cancer will respond to checkpoint inhibitor immunotherapy, and when the subject's cancer is determined to exhibit MSS, it is predicted that the cancer is less likely to respond to checkpoint inhibitor immunotherapy.
2. A method of predicting whether a subject's cancer will respond to checkpoint inhibitor immunotherapy, the method comprising:
   determining microsatellite instability status for a defined set of microsatellite repeat marker sequences in cells of the subject's cancer,
   wherein determining the microsatellite instability status of the subject's cancer comprises:
      (i) assaying loci in the defined set of microsatellite repeat marker sequences in cells of the subject's cancer for mutation, thereby generating microsatellite instability data; and
      (ii) processing the microsatellite instability data to output a categorical measure of microsatellite instability high (MSI-H) status or microsatellite stable (MSS) status for the subject's cancer;
      wherein when the subject's cancer is determined to exhibit MSI-H, it is predicted that the cancer will respond to checkpoint inhibitor immunotherapy, and when the subject's cancer is determined to exhibit MSS, it is predicted that the cancer is less likely to respond to checkpoint inhibitor immunotherapy.
3. The method of paragraph 1 or paragraph 2, wherein processing the microsatellite instability data to output a categorical measure of microsatellite instability high (MSI-H) or microsatellite stable (MSS) comprises applying a quantitative model relating the predictive capability of the markers in the set or a subset thereof to known MSI status.
4. The method of paragraph 3, wherein the quantitative model is selected from a continuous measure, regression or weighted scoring of markers, a combinatorial or decision-tree model, and a machine learning model.
5. The method of paragraph 4, wherein the continuous measure comprises the proportion of unmutated or stable loci to mutated or unstable loci detected in the set.
6. The method of paragraph 4, wherein the quantitative model comprises a random-forest model or a deep neural network machine learning model.
7. The method of paragraph 3, wherein the quantitative model evaluates or incorporates consideration of one or more test characteristics selected from the group consisting of sensitivity, accuracy, correlation, probability, specificity, false-positive rate, false negative rate, positive predictive value, negative predictive value and area under the receiver-operator characteristic (AUROC).
8. The method of paragraph 7, wherein thresholds for the one or more test characteristics indicative of MSI-H or MSS are defined within parameters of the known test characteristics for a given clinical application.
9. The method of paragraph 1 or paragraph 2, wherein:
   when the cancer is colon adenocarcinoma (COAD), the set of microsatellite repeat marker sequences comprises a plurality up to 500 of the microsatellites set out in Table 1;
   when the cancer is esophageal carcinoma (ESCA), the set of microsatellite repeat marker sequences comprises a plurality up to 503 of the microsatellites set out in Table 2;
   when the cancer is glioblastoma multiforme (GBM), the set of microsatellite repeat marker sequences comprises a plurality up to 500 of the microsatellites set out in Table 3;
   when the cancer is lung adenocarcinoma (LUAD), the set of microsatellite repeat marker sequences comprises a plurality up to 500 of the microsatellites set out in Table 4;
   when the cancer is lung squamous cell carcinoma (LUSC), the set of microsatellite repeat marker sequences comprises a plurality up to 500 of the microsatellites set out in Table 5;
   when the cancer is rectum adenocarcinoma (READ), the set of microsatellite repeat marker sequences comprises a plurality up to 501 of the microsatellites set out in Table 6;
   when the cancer is stomach adenocarcinoma (STAD), the set of microsatellite repeat marker sequences comprises a plurality up to 500 of the microsatellites set out in Table 7;
   when the cancer is brain lower grade glioma (LGG), the set of microsatellite repeat marker sequences comprises a plurality up to 266 of the microsatellites set out in Table 8;
   when the cancer is prostate adenocarcinoma (PRAD), the set of microsatellite repeat marker sequences comprises a plurality up to 500 of the microsatellites set out in Table 9;
   when the cancer is cervical squamous cell carcinoma (CESC), the set of microsatellite repeat marker sequences comprises a plurality up to 500 of the microsatellites set out in Table 10;
   when the cancer is lymphoid neoplasm diffuse large B-cell lymphoma (DLBC), the set of microsatellite repeat marker sequences comprises a plurality up to 212 of the microsatellites set out in Table 11;
   when the cancer is uterine corpus endometrial carcinoma (UCEC), the set of microsatellite repeat marker sequences comprises a plurality up to 500 of the microsatellites set out in Table 12;
   when the cancer is kidney renal clear cell carcinoma (KIRC), the set of microsatellite repeat marker sequences comprises a plurality up to 508 of the microsatellites set out in Table 13;
   when the cancer is breast invasive carcinoma (BRCA), the set of microsatellite repeat marker sequences comprises a plurality up to 500 of the microsatellites set out in Table 14A; and
   when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.
10. The method of paragraph 1 or paragraph 2, wherein the set of microsatellite repeat marker sequences comprises a plurality up to 200 of the sequences set out in the respective Table for the subject's cancer type.
11. The method of paragraph 1 or paragraph 2, wherein the set of microsatellite repeat marker sequences comprises a plurality up to 300 of the sequences set out in the respective Table for the subject's cancer type.
12. The method of paragraph 1 or paragraph 2, wherein the set of microsatellite repeat marker sequences comprises a plurality up to the first 100 of the sequences set out in the respective Table for the subject's cancer type.
13. The method of paragraph 1 or paragraph 2, wherein the set of microsatellite repeat marker sequences comprises a plurality up to the first 200 of the sequences set out in the respective Table for the subject's cancer type.
14. The method of paragraph 1 or paragraph 2, wherein the set of microsatellite repeat marker sequences comprises a plurality up to the first 300 of the sequences set out in the respective Table for the subject's cancer type.
15. The method of any one of paragraphs 1-14, wherein the checkpoint inhibitor immunotherapy is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).
16. The method of any one of paragraphs 1-15, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).
17. The method of paragraph 1 or paragraph 2, wherein the processing of MSI-H or MSS is determined by a threshold value for the set of microsatellite marker sequences.
18. The method of paragraph 17, wherein the threshold for the set of microsatellite marker sequences is determined using the percentage of mutated microsatellite markers in the set to calculate the area under the receiver operating characteristic (AUROC).
19. The method of paragraph 18, wherein the smallest number of markers in the set necessary to reach a selected AUROC value is the threshold.
20. The method of paragraph 19, wherein the selected AUROC value is a value between 0.6 and 0.99, inclusive.
21. The method of paragraph 19, wherein the selected AUROC value is 0.9 or more.
22. The method of any one of paragraphs 1-21, wherein the checkpoint inhibitor immunotherapy is an inhibitor of PD-1 or PD-L1.
23. The method of any one of paragraphs 1-22, wherein the checkpoint inhibitor immunotherapy comprises a checkpoint inhibitor antibody.
24. The method of paragraph 2, wherein assaying loci in the defined set of microsatellite repeat marker sequences in cells of the subject's cancer for mutation comprises polynucleotide sequencing, measurement of total mutational burden, immunohistochemical analysis, and/or polymerase chain reaction.
25. The method of paragraph 24, wherein polynucleotide sequencing comprises whole genome sequencing, whole exome sequencing, or targeted gene capture sequencing.
26. The method of paragraph 1 or paragraph 2, wherein microsatellite mutations correspond to those identified in reference human genome GRCh37/hg19 translated to a different build of the human reference genome.
27. A method of determining microsatellite instability (MSI) in a subject's cancer, the method comprising
   assaying mutation status for a set of microsatellite repeat marker sequences in cells of the subject's cancer, wherein when the set of microsatellites exhibits mutation at or above a threshold number of microsatellites in the set, the subject's cancer is determined to exhibit high microsatellite instability (MSI-H), and when the set of microsatellites exhibits mutation below the threshold number of microsatellites in the set, the subject's cancer is determined to exhibit microsatellite stability (MSS);
   wherein the threshold for the set is determined using the percentage of mutated microsatellite markers in the set to calculate the area under the receiver operating characteristic (AUROC), and wherein the smallest number of markers in the set necessary to reach an AUROC value selected in the range of 0.6 to 0.99, inclusive, is the threshold;
   and wherein:
   when the cancer is colon adenocarcinoma (COAD), the set of microsatellite repeat marker sequences comprises a plurality up to 500 of the microsatellites set out in Table 1;
   when the cancer is esophageal carcinoma (ESCA), the set of microsatellite repeat marker sequences comprises a plurality up to 503 of the microsatellites set out in Table 2;
   when the cancer is glioblastoma multiforme (GBM), the set of microsatellite repeat marker sequences comprises a plurality up to 500 of the microsatellites set out in Table 3;
   when the cancer is lung adenocarcinoma (LUAD), the set of microsatellite repeat marker sequences comprises a plurality up to 500 of the microsatellites set out in Table 4;
   when the cancer is lung squamous cell carcinoma (LUSC), the set of microsatellite repeat marker sequences comprises a plurality up to 500 of the microsatellites set out in Table 5;
   when the cancer is rectum adenocarcinoma (READ), the set of microsatellite repeat marker sequences comprises a plurality up to 501 of the microsatellites set out in Table 6;
   when the cancer is stomach adenocarcinoma (STAD), the set of microsatellite repeat marker sequences comprises a plurality up to 500 of the microsatellites set out in Table 7;
   when the cancer is brain lower grade glioma (LGG), the set of microsatellite repeat marker sequences comprises a plurality up to 266 of the microsatellites set out in Table 8;
   when the cancer is prostate adenocarcinoma (PRAD), the set of microsatellite repeat marker sequences comprises a plurality up to 500 of the microsatellites set out in Table 9;
   when the cancer is cervical squamous cell carcinoma (CESC), the set of microsatellite repeat marker sequences comprises a plurality up to 500 of the microsatellites set out in Table 10;
   when the cancer is lymphoid neoplasm diffuse large B-cell lymphoma (DLBC), the set of microsatellite repeat marker sequences comprises a plurality up to 212 of the microsatellites set out in Table 11;
   when the cancer is uterine corpus endometrial carcinoma (UCEC), the set of microsatellite repeat marker sequences comprises a plurality up to 500 of the microsatellites set out in Table 12;
   when the cancer is kidney renal clear cell carcinoma (KIRC), the set of microsatellite repeat marker sequences comprises a plurality up to 508 of the microsatellites set out in Table 13;
   when the cancer is breast invasive carcinoma (BRCA), the set of microsatellite repeat marker sequences comprises a plurality up to 500 of the microsatellites set out in Table 14A; and
   when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.
28. The method of paragraph 27, wherein the threshold is an AUROC value of 0.9.
29. The method of paragraph 27, wherein the set of microsatellite repeat marker sequences comprises a plurality up to 200 of the sequences set out in the respective Table for the subject's cancer type.
30. The method of paragraph 27, wherein the set of microsatellite repeat marker sequences comprises a plurality up to 300 of the sequences set out in the respective Table for the subject's cancer type.
31. The method of paragraph 27, wherein the set of microsatellite repeat marker sequences comprises a plurality up to the first 100 of the sequences set out in the respective Table for the subject's cancer type.
32. The method of paragraph 27, wherein the set of microsatellite repeat marker sequences comprises a plurality up to the first 200 of the sequences set out in the respective Table for the subject's cancer type.
33. The method of paragraph 27, wherein the set of microsatellite repeat marker sequences comprises a plurality up to the first 300 of the sequences set out in the respective Table for the subject's cancer type.
34. The method of any one of paragraphs 27-33, wherein if the cancer is determined to be MSI-H, the subject is administered an effective amount of a checkpoint inhibitor immunotherapy.
35. The method of any one of paragraphs 27-33, wherein if the cancer is determined to not be MSI-H, the subject is administered an effective amount of a non-checkpoint inhibitor cancer therapeutic.
36. The method of paragraph 34, wherein the checkpoint inhibitor immunotherapy is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).
37. The method of any one of paragraphs 27-36, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).
38. The method of any one of paragraphs 27-37, wherein the checkpoint inhibitor immunotherapy is an inhibitor of PD-1 or PD-L1.
39. The method of any one of paragraphs 27-38, wherein the checkpoint inhibitor immunotherapy comprises a checkpoint inhibitor antibody.
40. The method of any one of paragraphs 27-39, wherein assaying mutation status comprises polynucleotide sequencing, measurement of tumor mutational burden, inference of mutational signatures, immunohistochemical analysis, and/or polymerase chain reaction.
41. The method of paragraph 40, wherein polynucleotide sequencing comprises whole genome sequencing, whole exome sequencing, or targeted gene capture sequencing.
42. The method of any one of paragraphs 27-41, wherein microsatellite mutations correspond to those identified in reference human genome GRCh37/hg19 translated to a different build of the human reference genome.
43. A method of treating cancer in a subject in need thereof, the method comprising determining microsatellite instability status for cells of a subject's cancer by a method of any one of paragraphs 1-42, and, when the microsatellite instability status is determined to be MSI-H, administering a checkpoint inhibitor, or, when the microsatellite instability status is determined to be MSS, administering a non-checkpoint inhibitor cancer therapeutic.
44. The method of paragraph 43, wherein the checkpoint inhibitor is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).
45. The method of paragraph 43, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).
46. The method of paragraph 43, wherein the checkpoint inhibitor is an inhibitor of PD-1 or PD-L1.
47. The method of paragraph 43, wherein the checkpoint inhibitor comprises a checkpoint inhibitor antibody.
48. The method of paragraph 43, wherein the non-checkpoint inhibitor cancer therapy comprises one or more of angiostatin K1-3, DL-a-Difluoromethyl-ornithine, endostatin, fumagillin, genistein, minocycline, staurosporine, and (±)-thalidomide; a DNA intercalator/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, Dolastatin 15, Nocodazole, Paclitaxel, Podophyllotoxin, Rhizoxin, Vinblastine, Vincristine, Vindesine, and Vinorelbine (Navelbine); a neoantigen; and an unclassified antitumor agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™) idelalisib (Zydelig®), crizotinib (Xalkori®), erlotinib (Tarceva®), afatinib dimaleate (Gilotrif®), ceritinib (LDK378/Zykadia), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), a targeted therapy such as toremifene (Fareston®), fulvestrant (Faslodex®), anastrozole (Arimidex®), exemestane (Aromasin®), letrozole (Ferrara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®), an epigenetic targeted drug such as HDAC inhibitors, azacitidine (Vidaza®), decitabine (Dacogen®), vorinostat (Zolinza®), romidepsin (Istodax®), ruxolitinib (Jakafi®), kinase inhibitors, DNA methyltransferase inhibitors, histone demethylase inhibitors, or histone methylation inhibitors, and any derivative thereof.

49. A method of treating cancer in a subject in need thereof, wherein the subject's cancer has been determined to have a microsatellite instability status of MSI-H as determined by the method of any one of paragraphs 1-42, the method comprising administering an effective amount of a checkpoint inhibitor to the subject.
50. The method of paragraph 49, wherein the checkpoint inhibitor is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).
51. The method of paragraph 49, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).
52. The method of paragraph 49, wherein the checkpoint inhibitor is an inhibitor of PD-1 or PD-L1.
53. The method of paragraph 49, wherein the checkpoint inhibitor comprises a checkpoint inhibitor antibody.
54. The method of paragraph 49, wherein the non-checkpoint inhibitor cancer therapy comprises one or more of angiostatin K1-3, DL-α-Difluoromethyl-ornithine, endostatin, fumagillin, genistein, minocycline, staurosporine, and (±)-thalidomide; a DNA intercalator/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, Dolastatin 15, Nocodazole, Paclitaxel, Podophyllotoxin, Rhizoxin, Vinblastine, Vincristine, Vindesine, and Vinorelbine (Navelbine); a neoantigen; and an unclassified antitumor agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™) idelalisib (Zydelig®), crizotinib (Xalkori®), erlotinib (Tarceva®), afatinib dimaleate (Gilotrif®), ceritinib (LDK378/Zykadia), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), a targeted therapy such as toremifene (Fareston®), fulvestrant (Faslodex®), anastrozole (Arimidex®), exemestane (Aromasin®), letrozole (Ferrara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®), an epigenetic targeted drug such as HDAC inhibitors, azacitidine (Vidaza®), decitabine (Dacogen®), vorinostat (Zolinza®), romidepsin (Istodax®), ruxolitinib (Jakafi®), kinase inhibitors, DNA methyltransferase inhibitors, histone demethylase inhibitors, or histone methylation inhibitors, and any derivative thereof.
55. A method of predicting whether a subject's cancer will respond to checkpoint inhibitor immunotherapy, the method comprising:
   assaying mutation status for a set of microsatellite repeat marker sequences in cells of the subject's cancer, wherein when mutations are present in at least a threshold number of markers in the set, the set provides at least 95% sensitivity and at least 95% specificity for predicting high microsatellite instability (MSI-H) for the subject's tumor,
   wherein:
   when the cancer is colon adenocarcinoma (COAD), the microsatellite repeat marker sequences comprise a plurality of microsatellite repeat markers in Table 1 and the threshold number is 50%;
   when the cancer is esophageal carcinoma (ESCA), the microsatellite repeat marker sequences comprise a plurality of microsatellite repeat markers in Table 2 and the threshold number is 50%;
   when the cancer is glioblastoma multiforme (GBM), the microsatellite repeat marker sequences comprise a plurality of microsatellite repeat markers in Table 3 and the threshold number is 50%;
   when the cancer is lung adenocarcinoma (LUAD), the microsatellite repeat marker sequences comprise a plurality of microsatellite repeat markers in Table 4 and the threshold number is 50%;
   when the cancer is lung squamous cell carcinoma (LUSC), the microsatellite repeat marker sequences comprise a plurality of microsatellite repeat markers in Table 5 and the threshold number is 50%;
   when the cancer is rectum adenocarcinoma (READ), the microsatellite repeat marker sequences comprise a plurality of microsatellite repeat markers in Table 6 and the threshold number is 50%;
   when the cancer is stomach adenocarcinoma (STAD), the microsatellite repeat marker sequences comprise a plurality of microsatellite repeat markers in Table 7 and the threshold number is 25%;
   when the cancer is brain lower grade glioma (LGG), the microsatellite repeat marker sequences comprise a plurality of microsatellite repeat markers in Table 8 and the threshold number is 12.5%;
   when the cancer is prostate adenocarcinoma (PRAD), the microsatellite repeat marker sequences comprise a plurality of microsatellite repeat markers in Table 9 and the threshold number is 16.7%;
   when the cancer is cervical squamous cell carcinoma (CESC), the microsatellite repeat marker sequences comprise a plurality of microsatellite repeat markers in Table 10 and the threshold number is 45.8%;
   when the cancer is lymphoid neoplasm diffuse large B-cell lymphoma (DLBC), the microsatellite repeat marker sequences comprise a plurality of microsatellite repeat markers in Table 11 and the threshold number is 25%;
   when the cancer is uterine corpus endometrial carcinoma (UCEC), the microsatellite repeat marker sequences comprise a plurality of microsatellite repeat markers in Table 12 and the threshold number is 12.9%;
   when the cancer is kidney renal clear cell carcinoma (KIRC), the microsatellite repeat marker sequences comprise a plurality of microsatellite repeat markers in Table 13 and the threshold number is 7.5%;
   when the cancer is breast invasive carcinoma (BRCA), the microsatellite repeat marker sequences comprise a plurality of microsatellite repeat markers in Table 14A and the threshold number is 3.6%; and
   when the cancer is uterine corpus endometrial carcinoma (UCEC), colon adenocarcinoma (COAD), rectum adenocarcinoma (READ), or stomach adenocarcinoma (STAD), the microsatellite repeat marker sequences comprise a plurality of microsatellite repeat markers in Table 21 and the threshold number is at least 12.9% (e.g., 12.9%, 25%, 50%);
   and wherein
   when the subject's cancer exhibits greater than or equal to the threshold number of mutations for that tumor type in the set, the subject's cancer is determined to be MSI-H, and is predicted to be sensitive to checkpoint inhibitor immunotherapy.
56. The method of paragraph 55, wherein the checkpoint inhibitor immunotherapy is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).
57. The method of paragraph 55 or 56, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).
58. The method of any one of paragraphs 55-57, wherein the checkpoint inhibitor immunotherapy is an inhibitor of PD-1 or PD-L1.
59. The method of any one of paragraphs 55-58, wherein the checkpoint inhibitor immunotherapy comprises a checkpoint inhibitor antibody.
60. The method of any one of paragraphs 55-59, wherein assaying mutation status comprises polynucleotide sequencing, measurement of tumor mutational burden, inference of mutational signatures, immunohistochemical analysis, and/or polymerase chain reaction.
61. The method of paragraph 60, wherein polynucleotide sequencing comprises whole genome sequencing, whole exome sequencing, or targeted gene capture sequencing.
62. The method of any one of paragraphs 55-61, wherein microsatellite mutations correspond to those identified in reference human genome GRCh37/hg19 translated to a different build of the human reference genome.
63. The method of any one of paragraphs 55-62, wherein if the subject is predicted to be sensitive to the checkpoint inhibitor immunotherapy, the subject is administered an effective amount of the checkpoint inhibitor immunotherapy.
64. The method of any one of paragraphs 55-62, wherein if the subject is predicted to not be sensitive to the checkpoint inhibitor immunotherapy, the subject is administered an effective amount of a non-checkpoint inhibitor cancer therapeutic.
65. A method of determining microsatellite instability (MSI) in a subject's cancer, the method comprising:
   assaying mutation status for a set of microsatellite repeat marker sequences in the cells of the subject's cancer, wherein when mutations are present in at least a threshold number of markers in the set, the set provides at least 95% sensitivity and at least 95% specificity for predicting high microsatellite instability (MSI-H) for the subject's tumor,
   wherein:
   when the cancer is colon adenocarcinoma (COAD), the microsatellite repeat marker sequences comprise a plurality of microsatellite repeat markers in Table 1 and the threshold number is 50%;
   when the cancer is esophageal carcinoma (ESCA), the microsatellite repeat marker sequences comprise a plurality of microsatellite repeat markers in Table 2 and the threshold number is 50%;
   when the cancer is glioblastoma multiforme (GBM), the microsatellite repeat marker sequences comprise a plurality of microsatellite repeat markers in Table 3 and the threshold number is 50%;
   when the cancer is lung adenocarcinoma (LUAD), the microsatellite repeat marker sequences comprise a plurality of microsatellite repeat markers in Table 4 and the threshold number is 50%;
   when the cancer is lung squamous cell carcinoma (LUSC), the microsatellite repeat marker sequences comprise a plurality of microsatellite repeat markers in Table 5 and the threshold number is 50%;
   when the cancer is rectum adenocarcinoma (READ), the microsatellite repeat marker sequences comprise a plurality of microsatellite repeat markers in Table 6 and the threshold number is 50%;
   when the cancer is stomach adenocarcinoma (STAD), the microsatellite repeat marker sequences comprise a plurality of microsatellite repeat markers in Table 7 and the threshold number is 25%;
   when the cancer is brain lower grade glioma (LGG), the microsatellite repeat marker sequences comprise a plurality of microsatellite repeat markers in Table 8 and the threshold number is 12.5%;
   when the cancer is prostate adenocarcinoma (PRAD), the microsatellite repeat marker sequences comprise a plurality of microsatellite repeat markers in Table 9 and the threshold number is 16.7%;
   when the cancer is cervical squamous cell carcinoma (CESC), the microsatellite repeat marker sequences comprise a plurality of microsatellite repeat markers in Table 10 and the threshold number is 45.8%;
   when the cancer is lymphoid neoplasm diffuse large B-cell lymphoma (DLBC), the microsatellite repeat marker sequences comprise a plurality of microsatellite repeat markers in Table 11 and the threshold number is 25%;
   when the cancer is uterine corpus endometrial carcinoma (UCEC), the microsatellite repeat marker sequences comprise a plurality of microsatellite repeat markers in Table 12 and the threshold number is 12.9%;
   when the cancer is kidney renal clear cell carcinoma (KIRC), the microsatellite repeat marker sequences comprise a plurality of microsatellite repeat markers in Table 13 and the threshold number is 7.5%;
   when the cancer is breast invasive carcinoma (BRCA), the microsatellite repeat marker sequences comprise a plurality of microsatellite repeat markers in Table 14A and the threshold number is 3.6%; and
   when the cancer is uterine corpus endometrial carcinoma (UCEC), colon adenocarcinoma (COAD), rectum adenocarcinoma (READ), or stomach adenocarcinoma (STAD), the microsatellite repeat marker sequences comprise a plurality of microsatellite repeat markers in Table 21 and the threshold number is at least 12.9% (e.g., 12.9%, 25%, 50%); and
   wherein, cancer that exhibits greater than or equal to the threshold number of mutations for that tumor type in the set is determined to be MSI-H, and
   cancer that exhibits fewer than the threshold number of mutations for that tumor type in the set, the subject's cancer is determined to be microsatellite stable or not MSI-H.
66. The method of paragraph 65, wherein assaying mutation status comprises polynucleotide sequencing, measurement of tumor mutational burden, inference of mutational signatures, immunohistochemical analysis, and/or polymerase chain reaction.
67. The method of paragraph 66, wherein polynucleotide sequencing comprises whole genome sequencing, whole exome sequencing, or targeted gene capture sequencing.
68. The method of any one of paragraphs 65-67, wherein microsatellite mutations correspond to those identified in reference human genome GRCh37/hg19 translated to a different build of the human reference genome.
69. The method of any one of paragraphs 65-68, wherein if the cancer is determined to be MSI-H, the subject is administered an effective amount of a checkpoint inhibitor immunotherapy.
70. The method of any one of paragraphs 65-68, wherein if the cancer is determined to not be MSI-H, the subject is administered an effective amount of a non-checkpoint inhibitor cancer therapeutic.
71. A method of treating cancer in a subject in need thereof, the method comprising:
   assaying mutation status for a set of microsatellite repeat marker sequences in cells of the subject's cancer, wherein when mutations are present in at least a threshold number of markers in the set, and the set provides at least 95% sensitivity and at least 95% specificity for predicting high microsatellite instability (MSI-H) for the subject's tumor,
   wherein:
   when the cancer is colon adenocarcinoma (COAD), the microsatellite repeat marker sequences comprise a plurality of microsatellite repeat markers in Table 1 and the threshold number is 50%;
   when the cancer is esophageal carcinoma (ESCA), the microsatellite repeat marker sequences comprise a plurality of microsatellite repeat markers in Table 2 and the threshold number is 50%;
   when the cancer is glioblastoma multiforme (GBM), the microsatellite repeat marker sequences comprise a plurality of microsatellite repeat markers in Table 3 and the threshold number is 50%;
   when the cancer is lung adenocarcinoma (LUAD), the microsatellite repeat marker sequences comprise a plurality of microsatellite repeat markers in Table 4 and the threshold number is 50%;
   when the cancer is lung squamous cell carcinoma (LUSC), the microsatellite repeat marker sequences comprise a plurality of microsatellite repeat markers in Table 5 and the threshold number is 50%;
   when the cancer is rectum adenocarcinoma (READ), the microsatellite repeat marker sequences comprise a plurality of microsatellite repeat markers in Table 6 and the threshold number is 50%;
   when the cancer is stomach adenocarcinoma (STAD), the microsatellite repeat marker sequences comprise a plurality of microsatellite repeat markers in Table 7 and the threshold number is 25%;
   when the cancer is brain lower grade glioma (LGG), the microsatellite repeat marker sequences comprise a plurality of microsatellite repeat markers in Table 8 and the threshold number is 12.5%;
   when the cancer is prostate adenocarcinoma (PRAD), the microsatellite repeat marker sequences comprise a plurality of microsatellite repeat markers in Table 9 and the threshold number is 16.7%;
   when the cancer is cervical squamous cell carcinoma (CESC), the microsatellite repeat marker sequences comprise a plurality of microsatellite repeat markers in Table 10 and the threshold number is 45.8%;
   when the cancer is lymphoid neoplasm diffuse large B-cell lymphoma (DLBC), the microsatellite repeat marker sequences comprise a plurality of microsatellite repeat markers in Table 11 and the threshold number is 25%;
   when the cancer is uterine corpus endometrial carcinoma (UCEC), the microsatellite repeat marker sequences comprise a plurality of microsatellite repeat markers in Table 12 and the threshold number is 12.9%;
   when the cancer is kidney renal clear cell carcinoma (KIRC), the microsatellite repeat marker sequences comprise a plurality of microsatellite repeat markers in Table 13 and the threshold number is 7.5%;
   when the cancer is breast invasive carcinoma (BRCA), the microsatellite repeat marker sequences comprise a plurality of microsatellite repeat markers in Table 14A and the threshold number is 3.6%; and
   when the cancer is uterine corpus endometrial carcinoma (UCEC), colon adenocarcinoma (COAD), rectum adenocarcinoma (READ), or stomach adenocarcinoma (STAD), the microsatellite repeat marker sequences comprise a plurality of microsatellite repeat markers in Table 21 and the threshold number is at least 12.9% (e.g., 12.9%, 25%, 50%); and
   wherein
   when the subject's cancer exhibits greater than or equal to the threshold number of mutations for that tumor type in the set, administering a checkpoint inhibitor immunotherapy, and
   when the subject's cancer exhibits fewer than the threshold number of mutations for that tumor type in the set, administering a non-checkpoint inhibitor cancer therapy.
72. The method of paragraph 71, wherein assaying mutation status comprises polynucleotide sequencing, measurement of tumor mutational burden, inference of mutational signatures, immunohistochemical analysis, and/or polymerase chain reaction.
73. The method of paragraph 72, wherein polynucleotide sequencing comprises whole genome sequencing, whole exome sequencing, or targeted gene capture sequencing.
74. The method of any one of paragraphs 71-73, wherein microsatellite mutations correspond to those identified in reference human genome GRCh37/hg19 translated to a different build of the human reference genome.
75. Use of a checkpoint inhibitor in a method for the treatment of cancer, the method comprising:
   (a) receiving microsatellite instability data for a defined set of microsatellite repeat marker sequences in cells of the subject's cancer; and
   (b) processing the microsatellite instability data to output a categorical measure of microsatellite instability high (MSI-H) or microsatellite stable (MSS);
   wherein the checkpoint inhibitor is administered when the subject's cancer is determined to exhibit MSI-H.
76. Use of a checkpoint inhibitor in a method for the treatment of cancer, the method comprising:
   determining microsatellite instability status for a defined set of microsatellite repeat marker sequences in cells of the subject's cancer,
   wherein determining the microsatellite instability status of the subject's cancer comprises:
      (i) assaying loci in the defined set of microsatellite repeat marker sequences in cells of the subject's cancer for mutation, thereby generating microsatellite instability data; and
      (ii) processing the microsatellite instability data to output a categorical measure of microsatellite instability high (MSI-H) status or microsatellite stable (MSS) status for the subject's cancer;
      wherein the checkpoint inhibitor is administered when the subject's cancer is determined to exhibit MSI-H.
77. The use of paragraph 75 or paragraph 76, wherein processing the microsatellite instability data to output a categorical measure of microsatellite instability high (MSI-H) or microsatellite stable (MSS) comprises applying a quantitative model relating the predictive capability of the markers in the set or a subset thereof to known MSI status.
78. The use of paragraph 77, wherein the quantitative model is selected from a continuous measure, regression or weighted scoring of markers, a combinatorial or decision-tree model, and a machine learning model.
79. The use of paragraph 78, wherein the continuous measure comprises the proportion of unmutated or stable loci to mutated or unstable loci detected in the set.
80. The use of paragraph 78, wherein the quantitative model comprises a random-forest model or a deep neural network machine learning model.
81. The use of paragraph 77, wherein the quantitative model evaluates or incorporates consideration of one or more test characteristics selected from the group consisting of sensitivity, accuracy, correlation, probability, specificity, false-positive rate, false negative rate, positive predictive value, negative predictive value and area under the receiver-operator characteristic (AUROC).
82. The use of paragraph 81, wherein thresholds for the one or more test characteristics indicative of MSI-H or MSS are defined within parameters of the known test characteristics for a given clinical application.
83. The use of paragraph 75 or paragraph 76, wherein:
   when the cancer is colon adenocarcinoma (COAD), the set of microsatellite repeat marker sequences comprises a plurality up to 500 of the microsatellites set out in Table 1;
   when the cancer is esophageal carcinoma (ESCA), the set of microsatellite repeat marker sequences comprises a plurality up to 503 of the microsatellites set out in Table 2;
   when the cancer is glioblastoma multiforme (GBM), the set of microsatellite repeat marker sequences comprises a plurality up to 500 of the microsatellites set out in Table 3;
   when the cancer is lung adenocarcinoma (LUAD), the set of microsatellite repeat marker sequences comprises a plurality up to 500 of the microsatellites set out in Table 4;
   when the cancer is lung squamous cell carcinoma (LUSC), the set of microsatellite repeat marker sequences comprises a plurality up to 500 of the microsatellites set out in Table 5;
   when the cancer is rectum adenocarcinoma (READ), the set of microsatellite repeat marker sequences comprises a plurality up to 501 of the microsatellites set out in Table 6;
   when the cancer is stomach adenocarcinoma (STAD), the set of microsatellite repeat marker sequences comprises a plurality up to 500 of the microsatellites set out in Table 7;
   when the cancer is brain lower grade glioma (LGG), the set of microsatellite repeat marker sequences comprises a plurality up to 266 of the microsatellites set out in Table 8;
   when the cancer is prostate adenocarcinoma (PRAD), the set of microsatellite repeat marker sequences comprises a plurality up to 500 of the microsatellites set out in Table 9;
   when the cancer is cervical squamous cell carcinoma (CESC), the set of microsatellite repeat marker sequences comprises a plurality up to 500 of the microsatellites set out in Table 10;
   when the cancer is lymphoid neoplasm diffuse large B-cell lymphoma (DLBC), the set of microsatellite repeat marker sequences comprises a plurality up to 212 of the microsatellites set out in Table 11;
   when the cancer is uterine corpus endometrial carcinoma (UCEC), the set of microsatellite repeat marker sequences comprises a plurality up to 500 of the microsatellites set out in Table 12;
   when the cancer is kidney renal clear cell carcinoma (KIRC), the set of microsatellite repeat marker sequences comprises a plurality up to 508 of the microsatellites set out in Table 13;
   when the cancer is breast invasive carcinoma (BRCA), the set of microsatellite repeat marker sequences comprises a plurality up to 500 of the microsatellites set out in Table 14A; and
   when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.
84. The use of paragraph 75 or paragraph 76, wherein the set of microsatellite repeat marker sequences comprises a plurality up to 200 of the sequences set out in the respective Table for the subject's cancer type.
85. The use of paragraph 75 or paragraph 76, wherein the set of microsatellite repeat marker sequences comprises a plurality up to 300 of the sequences set out in the respective Table for the subject's cancer type.
86. The use of paragraph 75 or paragraph 76, wherein the set of microsatellite repeat marker sequences comprises a plurality up to the first 100 of the sequences set out in the respective Table for the subject's cancer type.
87. The use of paragraph 75 or paragraph 76, wherein the set of microsatellite repeat marker sequences comprises a plurality up to the first 200 of the sequences set out in the respective Table for the subject's cancer type.
88. The use of paragraph 75 or paragraph 76, wherein the set of microsatellite repeat marker sequences comprises a plurality up to the first 300 of the sequences set out in the respective Table for the subject's cancer type.
89. The use of any one of paragraphs 75-88, wherein the checkpoint inhibitor immunotherapy is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).
90. The use of any one of paragraphs 75-89, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).
91. The use of paragraph 75 or paragraph 76, wherein the processing of MSI-H or MSS is determined by a threshold value for the set of microsatellite marker sequences.
92. The use of paragraph 91, wherein the threshold for the set of microsatellite marker sequences is determined using the percentage of mutated microsatellite markers in the set to calculate the area under the receiver operating characteristic (AUROC).
93. The use of paragraph 92, wherein the smallest number of markers in the set necessary to reach a selected AUROC value is the threshold.
94. The use of paragraph 93, wherein the selected AUROC value is a value between 0.6 and 0.99, inclusive.
95. The use of paragraph 93, wherein the selected AUROC value is 0.9 or more.
96. The use of any one of paragraphs 75-95, wherein the checkpoint inhibitor immunotherapy is an inhibitor of PD-1 or PD-L1.
97. The use of any one of paragraphs 75-96, wherein the checkpoint inhibitor immunotherapy comprises a checkpoint inhibitor antibody.
98. The use of paragraph 76, wherein assaying loci in the defined set of microsatellite repeat marker sequences in cells of the subject's cancer for mutation comprises polynucleotide sequencing, measurement of total mutational burden, immunohistochemical analysis, and/or polymerase chain reaction.
99. The use of paragraph 98, wherein polynucleotide sequencing comprises whole genome sequencing, whole exome sequencing, or targeted gene capture sequencing.
100. The use of paragraph 75 or paragraph 76, wherein microsatellite mutations correspond to those identified in reference human genome GRCh37/hg19 translated to a different build of the human reference genome.
101. A diagnostic kit for determining microsatellite instability in a cancer, the kit comprising reagents that permit the detection of microsatellite mutation in a set of microsatellite repeat marker sequences in the cancer, wherein:
   when the cancer is colon adenocarcinoma (COAD), the set of microsatellite repeat marker sequences comprises a plurality up to 500 of the microsatellites set out in Table 1;
   when the cancer is esophageal carcinoma (ESCA), the set of microsatellite repeat marker sequences comprises a plurality up to 503 of the microsatellites set out in Table 2;
   when the cancer is glioblastoma multiforme (GBM), the set of microsatellite repeat marker sequences comprises a plurality up to 500 of the microsatellites set out in Table 3;
   when the cancer is lung adenocarcinoma (LUAD), the set of microsatellite repeat marker sequences comprises a plurality up to 500 of the microsatellites set out in Table 4;
   when the cancer is lung squamous cell carcinoma (LUSC), the set of microsatellite repeat marker sequences comprises a plurality up to 500 of the microsatellites set out in Table 5;
   when the cancer is rectum adenocarcinoma (READ), the set of microsatellite repeat marker sequences comprises a plurality up to 501 of the microsatellites set out in Table 6;
   when the cancer is stomach adenocarcinoma (STAD), the set of microsatellite repeat marker sequences comprises a plurality up to 500 of the microsatellites set out in Table 7;
   when the cancer is brain lower grade glioma (LGG), the set of microsatellite repeat marker sequences comprises a plurality up to 266 of the microsatellites set out in Table 8;
   when the cancer is prostate adenocarcinoma (PRAD), the set of microsatellite repeat marker sequences comprises a plurality up to 500 of the microsatellites set out in Table 9;
   when the cancer is cervical squamous cell carcinoma (CESC), the set of microsatellite repeat marker sequences comprises a plurality up to 500 of the microsatellites set out in Table 10;
   when the cancer is lymphoid neoplasm diffuse large B-cell lymphoma (DLBC), the set of microsatellite repeat marker sequences comprises a plurality up to 212 of the microsatellites set out in Table 11;
   when the cancer is uterine corpus endometrial carcinoma (UCEC), the set of microsatellite repeat marker sequences comprises a plurality up to 500 of the microsatellites set out in Table 12;
   when the cancer is kidney renal clear cell carcinoma (KIRC), the set of microsatellite repeat marker sequences comprises a plurality up to 508 of the microsatellites set out in Table 13;
   when the cancer is breast invasive carcinoma (BRCA), the set of microsatellite repeat marker sequences comprises a plurality up to 500 of the microsatellites set out in Table 14A; and
   when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.
102. The diagnostic kit for determining microsatellite instability of paragraph 101, wherein the set of microsatellite repeat marker sequences comprises a plurality up to 200 of the sequences set out in the respective Table for the subject's cancer type.
103. The diagnostic kit for determining microsatellite instability of paragraph 101, wherein the set of microsatellite repeat marker sequences comprises a plurality up to 300 of the sequences set out in the respective Table for the subject's cancer type.
104. The diagnostic kit for determining microsatellite instability of paragraph 101, wherein the set of microsatellite repeat marker sequences comprises a plurality up to the first 100 of the sequences set out in the respective Table for the subject's cancer type.
105. The diagnostic kit for determining microsatellite instability of paragraph 101, wherein the set of microsatellite repeat marker sequences comprises a plurality up to the first 200 of the sequences set out in the respective Table for the subject's cancer type.
106. The diagnostic kit for determining microsatellite instability of paragraph 101, wherein the set of microsatellite repeat marker sequences comprises a plurality up to the first 300 of the sequences set out in the respective Table for the subject's cancer type.
107. The diagnostic kit of any one of paragraphs 101-106, wherein the reagents comprise PCR primers that permit amplification of members of the set of microsatellite repeat marker sequences.
108. The diagnostic kit of paragraph 107, wherein the kit comprises an array of reagents that permit the detection of microsatellite mutation on a solid support.
109. An array for detecting microsatellite instability in a cancer, the array comprising nucleic acids that permit the detection of microsatellite mutation in a set of microsatellite repeat marker sequences in the cancer, wherein the nucleic acids are linked to a solid support.
110. The array of paragraph 109, wherein the nucleic acids are complementary to at least a portion of the microsatellite sequences.
111. The array of paragraph 109 or paragraph 110 wherein:
   when the cancer is colon adenocarcinoma (COAD), the set of microsatellite repeat marker sequences comprises a plurality up to 500 of the microsatellites set out in Table 1;
   when the cancer is esophageal carcinoma (ESCA), the set of microsatellite repeat marker sequences comprises a plurality up to 503 of the microsatellites set out in Table 2;
   when the cancer is glioblastoma multiforme (GBM), the set of microsatellite repeat marker sequences comprises a plurality up to 500 of the microsatellites set out in Table 3;
   when the cancer is lung adenocarcinoma (LUAD), the set of microsatellite repeat marker sequences comprises a plurality up to 500 of the microsatellites set out in Table 4;
   when the cancer is lung squamous cell carcinoma (LUSC), the set of microsatellite repeat marker sequences comprises a plurality up to 500 of the microsatellites set out in Table 5;
   when the cancer is rectum adenocarcinoma (READ), the set of microsatellite repeat marker sequences comprises a plurality up to 501 of the microsatellites set out in Table 6;
   when the cancer is stomach adenocarcinoma (STAD), the set of microsatellite repeat marker sequences comprises a plurality up to 500 of the microsatellites set out in Table 7;
   when the cancer is brain lower grade glioma (LGG), the set of microsatellite repeat marker sequences comprises a plurality up to 266 of the microsatellites set out in Table 8;
   when the cancer is prostate adenocarcinoma (PRAD), the set of microsatellite repeat marker sequences comprises a plurality up to 500 of the microsatellites set out in Table 9;
   when the cancer is cervical squamous cell carcinoma (CESC), the set of microsatellite repeat marker sequences comprises a plurality up to 500 of the microsatellites set out in Table 10;
   when the cancer is lymphoid neoplasm diffuse large B-cell lymphoma (DLBC), the set of microsatellite repeat marker sequences comprises a plurality up to 212 of the microsatellites set out in Table 11;
   when the cancer is uterine corpus endometrial carcinoma (UCEC), the set of microsatellite repeat marker sequences comprises a plurality up to 500 of the microsatellites set out in Table 12;
   when the cancer is kidney renal clear cell carcinoma (KIRC), the set of microsatellite repeat marker sequences comprises a plurality up to 508 of the microsatellites set out in Table 13;
   when the cancer is breast invasive carcinoma (BRCA), the set of microsatellite repeat marker sequences comprises a plurality up to 500 of the microsatellites set out in Table 14A; and
   when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.
112. The array of any one of paragraphs 109-111, wherein the set of microsatellite repeat marker sequences comprises a plurality up to 200 of the sequences set out in the respective Table for the subject's cancer type.
113. The array of any one of paragraphs 109-111, wherein the set of microsatellite repeat marker sequences comprises a plurality up to 300 of the sequences set out in the respective Table for the subject's cancer type.
114. The array of any one of paragraphs 109-111, wherein the set of microsatellite repeat marker sequences comprises a plurality up to the first 100 of the sequences set out in the respective Table for the subject's cancer type.
115. The array of any one of paragraphs 109-111, wherein the set of microsatellite repeat marker sequences comprises a plurality up to the first 200 of the sequences set out in the respective Table for the subject's cancer type.
116. The array of any one of paragraphs 109-111, wherein the set of microsatellite repeat marker sequences comprises a plurality up to the first 300 of the sequences set out in the respective Table for the subject's cancer type.
117. The array of any one of paragraphs 109-111, wherein the nucleic acids comprise PCR primers that permit amplification of members of the set of microsatellite repeat marker sequences.

### EXAMPLES

### Example 1: Optimal Diagnostic Markers for MSI

Microsatellite instability (MSI) predicts oncological response to checkpoint blockade immunotherapies. Although microsatellite mutation is pathognomonic for the condition, loci have unequal diagnostic value for predicting MSI within and across cancer types.

METHODS SUMMARY: To better inform molecular diagnosis of MSI, 9,438 tumor-normal exome pairs and 901 whole genome sequence pairs from 33 different cancer types were examined, and genome-wide microsatellite instability events were cataloged. Using a statistical framework, microsatellite mutations were identified that were predictive of MSI within and across cancer types. The diagnostic accuracy of different subsets of maximally informative markers was estimated computationally using a dedicated validation set.

RESULTS SUMMARY: Twenty-five cancer types exhibited hypermutated states consistent with MSI. Recurrently mutated microsatellites associated with MSI were identifiable in 15 cancer types, but were largely specific to individual cancer types. Cancer-specific microsatellite panels of one to seven loci were needed to attain 95% diagnostic sensitivity and specificity for 11 cancer types, and in eight of the cancer types, 100% sensitivity and specificity were achieved. Breast cancer required 800 loci to achieve comparable performance, and recurrent microsatellite mutations were not identified supporting reliable MSI diagnosis in ovarian tumors. Features associated with informative microsatellites were cataloged.

CONCLUSION SUMMARY: Most microsatellites informative for MSI are specific to particular cancer types, requiring the use of tissue-specific loci for optimal diagnosis. Limited numbers of markers are needed to provide accurate MSI diagnosis in most tumor types, but it is challenging to diagnose breast and ovarian cancers using pre-defined microsatellite locus panels.

### Background

Microsatellite instability (MSI) is a molecular tumor phenotype that is indicative of genomic hypermutability, usually reflecting inactivation of the mismatch repair (MMR) system. MSI is marked by spontaneous gains or losses of nucleotides from repetitive DNA tracts, resulting in new alleles of differing length that serve as the basis for its clinical diagnosis. Although classically associated with colorectal and endometrial tumors, MSI has now been recognized in most cancer types with varying prevalence and is accompanied by a generally increased rate of mutations genome-wide. Testing for MSI has subsequently emerged as a pan-cancer biomarker of therapeutic response to PDL-1 and PD-1 immune checkpoint inhibitors, where the MSI positive (microsatellite high, or MSI-H) phenotype is believed to serve as an indicator of mutation-associated neoantigens that permit a more robust T lymphocyte response than for MSI negative (microsatellite stable, or MSS) cases.

Molecular diagnosis of MSI in clinical practice is most commonly achieved using multiplexed PCR of defined microsatellite loci, followed by capillary electrophoresis to qualitatively detect new alleles (MSI-PCR). Quantitative next-generation sequencing (NGS) methods have been developed to identify MSI by assessing overall microsatellite mutation frequency at repetitive loci that are either directly targeted or incidentally captured by targeted gene enrichment oncology panels. Nevertheless, both conventional and NGS approaches interrogate markedly limited subsets of the millions of microsatellite markers available in the human genome: only five loci are included in standard MSI-PCR, whereas dozens to hundreds of sites are examined by typical NGS approaches.

Studies have revealed tissue-specific signatures of microsatellite mutation, such that alterations in specific loci can occur with disparate frequencies in tumors from different tissues. Consequently, microsatellites that are diagnostic for MSI potentially have unequal prognostic value within and across tumor types, such that loci useful in one cancer type may not yield accurate diagnoses in others. Supporting this hypothesis, standard MSI-PCR markers were developed for use in colon cancers and can exhibit poor performance in other malignancies.

The choice of markers is therefore critical to maximize sensitivity and specificity of molecular MSI diagnosis, regardless of whether testing is performed by conventional or NGS methods. Nevertheless, little effort to date has focused on using systematic, genome-scale analysis to identify optimal microsatellite loci for diagnosing MSI. Here, tumor-normal pairs of exome and whole genome data from 33 different cancer types were systematically evaluated in order to ascertain the most informative microsatellites for predicting MSI.

### Methods:

### Sequence Data and Identification of Microsatellite Loci

Genomic microsatellite loci were identified as previously, with some modifications; see e.g., Hause et al. Nat Med. 2016, 22:1342-50, the contents of which are incorporated herein by reference in its entirety. Briefly, microsatellites were defined in the human genome (GRCh37/hg19; see e.g., Table 18 for sequence references) as repeating subunits of 1-5 bp in length and comprising ≥5 repeats using MISA. Adjacent microsatellites within ten base pairs of each other were termed ‘complex’ (c*) single loci if comprised of tracts with different repeating subunit lengths or ‘compound’ (c) single loci for those having the same repeat length. This analysis defined 19,035,602 loci, of which the 18,882,838 present on autosomes and chromosome X were retained. Repeat features were annotated using ANNOVAR (24 Feb. 2014 release).

Sequence alignments of tumors and patient-matched normal specimens from exome and whole genome sequencing projects were obtained from TCGA Research Network (see e.g., cancergenome.nih.gov/). Alignments were standardized prior to analysis by converting alignments to FASTQ files using PICARD v1.98, re-aligning to GRCh37/hg19 using BWA-MEM v0.7.12, and indexing with SamTools v1.1.

### Cataloging Microsatellite Instability Events.

For each tumor and normal specimen, the number of sequence reads supporting different tract lengths at each locus were quantified using mSINGS (Git Commit ID a7e9ea9).

To identify instability events, multinomial distributions of allele lengths for tumor at each locus were compared to the joint multinomial distribution of allele lengths across tumor and normal at the site by:

(X_{wildtype_allele},X_{alternative_allele_1}, . . . X_{alternative_allele_i})˜Mult(n,p)

where which n refers to the number of reads at a site and p refers to the proportion supporting each alternative allelic length. “Unstable” microsatellites (those evidencing somatic mutations) were defined as those with nominally significant differences (p<0.05) by likelihood ratio (G) tests without continuity correction. Rates for calling false positive instability at this heuristic threshold were estimated as <3% at all sites having ≥2 reads in tumor and ≥1 read in its paired normal by simulating and comparing two distributions of 1,000 normal sites with median observed multinomial distributions of allele lengths by:

Mult(n=read_depth,P=0.9_{wildtype_allele},0.1_{alternative_alleles})

To confidently define “stable” sites in tumor (those lacking somatic mutations), sites were simulated from empirically observed multinomial read distributions for highly unstable sites in cases having >30 read coverage in both tumor and normal by:

Mult(1000,p=0.4_{wildtype_allele},0.6_{alternative_alleles})

Down-sampling analyses estimated that ≥18 reads per site yielded 95% power for identifying an unstable locus, providing strong evidence to conclude that a site was “stable” if no difference in allelic distribution was observed at that coverage. Sites with 5-18 reads in both tumor and paired normal but no indication of instability (p>0.05), and those covered by fewer than five reads in either tumor or normal samples were marked as “missing data”.

As a quality control measure, samples were excluded having ≥75% missing data, leaving 9,438 tumor-normal exome pairs and 901 whole genome pairs for subsequent analysis. Similarly, individual loci were excluded for which ≥75% of specimens evidenced missing data.

### Gaussian Mixture Model to Classify MSI Status

The overall frequency of microsatellite mutations was quantified for each tumor as the fraction of unstable sites over total callable sites, and given the skewed nature of the data, log₁₀ transformation was performed. For each cancer type, a Gaussian mixture model was then fit to these values using MCLUST v5.4.5 with one or two mixture components with equal variance. If the two-component model could be validly applied to a cancer type, individual tumors were classified as MSS (lower mode), MSI-H (higher mode), or indeterminate (uncertainty value >0.1). If distributions were instead consistent with a single component, all tumors of that cancer type were classified as MSS.

### Identifying and Modeling Predictive Abilities of Informative Markers

After excluding “intermediate” MSI classifications, 80% of tumors in each cancer type were randomly assigned into training sets and 20% into validation sets. Using the training sets for each tissue type, microsatellites were identified that were most frequently mutated in MSI-H relative to MSS tumors by Fisher's exact test. The proportions of stable and unstable sites between MSS and MSI-H tumors were compared (excluding missing data), allowing loci to be rank-ordered by p value. For each tissue type, subsets of the top n most informative loci (ranging from 1 to 2,000 markers) were then selected and the percentage of unstable loci for each sample (excluding missing data from both numerator and denominator) was calculated. These values were used to calculate the area under the receiver operating characteristic (AUROC) using pROC v1.15.3. The optimal percentage on the receiver operating characteristic curve was identified by Youden's J statistic. This value was subsequently used as the threshold to assign MSS or MSI-H classification to each sample based on the fraction of mutated markers identified from the simulated panel and to determine sensitivity and specificity for each tissue type.

To account for the possibility of more complex trans-genomic interactions between sites not captured by an additive model of instability, machine learning approaches were explored including random forest and boosted trees. Iterative reduction of marker features was performed using both Fisher exact test p values and Shapley feature importance values in the training dataset. However, these approaches did not meaningfully outperform the simpler, additive model.

### Identification of MSI-Associated Microsatellite Mutations Common to Multiple Tissue Types

The top 2,000 loci most strongly associated with MSI-H status for each tissue type were examined for cross-performance across tissue types by hierarchical clustering of normalized p values. Normalization was required to account for differences in the uneven sample sizes, and was accomplished by log 10-transformation of raw p values, followed by scaling on a per-tissue basis from a range of zero (least significant) to one (most significant). A heatmap was generated using superheat v0.1.0, and pairwise and cophenetic distances between tissue types were subsequently calculated and compared using the base-R dist, cophenetic, and cor functions.

### Features of Informative Microsatellites

Enrichment of particular locus features (annotated genic context of the repeat, repeat class, and number of repeat subunits in deciles) was examined among individual microsatellite associations with MSI-H status using linear regression. Intergenic annotation, pentanucleotide repeats, and a length of 5-11 repeats, respectively, were arbitrarily selected as reference levels for these analyses. Whole genome data were used for this analysis, as they more comprehensively represented feature annotations across coding and noncoding regions than exome data.

### Results:

### Prevalence of Microsatellite Mutation and MSI-H Tumors Vary Across Cancer Types

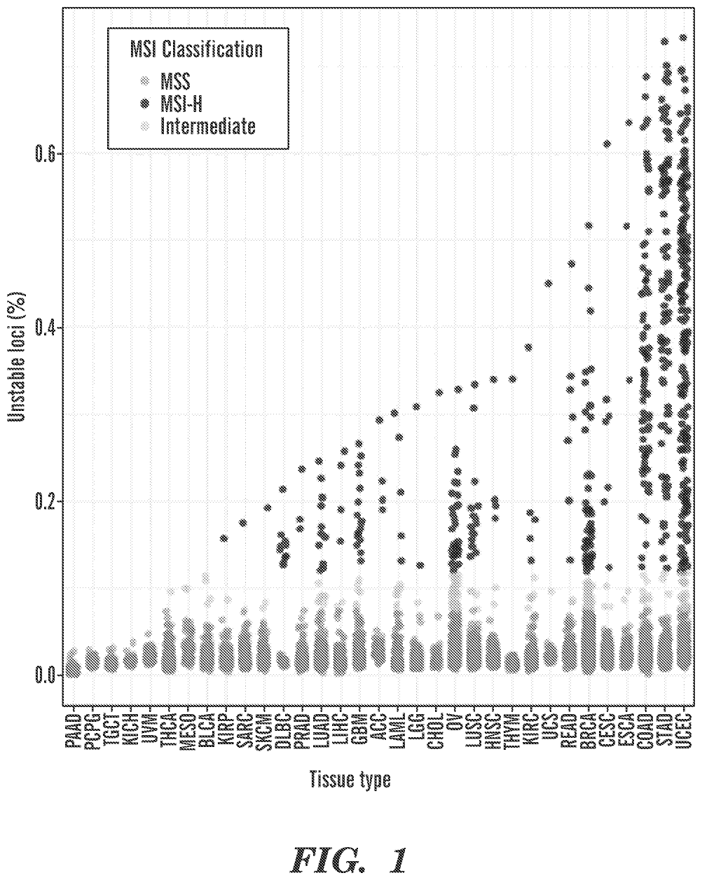

To ascertain the relative degree of genomic instability within and across cancer types, the overall frequency of microsatellite mutations was assessed within each of 33 cancer types using paired tumor-normal exome sequencing data. For each cancer type, Gaussian mixture modeling was used to circumscribe subpopulations of tumors having high burdens of microsatellite mutation, corresponding to MSI-H cases (see e.g., FIG. 1A, Table 1; see e.g., Supplemental Table 1 in Appendix A of U.S. Provisional Application No. 63/044,029 filed Jun. 25, 2020, the contents of which are incorporated herein by reference in their entirety). Comparison of MSI classifications established by this approach showed high agreement with determinations made by “gold-standard” MSI-PCR (accuracy 97.8%, 95% CI 95.5%-98.35%) or MOSAIC genome-scale analysis (accuracy 98.8%, 95% CI 98.5%-99.1%), supporting their validity.

TABLE 16
Tumor Exome Sequences and inferred MSI diagnoses

| Cancer Type (TCGA | Fraction of Cases (Count) | | | | | |
|---|---|---|---|---|---|---|
| Code)* | MSS | | Intermediate | | MSI-H | |
| BLCA | 0.99 | (375) | 0.01 | (4) | 0 | (0) |
| BRCA | 0.9 | (866) | 0.04 | (43) | 0.06 | (53) |
| CESC | 0.97 | (206) | 0.005 | (1) | 0.02 | (5) |
| CHOL | 0.98 | (49) | 0 | (0) | 0.02 | (1) |
| COAD | 0.79 | (309) | 0.02 | (8) | 0.19 | (74) |
| DLBC | 0.77 | (36) | 0 | (0) | 0.23 | (11) |
| ESCA | 0.98 | (145) | 0 | (0) | 0.02 | (3) |
| GBM | 0.95 | (356) | 0.01 | (4) | 0.04 | (14) |
| HNSC | 0.99 | (506) | 0.004 | (2) | 0.01 | (4) |
| KICH | 1 | (64) | 0 | (0) | 0 | (0) |
| KIRC | 0.97 | (321) | 0.02 | (5) | 0.02 | (5) |
| KIRP | 0.99 | (248) | 0.004 | (1) | 0.004 | (1) |
| LAML | 0.92 | (113) | 0.07 | (8) | 0.02 | (2) |
| LGG | 1 | (494) | 0 | (0) | 0.004 | (2) |
| LIHC | 0.99 | (329) | 0.003 | (1) | 0.01 | (4) |
| LUAD | 0.97 | (525) | 0.01 | (7) | 0.02 | (10) |
| LUSC | 0.96 | (436) | 0.01 | (4) | 0.03 | (12) |
| MESO | 0.98 | (59) | 0.02 | (1) | 0 | (0) |
| OV | 0.87 | (339) | 0.09 | (34) | 0.05 | (18) |
| PAAD | 1 | (174) | 0 | (0) | 0 | (0) |
| PCPG | 1 | (184) | 0 | (0) | 0 | (0) |
| PRAD | 0.99 | (455) | 0 | (0) | 0.01 | (3) |
| READ | 0.94 | (133) | 0.01 | (2) | 0.05 | (7) |
| SARC | 0.99 | (186) | 0.01 | (1) | 0.01 | (1) |
| SKCM | 0.99 | (439) | 0.005 | (2) | 0.002 | (1) |
| STAD | 0.79 | (328) | 0.005 | (2) | 0.2 | (84) |
| TGCT | 1 | (156) | 0 | (0) | 0 | (0) |
| THCA | 1 | (454) | 0.002 | (1) | 0 | (0) |
| THYM | 0.99 | (118) | 0 | (0) | 0.01 | (1) |
| UCEC | 0.57 | (255) | 0.04 | (17) | 0.4 | (179) |
| UCS | 0.96 | (55) | 0.02 | (1) | 0.02 | (1) |
| UVM | 1 | (80) | 0 | (0) | 0 | (0) |

In Table 16, “*” indicates the following TCGA codes: BLCA, Bladder Urothelial Carcinoma; BRCA, Breast invasive carcinoma; CESC, Cervical squamous cell carcinoma and endocervical adenocarcinoma; CHOL, Cholangiocarcinoma; COAD, Colon adenocarcinoma; DLBC, Lymphoid Neoplasm Diffuse Large B-cell Lymphoma; ESCA, Esophageal carcinoma; GBM, Glioblastoma multiforme; HNSC, Head and Neck squamous cell carcinoma; KICH, Kidney Chromophobe; KIRC, Kidney renal clear cell carcinoma; KIRP, Kidney renal papillary cell carcinoma; LAML, Acute Myeloid Leukemia; LGG, Brain Lower Grade Glioma; LIHC, Liver hepatocellular carcinoma; LUAD, Lung adenocarcinoma; LUSC, Lung squamous cell carcinoma; MESO, Mesothelioma; OV, Ovarian serous cystadenocarcinoma; PAAD, Pancreatic adenocarcinoma; PCPG, Pheochromocytoma and Paraganglioma; PRAD, Prostate adenocarcinoma; READ, Rectum adenocarcinoma; SARC, Sarcoma; SKCM, Skin Cutaneous Melanoma; STAD, Stomach adenocarcinoma; TGCT, Testicular Germ Cell Tumors; THCA, Thyroid carcinoma; THYM, Thymoma; UCEC, Uterine Corpus Endometrial Carcinoma; UCS, Uterine Carcinosarcoma; UVM, Uveal Melanoma.

Twenty-five of 32 cancer types evidenced one or more hypermutated tumors consistent with an MSI-H phenotype, ranging in incidence from 0.2% to 40%, similar to other reports; see e.g., Hause et al. Nat Med. 2016, 22:1342-50; Cortes-Ciriano et al. Nat Commun. 2017, 8:15180; Bonneville et al. JCO Precision Oncology. 2017, 1-15; the contents of each of which are incorporated herein by reference in their entireties. The total fraction of mutated microsatellites in MSI-H tumors varied considerably across cancer types, with the greatest microsatellite mutation burdens occurring in stomach, colon, and endometrial tumors (see e.g., FIG. 1).

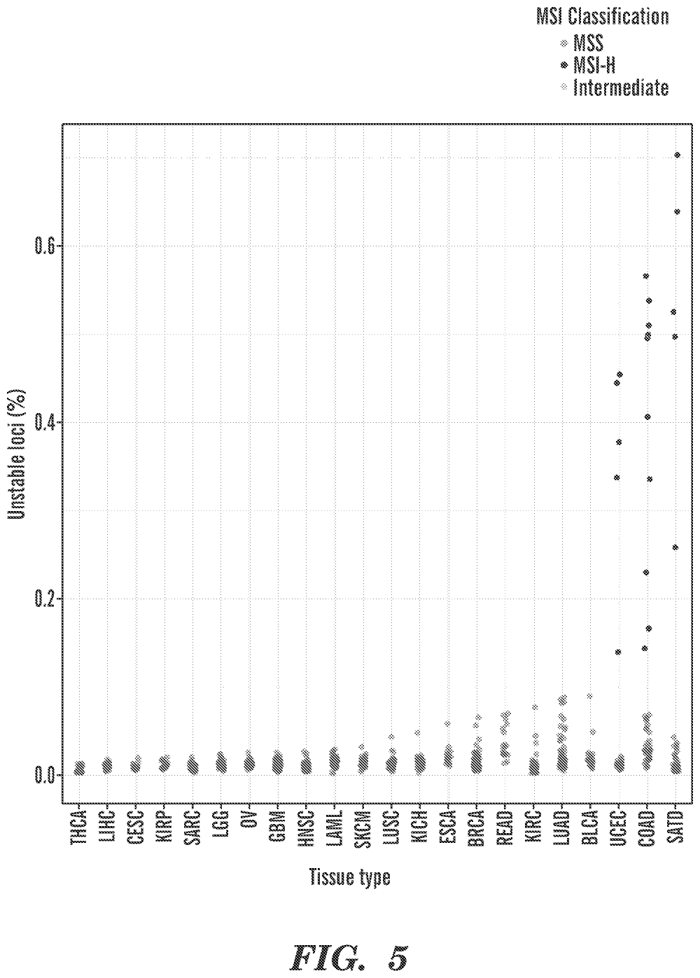

Parallel analyses were performed using whole genome data (see e.g., FIG. 5, Supplemental Table 2 in Appendix A of U.S. Provisional Application No. 63/044,029 filed Jun. 25, 2020, the contents of which are incorporated herein by reference in their entirety), which included intergenic regions but had lower sequencing read depths and were available for fewer cases and cancer types. Although data were sparser, results from whole genome analysis were consistent with those from exome data.

### Informative Microsatellite Markers Differ Between Cancer Types

Next, microsatellite mutations most predictive of MSI were identified by cataloging events occurring significantly in MSI-H relative to MSS tumors of each cancer type. This analysis was restricted to cancers for which locus performance could be evaluated in both testing and validation sets, permitting examination of 15 cancer types by exome data alone (see e.g., Supplemental Table 3 in Appendix A of U.S. Provisional Application No. 63/044,029 filed Jun. 25, 2020, the contents of which are incorporated herein by reference in their entirety).

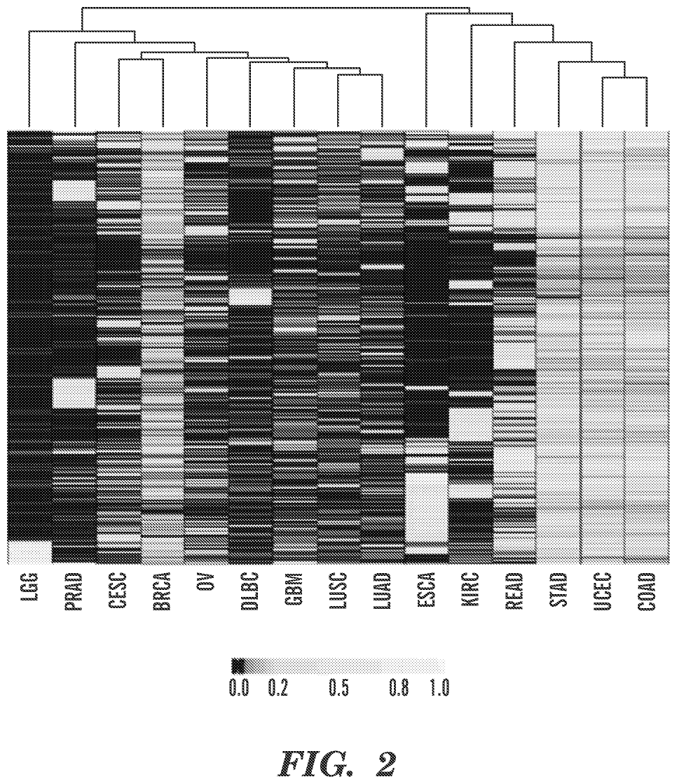

Hierarchical clustering (see e.g., FIG. 2) revealed that cancer types displayed markedly different, and largely non-overlapping subsets of informative microsatellites from across the genome. Correlation between measurements of pairwise (see e.g., Table 19) and cophenetic (see e.g., Table 20) distance matrices of locus informativity was 0.82, indicating that clustering accurately represents a large degree of variation in patterns of MSI-associated mutation across cancers. Endometrial and colon tumors were most similar by these metrics, sharing 34.6% of informative sites (4,767 loci), whereas esophageal carcinoma and brain lower grade glioma were most disparate, having only 5% informative microsatellites (79 loci) in common. Similarities in microsatellite mutation patterns were apparent among rectal, stomach, colon and uterine corpus endometrial tumors, as well as between lung adenocarcinoma and squamous cell carcinoma (see e.g., FIG. 2, Supplemental Table 3 in Appendix A of U.S. Provisional Application No. 63/044,029 filed Jun. 25, 2020, the contents of which are incorporated herein by reference in their entirety). However, most cancer types evidenced distinct profiles of microsatellite mutations. Thus, the great majority of loci whose mutation correlates with an MSI-H phenotype are specific to particular cancer types.

Predictive Value of Microsatellites for MSI Diagnosis within and Across Cancer Types

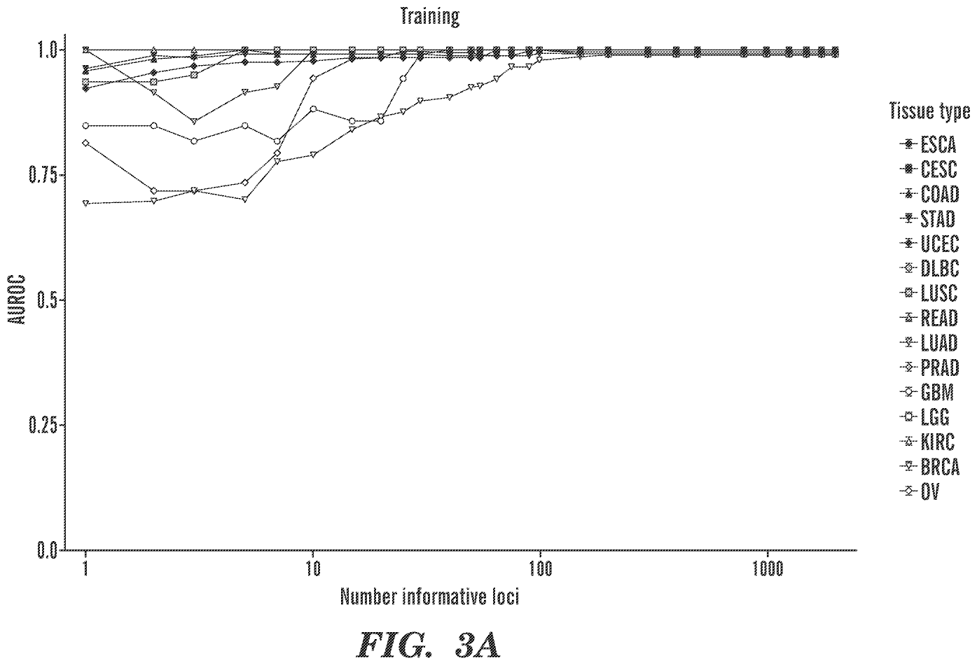

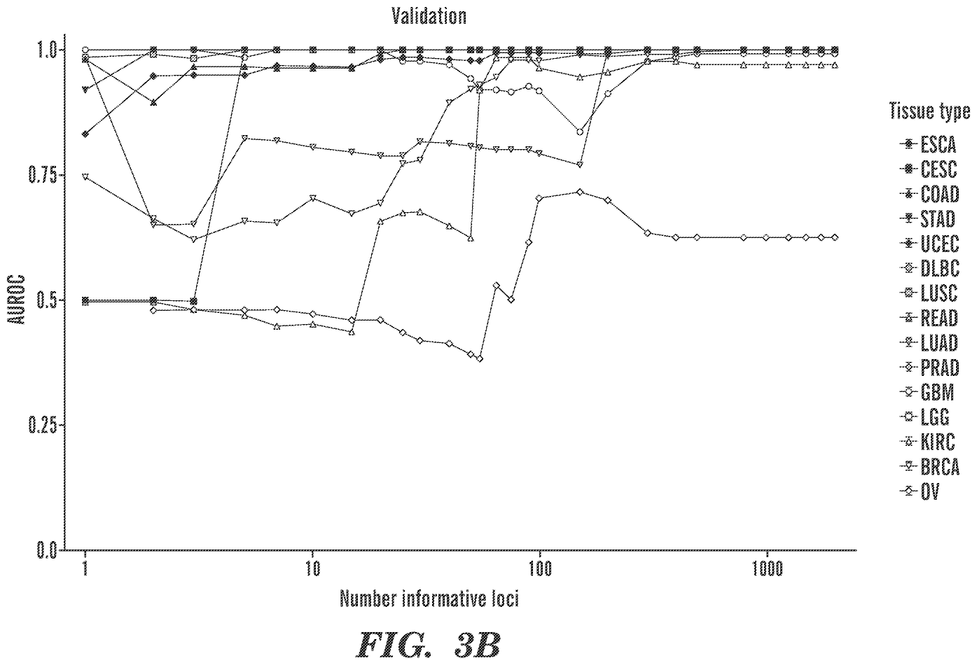

Subsets of one to 2,000 of the most highly informative loci identified per cancer type were used to computationally evaluate their performance for diagnosing MSI within their tumor type of origin, using an independent set of tumor samples for validation (see e.g., FIG. 3). Specimens were classified as MSS or MSI-H based on the fraction of mutated microsatellites observed in a given panel, with optimal thresholds for discriminating these classifications determined empirically (see e.g., Table 17). These classifications were compared against determinations made from exome-wide analysis (see e.g., Table 16) to assess their accuracy.

The number of markers needed to achieve MSI diagnosis was initially determined with at least 95% sensitivity and specificity (see e.g., FIG. 3, Table 17). The number of requisite markers varied considerably across tumor types. For 11 of the 15 cancer types, seven or fewer markers provided the requisite performance characteristics, and in eight cancers MSI could be diagnosed with 100% specificity and sensitivity. Diagnosis of MSI in endometrial tumors required 20 markers, while 65 were needed for classification of kidney renal clear cell cancers. In contrast, MSI determination in breast cancer required 800 markers to achieve comparable performance. MSI diagnosis in ovarian tumors, while favorable for the training set (see e.g., FIG. 3A), did not permit reliable diagnosis in the validation set using any number of markers considered (see e.g., FIG. 3B, Table 17).

The desired balance of sensitivity and specificity can vary by clinical application, and relates to the number of markers examined. Therefore, the predictive capacity of various numbers of markers was additionally determined as measured by the AUROC and the number of makers required to achieve an area under the curve (AUC) of 0.9 or greater (see e.g., Table 17). Although the number of markers required for most cancers by this metric remained similar, decreases for breast (from 800 to 50), kidney renal clear cell (65 to 55), endometrial (20 to 2), and stomach (2 to 1) were observed.

A small number of loci were informative for MSI across multiple cancer types. Therefore, informative microsatellites were examined in endometrial, colon, rectal, and stomach cancers, which collectively showed closely related mutational patterns, in order to determine whether a common marker panel could be used to diagnose MSI in those tumors. After Bonferroni correction, 37 shared microsatellites were independently associated with MSI-H status in each of those four cancer types (see e.g., Table 21). The 37-marker panel demonstrated favorable performance characteristics for the four specific cancer types (0.98 AUC, sensitivity 94.3%, specificity 97.7%), but did not outperform respective tissue-type specific marker panels (see e.g., Table 17) and functioned poorly when applied to other cancers. For example, AUC was 0.45 when the panel was applied to lung squamous cell carcinoma and lung adenocarcinoma, compared with AUC 0.95 for a similarly sized panel specific to those cancer types.

TABLE 17
Performance characteristics of tissue-specific microsatellite
panels for MSI diagnosis in validation set; “*” indicates
that the cancer did not achieve cutoff for test-characteristic
threshold with the maximum number of loci tested.

| | Smallest panel achieving sensitivity and specificity of 95% | | | | |
|---|---|---|---|---|---|
| | | Min unstable | | | |
| Cancer | | markers for | | | |
| Type | | MSI-H | | | |
| (TCGA | # of | classification | | | |
| Code) | markers | (%) | AUC | Sensitivity | Specificity |
| COAD | 1 | 50.0% | 0.98 | 100.0% | 95.8% |
| ESCA | 1 | 50.0% | 1.00 | 100.0% | 100.0% |
| GBM | 1 | 50.0% | 1.00 | 100.0% | 100.0% |
| LUAD | 1 | 50.0% | 0.99 | 100.0% | 97.1% |
| LUSC | 1 | 50.0% | 0.99 | 100.0% | 97.1% |
| READ | 2 | 50.0% | 1.00 | 100.0% | 100.0% |
| STAD | 2 | 25.0% | 1.00 | 100.0% | 100.0% |
| LGG | 5 | 12.5% | 1.00 | 100.0% | 100.0% |
| PRAD | 5 | 16.7% | 1.00 | 100.0% | 100.0% |
| CESC | 7 | 45.8% | 1.00 | 100.0% | 100.0% |
| UCEC | 20 | 12.9% | 0.98 | 97.0% | 97.9% |
| KIRC | 65 | 7.5% | 0.99 | 100.0% | 98.5% |
| BRCA | 800 | 3.6% | 1.00 | 100.0% | 99.5% |
| OV* | 2000 | 2.0% | 0.63 | 100.0% | 47.8% |

| | Smallest panel achieving AUROC of 0.9 | | | | |
|---|---|---|---|---|---|
| | | Min unstable | | | |
| | | markers for | | | |
| | | MSI-H | | | |
| | # of | classification | | | |
| | markers | (%) | AUC | Sensitivity | Specificity |
| COAD | 1 | 50.0% | 0.98 | 100.0% | 95.8% |
| ESCA | 1 | 50.0% | 1.00 | 100.0% | 100.0% |
| GBM | 1 | 50.0% | 1.00 | 100.0% | 100.0% |
| LUAD | 1 | 50.0% | 0.99 | 100.0% | 97.1% |
| LUSC | 1 | 50.0% | 0.99 | 100.0% | 97.1% |
| READ | 2 | 50.0% | 1.00 | 100.0% | 100.0% |
| STAD | 1 | 50.0% | 0.92 | 84.2% | 100.0% |
| LGG | 5 | 12.5% | 1.00 | 100.0% | 100.0% |
| PRAD | 5 | 16.7% | 1.00 | 100.0% | 100.0% |
| CESC | 7 | 45.8% | 1.00 | 100.0% | 100.0% |
| DLBC | 7 | 25.0% | 1.00 | 100.0% | 100.0% |
| UCEC | 2 | 25.0% | 0.95 | 90.3% | 97.2% |
| KIRC | 55 | 4.6% | 0.93 | 100.0% | 86.6% |
| BRCA | 50 | 4.3% | 0.92 | 100.0% | 81.1% |
| OV* | 2000 | 2.0% | 0.63 | 100.0% | 47.8% |

### Properties of Informative Microsatellites

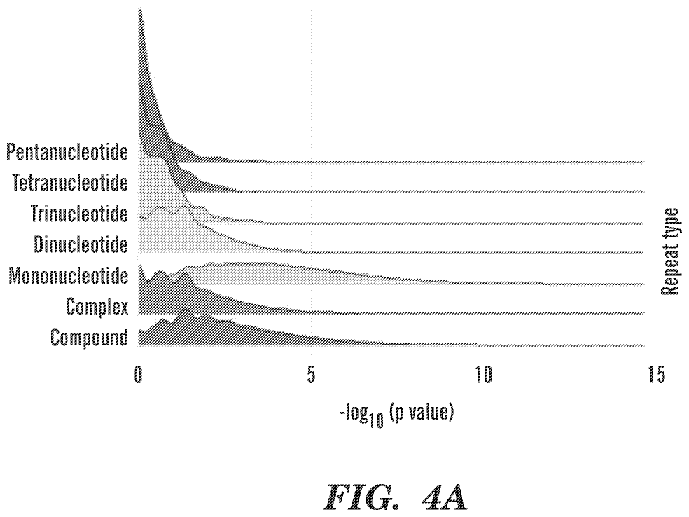

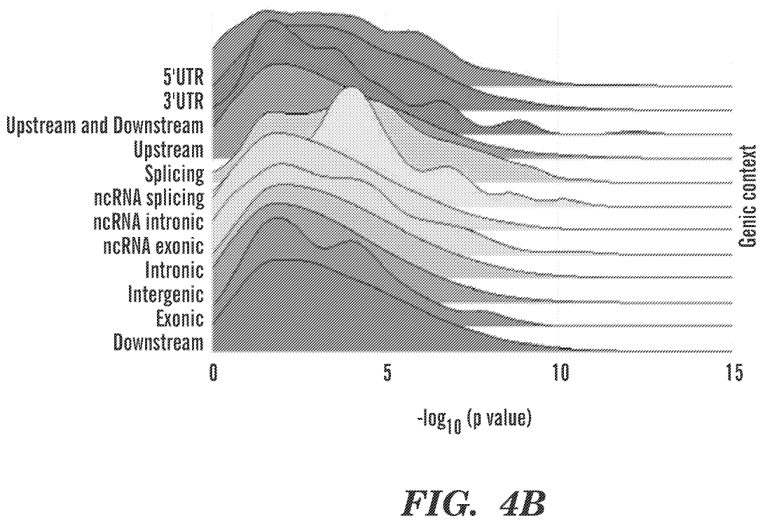

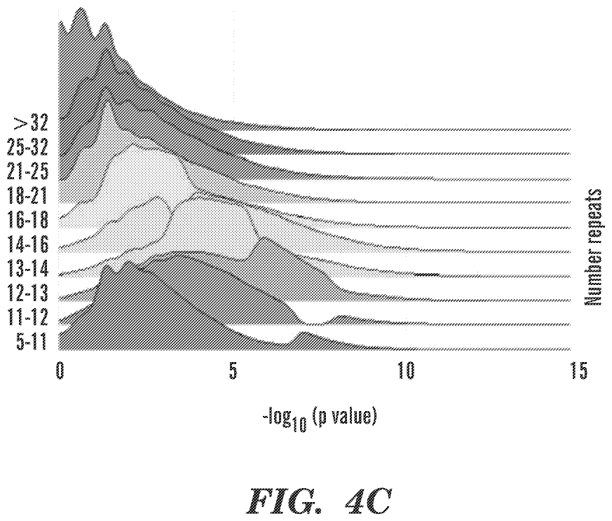

The sequence composition and genic feature annotations that were enriched in microsatellites having globally high informativity were examined for MSI-H tumors (see e.g., FIG. 4, Table 22). Whole genome data were examined in order to allow exploration of coding and noncoding regions; this approach showed agreement with analyses of exome sequencing where direct comparisons were possible (data not shown). Of the different types of repeat sequence classes examined (see e.g., FIG. 4A), mononucleotide microsatellite mutations correlated most highly with MSI-H, followed by complex repeats (which themselves may be composed of multiple mononucleotide repeats). MSI-informative markers were most significantly enriched in splice sites (see e.g., FIG. 4B), and conversely, intergenic regions and noncoding intronic annotations contained the fewest informative microsatellites elements. Notably, a non-linear correlation was found between the length of mononucleotide markers and their informativity (see e.g., FIG. 4C and FIG. 4D). Tracts comprised of 12 to 13 repeats proved most informative, with longer or shorter loci showing a decrease in informativity proportional to their distance from this maximum.

### CONCLUSIONS

Herein, genomic analyses were used to prioritize microsatellite markers that are most informative for diagnosing MSI by molecular methods. Whereas other work has cataloged loci that are frequently mutated in MSI-H tumors from the three or four cancer types where the phenotype occurs most often (see e.g., Hause et al., 2016, supra; Cortes-Ciriano et al., 2017, supra), herein microsatellite mutation occurrence was more broadly examined across cancer types and the performance of variously sized marker subsets was also evaluated for classifying MSI-H tumors in clinical practice.

Cancer types exhibit distinct patterns of microsatellite mutation overall, and herein it was found that microsatellites that are informative for diagnosing MSI also vary across cancer types (see e.g., FIG. 2). These results strongly argue that MSI diagnostic panels developed for particular tumor types should not be considered generally applicable across all cancers. Although a small subset of markers was cross-informative across a limited group of cancer types that shared similar mutational profiles (see e.g., Table 17), microsatellites tailored to a specific cancer type provided maximal diagnostic accuracy.

Accordingly, in determining markers with the highest diagnostic utility (see e.g., FIG. 4), it was observed that microsatellite mutations within functional elements including splice sites and exons were significantly enriched, supporting the notion that biological pressures are involved in selecting microsatellite mutations associated with MSI. It was also observed that mononucleotide microsatellites, loci occurring in splicing regions, and microsatellites comprised of 12 to 13 repeat subunits provided the greatest diagnostic benefit, with microsatellites of 18 or greater repeats being significantly associated with stability. This latter finding indicates that, unlike in in vitro systems, MSI phenotypes in vivo preferentially involve tracts of specific lengths.

The performance was evaluated of variously sized subsets of the most informative loci per cancer type in data sets withheld from those used to identify informative loci (see e.g., FIG. 3, Table 17). Due to the degree of missing microsatellite calls inherent to the dataset, existing performance estimates are likely to be conservative. Even so, eight of the 15 cancer types achieved 100% sensitivity and specificity using seven or fewer loci, and in two of those cancer types only a single marker was required. These modest testing requirements are compatible with MSI diagnosis by highly focused methods including conventional MSI-PCR or targeted NGS. Endometrial and kidney renal clear cell tumors required slightly more markers (20 and 65, respectively). In two extreme cases, breast cancer required 800 markers whereas reliable MSI diagnosis could not be achieved in ovarian cancer using pre-defined subsets of microsatellite markers. These findings are consistent with microsatellites following less stereotyped patterns of mutation for breast and ovarian cancers, possibly owing to instability events having more neutral fitness effects for breast and ovarian tumors and subsequently resulting in fewer recurrently mutated loci. In these cancer types, and possibly others, a measurement of overall genome-wide microsatellite mutation burden can be required to reliably establish MSI diagnosis (see e.g., FIG. 1).

Although MSI status is currently an approved diagnostic marker to indicate eligibility for PDL-1 and PD-1 inhibitor treatments, alternative methods for testing tumor susceptibility to those immunotherapies are now available. The most widely used of these alternative approaches is tumor mutation burden (TMB), a biomarker based on estimating the total substitution and indel lesions present in a cancer genome. Nevertheless, TMB determination requires sequencing large gene panels and is of contested clinical utility. Because immunotherapy response is particularly associated with insertion-deletion mutational load, MSI is considered a more reliable positive predictor of treatment outcomes even though it is unable to identify all cancers for which a favorable response can be achieved. Although MSI and TMB determinations frequently overlap, they provide distinct information. Given these considerations, MSI and TMB can be considered complementary, and dedicated testing for MSI can continue to provide utility as an inexpensive, primary screening method for immunotherapy response.

The tissue-specific diagnostic performance of loci identified herein could not be directly compared to microsatellite loci included in clinical MSI assays due to both the unknown sensitivity of NGS relative to MSI-PCR for detecting microsatellite mutations and inadequate read depths for those markers in both exome and whole genome data. It is noteworthy that the most informative markers identified by these analyses do not overlap with those utilized in standard clinical assays for MSI.

Nonstandard abbreviations include the following: MSI, microsatellite instability; MMR, mismatch repair; MSI-H, microsatellite instability high; MSS, microsatellite stable; NGS, Next-Generation DNA sequencing; TMB, tumor mutation burden; AUC, area under the curve; AUROC, area under the receiver operating characteristic. Human gene abbreviations include at least the following: PD-1, programmed cell death 1; PDL-1, programmed cell death 1 ligand 1. All gene abbreviations herein (e.g., in Tables 1-15) are used as known in art.

References include the following, the contents of each of which are incorporated herein by reference in their entireties:
1. Vilar E, Gruber S B. Microsatellite instability in colorectal cancer—the stable evidence. Nature reviews Clinical oncology. 2010; 7:153-62.
2. Murphy K M, Zhang S, Geiger T, Hafez M J, Bacher J, Berg K D, et al. Comparison of the microsatellite instability analysis system and the Bethesda panel for the determination of microsatellite instability in colorectal cancers. The Journal of molecular diagnostics: JMD. 2006; 8:305-11.
3. Baudrin L G, Deleuze J-F, How-Kit A. Molecular and Computational Methods for the Detection of Microsatellite Instability in Cancer. Front Oncol. 2018; 8: 621.
4. Zhang L. Immunohistochemistry versus microsatellite instability testing for screening colorectal cancer patients at risk for hereditary nonpolyposis colorectal cancer syndrome. Part II. The utility of microsatellite instability testing. The Journal of molecular diagnostics: JMD. 2008; 10:301-7.
5. Ribic C M, Sargent D J, Moore M J, Thibodeau S N, French A J, Goldberg R M, et al. Tumor microsatellite-instability status as a predictor of benefit from fluorouracil-based adjuvant chemotherapy for colon cancer. The New England journal of medicine. 2003; 349: 247-57.
6. Beamer L C, Grant M L, Espenschied C R, Blazer K R, Hampel H L, Weitzel J N, et al. Reflex immunohistochemistry and microsatellite instability testing of colorectal tumors for Lynch syndrome among US cancer programs and follow-up of abnormal results. Journal of clinical oncology: official journal of the American Society of Clinical Oncology. 2012; 30:1058-63.
7. Hause R J, Pritchard C C, Shendure J, Salipante S J. Classification and characterization of microsatellite instability across 18 cancer types. Nat Med. 2016; 22:1342-50.
8. Cortes-Ciriano I, Lee S, Park W-Y, Kim T-M, Park P J. A molecular portrait of microsatellite instability across multiple cancers. Nat Commun. 2017; 8:15180.
9. Bonneville R, Krook M A, Kautto E A, Miya J, Wing M R, Chen H-Z, et al. Landscape of Microsatellite Instability Across 39 Cancer Types. JCO Precision Oncology. 2017; 1-15.
10 Maruvka Y E, Mouw K W, Karlic R, Parasuraman P, Kamburov A, Polak P, et al. Analysis of somatic microsatellite indels identifies driver events in human tumors. Nat Biotechnol. 2017; 35:951-9.
11. Vanderwalde A, Spetzler D, Xiao N, Gatalica Z, Marshall J. Microsatellite instability status determined by next-generation sequencing and compared with PD-L1 and tumor mutational burden in 11,348 patients. Cancer Med. 2018; 7:746-56.
12. Mandal R, Samstein R M, Lee K-W, Havel J J, Wang H, Krishna C, et al. Genetic diversity of tumors with mismatch repair deficiency influences anti-PD-1 immunotherapy response. Science. 2019; 364:485-91.
13. Le D T, Uram J N, Wang H, Bartlett B R, Kemberling H, Eyring A D, et al. PD-1 Blockade in Tumors with Mismatch-Repair Deficiency. N Engl J Med. 2015; 372:2509-20.
14. Le D T, Durham J N, Smith K N, Wang H, Bartlett B R, Aulakh L K, et al. Mismatch repair deficiency predicts response of solid tumors to PD-1 blockade. Science. 2017; 357:409-13.
15. Chang L, Chang M, Chang H M, Chang F. Microsatellite Instability: A Predictive Biomarker for Cancer Immunotherapy. Appl Immunohistochem Mol Morphol. 2018; 26: e15-21.
16. Luksza M, Riaz N, Makarov V, Balachandran V P, Hellmann M D, Solovyov A, et al. A neoantigen fitness model predicts tumour response to checkpoint blockade immunotherapy. Nature. 2017; 551:517-20.
17. de la Chapelle A, Hampel H. Clinical relevance of microsatellite instability in colorectal cancer. Journal of clinical oncology: official journal of the American Society of Clinical Oncology. 2010; 28:3380-7.
18. Bacher J W, Flanagan L A, Smalley R L, Nassif N A, Burgart L J, Halberg R B, et al. Development of a fluorescent multiplex assay for detection of MSI-High tumors. Disease markers. 2004; 20:237-50.
19. Salipante S J, Scroggins S M, Hampel H L, Turner E H, Pritchard C C. Microsatellite instability detection by next generation sequencing. Clinical chemistry. 2014; 60:1192-9.
20. Huang M N, McPherson J R, Cutcutache I, Teh B T, Tan P, Rozen S G. MSIseq: Software for Assessing Microsatellite Instability from Catalogs of Somatic Mutations. Sci Rep. 2015; 5:13321.
21. Kautto E A, Bonneville R, Miya J, Yu L, Krook M A, Reeser J W, et al. Performance evaluation for rapid detection of pan-cancer microsatellite instability with MANTIS. Oncotarget. 2017; 8:7452-63.
22. Middha S, Zhang L, Nafa K, Jayakumaran G, Wong D, Kim H R, et al. Reliable Pan-Cancer Microsatellite Instability Assessment by Using Targeted Next-Generation Sequencing Data. JCO Precision Oncology. 2017; 1-17.
23. Waalkes A, Smith N, Penewit K, Hempelmann J, Konnick E Q, Hause R J, et al. Accurate Pan-Cancer Molecular Diagnosis of Microsatellite Instability by Single-Molecule Molecular Inversion Probe Capture and High-Throughput Sequencing. Clin Chem. 2018; 64:950-8.
24. Gray P N, Tsai P, Chen D, Wu S, Hoo J, Mu W, et al. TumorNext-Lynch-MMR: a comprehensive next generation sequencing assay for the detection of germline and somatic mutations in genes associated with mismatch repair deficiency and Lynch syndrome. Oncotarget. 2018; 9:20304-22.
25. Hempelmann J A, Scroggins S M, Pritchard C C, Salipante S J. MSIplus: Integrated Colorectal Cancer Molecular Testing by Next-Generation Sequencing. J Mol Diagn. 2015;
26. Willis J, Lefterova M I, Artyomenko A, Kasi P M, Nakamura Y, Mody K, et al. Validation of Microsatellite Instability Detection Using a Comprehensive Plasma-Based Genotyping Panel. Clin Cancer Res. 2019;
27. Trabucco S E, Gowen K, Maund S L, Sanford E, Fabrizio D A, Hall M J, et al. A Novel Next-Generation Sequencing Approach to Detecting Microsatellite Instability and Pan-Tumor Characterization of 1000 Microsatellite Instability-High Cases in 67,000 Patient Samples. J Mol Diagn. 2019; 21:1053-66.
28. Hampel H, Frankel W, Panescu J, Lockman J, Sotamaa K, Fix D, et al. Screening for Lynch syndrome (hereditary nonpolyposis colorectal cancer) among endometrial cancer patients. Cancer Res. 2006; 66:7810-7.
29. Faulkner R D, Seedhouse C H, Das-Gupta E P, Russell N H. BAT-25 and BAT-26, two mononucleotide microsatellites, are not sensitive markers of microsatellite instability in acute myeloid leukaemia. Br J Haematol. 2004; 124:160-5.
30. Evaluation of Genomic Applications in Practice and Prevention (EGAPP) Working Group. Recommendations from the EGAPP Working Group: genetic testing strategies in newly diagnosed individuals with colorectal cancer aimed at reducing morbidity and mortality from Lynch syndrome in relatives. Genet Med. 2009; 11:35-41.
31. Beier S, Thiel T, Münch T, Scholz U, Mascher M. MISA-web: a web server for microsatellite prediction. Bioinformatics. 2017; 33:2583-5.
32. Wang K, Li M, Hakonarson H. ANNOVAR: functional annotation of genetic variants from high-throughput sequencing data. Nucleic acids research. 2010; 38: e164.
33. Niu B, Ye K, Zhang Q, Lu C, Xie M, McLellan M D, et al. MSIsensor: microsatellite instability detection using paired tumor-normal sequence data. Bioinformatics. 2014; 30:1015-6.
34. Koole W, Schafer H S, Agami R, van Haaften G, Tijsterman M. A versatile microsatellite instability reporter system in human cells. Nucleic Acids Res. 2013; 41: e158.
35. Chalmers Z R, Connelly C F, Fabrizio D, Gay L, Ali S M, Ennis R, et al. Analysis of 100,000 human cancer genomes reveals the landscape of tumor mutational burden. Genome Med. 2017; 9:34.
36. Nagahashi M, Wakai T, Shimada Y, Ichikawa H, Kameyama H, Kobayashi T, et al. Genomic landscape of colorectal cancer in Japan: clinical implications of comprehensive genomic sequencing for precision medicine. Genome Med. 2016; 8:136.
37. Van Allen E M, Miao D, Schilling B, Shukla S A, Blank C, Zimmer L, et al. Genomic correlates of response to CTLA-4 blockade in metastatic melanoma. Science. 2015; 350:207-11.
38. Danilova L, Wang H, Sunshine J, Kaunitz G J, Cottrell T R, Xu H, et al. Association of PD-1/PD-L axis expression with cytolytic activity, mutational load, and prognosis in melanoma and other solid tumors. Proc Natl Acad Sci USA. 2016; 113: E7769-77.
39. Brown S D, Warren R L, Gibb E A, Martin S D, Spinelli J J, Nelson B H, et al. Neo-antigens predicted by tumor genome meta-analysis correlate with increased patient survival. Genome Res. 2014; 24:743-50.
40. Gonzalez-Cao M, Viteri S, Karachaliou N, Aguilar A, Garcia-Mosquera J J, Rosell R. Tumor mutational burden as predictive factor of response to immunotherapy. Transl Lung Cancer Res. 2018; 7: S358-61.
41. The Cancer Genome Atlas Program. available on the world wide web at cancer. gov/tcga33. (Accessed July 2020).
42. Long et al., Identifying Optimal Loci for the Molecular Diagnosis of Microsatellite Instability, Clin Chem. 2020 Oct. 1; 66(10):1310-1318.

TABLE 19
Pairwise Euclidean dissimilarity matrix of tissue-specific informative microsatellite loci based on tissue-scaled p-values.

| | UCEC | STAD | READ | PRAD | OV | LUSC | LUAD | LGG | KIRC | GBM | ESCA | DLBC | COAD | CESC | BRCA |
|---|---|---|---|---|---|---|---|---|---|---|---|---|---|---|---|
| UCEC | 0 | 10.7 | 13.8 | 18.2 | 17.2 | 16.6 | 16.6 | 20.8 | 17.4 | 16.8 | 17.1 | 18.9 | 9.3 | 15 | 12.4 |
| STAD | 10.7 | 0 | 16.1 | 19.3 | 19.5 | 18.7 | 18.4 | 22.8 | 19.3 | 18.7 | 19.6 | 21.8 | 12.5 | 18 | 15.4 |
| READ | 13.8 | 16.1 | 0 | 19.9 | 18.8 | 18.2 | 18.6 | 22.1 | 19.7 | 18.9 | 20.4 | 19.8 | 13.1 | 17.9 | 15.7 |
| PRAD | 18.2 | 19.3 | 19.9 | 0 | 16 | 13.7 | 14.6 | 18.2 | 18.7 | 15 | 21.2 | 15.9 | 19.2 | 16.2 | 15.4 |
| OV | 17.2 | 19.5 | 18.8 | 16 | 0 | 11.6 | 12.8 | 16.7 | 17 | 13.2 | 20.3 | 13 | 18.3 | 14.4 | 13.4 |
| LUSC | 16.6 | 18.7 | 18.2 | 13.7 | 11.6 | 0 | 9.9 | 14.4 | 15.5 | 10.4 | 19.4 | 10.6 | 17.8 | 12.4 | 11.8 |
| LUAD | 16.6 | 18.4 | 18.6 | 14.6 | 12.8 | 9.9 | 0 | 15.6 | 16.2 | 11.2 | 19.7 | 12.2 | 17.9 | 13.3 | 12.5 |
| LGG | 20.8 | 22.8 | 22.1 | 18.2 | 16.7 | 14.4 | 15.6 | 0 | 19.7 | 15.6 | 23 | 15.8 | 22 | 17.2 | 16.8 |
| KIRC | 17.4 | 19.3 | 19.7 | 18.7 | 17 | 15.5 | 16.2 | 19.7 | 0 | 16.4 | 21.9 | 17 | 18.8 | 17.2 | 15.8 |
| GBM | 16.8 | 18.7 | 18.9 | 15 | 13.2 | 10.4 | 11.2 | 15.6 | 16.4 | 0 | 20 | 12.4 | 18.3 | 13.8 | 12.6 |
| ESCA | 17.1 | 19.6 | 20.4 | 21.2 | 20.3 | 19.4 | 19.7 | 23 | 21.9 | 20 | 0 | 20.9 | 18.3 | 18.3 | 18.2 |
| DLBC | 18.9 | 21.8 | 19.8 | 15.9 | 13 | 10.6 | 12.2 | 15.8 | 17 | 12.4 | 20.9 | 0 | 19.8 | 14.3 | 13.8 |
| COAD | 9.3 | 12.5 | 13.1 | 19.2 | 18.3 | 17.8 | 17.9 | 22 | 18.8 | 18.3 | 18.3 | 19.8 | 0 | 16.5 | 14 |
| CESC | 15 | 18 | 17.9 | 16.2 | 14.4 | 12.4 | 13.3 | 17.2 | 17.2 | 13.8 | 18.3 | 14.3 | 16.5 | 0 | 13.1 |
| BRCA | 12.4 | 15.4 | 15.7 | 15.4 | 13.4 | 11.8 | 12.5 | 16.8 | 15.8 | 12.6 | 18.2 | 13.8 | 14 | 13.1 | 0 |

TABLE 20
Pairwise Cophenetic dissimilarity matrix of tissue-specific informative microsatellite loci based on tissue-scaled p-values.

| | UCEC | STAD | READ | PRAD | OV | LUSC | LUAD | LGG | KIRC | GBM | ESCA | DLBC | COAD | CESC | BRCA |
|---|---|---|---|---|---|---|---|---|---|---|---|---|---|---|---|
| UCEC | 0 | 12.5 | 16.1 | 23 | 23 | 23 | 23 | 23 | 19.7 | 23 | 21.9 | 23 | 9.3 | 23 | 23 |
| STAD | 12.5 | 0 | 16.1 | 23 | 23 | 23 | 23 | 23 | 19.7 | 23 | 21.9 | 23 | 12.5 | 23 | 23 |
| READ | 16.1 | 16.1 | 0 | 23 | 23 | 23 | 23 | 23 | 19.7 | 23 | 21.9 | 23 | 16.1 | 23 | 23 |
| PRAD | 23 | 23 | 23 | 0 | 16.2 | 16.2 | 16.2 | 18.2 | 23 | 16.2 | 23 | 16.2 | 23 | 16.2 | 16.2 |
| OV | 23 | 23 | 23 | 16.2 | 0 | 13.2 | 13.2 | 18.2 | 23 | 13.2 | 23 | 13.2 | 23 | 14.4 | 14.4 |
| LUSC | 23 | 23 | 23 | 16.2 | 13.2 | 0 | 9.9 | 18.2 | 23 | 11.2 | 23 | 12.4 | 23 | 14.4 | 14.4 |
| LUAD | 23 | 23 | 23 | 16.2 | 13.2 | 9.9 | 0 | 18.2 | 23 | 11.2 | 23 | 12.4 | 23 | 14.4 | 14.4 |
| LGG | 23 | 23 | 23 | 18.2 | 18.2 | 18.2 | 18.2 | 0 | 23 | 18.2 | 23 | 18.2 | 23 | 18.2 | 18.2 |
| KIRC | 19.7 | 19.7 | 19.7 | 23 | 23 | 23 | 23 | 23 | 0 | 23 | 21.9 | 23 | 19.7 | 23 | 23 |
| GBM | 23 | 23 | 23 | 16.2 | 13.2 | 11.2 | 11.2 | 18.2 | 23 | 0 | 23 | 12.4 | 23 | 14.4 | 14.4 |
| ESCA | 21.9 | 21.9 | 21.9 | 23 | 23 | 23 | 23 | 23 | 21.9 | 23 | 0 | 23 | 21.9 | 23 | 23 |
| DLBC | 23 | 23 | 23 | 16.2 | 13.2 | 12.4 | 12.4 | 18.2 | 23 | 12.4 | 23 | 0 | 23 | 14.4 | 14.4 |
| COAD | 9.3 | 12.5 | 16.1 | 23 | 23 | 23 | 23 | 23 | 19.7 | 23 | 21.9 | 23 | 0 | 23 | 23 |
| CESC | 23 | 23 | 23 | 16.2 | 14.4 | 14.4 | 14.4 | 18.2 | 23 | 14.4 | 23 | 14.4 | 23 | 0 | 13.1 |
| BRCA | 23 | 23 | 23 | 16.2 | 14.4 | 14.4 | 14.4 | 18.2 | 23 | 14.4 | 23 | 14.4 | 23 | 13.1 | 0 |

TABLE 21
Coordinates of cross-informative microsatellites for diagnosing MSI in endometrial, colon, rectal, and stomach cancers.

| | Start | End | | Start | End | | Start | End |
|---|---|---|---|---|---|---|---|---|
| Chromosome | Position | Position | Chromosome | Position | Position | Chromosome | Position | Position |
| 1 | 149900985 | 149901002 | 16 | 3808052 | 3808066 | 6 | 102130410 | 102130422 |
| 1 | 222904869 | 222904881 | 16 | 9010826 | 9010844 | 6 | 122733490 | 122733508 |
| 10 | 112360315 | 112360333 | 17 | 29508819 | 29508836 | 6 | 36452603 | 36452620 |
| 10 | 35314130 | 35314161 | 19 | 6026288 | 6026302 | 6 | 46636556 | 46636581 |
| 10 | 64022582 | 64022593 | 2 | 141294271 | 141294299 | 6 | 49415351 | 49415368 |
| 11 | 118885668 | 118885701 | 2 | 203676382 | 203676403 | 7 | 97939975 | 97939988 |
| 12 | 110834030 | 110834049 | 2 | 32095023 | 32095056 | 8 | 100160282 | 100160300 |
| 12 | 125587478 | 125587491 | 2 | 8998775 | 8998788 | 8 | 33356825 | 33356839 |
| 12 | 85285920 | 85285938 | 3 | 41877473 | 41877487 | 9 | 52625 | 52641 |
| 12 | 89853387 | 89853405 | 4 | 146031236 | 146031253 | 9 | 75420448 | 75420463 |
| 13 | 79216356 | 79216381 | 5 | 172578549 | 172578567 | X | 106092450 | 106092473 |
| 15 | 55841195 | 55841209 | 5 | 36123065 | 36123135 | X | 19380975 | 19380993 |
| 16 | 10567751 | 10567778 | | | | | | |

TABLE 22
Covariates associated with instability of MSI-informative microsatellites. “*” indicates that the
Positive coefficients values are associated with microsatellite instability; negative values are associated
with microsatellite stability “**” indicates that the category was used as reference for statistical comparisons.

| Feature Category | Feature | Coefficient* | 97.5% confidence interval | p value |
|---|---|---|---|---|
| Coding context | Intergenic | Not applicable** | | |
| | Upstream | 0.26330362 | 0.201082791 to 0.32552445 | 1.1E−16 |
| | Downstream | 0.24775092 | 0.19523743 to 0.3002644 | 2.32E−20 |
| | Exonic | 0.63116873 | 0.312458773 to 0.94987869 | 0.000104 |
| | Intronic | 0.22357372 | 0.214076632 to 0.23307081 | 0 |
| | ncRNA exonic | 0.24869416 | 0.139176607 to 0.35821171 | 8.56E−06 |
| | ncRNA intronic | 0.06674437 | 0.045628207 to 0.08786053 | 5.83E−10 |
| | ncRNA splicing | 0.80560147 | 0.355024088 to 1.25617886 | 0.000458 |
| | Splicing | 1.095599 | 0.889927325 to 1.30127068 | 1.63E−25 |
| | Upstream and Downstream | 0.20875388 | −0.103550755 to 0.52105852 | 0.190162 |
| | 3′ UTR | 0.54949742 | 0.502719112 to 0.59627574 | 3.1E−117 |
| | 5′ UTR | 0.64615781 | 0.442216539 to 0.85009909 | 5.31E−10 |
| | 5′ UTR5 and 3′ UTR | 0.0103701 | −3.508426006 to 3.52916621 | 0.995391 |
| Repeat class | Compound repeat | 1.46871369 | 1.301270946 to 1.63615644 | 3.18E−66 |
| | Complex repeat | 0.92271293 | 0.751505079 to 1.09392078 | 4.44E−26 |
| | Mononucleotide repeat | 1.44146313 | 1.272848876 to 1.61007737 | 5.33E−63 |
| | Dinucleotide repeat | 0.15687561 | −0.012360733 to 0.32611194 | 0.069246 |
| | Trinucleotide repeat | −0.118346 | −0.4131167 to 0.17642471 | 0.431342 |
| | Tetranucleotide Repeat | 0.24265869 | 0.054478379 to 0.43083901 | 0.011492 |
| | Pentanucleotide Repeat | Not applicable** | | |
| Number of repeats | 5-11 | Not applicable** | | |
| | 11-12 | 0.80031813 | 0.779980199 to 0.82065606 | 0 |
| | 12-13 | 1.77745115 | 1.757111464 to 1.79779084 | 0 |
| | 13-14 | 1.59183987 | 1.571499926 to 1.61217982 | 0 |
| | 14-16 | 1.50008182 | 1.479731404 to 1.52043224 | 0 |
| | 16-18 | 0.31325393 | 0.2920993 to 0.33440855 | 4.6E−185 |
| | 18-21 | −0.1869956 | −0.211463299 to −0.162528 | 1.03E−50 |
| | 21-25 | −0.2282874 | −0.255785148 to −0.2007896 | 1.61E−59 |
| | 25-32 | −0.5444188 | −0.572807037 to −0.5160306 | 0 |
| | >32 | −1.2434585 | −1.272243027 to −1.2146739 | 0 |

### Example 2: Supplemental Information

For full versions of the Supplemental Tables 1-3 described in EXAMPLE 1 above, please see APPENDIX A of U.S. Provisional Application No. 63/044,029 filed Jun. 25, 2020, the contents of which are incorporated herein by reference in their entirety.

Please see APPENDIX B of U.S. Provisional Application No. 63/044,029 filed Jun. 25, 2020, the contents of which are incorporated herein by reference in their entirety, for a table of microsatellite sequences most significantly associated with MSI-H classification for each cancer type. The location of each microsatellite is listed according to hg19 coordinates; and the p value for significance is indicated for each specified cancer type. Blank cells indicate non-significant values. See also Tables 1-15 herein.

1. A method of predicting whether a subject's cancer will respond to checkpoint inhibitor immunotherapy, the method comprising:
(a) receiving microsatellite instability data for a defined set of microsatellite repeat marker sequences in cells of the subject's cancer; and
(b) processing the microsatellite instability data to output a categorical measure of microsatellite instability high (MSI-H) or microsatellite stable (MSS);
wherein when the subject's cancer is determined to exhibit MSI-H, it is predicted that the cancer will respond to checkpoint inhibitor immunotherapy, and when the subject's cancer is determined to exhibit MSS, it is predicted that the cancer is less likely to respond to checkpoint inhibitor immunotherapy.

2. (canceled)

3. The method of claim 1, wherein processing the microsatellite instability data to output a categorical measure of microsatellite instability high (MSI-H) or microsatellite stable (MSS) comprises applying a quantitative model relating the predictive capability of the markers in the set or a subset thereof to known MSI status.

4. The method of claim 3, wherein the quantitative model:
is selected from a continuous measure, regression or weighted scoring of markers, a combinatorial or decision-tree model, and a machine learning model;
comprises a random-forest model or a deep neural network machine learning model; or
evaluates or incorporates consideration of one or more test characteristics selected from the group consisting of sensitivity, accuracy, correlation, probability, specificity, false-positive rate, false negative rate, positive predictive value, negative predictive value and area under the receiver-operator characteristic (AUROC).

5. The method of claim 4, wherein the continuous measure comprises the proportion of unmutated or stable loci to mutated or unstable loci detected in the set.

6-7. (canceled)

8. The method of claim 4, wherein thresholds for the one or more test characteristics indicative of MSI-H or MSS are defined within parameters of the known test characteristics for a given clinical application.

9. The method of claim 1, wherein:
when the cancer is colon adenocarcinoma (COAD), the set of microsatellite repeat marker sequences comprises a plurality up to 500 of the microsatellites set out in Table 1;
when the cancer is esophageal carcinoma (ESCA), the set of microsatellite repeat marker sequences comprises a plurality up to 503 of the microsatellites set out in Table 2;
when the cancer is glioblastoma multiforme (GBM), the set of microsatellite repeat marker sequences comprises a plurality up to 500 of the microsatellites set out in Table 3;
when the cancer is lung adenocarcinoma (LUAD), the set of microsatellite repeat marker sequences comprises a plurality up to 500 of the microsatellites set out in Table 4;
when the cancer is lung squamous cell carcinoma (LUSC), the set of microsatellite repeat marker sequences comprises a plurality up to 500 of the microsatellites set out in Table 5;
when the cancer is rectum adenocarcinoma (READ), the set of microsatellite repeat marker sequences comprises a plurality up to 501 of the microsatellites set out in Table 6;
when the cancer is stomach adenocarcinoma (STAD), the set of microsatellite repeat marker sequences comprises a plurality up to 500 of the microsatellites set out in Table 7;
when the cancer is brain lower grade glioma (LGG), the set of microsatellite repeat marker sequences comprises a plurality up to 266 of the microsatellites set out in Table 8;
when the cancer is prostate adenocarcinoma (PRAD), the set of microsatellite repeat marker sequences comprises a plurality up to 500 of the microsatellites set out in Table 9;
when the cancer is cervical squamous cell carcinoma (CESC), the set of microsatellite repeat marker sequences comprises a plurality up to 500 of the microsatellites set out in Table 10;
when the cancer is lymphoid neoplasm diffuse large B-cell lymphoma (DLBC), the set of microsatellite repeat marker sequences comprises a plurality up to 212 of the microsatellites set out in Table 11;
when the cancer is uterine corpus endometrial carcinoma (UCEC), the set of microsatellite repeat marker sequences comprises a plurality up to 500 of the microsatellites set out in Table 12;
when the cancer is kidney renal clear cell carcinoma (KIRC), the set of microsatellite repeat marker sequences comprises a plurality up to 508 of the microsatellites set out in Table 13;
when the cancer is breast invasive carcinoma (BRCA), the set of microsatellite repeat marker sequences comprises a plurality up to 500 of the microsatellites set out in Table 14A; and
when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.

10-11. (canceled)

12. The method of claim 1, wherein the set of microsatellite repeat marker sequences comprises a plurality up to the first 100 of the sequences set out in the respective Table for the subject's cancer type.

13-16. (canceled)

17. The method of claim 1, wherein the processing of MSI-H or MSS is determined by a threshold value for the set of microsatellite marker sequences.

18. The method of claim 17, wherein the threshold for the set of microsatellite marker sequences is determined using the percentage of mutated microsatellite markers in the set to calculate the area under the receiver operating characteristic (AUROC).

19. The method of claim 18, wherein the smallest number of markers in the set necessary to reach a selected AUROC value is the threshold.

20. The method of claim 19, wherein the selected AUROC value is a value between 0.6 and 0.99, inclusive.

21. The method of claim 19, wherein the selected AUROC value is 0.9 or more.

22-25. (canceled)

26. The method of claim 1, wherein microsatellite mutations correspond to those identified in reference human genome GRCh37/hg19 translated to a different build of the human reference genome.

27-42. (canceled)

43. A method of treating cancer in a subject in need thereof, the method comprising determining microsatellite instability status for cells of a subject's cancer by a method of claim 1, and, when the microsatellite instability status is determined to be MSI-H, administering a checkpoint inhibitor, or, when the microsatellite instability status is determined to be MSS, administering a non-checkpoint inhibitor cancer therapeutic.

44. The method of claim 43, wherein the checkpoint inhibitor is an inhibitor of a checkpoint molecule selected from the group consisting of: PD-1 or PD-L1, CTLA4, Adenosine A2A receptor (A2AR), CD276, CD39, CD73, B7 family immune checkpoint molecules, V-set domain-containing T-cell activation inhibitor 1 (B7H4), B and T Lymphocyte Attenuator (BTLA), Indoleamine 2,3-dioxygenase (IDO), Killer-cell Immunoglobulin-like Receptor (KIR), Lymphocyte Activation Gene-3 (LAG3), nicotinamide adenine dinucleotide phosphate NADPH oxidase isoform 2 (NOX2), T-cell Immunoglobulin domain and Mucin domain 3 (TIM-3), T cell immunoreceptor with Ig and ITIM domains (TIGIT), V-domain Ig suppressor of T cell activation (VISTA), and Sialic acid-binding immunoglobulin-type lectin 7 (SIGLEC7).

45. The method of claim 43, wherein the checkpoint inhibitor is selected from the group consisting of pembrolizumab (Keytruda®), nivolumab (Opdivo®), cemiplimab (Libtayo®), spartalizumab, camrelizumab, sintilimab, tislelizumab, toripalimab, dostarlimab, INVMGA00012, AMP-224, AMP-514, atezolizumab (Tecentriq®), avelumab (Bavencio®), survalumab (Imfinzi®), KN035, CK-301, AUNP12, CA-170, BMS-986189, and ipilimumab (Yervoy®).

46-47. (canceled)

48. The method of claim 43, wherein the non-checkpoint inhibitor cancer therapy comprises one or more of angiostatin K1-3, DL-a-Difluoromethyl-ornithine, endostatin, fumagillin, genistein, minocycline, staurosporine, and (±)-thalidomide; a DNA intercalator/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid, all trans (Vitamin A acid), 9-cis-Retinoic Acid, 13-cis-Retinoic acid, Retinol (Vitamin A), Tamoxifen, and Troglitazone; a microtubule inhibitor, such as Colchicine, Dolastatin 15, Nocodazole, Paclitaxel, Podophyllotoxin, Rhizoxin, Vinblastine, Vincristine, Vindesine, and Vinorelbine (Navelbine); a neoantigen; and an unclassified antitumor agent, such as 17-(Allylamino)-17-demethoxygeldanamycin, 4-Amino-1, 8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™), idelalisib (Zydelig®), crizotinib (Xalkori®), erlotinib (Tarceva®), afatinib dimaleate (Gilotrif®), ceritinib (LDK378/Zykadia), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodaq™), lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®), an epigenetic targeted drug such as HDAC inhibitors, azacitidine (Vidaza®), decitabine (Dacogen®), vorinostat (Zolinza®), romidepsin (Istodax®), ruxolitinib (Jakafi®), kinase inhibitors, DNA methyltransferase inhibitors, histone demethylase inhibitors, or histone methylation inhibitors, and any derivative thereof.

49-54. (canceled)

55. The method of claim 1,
wherein when mutations are present in at least a threshold number of markers in the set, the set provides at least 95% sensitivity and at least 95% specificity for predicting high microsatellite instability (MSI-H) for the subject's tumor,
wherein:
when the cancer is colon adenocarcinoma (COAD), the microsatellite repeat marker sequences comprise a plurality of microsatellite repeat markers in Table 1 and the threshold number is 50%;
when the cancer is esophageal carcinoma (ESCA), the microsatellite repeat marker sequences comprise a plurality of microsatellite repeat markers in Table 2 and the threshold number is 50%;
when the cancer is glioblastoma multiforme (GBM), the microsatellite repeat marker sequences comprise a plurality of microsatellite repeat markers in Table 3 and the threshold number is 50%;
when the cancer is lung adenocarcinoma (LUAD), the microsatellite repeat marker sequences comprise a plurality of microsatellite repeat markers in Table 4 and the threshold number is 50%;
when the cancer is lung squamous cell carcinoma (LUSC), the microsatellite repeat marker sequences comprise a plurality of microsatellite repeat markers in Table 5 and the threshold number is 50%;
when the cancer is rectum adenocarcinoma (READ), the microsatellite repeat marker sequences comprise a plurality of microsatellite repeat markers in Table 6 and the threshold number is 50%;
when the cancer is stomach adenocarcinoma (STAD), the microsatellite repeat marker sequences comprise a plurality of microsatellite repeat markers in Table 7 and the threshold number is 25%;
when the cancer is brain lower grade glioma (LGG), the microsatellite repeat marker sequences comprise a plurality of microsatellite repeat markers in Table 8 and the threshold number is 12.5%;
when the cancer is prostate adenocarcinoma (PRAD), the microsatellite repeat marker sequences comprise a plurality of microsatellite repeat markers in Table 9 and the threshold number is 16.7%;
when the cancer is cervical squamous cell carcinoma (CESC), the microsatellite repeat marker sequences comprise a plurality of microsatellite repeat markers in Table 10 and the threshold number is 45.8%;
when the cancer is lymphoid neoplasm diffuse large B-cell lymphoma (DLBC), the microsatellite repeat marker sequences comprise a plurality of microsatellite repeat markers in Table 11 and the threshold number is 25%;
when the cancer is uterine corpus endometrial carcinoma (UCEC), the microsatellite repeat marker sequences comprise a plurality of microsatellite repeat markers in Table 12 and the threshold number is 12.9%;
when the cancer is kidney renal clear cell carcinoma (KIRC), the microsatellite repeat marker sequences comprise a plurality of microsatellite repeat markers in Table 13 and the threshold number is 7.5%;
when the cancer is breast invasive carcinoma (BRCA), the microsatellite repeat marker sequences comprise a plurality of microsatellite repeat markers in Table 14A and the threshold number is 3.6%; and
when the cancer is uterine corpus endometrial carcinoma (UCEC), colon adenocarcinoma (COAD), rectum adenocarcinoma (READ), or stomach adenocarcinoma (STAD), the microsatellite repeat marker sequences comprise a plurality of microsatellite repeat markers in Table 21 and the threshold number is at least 12.9%;
and wherein
when the subject's cancer exhibits greater than or equal to the threshold number of mutations for that tumor type in the set, the subject's cancer is determined to be MSI-H, and is predicted to be sensitive to checkpoint inhibitor immunotherapy.

56-100. (canceled)

101. A diagnostic kit for determining microsatellite instability in a cancer, the kit comprising reagents that permit the detection of microsatellite mutation in a set of microsatellite repeat marker sequences in the cancer, wherein:
when the cancer is colon adenocarcinoma (COAD), the set of microsatellite repeat marker sequences comprises a plurality up to 500 of the microsatellites set out in Table 1;
when the cancer is esophageal carcinoma (ESCA), the set of microsatellite repeat marker sequences comprises a plurality up to 503 of the microsatellites set out in Table 2;
when the cancer is glioblastoma multiforme (GBM), the set of microsatellite repeat marker sequences comprises a plurality up to 500 of the microsatellites set out in Table 3;
when the cancer is lung adenocarcinoma (LUAD), the set of microsatellite repeat marker sequences comprises a plurality up to 500 of the microsatellites set out in Table 4;
when the cancer is lung squamous cell carcinoma (LUSC), the set of microsatellite repeat marker sequences comprises a plurality up to 500 of the microsatellites set out in Table 5;
when the cancer is rectum adenocarcinoma (READ), the set of microsatellite repeat marker sequences comprises a plurality up to 501 of the microsatellites set out in Table 6;
when the cancer is stomach adenocarcinoma (STAD), the set of microsatellite repeat marker sequences comprises a plurality up to 500 of the microsatellites set out in Table 7;
when the cancer is brain lower grade glioma (LGG), the set of microsatellite repeat marker sequences comprises a plurality up to 266 of the microsatellites set out in Table 8;
when the cancer is prostate adenocarcinoma (PRAD), the set of microsatellite repeat marker sequences comprises a plurality up to 500 of the microsatellites set out in Table 9;
when the cancer is cervical squamous cell carcinoma (CESC), the set of microsatellite repeat marker sequences comprises a plurality up to 500 of the microsatellites set out in Table 10;
when the cancer is lymphoid neoplasm diffuse large B-cell lymphoma (DLBC), the set of microsatellite repeat marker sequences comprises a plurality up to 212 of the microsatellites set out in Table 11;
when the cancer is uterine corpus endometrial carcinoma (UCEC), the set of microsatellite repeat marker sequences comprises a plurality up to 500 of the microsatellites set out in Table 12;
when the cancer is kidney renal clear cell carcinoma (KIRC), the set of microsatellite repeat marker sequences comprises a plurality up to 508 of the microsatellites set out in Table 13;
when the cancer is breast invasive carcinoma (BRCA), the set of microsatellite repeat marker sequences comprises a plurality up to 500 of the microsatellites set out in Table 14A; and
when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.

102-108. (canceled)

109. An array for detecting microsatellite instability in a cancer, the array comprising nucleic acids that permit the detection of microsatellite mutation in a set of microsatellite repeat marker sequences in the cancer, wherein the nucleic acids are linked to a solid support, wherein the nucleic acids are complementary to at least a portion of the microsatellite sequences, and wherein:
when the cancer is colon adenocarcinoma (COAD), the set of microsatellite repeat marker sequences comprises a plurality up to 500 of the microsatellites set out in Table 1;
when the cancer is esophageal carcinoma (ESCA), the set of microsatellite repeat marker sequences comprises a plurality up to 503 of the microsatellites set out in Table 2;
when the cancer is glioblastoma multiforme (GBM), the set of microsatellite repeat marker sequences comprises a plurality up to 500 of the microsatellites set out in Table 3;
when the cancer is lung adenocarcinoma (LUAD), the set of microsatellite repeat marker sequences comprises a plurality up to 500 of the microsatellites set out in Table 4;
when the cancer is lung squamous cell carcinoma (LUSC), the set of microsatellite repeat marker sequences comprises a plurality up to 500 of the microsatellites set out in Table 5;
when the cancer is rectum adenocarcinoma (READ), the set of microsatellite repeat marker sequences comprises a plurality up to 501 of the microsatellites set out in Table 6;
when the cancer is stomach adenocarcinoma (STAD), the set of microsatellite repeat marker sequences comprises a plurality up to 500 of the microsatellites set out in Table 7;
when the cancer is brain lower grade glioma (LGG), the set of microsatellite repeat marker sequences comprises a plurality up to 266 of the microsatellites set out in Table 8;
when the cancer is prostate adenocarcinoma (PRAD), the set of microsatellite repeat marker sequences comprises a plurality up to 500 of the microsatellites set out in Table 9;
when the cancer is cervical squamous cell carcinoma (CESC), the set of microsatellite repeat marker sequences comprises a plurality up to 500 of the microsatellites set out in Table 10;
when the cancer is lymphoid neoplasm diffuse large B-cell lymphoma (DLBC), the set of microsatellite repeat marker sequences comprises a plurality up to 212 of the microsatellites set out in Table 11;
when the cancer is uterine corpus endometrial carcinoma (UCEC), the set of microsatellite repeat marker sequences comprises a plurality up to 500 of the microsatellites set out in Table 12;
when the cancer is kidney renal clear cell carcinoma (KIRC), the set of microsatellite repeat marker sequences comprises a plurality up to 508 of the microsatellites set out in Table 13;
when the cancer is breast invasive carcinoma (BRCA), the set of microsatellite repeat marker sequences comprises a plurality up to 500 of the microsatellites set out in Table 14A; and
when the cancer is uterine corpus endometrial carcinoma (UCEC), colon adenocarcinoma (COAD), rectum adenocarcinoma (READ), or stomach adenocarcinoma (STAD), the set of microsatellite repeat marker sequences comprises a plurality up to 37 of the microsatellites set out in Table 21.

110-117. (canceled)
